(19) United States
(12) Patent Application Publication
(10) Pub. No.: US 2025/0188103 A1
(43) Pub. Date: Jun. 12, 2025

(54) HETEROCYCLIC GLP-1 AGONISTS

(71) Applicant: Gasherbrum Bio, Inc., South San Francisco, CA (US)

(72) Inventors: Xichen LIN, Shanghai (CN); Qinghua MENG, Shanghai (CN); Weiqiang XING, Shanghai (CN); Zhongmiao XU, Shanghai (CN)

(21) Appl. No.: 18/845,381

(22) PCT Filed: Mar. 8, 2023
(86) PCT No.: PCT/CN2023/080254

§ 371 Date: Sep. 9, 2024

(30) Foreign Application Priority Data
Mar. 9, 2022 (WO) PCT/CN2022/079865
Dec. 22, 2022 (WO) PCT/CN2022/141072

(51) Int. Cl. C07F 9/6561; A61K 31/675

(52) U.S. Cl. CPC C07F 9/6561; A61K 31/675; C07B 2200/05

(57) ABSTRACT

This disclosure relates to GLP-1 agonists, pharmaceutical compositions, and methods of use thereof.

### CROSS REFERENCE TO RELATED APPLICATIONS

The application claims the benefit of International Patent Application Number PCT/CN2022/079865, filed Mar. 9, 2022, and PCT/CN2022/141072, filed Dec. 22, 2022, each of which is incorporated herein by reference in its entirety.

### TECHNICAL FIELD

This disclosure relates to GLP-1 agonists, pharmaceutical compositions, and methods of use thereof.

### BACKGROUND

Incretin metabolic hormones, including glucagon-like peptide-1 (GLP-1) and glucose-dependent insulinotropic polypeptide (GIP), are important in the regulation of glucose homeostasis. Medicaments targeting this family of intestinal peptides, such as GLP-1 agonists, have been shown to suppress glucagon production, decrease gastric motility, and increase satiety.

Diabetes mellitus refers to a group of metabolic disorders characterized by persistent hyperglycemia. The most common form, type 2 diabetes mellitus (T2DM) is an acquired condition that accounts for more than 90% of diabetes cases. Typical onset occurs in obese or otherwise sedentary adults and begins with insulin resistance. Though lifestyle changes can be useful in management of this disorder, patients with T2DM may be required to take anti-diabetic medications, including dipeptidyl peptidase-4 inhibitors, SGLT2 inhibitors, and sulfonylureas, among others.

In healthy individuals, the incretin hormones glucose-dependent insulinotropic polypeptide (GIP) and glucagon-like peptide 1 (GLP-1) provide tandem modulation of insulin secretory response to glucose ingestion. While this incretin effect is significantly diminished (if at all present) in cases of T2DM, GLP-1 retains insulinotropic properties, even as endocrine pancreatic response to GIP is effectively halted. As such, incretin mimetics and other GLP-1-based therapies can help stimulate insulin production in T2DM patients.

### SUMMARY

The present application describes heterocyclic GLP-1 agonists, as well as pharmaceutical compositions comprising the compounds disclosed herein. Also provided are methods for treating GLP-1-associated diseases, disorders, and conditions.

Accordingly, provided herein are compounds of Formula I:

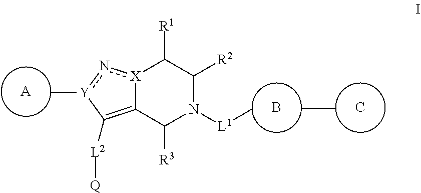

or a pharmaceutically acceptable salt, deuterated analog, or solvate thereof, wherein:
X is C and Y is N, or Y is C and X is N and the dashed lines represent an optional bond such that the moiety

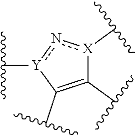

is

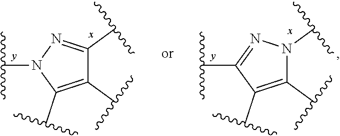 [SMILES: *c1nn(*)c(*)c1* ; *c1nn(*)c(*)c1*]

where bond y is attached to Y and bond x is attached to X;
Ring A is C₆₋₁₀ aryl, C₅₋₁₀ cycloalkyl, 5-10 membered heterocyclyl, or 5-10 membered heteroaryl, each of which is optionally substituted with from 1-5 substituents each independently selected from the group consisting of halo, C₁₋₆ alkyl, C₁₋₆ haloalkyl, C₁₋₆ alkoxy, and C₃₋₆ cycloalkyl;
R¹, R², and R³ are each independently selected from the group consisting of H and C₁₋₆ alkyl which is optionally substituted with from 1-6 substituents each independently selected from the group consisting of halo, —OH, and C₁₋₆ alkoxy;
L¹ is selected from the group consisting of —C(═O)—, —CH₂—, —CH(C₁₋₆ alkyl)-, and —S(═O)₂;
Ring B is selected from the group consisting of

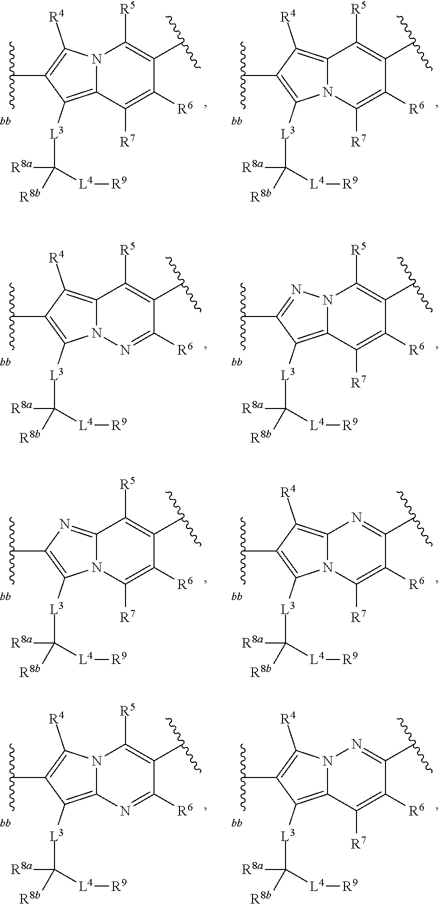

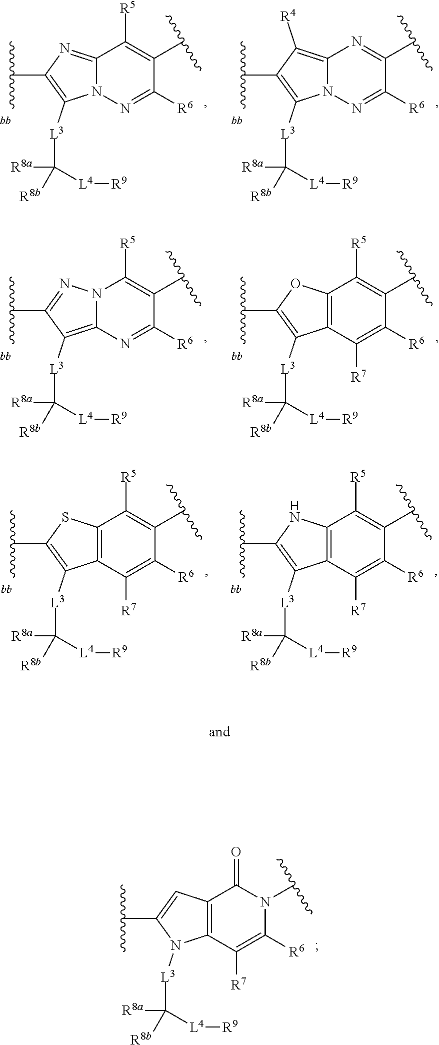

wherein bb represents point of attachment to L¹;
R⁴, R⁵, R⁶, and R⁷ are independently selected from the group consisting of H, halo, and C₁₋₆ alkyl;
L³ is a bond or C₁₋₃ alkylene;
L⁴ is a bond or C₁₋₅ alkylene;
R^{8a} and R^{8b} are independently selected from the group consisting of H and C₁₋₆ alkyl optionally substituted with one or more substituents independently selected from the group consisting of halo and C₃₋₁₅ cycloalkyl;
or R^{8a} and R^{8b} taken together with the carbon atom to which each is attached forms a C₃₋₁₅ cycloalkyl ring which is optionally substituted with from 1-3 independently selected C₁₋₆ alkyl, wherein the C₁₋₆ alkyl is optionally substituted with from 1-6 independently selected from R^{f};
or the moiety -L³-C(R^{8a}R^{8b})-L⁴-R⁹ is a phenyl substituted with C(═O)OH, and optionally 1-4 additional substituents independently selected from R^{f};
R⁹ is selected from the group consisting of C(═O)OH, C(═O)(OC₁₋₆ alkyl), C(═O)NR^{9a}R^{9b}, (IX-1), (IX-2), (IX-3), and (IX-4):

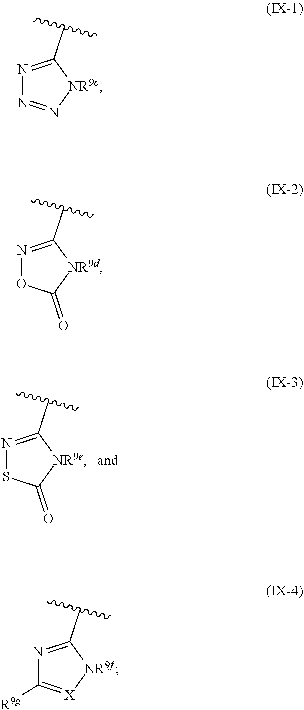

R^{9a} is H or C₁₋₆ alkyl;
R^{9b} is H, C₁₋₆ alkyl, C(═O)(C₁₋₆ alkyl), S(O)₀₋₂(C₁₋₆ alkyl), or cyano;
R^{9c}, R^{9d}, R^{9e}, R^{9f}, and R^{9g} are each independently selected from the group consisting of H, C₁₋₆ alkyl optionally substituted with from 1-6 independently selected halo and C₁₋₆ alkoxy, and C(═O)(C₁₋₆ alkyl);
Ring C is selected from the group consisting of 3-12 membered heterocyclyl, C₃₋₁₅ cycloalkyl, and 5-10 membered heteroaryl, each of which is optionally substituted with from 1-3 R^{Ca};
each R^{Ca} is independently selected from the group consisting of halo, C₁₋₆ alkyl, C₁₋₆ haloalkyl, C₁₋₆ alkoxy, and NR^{c}R^{d};
or a pair of R^{Ca} on the same or different ring atoms, taken together with the ring atom(s) to which each is attached, forms a carbocyclic ring including from 3-8 ring atoms;
L² is selected from the group consisting of

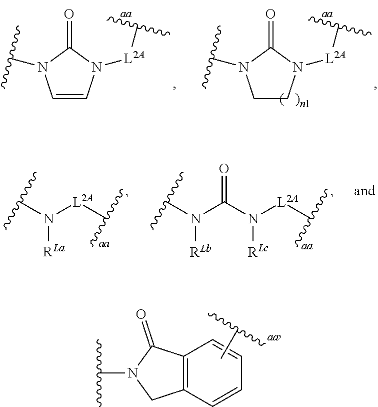

wherein aa represents the point of attachment to Q;
n1 is an integer from 1-3;
L^{2A} is a bond or C₁₋₁₀ alkylene;
R^{La} is selected from the group consisting of H, C₁₋₆ alkyl, and C(═O)(C₁₋₁₆ alkyl);
each of R^{Lb} and R^{L}c is independently selected from the group consisting of H and C₁₋₆ alkyl;
Q is selected from the group consisting of C₁₋₁₀ alkyl; C₃₋₁₅ cycloalkyl; 3-12 membered heterocyclyl; 5-10 membered heteroaryl; and C₆₋₁₀ aryl, wherein Q is substituted with —P(═O)R^{a}R^{b}, and optionally substituted with from 1-6 additional substituents independently selected from R^{Q};
each R^{Q} is independently selected from the group consisting of
(a) halo;
(b) cyano;
(c) OH or oxo;
(d) —NR^{c}R^{d};
(e) —C(═O)NR^{c}R^{d} or —S(O)₂NR^{c}R^{d};
(f) —S(═O)₀₋₂R^{c};
(g) C₁₋₆ alkyl optionally substituted with from 1-6 independently selected R^{f};
(h) C₁₋₆ alkoxy optionally substituted with from 1-6 independently selected R^{f};
(i) 3-12 membered heterocyclyl optionally substituted with from 1-6 independently selected R^{g};
(j) C₆₋₁₀ aryl optionally substituted with from 1-6 independently selected R^{g};
(k) 5-10 membered heteroaryl optionally substituted with from 1-6 independently selected R^{g};
(l) C₃₋₈ cycloalkyl optionally substituted with from 1-6 independently selected R^{g};
(m) —P(═O)R^{a}R^{b}; and
(n) —(CR^{h}R^{h})_{q1}—S(O)₂-L^{e}-R^{e}, wherein q1 is 1, 2, or 3;
or Q is C₃₋₁₅ cycloalkyl; 3-12 membered heterocyclyl; 5-10 membered heteroaryl; and C₆₋₁₀ aryl, each of which is optionally substituted with from 1-6 substituents each independently selected from the group consisting of R^{QA} and R^{QB};
provided that one or both of (aa) or (bb) applies:
(aa) Q is substituted with at least one R^{QB}; or
(bb) Q is 4-12 membered heterocyclyl or 7-10 membered bicyclic heteroaryl, wherein Q comprises an endocyclic S(O)₂ group;
each R^{QA} is independently selected from the group consisting of
(a) halo;
(b) cyano;
(c) OH or oxo;
(d) —NR^{a}R^{b};
(e) —C(═O)NR^{c}R^{d};
(f) C₁₋₆ alkyl optionally substituted with from 1-6 independently selected R^{f};
(g) C₁₋₆ alkoxy optionally substituted with from 1-6 independently selected R^{f};
(h) 3-12 membered heterocyclyl optionally substituted with from 1-6 independently selected R^{g};
(i) C₆₋₁₀ aryl optionally substituted with from 1-6 independently selected R^{g};
(j) 5-10 membered heteroaryl optionally substituted with from 1-6 independently selected R^{g}; and
(k) C₃₋₈ cycloalkyl optionally substituted with from 1-6 independently selected R^{g};
each R^{QB} is independently selected from the group consisting of:
(a) -L^{a}-S(O)₂-L^{b}-R^{c}; and
(b) 4-12 membered heterocyclyl or 7-10 membered bicyclic heteroaryl, each of which comprises an endocyclic S(O)₂ group, wherein the heterocyclyl or heteroaryl is optionally substituted with from 1-6 independently selected R^{g};
L^{a} is selected from the group consisting of —N(H)—, —N(R^{c})—, and —(CR^{h}R^{h})_{q1}—;
L^{b} is selected from the group consisting of —O—, —N(H)—, —N(R^{c})—, and —(CR^{h}R^{h})_{q2}—;
q1 and q2 are independently 0, 1, 2, 3, or 4;
provided that when L^{a} is —(CR^{h}R^{h})_{q1}— and q1 is 0, then -L^{b}-R^{e} is other than unsubstituted C₁₋₃ alkyl;
R^{a} and R^{b} are independently selected from the group consisting of C₁₋₆ alkyl which is optionally substituted with from 1-6 substituents each independently selected from the group consisting of C₁₋₆ alkoxy, C₃₋₆ cycloalkyl, and halo; C₃₋₆ cycloalkyl optionally substituted with from 1-3 substituents each independently selected from the group consisting of C₁₋₃ alkyl and halo; and C₆₋₁₀ aryl optionally substituted with from 1-3 independently selected C₁₋₃ alkyl; or
R^{a} and R^{b} taken together with the phosphorous atom to which each is attached forms a ring including from 5-8 ring atoms, wherein from 0-2 ring atoms (in addition to the phosphorous attached to R^{a} and R^{b}) are heteroatoms each independently selected from the group consisting of O, S, and N, wherein the ring is optionally substituted with from 1-3 independently selected C₁₋₆ alkyl;
each R^{c} and R^{d} are independently selected from the group consisting of H, C₁₋₆ alkyl, C₃₋₆ cycloalkyl, C(═O)(C₁₋₆ alkyl), C(═O)(C₃₋₆ cycloalkyl), C(═O)O(C₁₋₆ alkyl), S(O)₁₋₂(C₁₋₆ alkyl), and S(O)₁₋₂(C₃₋₆ cycloalkyl), wherein the C₁₋₆ alkyl, C(═O)(C₁₋₆ alkyl), C(═O)(C₃₋₆ cycloalkyl), C(═O)O(C₁₋₆ alkyl), S(O)₁₋₂(C₁₋₆ alkyl), and S(O)₁₋₂(C₃₋₆ cycloalkyl) are each optionally substituted with from 1-6 substituents independently selected from the group consisting of —OH, halo, and C₁₋₆ alkoxy;
R^{e} is H, or R^{e} is selected from the group consisting of C₁₋₆ alkyl, C₁₋₆ haloalkyl, C₁₋₆ alkoxy, C₁₋₆ haloalkoxy, C₃₋₆ cycloalkyl, and 3-8 membered heterocyclyl, each of which is optionally substituted with from 1-3 substituents each independently selected from the group consisting of halo, cyano, C₁₋₃ alkyl, C₁₋₃ haloalkyl, —OH, NH₂, NH(C₁₋₃ alkyl), N(C₁₋₃ alkyl)₂, C₁₋₃ alkoxy, and C₁₋₃ haloalkoxy;
L^{e} is a bond, NR^{c}, or O;
each R^{f} is independently selected from the group consisting of halo, —OH, NR^{c}R^{d}, C₁₋₆ alkoxy, C₁₋₆ haloalkoxy, and 3-12 membered heterocyclyl which is optionally substituted with from 1-4 substituents each independently selected from the group consisting of —OH, C₁₋₆ alkyl, and 3-12 membered heterocyclyl;
each R^{g} is independently selected from the group consisting of C₁₋₆ alkyl, C₁₋₆ haloalkyl, C₁₋₆ alkoxy, C₁₋₆ haloalkoxy, NR^{c}R^{d}, cyano, halo, C₃₋₆ cycloalkyl, and 3 to 12 membered heterocyclyl optionally substituted with one or more substituents each independently selected from the group consisting of C₁₋₆ alkyl and C(═O)C₁₋₆ alkyl; and
each occurrence of R^{h} is independently selected from the group consisting of H, C₁₋₆ alkyl, C₁₋₆ haloalkyl, C₃₋₆ cycloalkyl, and halo; or
a pair of R^{h} on the same or different carbon atom(s), taken together with the carbon atom(s) connecting them forms C₃₋₆ cycloalkyl or 4-8 membered heterocyclyl, each of which is optionally substituted with from 1-3 substituents each independently selected from the group consisting of halo, cyano, C₁₋₃ alkyl, C₁₋₃ haloalkyl, —OH, C₁₋₃ alkoxy, and C₁₋₃ haloalkoxy.

Also provided herein are pharmaceutical compositions comprising a compound of Formula I, or a pharmaceutically acceptable salt, deuterated analog, or solvate thereof, and a pharmaceutically acceptable excipient.

Also provided herein are methods for treating type 2 diabetes mellitus in a patient in need thereof, the methods comprising administering to the patient a therapeutically effective amount of a compound of Formula I, or a pharmaceutically acceptable salt, deuterated analog, or solvate thereof, or a pharmaceutical composition thereof.

Also provided herein are methods for treating type 2 diabetes mellitus in a patient, the methods comprising administering to a patient identified or diagnosed as having type 2 diabetes mellitus a therapeutically effective amount of a compound of Formula I, or a pharmaceutically acceptable salt, deuterated analog, or solvate thereof, or a pharmaceutical composition thereof.

Also provided herein are methods for treating diabetes mellitus in a patient, the methods comprising determining that the patient has type 2 diabetes mellitus; and administering to the patient a therapeutically effective amount of a compound of Formula I, or a pharmaceutically acceptable salt, deuterated analog, or solvate thereof, or a pharmaceutical composition thereof. In some embodiments, the step of determining that the patient has type 2 diabetes mellitus includes performing an assay to determine the level of an analyte in a sample from the patient, wherein the analyte is selected from the group consisting of hemoglobin A1c (HbA1c), fasting plasma glucose, non-fasting plasma glucose, or any combination thereof. In some embodiments, the level of HbA1c is greater than or about 6.5%. In some embodiments, the level of fasting plasma glucose is greater than or about 126 mg/dL. In some embodiments, the level of non-fasting plasma glucose is greater than or about 200 mg/dL.

In some embodiments, the methods further comprise obtaining a sample from the patient. In some embodiments, the sample is a body fluid sample. In some embodiments, the patient is about 40 to about 70 years old and is overweight or obese. In some embodiments, the patient has a body mass index (BMI) greater than or about 22 kg/m². In some embodiments, the patient has a BMI greater than or about 30 kg/m².

In some embodiments, the methods for the treatment of type 2 diabetes mellitus comprise a reduction in fasting plasma glucose levels. In some embodiments, the fasting plasma glucose levels are reduced to about or below 100 mg/dL.

In some embodiments, the methods for the treatment of type 2 diabetes mellitus comprise a reduction in HbA1c levels. In some embodiments, the HbA1c levels are reduced to about or below 5.7%.

In some embodiments, the methods for the treatment of type 2 diabetes mellitus comprise a reduction in glucagon levels.

In some embodiments, the methods for the treatment of type 2 diabetes mellitus comprise an increase in insulin levels.

In some embodiments, the methods for the treatment of type 2 diabetes mellitus comprise a decrease in BMI. In some embodiments, the BMI is decreased to about or below 25 kg/m².

In some embodiments, the compound of Formula I, or a pharmaceutically acceptable salt, deuterated analog, or solvate thereof, or a pharmaceutical composition thereof, is administered orally.

In some embodiments, the methods of treatment for type 2 diabetes mellitus further comprise administering an additional therapy or therapeutic agent to the patient. In some embodiments, the additional therapy or therapeutic agent is selected from the group consisting of an anti-diabetic agent, an anti-obesity agent, a GLP-1 receptor agonist, an agent to treat non-alcoholic steatohepatitis (NASH), gastric electrical stimulation, dietary monitoring, physical activity, or any combinations thereof. In some embodiments, the anti-diabetic agent is selected from the group consisting of a biguanide, a sulfonylurea, a glitazar, a thiazolidinedione, a dipeptidyl peptidase 4 (DPP-4) inhibitor, a meglitinide, a sodium-glucose linked transporter 2 (SGLT2) inhibitor, a glitazone, a GRP40 agonist, a glucose-dependent insulinotropic peptide (GIP), an insulin or insulin analogue, an alpha glucosidase inhibitor, a sodium-glucose linked transporter 1 (SGLT1) inhibitor, or any combinations thereof. In some embodiments, the biguanide is metformin. In some embodiments, the anti-obesity agent is selected from the group consisting of neuropeptide Y receptor type 2 (NPYR2) agonist, a NPYR1 or NPYR5 antagonist, a human proislet peptide (HIP), a cannabinoid receptor type 1 (CB1R) antagonist, a lipase inhibitor, a melanocortin receptor 4 agonist, a farnesoid X receptor (FXR) agonist, phentermine, zonisamide, a norepinephrine/dopamine reuptake inhibitor, a GDF-15 analog, an opioid receptor antagonist, a cholecystokinin agonist, a serotonergic agent, a methionine aminopeptidase 2 (MetAP2) inhibitor, diethylpropion, phendimetrazine, benzphetamine, a fibroblast growth factor receptor (FGFR) modulator, an AMP-activated protein kinase (AMPK) activator, or any combinations thereof. In some embodiments, the GLP-1 receptor agonist is selected from the group consisting of liraglutide, exenatide, dulaglutide, albiglutide, taspoglutide, lixisenatide, semaglutide, or any combinations thereof. In some embodiments, the agent to treat NASH is selected from the group consisting of an FXR agonist, PF-05221304, a synthetic fatty acid-bile conjugate, an anti-lysyl oxidase homologue 2 (LOXL2) monoclonal antibody, a caspase inhibitor, a MAPK5 inhibitor, a galectin 3 inhibitor, a fibroblast growth factor 21 (FGF21) agonist, a niacin analogue, a leukotriene D4 (LTD4) receptor antagonist, an acetyl-CoA carboxylase (ACC) inhibitor, a ketohexokinase (KHK) inhibitor, an ileal bile acid transporter (IBAT) inhibitor, an apoptosis signal-regulating kinase 1 (ASK1) inhibitor, or any combinations thereof. In some embodiments, the compound of Formula I, or a pharmaceutically acceptable salt, deuterated analog, or solvate thereof, or a pharmaceutical composition thereof, and the additional therapeutic agent are administered as separate dosages sequentially in any order.

Also provided herein are methods for modulating insulin levels in a patient in need of such modulating, the method comprising administering to the patient an effective amount of a compound of Formula I, or a pharmaceutically acceptable salt, deuterated analog, or solvate thereof, or a pharmaceutical composition thereof. In some embodiments, the modulation results in an increase of insulin levels.

Also provided herein are methods for modulating glucose levels in a patient in need of such modulating, the method comprising administering to the patient an effective amount of a compound of Formula I, or a pharmaceutically acceptable salt, deuterated analog, or solvate thereof, or a pharmaceutical composition thereof. In some embodiments, the modulation results in a decrease of glucose levels.

Also provided herein are methods for treating a GLP-1 associated disease, disorder, or condition, the method comprising administering to a patient in need thereof an effective amount of a compound of Formula I, or a pharmaceutically acceptable salt, deuterated analog, or solvate thereof, or a pharmaceutical composition thereof. In some embodiments, the disease, disorder, or condition is selected from the group consisting of type 1 diabetes mellitus, type 2 diabetes mellitus, early onset type 2 diabetes mellitus, idiopathic type 1 diabetes mellitus (Type 1b), youth-onset atypical diabetes (YOAD), maturity onset diabetes of the young (MODY), latent autoimmune diabetes in adults (LADA), obesity (including hypothalamic obesity and monogenic obesity), weight gain from use of other agents, idiopathic intracranial hypertension, Wolfram syndrome, gout, excessive sugar craving, hypertriglyceridemia, dyslipidemia, malnutrition-related diabetes, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, traumatic brain injury, peripheral vascular disease, endothelial dysfunction, impaired vascular compliance, vascular restenosis, thrombosis, hypertension, pulmonary hypertension, restenosis after angioplasty, intermittent claudication, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, macular degeneration, cataract, glomerulosclerosis, arthritis, osteoporosis, treatment of addiction, cocaine dependence, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), ulcerative colitis, inflammatory bowel disease, colitis, irritable bowel syndrome, Crohn's disease, short bowel syndrome, Parkinson's, Alzheimer's disease, impaired cognition, schizophrenia, Polycystic Ovary Syndrome (PCOS), or any combination thereof. In some embodiments, the disease, disorder, or condition is selected from the group consisting of type 2 diabetes mellitus, early onset type 2 diabetes mellitus, obesity, weight gain from use of other agents, gout, excessive sugar craving, hypertriglyceridemia, dyslipidemia, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), short bowel syndrome, Parkinson's disease, Polycystic Ovary Syndrome (PCOS), or any combination thereof. In some embodiments, the disease, disorder, or condition includes, but is not limited to type 2 diabetes mellitus, early onset type 2 diabetes mellitus, obesity, weight gain from use of other agents, gout, excessive sugar craving, hypertriglyceridemia, dyslipidemia, gestational diabetes, adipocyte dysfunction, visceral adipose deposition, myocardial infarction, peripheral arterial disease, stroke, transient ischemic attacks, hyperglycemia, post-prandial lipemia, metabolic acidosis, ketosis, hyperinsulinemia, impaired glucose metabolism, insulin resistance, hepatic insulin resistance, chronic renal failure, syndrome X, angina pectoris, diabetic nephropathy, impaired glucose tolerance, diabetic neuropathy, diabetic retinopathy, skin and connective tissue disorders, foot ulcerations, or a combination thereof.

### DETAILED DESCRIPTION

### Definitions

The following description sets forth exemplary embodiments of the present technology. It should be recognized, however, that such description is not intended as a limitation on the scope of the present disclosure but is instead provided as a description of exemplary embodiments.

As used in the present specification, the following words, phrases and symbols are generally intended to have the meanings as set forth below, except to the extent that the context in which they are used indicates otherwise.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

As used herein, the term “halo” or “halogen” means —F (sometimes referred to herein as “fluoro” or “fluoros”), —Cl (sometimes referred to herein as “chloro” or “chloros”), —Br (sometimes referred to herein as “bromo” or “bromos”), and —I (sometimes referred to herein as “iodo” or “iodos”).

As used herein, the term “alkyl” refers to saturated linear or branched-chain monovalent hydrocarbon radicals, containing the indicated number of carbon atoms. For example, “C₁₋₆ alkyl” refers to saturated linear or branched-chain monovalent hydrocarbon radicals of one to six carbon atoms. Non-limiting examples of alkyl include methyl, ethyl, 1-propyl, isopropyl, 1-butyl, isobutyl, sec-butyl, tert-butyl, 2-methyl-2-propyl, pentyl, neopentyl, and hexyl.

As used herein, the term “alkylene” refers to a divalent alkyl containing the indicated number of carbon atoms. For example, “C₁₋₃ alkylene” refers to a divalent alkyl having one to three carbon atoms (e.g., —CH₂—, —CH(CH₃)—, —CH₂CH₂—, or —CH₂CH₂CH₂—).

As used herein, the term “alkenyl” refers to a linear or branched mono-unsaturated hydrocarbon chain, containing the indicated number of carbon atoms. For example, “C₂₋₆ alkenyl” refers a linear or branched mono unsaturated hydrocarbon chain of two to six carbon atoms. Non-limiting examples of alkenyl include ethenyl, propenyl, butenyl, or pentenyl.

As used herein, the term “alkynyl” refers to a linear or branched di-unsaturated hydrocarbon chain, containing the indicated number of carbon atoms. For example, “C₂₋₆ alkynyl” refers to a linear or branched di-unsaturated hydrocarbon chain having two to six carbon atoms. Non-limiting examples of alkynyl include ethynyl, propynyl, butynyl, or pentynyl.

As used herein, the term “cycloalkyl” refers to a saturated or partially saturated cyclic hydrocarbon, containing the indicated number of carbon atoms. For example, “C₃₋₆ cycloalkyl” refers to a saturated or partially saturated cyclic hydrocarbon having three to six ring carbon atoms. Non-limiting examples of cycloalkyl include cyclopropyl, cyclobutyl, cyclopentyl, and cyclohexyl. Cycloalkyl may include multiple fused and/or bridged rings. Non-limiting examples of fused/bridged cycloalkyl includes: bicyclo[1.1.0]butane, bicyclo[2.1.0]pentane, bicyclo[1.1.1]pentane, bicyclo[3.1.0]hexane, bicyclo[2.1.1]hexane, bicyclo[3.2.0]heptane, bicyclo[4.1.0]heptane, bicyclo[2.2.1]heptane, bicyclo[3.1.1]heptane, bicyclo[4.2.0]octane, bicyclo[3.2.1]octane, bicyclo[2.2.2]octane, and the like. Cycloalkyl also includes spirocyclic rings (e.g., spirocyclic bicycle wherein two rings are connected through just one atom). Non-limiting examples of spirocyclic cycloalkyls include spiro[2.2]pentane, spiro[2.5]octane, spiro[3.5]nonane, spiro[3.5]nonane, spiro[3.5]nonane, spiro[4.4]nonane, spiro[2.6]nonane, spiro[4.5]decane, spiro[3.6]decane, spiro[5.5]undecane, and the like.

As used herein, the term “heterocyclyl” refers to a mono-, bi-, tri-, or polycyclic nonaromatic ring system containing indicated number of ring atoms (e.g., 3-8 membered monocyclic, 8-12 membered bicyclic, or 11-14 membered tricyclic ring system) having 1-3 heteroatoms if monocyclic, 1-6 heteroatoms if bicyclic, or 1-9 heteroatoms if tricyclic or polycyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocyclyl groups include piperazinyl, pyrrolidinyl, dioxanyl, morpholinyl, tetrahydrofuranyl, and the like. Heterocyclyl may include multiple fused and bridged rings. Non-limiting examples of fused/bridged heterocyclyl includes: 2-azabicyclo[1.1.0]butane, 2-azabicyclo[2.1.0]pentane, 2-azabicyclo[1.1.1]pentane, 3-azabicyclo[3.1.0]hexane, 5-azabicyclo[2.1.1]hexane, 3-azabicyclo[3.2.0]heptane, octahydrocyclopenta[c]pyrrole, 3-azabicyclo[4.1.0]heptane, 7-azabicyclo[2.2.1]heptane, 6-azabicyclo[3.1.1]heptane, 7-azabicyclo[4.2.0]octane, 2-azabicyclo[2.2.2]octane, 3-azabicyclo[3.2.1]octane, 2-oxabicyclo[1.1.0]butane, 2-oxabicyclo[2.1.0]pentane, 2-oxabicyclo[1.1.1]pentane, 3-oxabicyclo[3.1.0]hexane, 5-oxabicyclo[2.1.1]hexane, 3-oxabicyclo[3.2.0]heptane, 3-oxabicyclo[4.1.0]heptane, 7-oxabicyclo[2.2.1]heptane, 6-oxabicyclo[3.1.1]heptane, 7-oxabicyclo[4.2.0]octane, 2-oxabicyclo[2.2.2]octane, 3-oxabicyclo[3.2.1]octane, and the like. Heterocyclyl also includes spirocyclic rings (e.g., spirocyclic bicycle wherein two rings are connected through just one atom). Non-limiting examples of spirocyclic heterocyclyls include 2-azaspiro[2.2]pentane, 4-azaspiro[2.5]octane, 1-azaspiro[3.5]nonane, 2-azaspiro[3.5]nonane, 7-azaspiro[3.5]nonane, 2-azaspiro[4.4]nonane, 6-azaspiro[2.6]nonane, 1,7-diazaspiro[4.5]decane, 7-azaspiro[4.5]decane 2,5-diazaspiro[3.6]decane, 3-azaspiro[5.5]undecane, 2-oxaspiro[2.2]pentane, 4-oxaspiro[2.5]octane, 1-oxaspiro[3.5]nonane, 2-oxaspiro[3.5]nonane, 7-oxaspiro[3.5]nonane, 2-oxaspiro[4.4]nonane, 6-oxaspiro[2.6]nonane, 1,7-dioxaspiro[4.5]decane, 2,5-dioxaspiro[3.6]decane, 1-oxaspiro[5.5]undecane, 3-oxaspiro[5.5]undecane, 3-oxa-9-azaspiro[5.5]undecane and the like.

As used herein, the term “aryl” refers to a mono-, bi-, tri- or polycyclic hydrocarbon group containing the indicated numbers of carbon atoms, wherein at least one ring in the system is aromatic (e.g., C₆ monocyclic, C₁₀ bicyclic, or C₁₄ tricyclic aromatic ring system). Examples of aryl groups include phenyl, naphthyl, tetrahydronaphthyl, and the like.

As used herein, the term “heteroaryl” refers to a mono-, bi-, tri- or polycyclic group having indicated numbers of ring atoms (e.g., 5-6 ring atoms; e.g., 5, 6, 9, 10, or 14 ring atoms); wherein at least one ring in the system is aromatic (but does not have to be a ring which contains a heteroatom, e.g. tetrahydroisoquinolinyl, e.g., tetrahydroquinolinyl), and at least one ring in the system contains one or more heteroatoms independently selected from the group consisting of N, O, and S. Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridine, pyrazolo[4,3-b]pyridinyl, tetrazolyl, chromane, 2,3-dihydrobenzo[b][1,4]dioxine, benzo[d][1,3]dioxole, 2,3-dihydrobenzofuran, tetrahydroquinoline, 2,3-dihydrobenzo[b][1,4]oxathiine, isoindoline, and others.

As used herein, the term “haloalkyl” refers to an alkyl radical as defined herein, wherein one or more hydrogen atoms is replaced with one or more halogen atoms. Non-limiting examples include fluoromethyl, difluoromethyl, trifluoromethyl, 2-fluoroethyl, 2,2-difluoroethyl, 2,2,2-trifluoroethyl, chloromethyl, dichloromethyl, chloroethyl, trichloroethyl, bromomethyl, and iodomethyl.

As used herein, the term “alkoxy” refers to an —O-alkyl radical, wherein the radical is on the oxygen atom. For example, “C₁₋₆ alkoxy” refers to an —O—(C₁₋₆ alkyl) radical, wherein the radical is on the oxygen atom. Examples of alkoxy include methoxy, ethoxy, propoxy, isopropoxy, butoxy and tert-butoxy. Accordingly, as used herein, the term “haloalkoxy” refers to an —O-haloalkyl radical, wherein the radical is on the oxygen atom.

As used herein, “ [symbol] ” indicates an optional single or double bond, as allowed by valence. As used herein, “ [symbol] ” indicates the point of attachment to the parent molecule.

As used herein, the term “compound,” is meant to include all stereoisomers, geometric isomers, tautomers, and isotopes of the structures depicted. Compounds herein identified by name or structure as one particular tautomeric form are intended to include other tautomeric forms unless otherwise specified.

The term “tautomer” as used herein refers to compounds whose structures differ markedly in arrangement of atoms, but which exist in easy and rapid equilibrium, and it is to be understood that compounds provided herein may be depicted as different tautomers, and when compounds have tautomeric forms, all tautomeric forms are intended to be within the scope of the disclosure, and the naming of the compounds does not exclude any tautomer.

The term “GLP-1R” or “GLP-1 receptor” as used herein is meant to include, without limitation, nucleic acids, polynucleotides, oligonucleotides, sense and antisense polynucleotide strands, complementary sequences, peptides, polypeptides, proteins, homologous, and/or orthologous GLP-1R molecules, isoforms, precursors, mutants, variants, derivatives, splice variants, alleles, different species, and active fragments thereof.

The term “GLP-1 associated disease” as used herein is meant to include, without limitation, all those diseases, disorders, or conditions in which modulating glucagon-like peptide-1 (GLP-1) receptor signaling can alter the pathology and/or symptoms and/or progression of the disease, disorder, or condition.

The term “GLP-1 agonist” or “GLP-1 RA” as used herein refers to an agonist of the glucagon-like peptide-1 (GLP-1) receptor. GLP-1 RAs enhance glucose-dependent insulin secretion; suppress inappropriately elevated glucagon levels, both in fasting and postprandial states; and slow gastric emptying. Karla et al., Glucagon-like peptide-1 receptor agonists in the treatment of type 2 diabetes: Past, present, and future, Indian J Endocrinol Metab. 2016 March-April; 20(2): 254-267. GLP-1 RAs have been shown to treat type 2 diabetes. Examples of GLP-1 RAs include, but are not limited to, albiglutide (TANZEUM®), dulaglutide (LY2189265, TRULICITY®), efpeglenatide, exenatide (BYETTA®, BYDUREON®, Exendin-4), liraglutide (VICTOZA®, NN2211), lixisenatide (LYXUMIA®), semaglutide (OZEMPIC®), tirzepatide, ZP2929, NNC0113-0987, BPI-3016, and TT401. See, also, for example, additional GLP-1 receptor agonists described in U.S. Pat. Nos. 10,370,426; 10,308,700; 10,259,823; 10,208,019; 9,920,106; 9,839,664; 8,129,343; 8,536,122; 7,919,598; 6,414,126; 6,628,343; and RE45313.

The term “pharmaceutically acceptable” as used herein indicates that the compound, or salt or composition thereof is compatible chemically and/or toxicologically with the other ingredients comprising a formulation and/or the patient being treated therewith.

The term “pharmaceutically acceptable salt” of a given compound refers to salts that retain the biological effectiveness and properties of the given compound and which are not biologically or otherwise undesirable. “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include, e.g., hydrochloric acid, hydrobromic acid, sulfuric acid, nitric acid, phosphoric acid and the like. Salts derived from organic acids include, e.g., acetic acid, propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic and organic bases. Salts derived from inorganic bases include, by way of example only, sodium, potassium, lithium, aluminum, ammonium, calcium, and magnesium salts. Salts derived from organic bases include, but are not limited to, salts of NH₃, or primary, secondary, tertiary amines, such as salts derived from a N-containing heterocycle, a N-containing heteroaryl, or derived from an amine of formula N(R^{N})₃ (e.g., HN⁺(R^{N})₃ or (alkyl)N⁺(R^{N})₃) where each R^{N} is independently hydrogen, alkyl, alkenyl, alkynyl, haloalkyl, cycloalkyl, heterocyclyl, aryl, or heteroaryl, wherein each is optionally substituted, such as by one or more (e.g., 1-5 or 1-3) substituents (e.g., halo, cyano, hydroxy, amino, alkyl, alkenyl, alkynyl, haloalkyl, alkoxy, or haloalkoxy). Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

The term “therapeutic compound” as used herein is meant to include, without limitation, all compounds of Formula I, or pharmaceutically acceptable salts or solvates thereof (e.g., a compound of any one of Formula II, III, or IV, or a pharmaceutically acceptable salt, deuterated analog, or solvate thereof), and all compositions (e.g., pharmaceutical compositions) wherein a compound of Formula I, or a pharmaceutically acceptable salt, deuterated analog, or solvate thereof (e.g., a compound of any one of Formula II, III, or IV, or a pharmaceutically acceptable salt, deuterated analog, or solvate thereof) is a component of the composition.

A “solvate” is formed by the interaction of a solvent and a compound. Solvates of salts of the compounds described herein are also provided. Hydrates of the compounds described herein are also provided.

The term “deuterated analog” of compounds described herein in which one or more hydrogens is/are replaced by deuterium, such as a hydrogen on a carbon atom. The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen,” the position is understood to have hydrogen at its natural abundance isotopic composition. Accordingly, in the compounds of this disclosure any atom specifically designated as a deuterium (D) is meant to represent deuterium.

The term “administration” or “administering” refers to a method of giving a dosage of a compound or pharmaceutical composition to a vertebrate or invertebrate, including a mammal, a bird, a fish, or an amphibian. The method of administration can vary depending on various factors, e.g., the components of the pharmaceutical composition, the site of the disease, and the severity of the disease.

The terms “effective amount” or “effective dosage” or “pharmaceutically effective amount” or “therapeutically effective amount,” as used herein, refer to a sufficient amount of a chemical entity (e.g., a compound of Formula I, or a pharmaceutically acceptable salt, deuterated analog, or solvate thereof (e.g., a compound of any one of Formula II, III, or IV, or a pharmaceutically acceptable salt, deuterated analog, or solvate thereof)) being administered which will relieve to some extent one or more of the symptoms of the disease or condition being treated, and can include curing the disease. “Curing” means that the symptoms of active disease are eliminated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study. In some embodiments, a “therapeutically effective amount” of a compound as provided herein refers to an amount of the compound that is effective as a monotherapy or combination therapy.

The term “excipient” or “pharmaceutically acceptable excipient” means a pharmaceutically-acceptable material, composition, or vehicle, such as a liquid or solid filler, diluent, carrier, solvent, or encapsulating material. In some embodiments, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

The term “pharmaceutical composition” refers to a mixture of a compound of Formula I, or a pharmaceutically acceptable salt, deuterated analog, or solvate thereof (e.g., a compound of any one of Formula II, III, or IV or a pharmaceutically acceptable salt, deuterated analog, or solvate thereof) as described herein with other chemical components (referred to collectively herein as “excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to, rectal, oral, intravenous, aerosol, parenteral, ophthalmic, pulmonary, and topical administration.

The terms “treat,” “treating,” and “treatment,” in the context of treating a disease, disorder, or condition, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof.

The term “preventing”, as used herein, is the prevention of the onset, recurrence or spread, in whole or in part, of the disease or condition as described herein, or a symptom thereof.

The terms “subject,” “patient,” or “individual,” as used herein, are used interchangeably and refers to any animal, including mammals such as mice, rats, other rodents, rabbits, dogs, cats, swine, cattle, sheep, horses, primates, and humans. In some embodiments, the term refers to a subject, particularly a mammalian subject, for whom diagnosis, prognosis, or therapy is desired or needed. In some embodiments, the patient is a human. In some embodiments, the subject has experienced and/or exhibited at least one symptom of the disease, disorder, or condition to be treated and/or prevented.

The terms “treatment regimen” and “dosing regimen” are used interchangeably to refer to the dose and timing of administration of each therapeutic agent.

The term “pharmaceutical combination,” as used herein, refers to a pharmaceutical treatment resulting from the mixing or combining of more than one active ingredient and includes both fixed and non-fixed combinations of the active ingredients.

The term “combination therapy” as used herein refers to a dosing regimen of two different therapeutically active agents (i.e., the components or combination partners of the combination), wherein the therapeutically active agents are administered together or separately in a manner prescribed by a medical care taker or according to a regulatory agency as defined herein.

The term “modulation,” as used herein, refers to a regulation or an adjustment (e.g., increase or decrease) and can include, for example agonism, partial agonism or antagonism.

### Compounds

Provided herein are heterocyclic GLP-1 agonists for use in the management of T2DM and other conditions where activation of GLP-1 activity is useful.

In some embodiments, provided is a compound of Formula I:

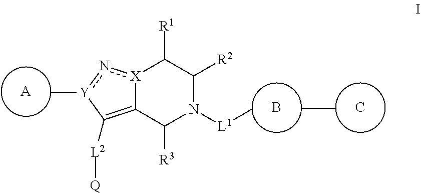

or a pharmaceutically acceptable salt, deuterated analog, or solvate thereof, wherein:
X is C and Y is N, or Y is C and X is N and the dashed lines represent an optional bond such that the moiety

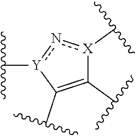

is

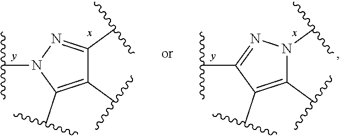 [SMILES: *c1nn(*)c(*)c1* ; *c1nn(*)c(*)c1*]

where bond y is attached to Y and bond x is attached to X;
Ring A is C₆₋₁₀ aryl, C₅₋₁₀ cycloalkyl, 5-10 membered heterocyclyl, or 5-10 membered heteroaryl, each of which is optionally substituted with from 1-5 substituents each independently selected from the group consisting of halo, C₁₋₆ alkyl, C₁₋₆ haloalkyl, C₁₋₆alkoxy, and C₃₋₆ cycloalkyl;
R¹, R², and R³ are each independently selected from the group consisting of H and C₁₋₆ alkyl which is optionally substituted with from 1-6 substituents each independently selected from the group consisting of halo, —OH, and C₁₋₆ alkoxy;
L¹ is selected from the group consisting of —C(═O)—, —CH₂—, —CH(C₁₋₆ alkyl)-, and —S(═O)₂;
Ring B is selected from the group consisting of

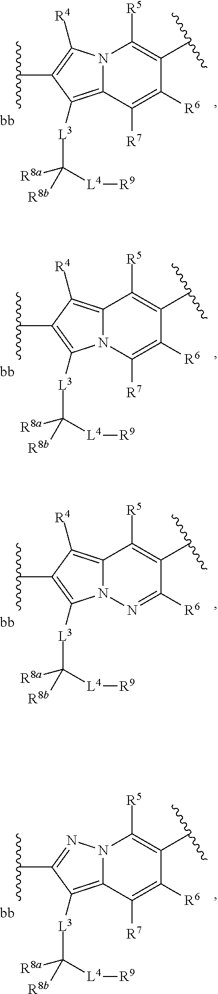

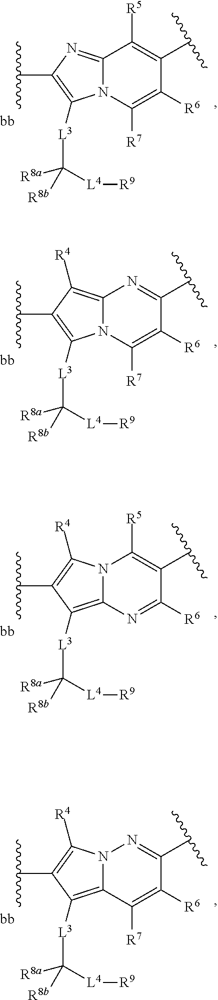

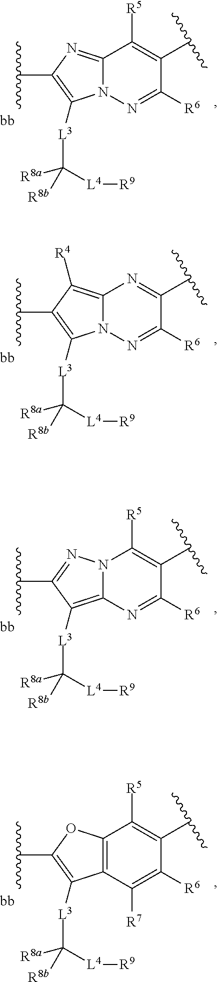

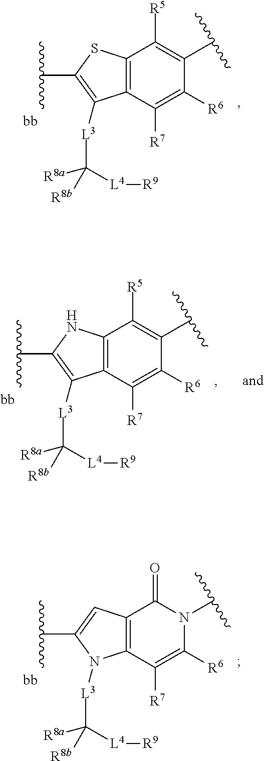

wherein bb represents point of attachment to L¹;
R⁴, R⁵, R⁶, and R⁷ are independently selected from the group consisting of H, halo, and C₁₋₆ alkyl;
L³ is a bond or C₁₋₃ alkylene;
L⁴ is a bond or C₁₋₅ alkylene;
R^{8a} and R^{8b} are independently selected from the group consisting of H and C₁₋₆ alkyl optionally substituted with one or more substituents independently selected from the group consisting of halo and C₃₋₁₅ cycloalkyl;
or R^{8a} and R^{8b} taken together with the carbon atom to which each is attached forms a C₃₋₁₅ cycloalkyl ring which is optionally substituted with from 1-3 independently selected C₁₋₆ alkyl, wherein the C₁₋₆ alkyl is optionally substituted with from 1-6 independently selected from R^{f};
or the moiety -L³-C(R^{8a}R^{8b})-L⁴-R⁹ is a phenyl substituted with C(═O)OH, and optionally 1-4 additional substituents independently selected from R^{f};
R⁹ is selected from the group consisting of C(═O)OH, C(═O)(OC₁₋₆ alkyl), C(═O)NR^{9a}R^{9b}, (IX-1), (IX-2), (IX-3), and (IX-4):

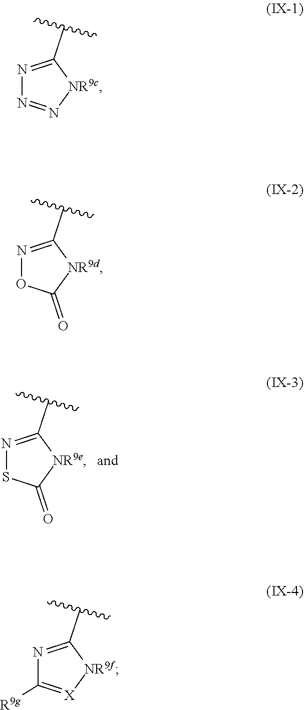

R^{9a} is H or C₁₋₆ alkyl;
R^{9b} is H, C₁₋₆ alkyl, C(═O)(C₁₋₆ alkyl), S(O)₀₋₂(C₁₋₆ alkyl), or cyano;
R^{9c}, R^{9d}, R^{9e}, R^{9f}, and R^{9g} are each independently selected from the group consisting of H, C₁₋₆ alkyl optionally substituted with from 1-6 independently selected halo and C₁₋₆ alkoxy, and C(═O)(C₁₋₆ alkyl);
Ring C is selected from the group consisting of 3-12 membered heterocyclyl, C₃₋₁₅ cycloalkyl, and 5-10 membered heteroaryl, each of which is optionally substituted with from 1-3 R^{Ca};
each R^{Ca} is independently selected from the group consisting of halo, C₁₋₆ alkyl, C₁₋₆ haloalkyl, C₁₋₆ alkoxy, and NR^{c}R^{d};
or a pair of R^{Ca} on the same or different ring atoms, taken together with the ring atom(s) to which each is attached, forms a carbocyclic ring including from 3-8 ring atoms;
L² is selected from the group consisting of

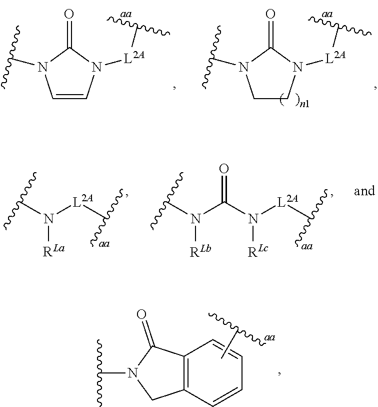

wherein aa represents the point of attachment to Q;
n1 is an integer from 1-3;
L^{2A} is a bond or C₁₋₁₀ alkylene;
R^{La} is selected from the group consisting of H, C₁₋₆ alkyl, and C(═O)(C₁₋₆ alkyl);
each of R^{Lb} and R^{L}c is independently selected from the group consisting of H and C₁₋₆ alkyl;
Q is selected from the group consisting of C₁₋₁₀ alkyl; C₃₋₁₅ cycloalkyl; 3-12 membered heterocyclyl; 5-10 membered heteroaryl; and C₆₋₁₀ aryl, wherein Q is substituted with —P(═O)R^{a}R^{b}, and optionally substituted with from 1-6 additional substituents independently selected from R^{Q};
each R^{Q} is independently selected from the group consisting of
(a) halo;
(b) cyano;
(c) OH or oxo;
(d) —NR^{c}R^{d};
(e) —C(═O)NR^{c}R^{d} or —S(O)₂NR^{c}R^{d};
(f) —S(═O)₀₋₂R^{c};
(g) C₁₋₆ alkyl optionally substituted with from 1-6 independently selected R^{f};
(h) C₁₋₆ alkoxy optionally substituted with from 1-6 independently selected R^{f};
(i) 3-12 membered heterocyclyl optionally substituted with from 1-6 independently selected R^{g};
(j) C₆₋₁₀ aryl optionally substituted with from 1-6 independently selected R^{g};
(k) 5-10 membered heteroaryl optionally substituted with from 1-6 independently selected R^{g};
(l) C₃₋₈ cycloalkyl optionally substituted with from 1-6 independently selected R^{g};
(m) —P(═O)R^{a}R^{b}; and
(n) —(CR^{h}R^{h})_{q1}—S(O)₂-L^{e}-R^{e}, wherein q1 is 1, 2, or 3;
or Q is C₃₋₁₅ cycloalkyl; 3-12 membered heterocyclyl; 5-10 membered heteroaryl; and C₆₋₁₀ aryl, each of which is optionally substituted with from 1-6 substituents each independently selected from the group consisting of R^{QA} and R^{QB};
provided that one or both of (aa) or (bb) applies:
(aa) Q is substituted with at least one R^{QB}; or
(bb) Q is 4-12 membered heterocyclyl or 7-10 membered bicyclic heteroaryl, wherein Q comprises an endocyclic S(O)₂ group;
each R^{QA} is independently selected from the group consisting of
(a) halo;
(b) cyano;
(c) OH or oxo;
(d) —NR^{a}R^{b};
(e) —C(═O)NR^{c}R^{d};
(f) C₁₋₆ alkyl optionally substituted with from 1-6 independently selected R^{f};
(g) C₁₋₆ alkoxy optionally substituted with from 1-6 independently selected R^{f};
(h) 3-12 membered heterocyclyl optionally substituted with from 1-6 independently selected R^{g};
(i) C₆₋₁₀ aryl optionally substituted with from 1-6 independently selected R^{g};
(j) 5-10 membered heteroaryl optionally substituted with from 1-6 independently selected R^{g}; and
(k) C₃₋₈ cycloalkyl optionally substituted with from 1-6 independently selected R^{g};
each R^{QB} is independently selected from the group consisting of:
   a) -L^{a}-S(O)₂-L^{b}-R^{e}; and
   b) 4-12 membered heterocyclyl or 7-10 membered bicyclic heteroaryl, each of which comprises an endocyclic S(O)₂ group, wherein the heterocyclyl or heteroaryl is optionally substituted with from 1-6 independently selected R^{g};
L^{a} is selected from the group consisting of —N(H)—, —N(R^{c})—, and —(CR^{h}R^{h})_{q1}—;
L^{b} is selected from the group consisting of —O—, —N(H)—, —N(R^{c})—, and —(CR^{h}R^{h})_{q2}—;
q1 and q2 are independently 0, 1, 2, 3, or 4;
provided that when L^{a} is —(CR^{h}R^{h})_{q1}— and q1 is 0, then -L^{b}-R^{e} is other than unsubstituted C₁₋₃ alkyl;
R^{a} and R^{b} are independently selected from the group consisting of C₁₋₆ alkyl which is optionally substituted with from 1-6 substituents each independently selected from the group consisting of C₁₋₆ alkoxy, C₃₋₆ cycloalkyl, and halo; C₃₋₆ cycloalkyl optionally substituted with from 1-3 substituents each independently selected from the group consisting of C₁₋₃ alkyl and halo; and C₆₋₁₀ aryl optionally substituted with from 1-3 independently selected C₁₋₃ alkyl; or
R^{a} and R^{b} taken together with the phosphorous atom to which each is attached forms a ring including from 5-8 ring atoms, wherein from 0-2 ring atoms (in addition to the phosphorous attached to R^{a} and R^{b}) are heteroatoms each independently selected from the group consisting of O, S, and N, wherein the ring is optionally substituted with from 1-3 independently selected C₁₋₆ alkyl;
each R^{c} and R^{d} are independently selected from the group consisting of H, C₁₋₆ alkyl, C₃₋₆ cycloalkyl, C(═O)(C₁₋₆ alkyl), C(═O)(C₃₋₆ cycloalkyl), C(═O)O(C₁₋₆ alkyl), S(O)₁₋₂(C₁₋₆ alkyl), and S(O)₁₋₂(C₃₋₆ cycloalkyl), wherein the C₁₋₆ alkyl, C(═O)(C₁₋₆ alkyl), C(═O)(C₃₋₆ cycloalkyl), C(═O)O(C₁₋₆ alkyl), S(O)₁₋₂(C₁₋₆ alkyl), and S(O)₁₋₂(C₃₋₆ cycloalkyl) are each optionally substituted with from 1-6 substituents independently selected from the group consisting of —OH, halo, and C₁₋₆ alkoxy;
R^{c} is H, or R^{c} is selected from the group consisting of C₁₋₆ alkyl, C₁₋₆ haloalkyl, C₁₋₆ alkoxy, C₁₋₆ haloalkoxy, C₃₋₆ cycloalkyl, and 3-8 membered heterocyclyl, each of which is optionally substituted with from 1-3 substituents each independently selected from the group consisting of halo, cyano, C₁₋₃ alkyl, C₁₋₃ haloalkyl, —OH, NH₂, NH(C₁₋₃ alkyl), N(C₁₋₃ alkyl)₂, C₁₋₃ alkoxy, and C₁₋₃ haloalkoxy;
L^{e} is a bond, NR^{c}, or O;
each R^{f} is independently selected from the group consisting of halo, —OH, NR^{c}R^{d}, C₁₋₆ alkoxy, C₁₋₆ haloalkoxy, and 3-12 membered heterocyclyl which is optionally substituted with from 1-4 substituents each independently selected from the group consisting of —OH, C₁₋₆ alkyl, and 3-12 membered heterocyclyl;
each R^{g} is independently selected from the group consisting of C₁₋₆ alkyl, C₁₋₆ haloalkyl, C₁₋₆ alkoxy, C₁₋₆ haloalkoxy, NR^{c}R^{d}, cyano, halo, C₃₋₆ cycloalkyl, and 3 to 12 membered heterocyclyl optionally substituted with one or more substituents each independently selected from the group consisting of C₁₋₆ alkyl and C(═O)C₁₋₆ alkyl; and
each occurrence of R^{h} is independently selected from the group consisting of H, C₁₋₆ alkyl, C₁₋₆ haloalkyl, C₃₋₆ cycloalkyl, and halo; or
a pair of R^{h} on the same or different carbon atom(s), taken together with the carbon atom(s) connecting them forms C₃₋₆ cycloalkyl or 4-8 membered heterocyclyl, each of which is optionally substituted with from 1-3 substituents each independently selected from the group consisting of halo, cyano, C₁₋₃ alkyl, C₁₋₃ haloalkyl, —OH, C₁₋₃ alkoxy, and C₁₋₃ haloalkoxy.

In some embodiments, the moiety

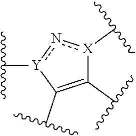

is

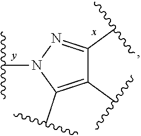 [SMILES: *c1nn(*)c(*)c1*]

where bond y is attached to Y and bond x is attached to X;

In some embodiments, the moiety

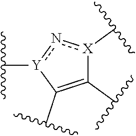

is

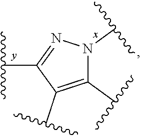 [SMILES: *c1nn(*)c(*)c1*]

where bond y is attached to Y and bond x is attached to X.

In some embodiments, provided is a compound of Formula II:

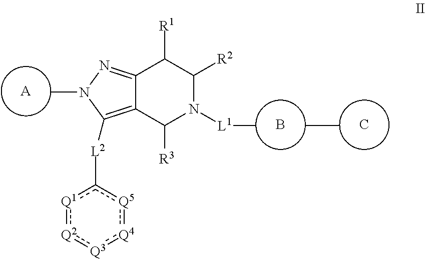

or a pharmaceutically acceptable salt thereof or a solvate thereof, wherein:
Q¹, Q², Q³, Q⁴, and Q⁵ are defined according to (AA) or (BB) below:
[symbol] (AA)
Q¹ and Q⁵ are independently selected from the group consisting of N, CH, and CR^{QC};
Q², Q³, and Q⁴ are independently selected from the group consisting of N, CH, CR^{QC}, and CR^{QD}, provided that at least one of Q², Q³, and Q⁴ is CR^{QD};
each [symbol] is a single bond or double bond, provided that the ring including Q¹-Q⁵ is aromatic;
[symbol] (BB)
Q¹ is a bond;
Q², Q³, Q⁴, and Q⁵ are independently selected from the group consisting of O, S, N, NH, NR^{c}, CH, CR^{QC}, and CR^{QD}, provided that at least one of Q², Q³, Q⁴, and Q⁵ is CR^{QD};
each [symbol] is a single bond or double bond, provided that the ring including Q¹-Q⁵ is aromatic;
R^{QD} is P(═O)R^{a}R^{b}, wherein R^{a} and R^{b} are independently selected from the group consisting of C₁₋₆ alkyl which is optionally substituted with from 1-6 substituents each independently selected from the group consisting of C₁₋₆ alkoxy, C₃₋₆ cycloalkyl, and halo; C₃₋₆ cycloalkyl optionally substituted with from 1-3 substituents each independently selected from the group consisting of C₁₋₃ alkyl and halo; and C₆₋₁₀ aryl optionally substituted with from 1-3 independently selected C₁₋₃ alkyl; or
R^{a} and R^{b} taken together with the phosphorous atom to which each is attached forms a ring including from 5-8 ring atoms, wherein from 0-2 ring atoms (in addition to the phosphorous attached to R^{a} and R^{b}) are heteroatoms each independently selected from the group consisting of O, S, and N, wherein the ring is optionally substituted with from 1-3 independently selected C₁₋₆ alkyl;
each R^{QC} is independently selected from the group consisting of (a) halo; (b) cyano; (c) OH; (d) —NR^{c}R^{d}; (e) C(═O)NR^{c}R^{d}; (f) S(═O)₀₋₂R^{c}; (g) C₁₋₆ alkyl optionally substituted with from 1-6 independently selected R^{f}; (h) C₁₋₆ alkoxy optionally substituted with from 1-6 substituents each independently selected from the group consisting of hydroxy, halo, and C₁₋₆ alkoxy; (i) 3-12 membered heterocyclyl optionally substituted with one or more substituents each independently selected from the group consisting of C₁₋₆ alkyl and C(═O)(C₁₋₆ alkyl); j) C₆₋₁₀ aryl optionally substituted with from 1-3 independently selected C(═O)(C₁₋₆ alkyl); and (k) 5-10 membered heteroaryl optionally substituted with from 1-6 independently selected R^{g};
or a pair of R^{QC} on adjacent carbon atoms, taken together with the atom to which each is attached, forms a ring including from 5-8 ring atoms, wherein from 0-2 ring atoms are heteroatoms each independently selected from the group consisting of O, N, and S, wherein said ring is optionally substituted with from 1-2 independently selected R^{h} groups;
L² is selected from the group consisting of

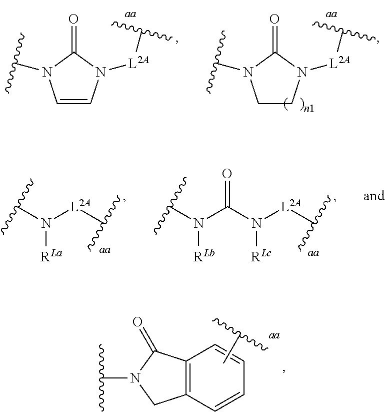

wherein aa represents the point of attachment to the ring containing Q¹-Q⁵;
n1 is an integer from 1-3;
L^{2A} is a bond or C₁₋₁₀ alkylene;
R^{La} is selected from the group consisting of H, C₁₋₆ alkyl, and C(═O)(C₁₋₆ alkyl);
each of R^{Lb} and R^{L}c is independently selected from the group consisting of H and C₁₋₆ alkyl;
Ring A is C₆₋₁₀ aryl, C₅₋₇ cycloalkyl, 5-7 membered heterocyclyl, or 5-10 membered heteroaryl, each of which is optionally substituted with from 1-5 substituents each independently selected from the group consisting of halo, C₁₋₆ alkyl, C₁₋₆ haloalkyl, C₁₋₆ alkoxy, and C₃₋₆ cycloalkyl;
R¹, R², and R³ are each independently selected from the group consisting of H and C₁₋₆ alkyl which is optionally substituted with from 1-6 substituents each independently selected from the group consisting of halo, —OH, and C₁₋₆ alkoxy;
L¹ is selected from the group consisting of —C(═O)—, —CH₂—, —CH(C₁₋₆ alkyl)-, and —S(═O)₂;
Ring B is selected from the group consisting of

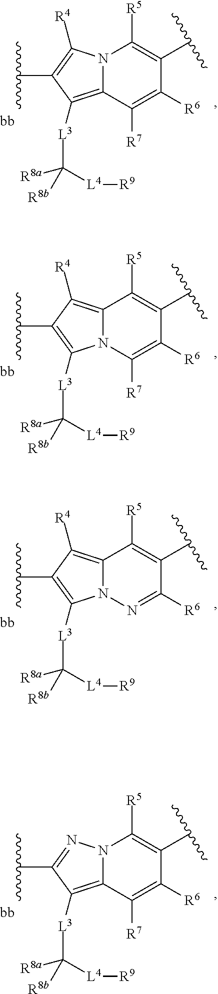

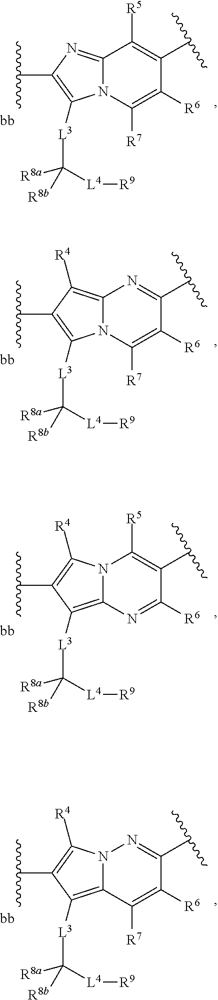

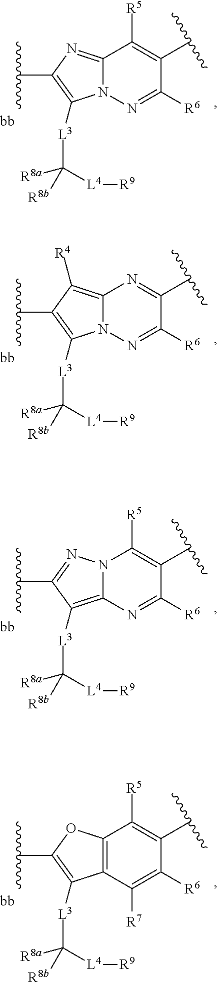

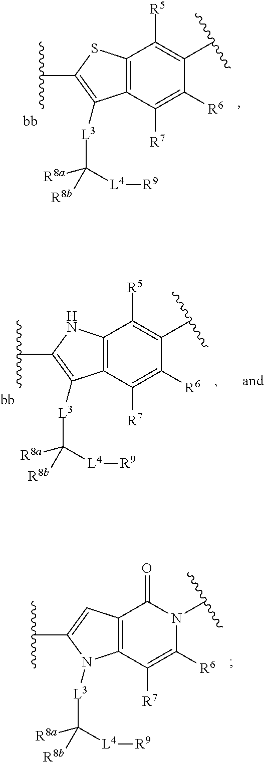

wherein bb represents point of attachment to L¹;
R⁴, R⁵, R⁶, and R⁷ are independently selected from the group consisting of H, halo, and C₁₋₆ alkyl;
L³ is a bond or C₁₋₃ alkylene;
L⁴ is a bond or C₁₋₅ alkylene;
R^{8a} and R^{8b} are independently selected from the group consisting of H and C₁₋₆ alkyl optionally substituted with one or more substituents independently selected from the group consisting of halo and C₃₋₁₅ cycloalkyl; or
R^{8a} and R^{8b} taken together with the carbon atom to which each is attached forms a C₃₋₁₅ cycloalkyl ring which is optionally substituted with from 1-3 independently selected C₁₋₆ alkyl, wherein the C₁₋₆ alkyl is optionally substituted with from 1-6 independently selected R^{f};
R⁹ is selected from the group consisting of C(═O)OH, C(═O)(OC₁₋₆ alkyl), C(═O)NR^{9a}R^{9b}, (IX-1), (IX-2), (IX-3), and (IX-4):

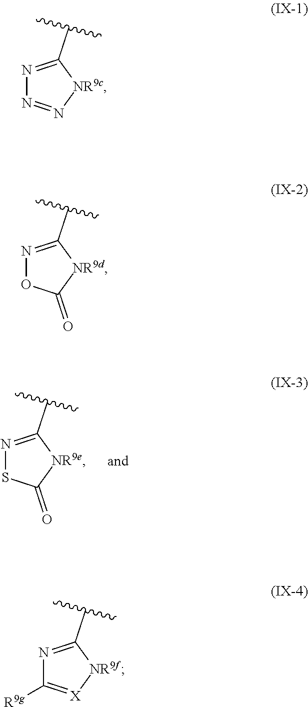

R^{9a} is H or C₁₋₆ alkyl;
R^{9b} is H, C₁₋₆ alkyl, C(═O)(C₁₋₆ alkyl), S(O)₀₋₂(C₁₋₆ alkyl), or cyano;
R^{9c}, R^{9d}, R^{9e}, R^{9f}, and R^{9g} are each independently selected from the group consisting of H; C₁₋₆ alkyl optionally substituted with from 1-6 independently selected halo and C₁₋₆ alkoxy; and C(═O)(C₁₋₆ alkyl);
Ring C is selected from the group consisting of 3-12 membered heterocyclyl; C₃₋₁₅ cycloalkyl; and 5-10 membered heteroaryl, each of which is optionally substituted with from 1-3 R^{Ca};
each R^{Ca} is independently selected from the group consisting of halo, C₁₋₆ alkyl, C₁₋₆ haloalkyl, C₁₋₆ alkoxy, and NR^{c}R^{d};
or a pair of R^{Ca} on the same or different ring atoms, taken together with the ring atom(s) to which each is attached, forms a carbocyclic ring including from 3-8 ring atoms;
each R^{c} and R^{d} are independently selected from the group consisting of H, C₁₋₆ alkyl, C(═O)(C₁₋₆ alkyl), C(═O)(C₃₋₆ cycloalkyl), C(═O)O(C₁₋₆ alkyl), S(O)₁₋₂(C₁₋₆ alkyl), and S(O)₁₋₂(C₃₋₆ cycloalkyl), wherein the C₁₋₆ alkyl, C(═O)(C₁₋₆ alkyl), C(═O)(C₃₋₆ cycloalkyl), C(═O)O(C₁₋₆ alkyl), S(O)₁₋₂(C₁₋₆ alkyl), and S(O)₁₋₂(C₃₋₆ cycloalkyl) are each optionally substituted with from 1-6 substituents independently selected from the group consisting of —OH, halo, and C₁₋₆ alkoxy;
R^{c} is H, C₁₋₆ alkyl, or C₁₋₆ haloalkyl;
each R^{f} is independently selected from the group consisting of halo, —OH, NR^{c}R^{d}, C₁₋₆ alkoxy, C₁₋₆ haloalkoxy, and 3-12 membered heterocyclyl which is optionally substituted with from 1-4 substituents each independently selected from the group consisting of —OH, C₁₋₆ alkyl, and 3-12 membered heterocyclyl;
each R^{g} is independently selected from the group consisting of C₁₋₆ alkyl, C₁₋₆ alkoxy, NR^{c}R^{d}, and 3 to 12 membered heterocyclyl optionally substituted with one or more substituents each independently selected from the group consisting of C₁₋₆ alkyl and C(═O)C₁₋₆ alkyl; and
each R^{h} is independently selected from the group consisting of halo, cyano, C₁₋₆ alkyl, C₁₋₆ haloalkyl, —OH, NH₂, NH(C₁₋₃ alkyl), N(C₁₋₃ alkyl)₂, C₁₋₃ alkoxy, and C₁₋₃ haloalkoxy.

In some embodiments, provided is a compound of Formula III:

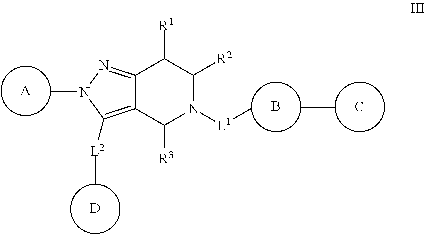

or a pharmaceutically acceptable salt, deuterated analog, or solvate thereof, wherein:
Ring D is selected from the group consisting of C₃₋₁₅ cycloalkyl; 3-12 membered heterocyclyl; 5-10 membered heteroaryl; and C₆₋₁₀ aryl, each of which is optionally substituted with from 1-6 substituents each independently selected from the group consisting of R^{QA} and R^{QB};
provided that one or both of (aa) or (bb) applies:
(aa) Ring D is substituted with at least one R^{QB}; or
(bb) Ring D is 4-12 membered heterocyclyl or 7-10 membered bicyclic heteroaryl, wherein Ring D comprises an endocyclic S(O)₂ group;
each R^{QA} is independently selected from the group consisting of
(a) halo;
(b) cyano;
(c) OH or oxo;
(d) —NR^{a}R^{b};
(e) —C(═O)NR^{c}R^{d};
(f) C₁₋₆ alkyl optionally substituted with from 1-6 independently selected R^{f};
(g) C₁₋₆ alkoxy optionally substituted with from 1-6 independently selected R^{f};
(h) 3-12 membered heterocyclyl optionally substituted with from 1-6 independently selected R^{g};
(i) C₆₋₁₀ aryl optionally substituted with from 1-6 independently selected R^{g};
(j) 5-10 membered heteroaryl optionally substituted with from 1-6 independently selected R^{g}; and
(k) C₃₋₈ cycloalkyl optionally substituted with from 1-6 independently selected R^{g}; each R^{QB} is independently selected from the group consisting of
   a) -L^{a}-S(O)₂-L^{b}-R^{c}; and
   b) 4-12 membered heterocyclyl or 7-10 membered bicyclic heteroaryl, each of which comprises an endocyclic S(O)₂ group, wherein the heterocyclyl or heteroaryl is optionally substituted with from 1-6 independently selected R^{g};
L^{a} is selected from the group consisting of —N(H)—, —N(R^{c})—, and —(CR^{h}R^{h})_{q1}—;
L^{b} is selected from the group consisting of —O—, —N(H)—, —N(R^{c})—, and —(CR^{h}R^{h})_{q2}—;
q1 and q2 are independently 0, 1, 2, 3, or 4;
provided that when L^{a} is —(CR^{h}R^{h})_{q1}— and q1 is 0, then -L^{b}-R^{c} is other than unsubstituted C₁₋₃ alkyl;
L² is selected from the group consisting of

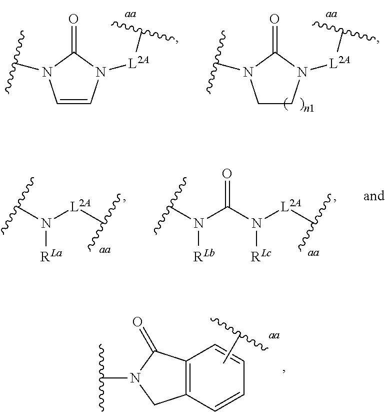

wherein aa represents the point of attachment to Ring D;
n1 is an integer from 1-3;
L^{2A} is a bond or C₁₋₁₀ alkylene;
R^{La} is selected from the group consisting of H, C₁₋₆ alkyl, and C(═O)(C₁₋₆ alkyl);
each of R^{Lb} and R^{Lc} is independently selected from the group consisting of H and C₁₋₆ alkyl;
Ring A is C₆₋₁₀ aryl, C₅₋₁₀ cycloalkyl, 5-10 membered heterocyclyl, or 5-10 membered heteroaryl, each of which is optionally substituted with from 1-5 substituents each independently selected from the group consisting of halo, C₁₋₆ alkyl, C₁₋₆ haloalkyl, C₁₋₆ alkoxy, and C₃₋₆ cycloalkyl;
R¹, R², and R³ are each independently selected from the group consisting of H and C₁₋₆ alkyl which is optionally substituted with from 1-6 substituents each independently selected from the group consisting of halo, —OH, and C₁₋₆ alkoxy;
L¹ is selected from the group consisting of —C(═O)—, —CH₂—, —CH(C₁₋₆ alkyl)-, and —S(═O)₂;
Ring B is selected from the group consisting of:

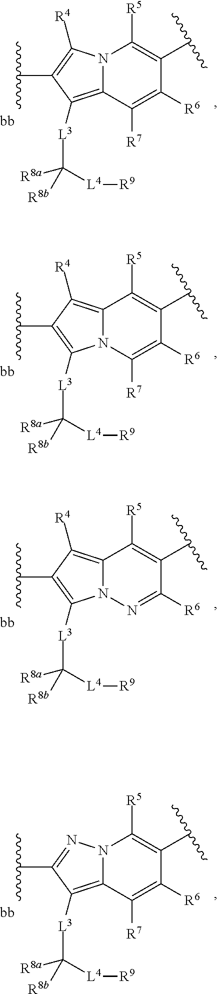

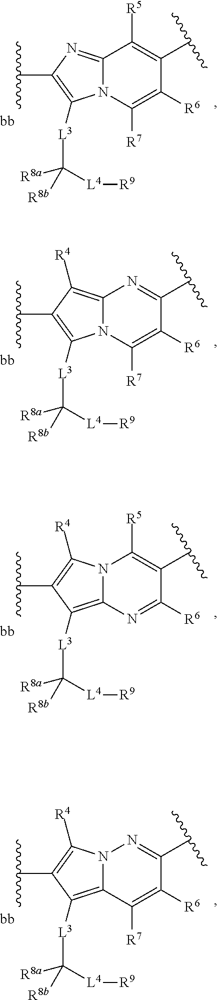

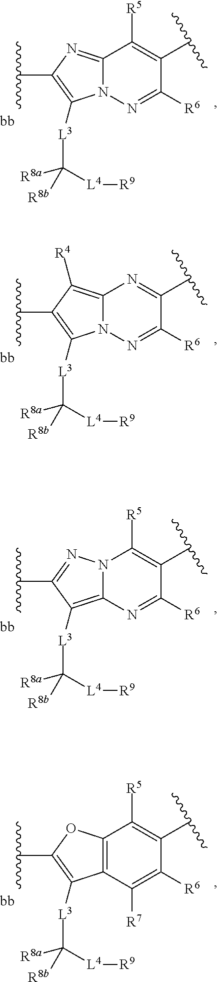

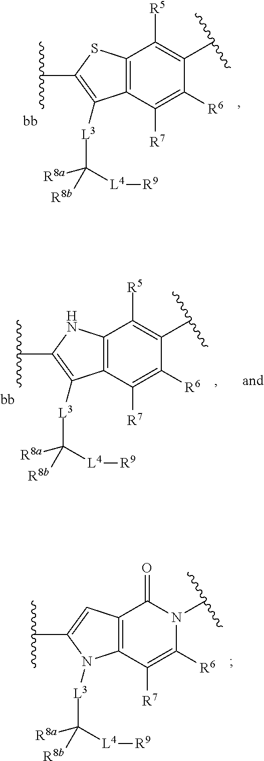

wherein bb represents point of attachment to L¹;
R⁴, R⁵, R⁶, and R⁷ are independently selected from the group consisting of H, halo, and C₁₋₆ alkyl;
L³ is a bond or C₁₋₃ alkylene;
L⁴ is a bond or C₁₋₅ alkylene;
R^{8a} and R^{8b} are independently selected from the group consisting of H and C₁₋₆ alkyl optionally substituted with one or more substituents independently selected from the group consisting of halo and C₃₋₁₅ cycloalkyl; or
R^{8a} and R^{8b} taken together with the carbon atom to which each is attached forms a C₃₋₁₅ cycloalkyl ring which is optionally substituted with from 1-3 independently selected C₁₋₆ alkyl, wherein the C₁₋₆ alkyl is optionally substituted with from 1-6 independently selected R^{f};
R⁹ is selected from the group consisting of C(═O)OH, C(═O)(OC₁₋₆ alkyl), C(═O)NR^{9a}R^{9b}, (IX-1), (IX-2), (IX-3), and (IX-4):

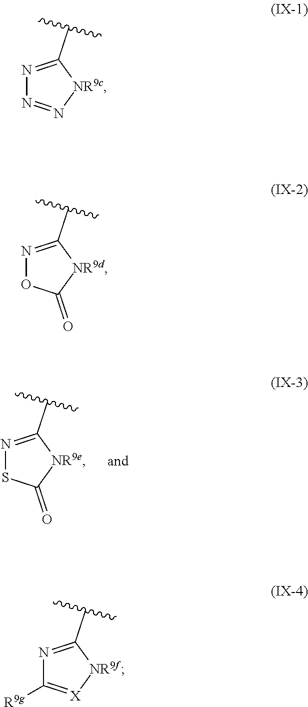

R^{9a} is H or C₁₋₆ alkyl;
R^{9b} is H, C₁₋₆ alkyl, C(═O)(C₁₋₆ alkyl), S(O)₀₋₂(C₁₋₆ alkyl), or cyano;
R^{9c}, R^{9d}, R^{9e}, R^{9f}, and R^{9g} are each independently selected from the group consisting of H; C₁₋₆ alkyl optionally substituted with from 1-6 independently selected halo and C₁₋₆ alkoxy; and C(═O)(C₁₋₆ alkyl);
Ring C is selected from the group consisting of 3-12 membered heterocyclyl; C₃₋₁₅ cycloalkyl; and 5-10 membered heteroaryl, each of which is optionally substituted with from 1-3 R^{Ca};
each R^{Ca} is independently selected from the group consisting of halo, C₁₋₆ alkyl, C₁₋₆ haloalkyl, C₁₋₆ alkoxy, and NR^{c}R^{d};
or a pair of R^{Ca} on the same or different ring atoms, taken together with the ring atom(s) to which each is attached, forms a carbocyclic ring including from 3-8 ring atoms;
each R^{a} and R^{b} are independently selected from the group consisting of H, C₁₋₆ alkyl, C₃₋₆ cycloalkyl, C(═O)(C₁₋₆ alkyl), C(═O)(C₃₋₆ cycloalkyl), and C(═O)O(C₁₋₆ alkyl), wherein the C₁₋₆ alkyl, C₃₋₆ cycloalkyl, C(═O)(C₁₋₆ alkyl), C(═O)(C₃₋₆ cycloalkyl), and C(═O)O(C₁₋₆ alkyl) are each optionally substituted with from 1-6 substituents independently selected from the group consisting of —OH, halo, and C₁₋₆ alkoxy;
each R^{c} and R^{d} are independently selected from the group consisting of H, C₁₋₆ alkyl, C₃₋₆ cycloalkyl, C(═O)(C₁₋₆ alkyl), C(═O)(C₃₋₆ cycloalkyl), C(═O)O(C₁₋₆ alkyl), S(O)₁₋₂(C₁₋₆ alkyl), and S(O)₁₋₂(C₃₋₆ cycloalkyl), wherein the C₁₋₆ alkyl, C₃₋₆ cycloalkyl, C(═O)(C₁₋₆ alkyl), C(═O)(C₃₋₆ cycloalkyl), C(═O)O(C₁₋₆ alkyl), S(O)₁₋₂(C₁₋₆ alkyl), and S(O)₁₋₂(C₃₋₆ cycloalkyl) are each optionally substituted with from 1-6 substituents independently selected from the group consisting of —OH, halo, and C₁₋₆ alkoxy;
R^{e} is H, or R^{c} is selected from the group consisting of C₁₋₆ alkyl, C₁₋₆ haloalkyl, C₁₋₆ alkoxy, C₁₋₆ haloalkoxy, C₃₋₆ cycloalkyl, and 3-8 membered heterocyclyl, each of which is optionally substituted with from 1-3 substituents each independently selected from the group consisting of halo, cyano, C₁₋₃ alkyl, C₁₋₃ haloalkyl, —OH, NH₂, NH(C₁₋₃ alkyl), N(C₁₋₃ alkyl)₂, C₁₋₃ alkoxy, and C₁₋₃ haloalkoxy;
each R^{f} is independently selected from the group consisting of halo, —OH, NR^{c}R^{d}, C₁₋₆ alkoxy, C₁₋₆ haloalkoxy, and 3-12 membered heterocyclyl which is optionally substituted with from 1-4 substituents each independently selected from the group consisting of —OH, C₁₋₆ alkyl, and 3-12 membered heterocyclyl;
each R^{g} is independently selected from the group consisting of C₁₋₆ alkyl, C₁₋₆ haloalkyl, C₁₋₆ alkoxy, C₁₋₆ haloalkoxy, NR^{c}R^{d}, cyano, halo, C₃₋₆ cycloalkyl, and 3 to 12 membered heterocyclyl optionally substituted with one or more substituents each independently selected from the group consisting of C₁₋₆ alkyl and C(═O)C₁₋₆ alkyl; and
each occurrence of R^{h} is independently selected from the group consisting of H, C₁₋₆ alkyl, C₁₋₆ haloalkyl, C₃₋₆ cycloalkyl, and halo; or
a pair of R^{h} on the same or different carbon atom(s), taken together with the carbon atom(s) connecting them forms C₃₋₆ cycloalkyl or 4-8 membered heterocyclyl, each of which is optionally substituted with from 1-3 substituents each independently selected from the group consisting of halo, cyano, C₁₋₃ alkyl, C₁₋₃ haloalkyl, —OH, C₁₋₃ alkoxy, and C₁₋₃ haloalkoxy.

In some embodiments, provided is a compound of Formula IV:

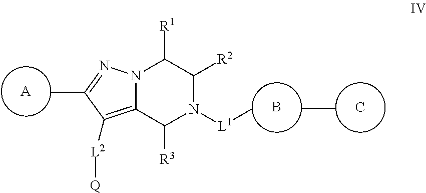

or a pharmaceutically acceptable salt, deuterated analog, or solvate thereof, wherein:
Ring A is C₆₋₁₀ aryl, C₅₋₁₀ cycloalkyl, 5-10 membered heterocyclyl, or 5-10 membered heteroaryl, each of which is optionally substituted with from 1-5 substituents each independently selected from the group consisting of halo, C₁₋₆ alkyl, C₁₋₆ haloalkyl, C₁₋₆ alkoxy, and C₃₋₆ cycloalkyl;
R¹, R², and R³ are each independently selected from the group consisting of H and C₁₋₆ alkyl which is optionally substituted with from 1-6 substituents each independently selected from the group consisting of halo, —OH, and C₁₋₆ alkoxy;
L¹ is selected from the group consisting of —C(═O)—, —CH₂—, —CH(C₁₋₆ alkyl)-, and —S(═O)₂;
Ring B is selected from the group consisting of:

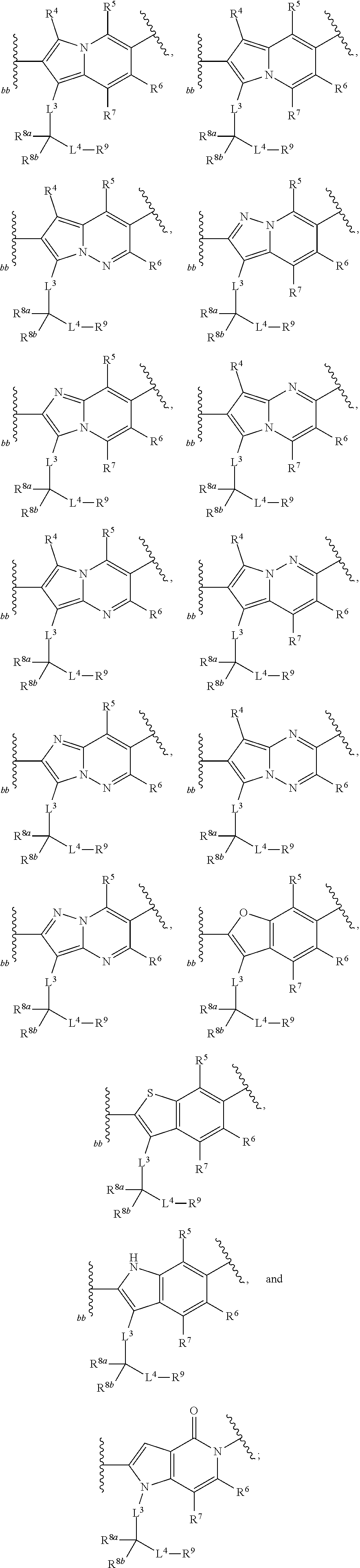

wherein bb represents point of attachment to L¹;
R⁴, R⁵, R⁶, and R⁷ are independently selected from the group consisting of H, halo, and C₁₋₆ alkyl;
L³ is a bond or C₁₋₃ alkylene;
L⁴ is a bond or C₁₋₅ alkylene;
R^{8a} and R^{8b} are independently selected from the group consisting of H and C₁₋₆ alkyl optionally substituted with one or more substituents independently selected from the group consisting of halo and C₃₋₁₅ cycloalkyl; or
R^{8a} and R^{8b} taken together with the carbon atom to which each is attached forms a C₃₋₁₅ cycloalkyl ring which is optionally substituted with from 1-3 independently selected C₁₋₆ alkyl, wherein the C₁₋₆ alkyl is optionally substituted with from 1-6 independently selected R^{f};
R⁹ is selected from the group consisting of C(═O)OH, C(═O)(OC₁₋₆ alkyl), C(═O)NR^{9a}R^{9b}, (IX-1), (IX-2), (IX-3), and (IX-4):

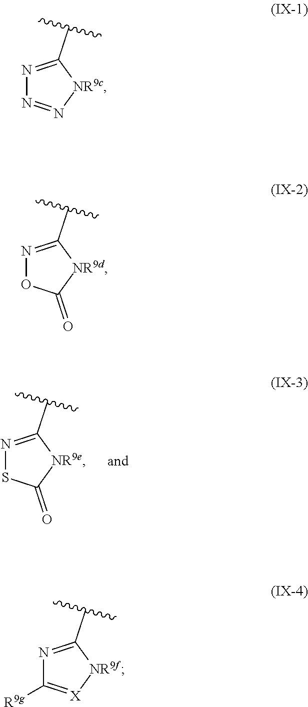

R^{9a} is H or C₁₋₆ alkyl;
R^{9b} is H, C₁₋₆ alkyl, C(═O)(C₁₋₆ alkyl), S(O)₀₋₂(C₁₋₆ alkyl), or cyano;
R^{9c}, R^{9d}, R^{9c}, R^{9f}, and R^{9g} are each independently selected from the group consisting of H; C₁₋₆ alkyl optionally substituted with from 1-6 independently selected halo and C₁₋₆ alkoxy; and C(═O)(C₁₋₆ alkyl);
Ring C is selected from the group consisting of 3-12 membered heterocyclyl; C₃₋₁₀ cycloalkyl; and 5-10 membered heteroaryl, each of which is optionally substituted with from 1-3 R^{Ca};
each R^{Ca} is independently selected from the group consisting of halo, C₁₋₆ alkyl, C₁₋₆ haloalkyl, C₁₋₆ alkoxy, and NR^{c}R^{d};
or a pair of R^{Ca} on the same or different ring atoms, taken together with the ring atom(s) to which each is attached, forms a carbocyclic ring including from 3-8 ring atoms;
L² is selected from the group consisting of

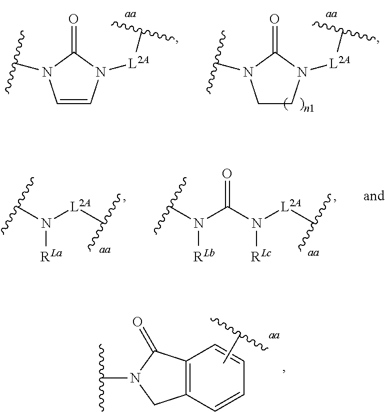

wherein aa represents the point of attachment to Q;
n1 is an integer from 1-3;
L^{2A} is a bond or C₁₋₁₀ alkylene;
R^{La} is selected from the group consisting of H, C₁₋₆ alkyl, and C(═O)(C₁₋₆ alkyl);
each of R^{Lb} and R^{Lc} is independently selected from the group consisting of H and C₁₋₆ alkyl;
Q is selected from the group consisting of C₁₋₁₀ alkyl; C₃₋₁₅ cycloalkyl; 3-12 membered heterocyclyl; 5-10 membered heteroaryl; and C₆₋₁₀ aryl, each of which is optionally substituted with from 1-6 independently selected R^{Q};
each R^{Q} is independently selected from the group consisting of
(a) halo;
(b) cyano;
(c) OH or oxo;
(d) —NR^{c}R^{d};
(e) —C(═O)NR^{c}R^{d} or —S(O)₂NR^{c}R^{d};
(f) —S(═O)₀₋₂R^{e};
(g) C₁₋₆ alkyl optionally substituted with from 1-6 independently selected R^{f};
(h) C₁₋₆ alkoxy optionally substituted with from 1-6 independently selected R^{f};
(i) 3-12 membered heterocyclyl optionally substituted with from 1-6 independently selected R^{g};
(j) C₆₋₁₀ aryl optionally substituted with from 1-6 independently selected R^{g};
(k) 5-10 membered heteroaryl optionally substituted with from 1-6 independently selected R^{g};
(l) C₃₋₈ cycloalkyl optionally substituted with from 1-6 independently selected R^{g};
(m) P(═O)R^{a}R^{b}; and
(n) —(CR^{h}R^{h})_{q1}—S(O)₂-L^{c}-R^{c}, wherein q1 is 1, 2, or 3;
R^{a} and R^{b} are independently selected from the group consisting of C₁₋₆ alkyl which is optionally substituted with from 1-6 substituents each independently selected from the group consisting of C₁₋₆ alkoxy, C₃₋₆ cycloalkyl, and halo; C₃₋₆ cycloalkyl optionally substituted with from 1-3 substituents each independently selected from the group consisting of C₁₋₃ alkyl and halo; and C₆₋₁₀ aryl optionally substituted with from 1-3 independently selected C₁₋₃ alkyl; or
R^{a} and R^{b} taken together with the phosphorous atom to which each is attached forms a ring including from 5-8 ring atoms, wherein from 0-2 ring atoms (in addition to the phosphorous attached to R^{a} and R^{b}) are heteroatoms each independently selected from the group consisting of O, S, and N, wherein the ring is optionally substituted with from 1-3 independently selected C₁₋₆ alkyl;
each R^{c} and R^{d} are independently selected from the group consisting of H, C₁₋₆ alkyl, C₃₋₆ cycloalkyl, C(═O)(C₁₋₆ alkyl), C(═O)(C₃₋₆ cycloalkyl), C(═O)O(C₁₋₆ alkyl), S(O)₁₋₂(C₁₋₆ alkyl), and S(O)₁₋₂(C₃₋₆ cycloalkyl), wherein the C₁₋₆ alkyl, C(═O)(C₁₋₆ alkyl), C(═O)(C₃₋₆ cycloalkyl), C(═O)O(C₁₋₆ alkyl), S(O)₁₋₂(C₁₋₆ alkyl), and S(O)₁₋₂(C₃₋₆ cycloalkyl) are each optionally substituted with from 1-6 substituents independently selected from the group consisting of —OH, halo, and C₁₋₆ alkoxy;
R^{e} is H, or R^{e} is selected from the group consisting of C₁₋₆ alkyl, C₁₋₆ haloalkyl, C₁₋₆ alkoxy, C₁₋₆ haloalkoxy, C₃₋₆ cycloalkyl, and 3-8 membered heterocyclyl, each of which is optionally substituted with from 1-3 substituents each independently selected from the group consisting of halo, cyano, C₁₋₃ alkyl, C₁₋₃ haloalkyl, —OH, NH₂, NH(C₁₋₃ alkyl), N(C₁₋₃ alkyl)₂, C₁₋₃ alkoxy, and C₁₋₃ haloalkoxy;
L^{e} is a bond, NR^{c}, or O;
each R^{f} is independently selected from the group consisting of halo, —OH, NR^{c}R^{d}, C₁₋₆ alkoxy, C₁₋₆ haloalkoxy, and 3-12 membered heterocyclyl which is optionally substituted with from 1-4 substituents each independently selected from the group consisting of —OH, C₁₋₆ alkyl, and 3-12 membered heterocyclyl;
each R^{g} is independently selected from the group consisting of C₁₋₆ alkyl, C₁₋₆ haloalkyl, C₁₋₆ alkoxy, C₁₋₆ haloalkoxy, NR^{c}R^{d}, cyano, halo, C₃₋₆ cycloalkyl, and 3 to 12 membered heterocyclyl optionally substituted with one or more substituents each independently selected from the group consisting of C₁₋₆ alkyl and C(═O)C₁₋₆ alkyl; and
each occurrence of R^{h} is independently selected from the group consisting of H, C₁₋₆ alkyl, C₁₋₆ haloalkyl, C₃₋₆ cycloalkyl, and halo; or
a pair of R^{h} on the same or different carbon atom(s), taken together with the carbon atom(s) connecting them forms C₃₋₆ cycloalkyl or 4-8 membered heterocyclyl, each of which is optionally substituted with from 1-3 substituents each independently selected from the group consisting of halo, cyano, C₁₋₃ alkyl, C₁₋₃ haloalkyl, —OH, C₁₋₃ alkoxy, and C₁₋₃ haloalkoxy.

In some embodiments, Ring A is C₆₋₁₀ aryl, optionally substituted with from 1-5 substituents each independently selected from the group consisting of halo, C₁₋₆ alkyl, C₁₋₆ haloalkyl, C₁₋₆ alkoxy, and C₃₋₆ cycloalkyl.

In some embodiments, Ring A is phenyl, optionally substituted with from 1-3 substituents each independently selected from the group consisting of fluoro, methyl, and cyclopropyl.

In some embodiments, R¹ and R² are H.

In some embodiments, R³ is C₁₋₆ alkyl.

In some embodiments, R³ is methyl.

In some embodiments, L¹ is —C(═O)—.

In some embodiments, Ring B is

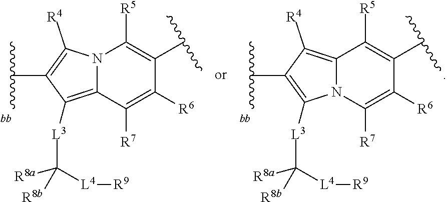

In some embodiments, Ring B is

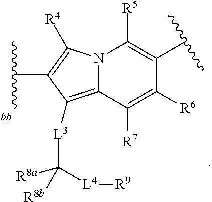

In some embodiments, Ring B is

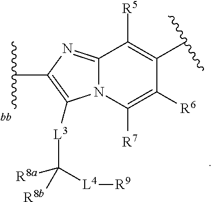

In some embodiments, Ring B is

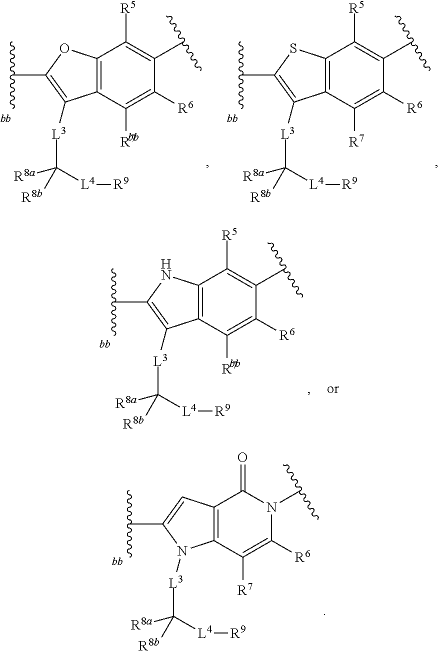

In some embodiments, Ring B is

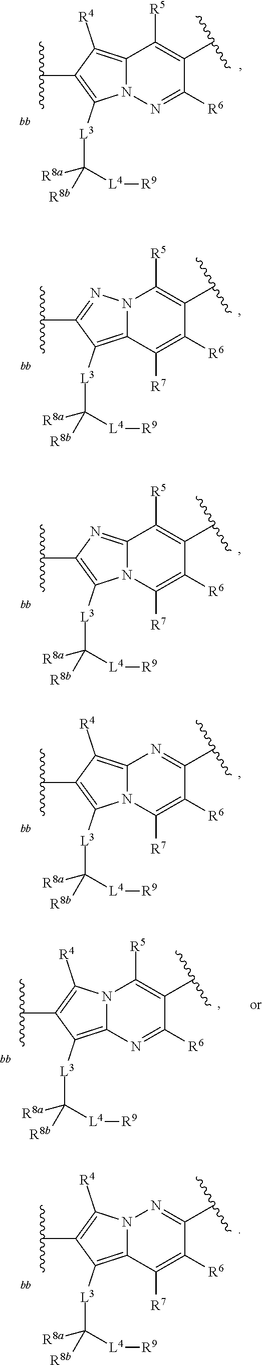

In some embodiments, Ring B is

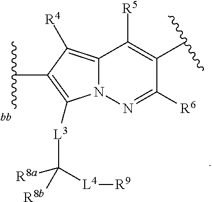

In some embodiments, Ring B is

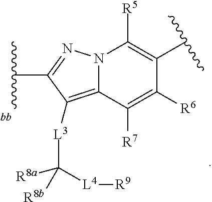

In some embodiments, Ring B is

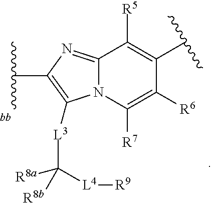

In some embodiments, Ring B is

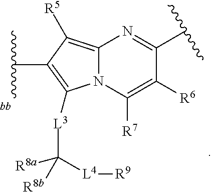

In some embodiments, Ring B is

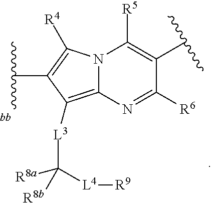

In some embodiments, Ring B is

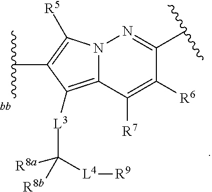

In some embodiments, Ring B is

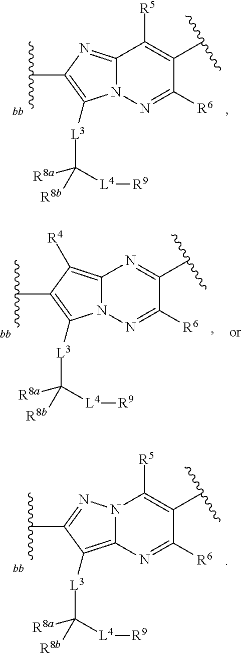

In some embodiments, Ring B is

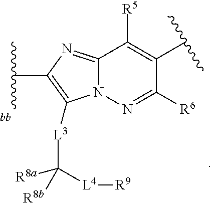

In some embodiments Ring B is

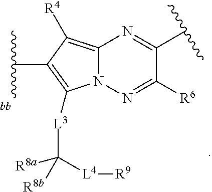

In some embodiments, Ring B is

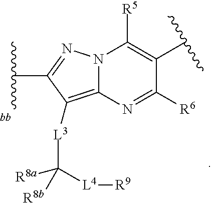

In some embodiments, Ring B is

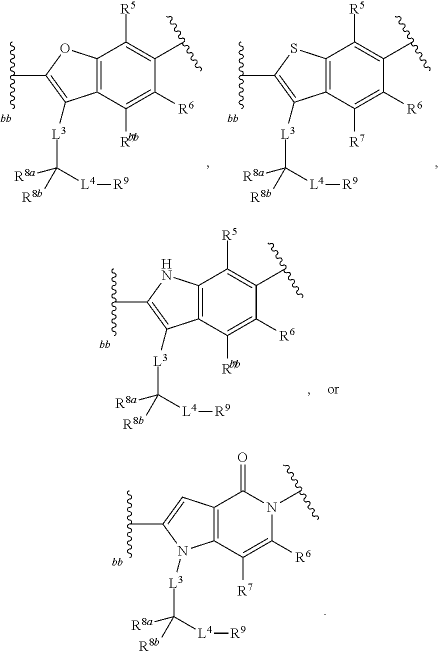

In some embodiments, Ring B is

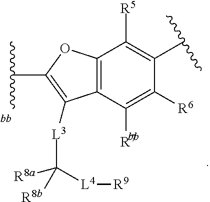

In some embodiments, Ring B is

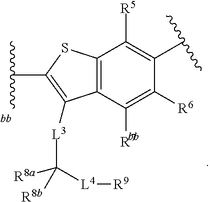

In some embodiments, Ring B is

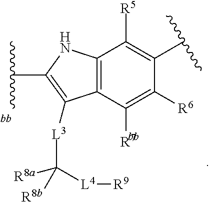

In some embodiments, Ring B is

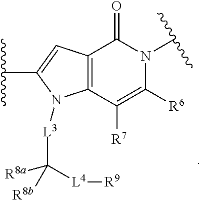

In some embodiments, R⁴, R⁵, R⁶, and R⁷ are H.

In some embodiments, L³ is a bond.

In some embodiments, R^{8a} and R^{8b} together with the carbon atom to which each is attached form a C₃₋₁₅ cycloalkyl.

In some embodiments, R^{8a} and R^{8b} together with the carbon atom to which each is attached form a cyclopropyl.

In some embodiments, R⁹ is

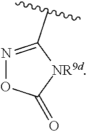

In some embodiments, R^{9d} is H.

In some embodiments, the moiety -L³-C(R^{8a}R^{8b})-L⁴-R⁹ is a phenyl substituted with C(═O)OH, and optionally 1-4 additional substituents independently selected from R^{f}. In some embodiments, the moiety -L³-C(R^{8a}R^{8b})-L⁴-R⁹ is a phenyl substituted with C(═O)OH. In some embodiments, the moiety -L³-C(R^{8a}R^{8b})-L⁴-R⁹ is

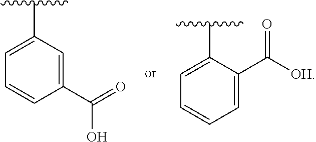 [SMILES: *c1cccc(C(=O)O)c1 ; "or" ; *c1ccccc1C(=O)O]

In some embodiments, Ring C is 3-12 membered heterocyclyl.

In some embodiments, Ring C is tetrahydropyranyl.

In some embodiments, L² is

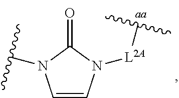

wherein aa represents the point of attachment to Q.

In some embodiments, L^{2A} is a bond.

In some embodiments, provided is a compound of Formula I or II, or a pharmaceutically acceptable salt or solvate thereof, comprising any one or more of the features delineated below.

In some embodiments, Q¹, Q², Q³, Q⁴, and Q⁵ are defined according to (AA).

In some embodiments, Q³ is CR^{QD}. In some embodiments, Q⁴ is N, CH, or CR^{QC}.

In some embodiments, Q⁴ is CR^{QD}. In some embodiments, Q³ is N, CH, or CR^{QC}.

In some embodiments, Q¹ is CH or CR^{QC}. In some embodiments, Q¹ is CH.

In some embodiments, Q² is CH or CR^{QC}. In some embodiments, Q² is CH.

In some embodiments, Q⁵ is CH or CR^{QC}. In some embodiments, Q⁵ is CH. In some embodiments, Q⁵ is CR^{QC}.

In some embodiments, Q³ is CR^{QD}; and each one of Q¹, Q², Q⁴, and Q⁵ is independently CH or CR^{QC}. In some embodiments, each one of Q¹, Q², Q⁴, and Q⁵ is CH.

In some embodiments (when Q³ is CR^{QD}; and each one of Q¹, Q², Q⁴, and Q⁵ is independently CH or CR^{QC}), one of Q¹, Q², Q⁴, and Q⁵ is CR^{QC}; and each remaining one of Q¹, Q², Q⁴, and Q⁵ is CH. In some embodiments, Q⁴ is CR^{QC}; and Q¹, Q², and Q⁵ are CH.

In some embodiments (when Q³ is CR^{QD}; and each one of Q¹, Q², Q⁴, and Q⁵ is independently CH or CR^{QC}), two of Q¹, Q², Q⁴, and Q⁵ are independently selected CR^{QC}; and each remaining one of Q¹, Q², Q⁴, and Q⁵ is CH.

In some embodiments, Q³ is CR^{QD}; one of Q¹, Q⁵, Q⁴, and Q⁵ is N; and each remaining one of Q¹, Q², Q⁴, and Q⁵ is independently CH or CR^{QC}. In some embodiments, Q⁴ is N. In some embodiments, Q¹, Q², and Q⁵ are CH.

In some embodiments, Q³ is CR^{QD}; and the

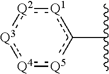

moiety is selected from the group consisting of:

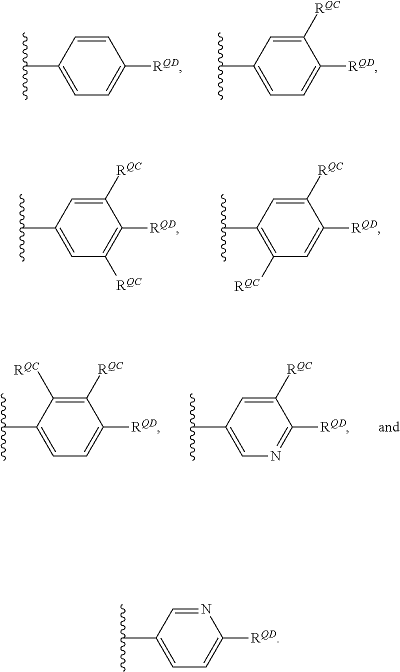

In some embodiments, Q³ is CR^{QD}; and the

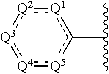

moiety is selected from the group consisting of:

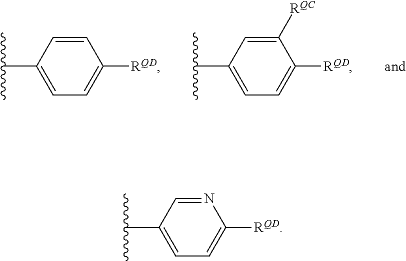

In some embodiments, the

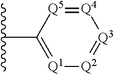

moiety is

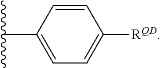

In some embodiments, the

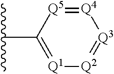

moiety is

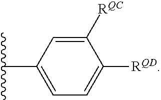

In some embodiments, Q³ is CR^{QD}; and the

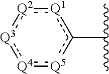

moiety is selected from the group consisting of:

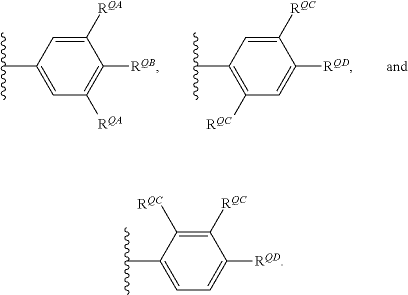

In some embodiments, the

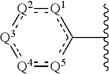

moiety is

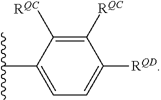

In some embodiments, the

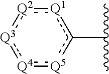

moiety can be

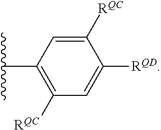

In some embodiments, Q⁴ is CR^{QD}; and each one of Q¹, Q², Q³, and Q⁵ is independently CH or CR^{QC}. In some embodiments, each one of Q¹, Q², Q³, and Q⁵ is CH.

In some embodiments (when Q⁴ is CR^{QD}; and each one of Q¹, Q², Q³, and Q⁵ is independently CH or CR^{QC}), one of Q¹, Q², Q³, and Q⁵ is CR^{QC}; and each remaining one of Q¹, Q², Q³, and Q⁵ is CH. In some embodiments, Q⁵ is CR^{QC}; and Q¹, Q², and Q³ are CH.

In some embodiments (when Q⁴ is CR^{QD}; and each one of Q¹, Q², Q³, and Q⁵ is independently CH or CR^{QC}), two of Q¹, Q², Q³, and Q⁵ are independently selected CR^{QC}; and each remaining one of Q¹, Q², Q³, and Q⁵ is CH. In some embodiments, Q² and Q³ are independently selected CR^{QC}; and Q¹ and Q⁵ are CH.

In some embodiments, Q⁴ is CR^{QD}; one of Q¹, Q¹, Q³, and Q⁵ is N; and each remaining one of Q¹, Q², Q³, and Q⁵ is independently CH or CR^{QC}.

In some embodiments, Q⁴ is CR^{QD}; and the

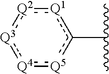

moiety is selected from the group consisting of:

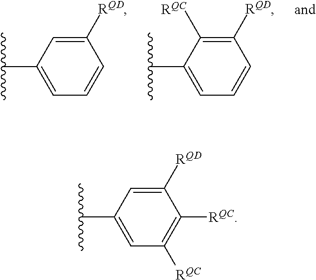

In some embodiments, the

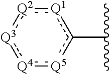

moiety is

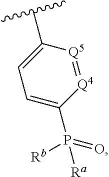

wherein Q⁴ and Q⁵ are independently selected from the group consisting of N, CH, and CR^{QC}. In some embodiments, each of Q⁴ and Q⁵ is CH. In some embodiments, Q⁴ is CR^{QC}; and Q⁵ is CH. In some embodiments, Q⁴ is N; and Q⁵ is CR^{QC} or CH. In certain embodiments, Q⁵ is CH.

In some embodiments, the

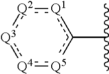

moiety is

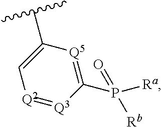

wherein Q², Q³, and Q⁵ are independently selected from the group consisting of N, CH, and CR^{QC}. In some embodiments, each of Q², Q³, and Q⁵ is CH. In some embodiments, Q⁵ is CR^{QC}; and each of Q² and Q³ is CH. In some embodiments, Q⁵ is CH; and Q² and Q³ are independently selected CR^{QC}.

In some embodiments, R^{QD} is P(═O)R^{a}R^{b}, wherein R^{a} and R^{b} are independently selected from the group consisting of C₁₋₆ alkyl and C₃₋₆ cycloalkyl. In some embodiments, CR^{QD} is P(═O)R^{a}R^{b}, wherein R^{a} and R^{b} are each independently C₁₋₆ alkyl. In some embodiments, CR^{QD} is P(═O)R^{a}R^{b}, wherein R^{a} and R^{b} are each independently C₁₋₃ alkyl.

In some embodiments, R^{a} and R^{b} are the same.

In some embodiments, R^{a} and R^{b} are each methyl (i.e., R^{QD} is P(═O)Me₂).

In some embodiments, R^{a} and R^{b} are each ethyl (i.e., R^{QD} is P(═O)Et₂).

In some embodiments, R^{a} and R^{b} are each propyl, such as isopropyl. For example, R^{QD} can be P(═O)iPr₂.

In some embodiments, R^{QD} is P(═O)R^{a}R^{b}; R^{a} and R^{b} are each independently C₁₋₆ alkyl; and R^{a} and R^{b} are different. In some embodiments, R^{a} is C₁₋₃ alkyl (e.g., methyl or ethyl); and R^{b} is C₄₋₆ alkyl (e.g., butyl such as tert-butyl). For example, R^{a} can be methyl; and R^{b} can be tert-butyl (i.e., R^{QD} can be P(═O)(Me)(tBu)). In some embodiments, R^{a} and R^{b} are independently selected C₁₋₃ alkyl, provided that R^{a} and R^{b} are different.

In some embodiments, R^{QD} is P(═O)R^{a}R^{b}, wherein R^{a} and R^{b} are each independently C₃₋₆ cycloalkyl. In certain of these embodiments, R^{a} and R^{b} are the same. For example, R^{a} and R^{b} can both be cyclopropyl (i.e., R^{QD} can be

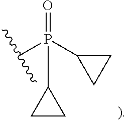 [SMILES: *P(=O)(C1CC1)C1CC1]

In some embodiments, R^{QD} is P(═O)R^{a}R^{b}, wherein R^{a} and R^{b} taken together with the phosphorous atom to which each is attached forms a ring including from 5-8 ring atoms, wherein from 0-2 ring atoms (in addition to the phosphorous attached to R^{a} and R^{b}) are heteroatoms each independently selected from the group consisting of: O, S, and N, wherein the ring is optionally substituted with from 1-3 independently selected C₁₋₆ alkyl.

In some embodiments, R^{QD} is P(═O)R^{a}R^{b}, wherein R^{a} and R^{b} taken together with the phosphorous atom to which each is attached forms a ring including from 5-6 ring atoms, wherein from 0-1 ring atom (in addition to the phosphorous attached to R^{a} and R^{b}) is a heteroatom selected from the group consisting of: O, S, and N, wherein the ring is optionally substituted with from 1-2 independently selected C₁₋₆ alkyl.

In some embodiments, R^{QD} can be

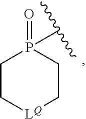

wherein L^{Q} is a bond, CH₂, O, S, NH, or N(C₁₋₆ alkyl). For example, R^{QD} can be or

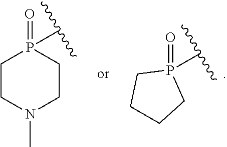

In some embodiments, each R^{QC} is selected from the group consisting of: halo, cyano, C₁₋₆ alkyl, C₁₋₆ alkoxy, OH, and NR^{c}R^{d}.

In some embodiments, each R^{QC} is selected from the group consisting of: halo, cyano, OH, and NR^{c}R^{d}.

In some embodiments, one occurrence of R^{QC} is halo. In certain of these embodiments, one occurrence of R^{QC} is —F.

In some embodiments, one occurrence of R^{QC} is —OH.

In some embodiments, one occurrence of R^{QC} is NR^{c}R^{d}.

In some embodiments, one occurrence of R^{QC} is NH(C₁₋₃ alkyl) (e.g., NHMe, NHEt, or NHiPr).

In some embodiments, one occurrence of R^{QC} is NH₂.

In some embodiments, one occurrence of R^{QC} is N(C₁₋₃ alkyl)₂ (e.g., NMe₂).

In some embodiments, one occurrence of R^{QC} is selected from the group consisting of NHC(═O)(C₁₋₆ alkyl), NHC(═O)(C₃₋₆ cycloalkyl), NHC(═O)O(C₁₋₆ alkyl), NHS(O)₁₋₂(C₁₋₆ alkyl), and NHS(O)₁₋₂(C₃₋₆ cycloalkyl).

In some embodiments, one occurrence of R^{QC} is NHC(═O)(C₁₋₃ alkyl), NHC(═O)(C₃₋₆ cycloalkyl) (e.g., NHC(═O)(cyclopropyl)), or NHS(O)₂(C₁₋₃ alkyl) (e.g., NHS(O)₂Me).

In some embodiments, one occurrence of R^{QC} is 5-6 membered heterocyclyl optionally substituted with one or more substituents each independently selected from the group consisting of C₁₋₆ alkyl and C(═O)(C₁₋₆ alkyl). In some embodiments, one occurrence of R^{QC} is morpholinyl.

In some embodiments, one occurrence of R^{QC} is C₁₋₆ alkoxy optionally substituted with from 1-6 substituents each independently selected from the group consisting of: hydroxy, halo, and C₁₋₆ alkoxy. In some embodiments, one occurrence of R^{QC} is C₁₋₆ alkoxy optionally substituted with from 1-6 (e.g., 1-3) independently selected halo. In some embodiments, R^{QC} can be —OMe, —OCF₃, or —OCHF₂. In some embodiments, R^{QC} is —OMe. In some embodiments, R^{QC} is —OCF₃. In some embodiments, R^{QC} is —OCHF₂.

In some embodiments, R^{QC} is C₁₋₆ alkyl optionally substituted with from 1-6 independently selected R^{f}. In some embodiments, R^{QC} is C₁₋₃ alkyl. In some embodiments, R^{QC} is methyl.

In some embodiments, one occurrence of R^{QC} is C₁₋₃ alkyl substituted with from 1-6 independently selected halo. In some embodiments, R^{QC} is CF₃, CHF₂, or CH₂F. In some embodiments, R^{QC} is CF₃ or CHF₂.

In some embodiments, one occurrence of R^{QC} is C₁₋₃ alkyl substituted with NR^{c}R^{d}. In some embodiments, R^{QC} is CH₂NHMe.

In some embodiments, each remaining R^{QC} when present is an independently selected halo, such as —F.

In some embodiments, a pair of R^{QC} on adjacent carbon atoms, taken together with the atom to which each is attached, forms a ring including from 5-6 ring atoms, wherein from 1-2 ring atoms are heteroatoms each independently selected from the group consisting of O, N, and S, wherein said ring is optionally substituted with from 1-2 independently selected R^{h} groups.

In some embodiments, a pair of R^{QC} on adjacent carbon atoms, taken together with the atom to which each is attached forms:

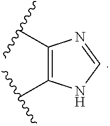 [SMILES: *c1nc[nH]c1*]

In some embodiments, a pair of R^{QC} on adjacent carbon atoms, taken together with the atom to which each is attached forms:

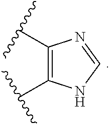 [SMILES: *c1nc[nH]c1*]

In some embodiments, each remaining R^{QC} when present is independently halo, cyano, or C₁₋₃ alkyl.

In some embodiments, L² is

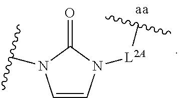

In some embodiments, L^{2A} is a bond. In some embodiments, L^{2A} is CH₂.

In some embodiments, L² is

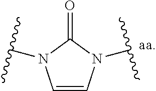 [SMILES: *n1ccn(*)c1=O]

In some embodiments, L² is

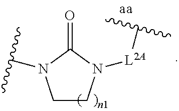

In some embodiments, n1 is 1. In certain other embodiments, n1 is 2 or 3. In some embodiments, L^{2A} is a bond. In some embodiments, L^{2A} is C₁₋₂ alkylene.

In some embodiments, L² is

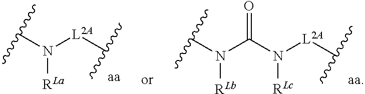

In some embodiments, L^{2A} is a bond. In some embodiments, L^{2A} is C₁₋₂ alkylene.

In some embodiments, L² is

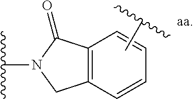

In some embodiments, Ring A is C₆₋₁₀ aryl or 5-10 membered heteroaryl, each of which is optionally substituted with from 1-4 substituents each independently selected from the group consisting of halo, C₁₋₆ alkyl, C₁₋₆ haloalkyl, and C₁₋₆ alkoxy.

In some embodiments, Ring A is phenyl or pyridyl, each of which is optionally substituted with from 2-4 substituents each independently selected from the group consisting of halo, C₁₋₆ alkyl, C₁₋₆ haloalkyl, and C₁₋₆ alkoxy.

In some embodiments, Ring A is phenyl, which is optionally substituted with from 2-4 substituents each independently selected from the group consisting of halo (e.g., —F) and C₁₋₆ alkyl (e.g., C₁₋₃ alkyl). In some embodiments, Ring A is

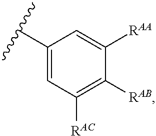

wherein R^{AA}, R^{AB}, and R^{AC} are independently halo or C₁₋₆ alkyl. In some embodiments, R^{AA} and R^{AC} are independently selected from C₁₋₃ alkyl (e.g., methyl); and/or R^{AB} is halo (e.g., —F).

In some embodiments, Ring A is pyridyl, which is optionally substituted with from 2-4 substituents each independently selected from the group consisting of halo and C₁₋₆ alkyl. In some embodiments, Ring A is 4-pyridyl, which is optionally substituted with from 2-4 substituents each independently selected from the group consisting of halo and C₁₋₆ alkyl. In some embodiments, Ring A is 3-pyridyl, which is optionally substituted with from 2-4 substituents each independently selected from the group consisting of halo and C₁₋₆ alkyl. In some embodiments, Ring A is 2-pyridyl, which is optionally substituted with from 2-4 substituents each independently selected from the group consisting of halo and C₁₋₆ alkyl.

In some embodiments, R¹ is H. In some embodiments, R¹ is C₁₋₆ alkyl.

In some embodiments, R² is H. In some embodiments, R² is C₁₋₆ alkyl.

In some embodiments, R³ is H. In some embodiments, R³ is C₁₋₆ alkyl (e.g., C₁₋₃ alkyl such as methyl).

In some embodiments, R¹, R², and R³ are H. In some embodiments, R¹ and R² are H; and R³ is C₁₋₆ alkyl. In some embodiments, R³ is methyl.

In some embodiments, R¹ is H; and R² and R³ are independently selected C₁₋₆ alkyl.

In some embodiments, L¹ is C(═O).

In some embodiments, L¹ is —CH₂— or —CH(C₁₋₆ alkyl)-.

In some embodiments, L¹ is —S(═O)₂.

In some embodiments, R⁴, R⁵, and R⁶ are each H or halo. In some embodiments, R⁴, R⁵, and R⁶ are each H or —F. In some embodiments, R⁴, R⁵, and R⁶ are each H. As another non-limiting example, R⁴ and R⁵ are H; and R⁶ is —F. In some embodiments, R⁷ is H. In some embodiments, R⁷ is —F.

In some embodiments, R⁴, R⁵, R⁶, and R⁷, when present, are each independently selected from the group consisting of —H and halo. In some embodiments, each of R⁴, R⁵, R⁶, and R⁷ when present are each independently selected from the group consisting of —H and —F.

In some embodiments, at least one of L³ and L⁴ is a bond. In some embodiments, both of L³ and L⁴ are bonds.

In some embodiments, L³ is a bond; and L⁴ is C₁₋₂ alkylene.

In some embodiments, L⁴ is a bond; and L³ is C₁₋₂ alkylene.

In some embodiments, L³ and L⁴ are each independently C₁₋₂ alkylene.

In some embodiments, R^{8a} and R^{8b} taken together with the carbon atom to which each is attached forms a C₃₋₈ cycloalkyl ring which is optionally substituted with from 1-2 independently selected C₁₋₆ alkyl, wherein the C₁₋₆ alkyl is optionally substituted with from 1-3 independently selected R^{f}.

In some embodiments, R^{8a} and R^{8b} taken together with the carbon atom to which each is attached forms a C₃₋₅ cycloalkyl ring which is optionally substituted with from 1-2 independently selected C₁₋₆ alkyl, wherein the C₁₋₆ alkyl is optionally substituted with from 1-3 independently selected R^{f}.

In some embodiments, R^{8a} and R^{8b} taken together with the carbon atom to which each is attached forms a C₃₋₄ cycloalkyl ring which is optionally substituted with from 1-2 independently selected C₁₋₆ alkyl.

In some embodiments, R^{8a} and R^{8b} taken together with the carbon atom to which each is attached forms:

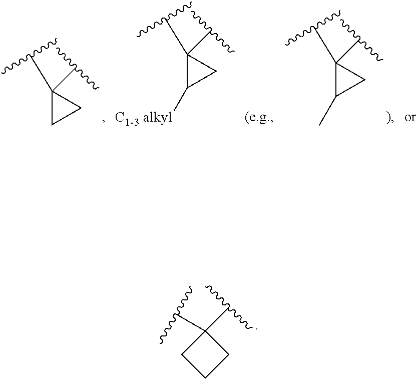

In some embodiments, R^{8a} and R^{8b} taken together with the carbon atom to which each is attached forms:

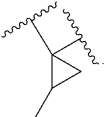 [SMILES: *C1(*)CC1C]

In some embodiments, R^{8a} and R^{8b} are each independently selected from the group consisting of H and C₁₋₆ alkyl. For example, R^{8a} and R^{8b} can both be H. In some embodiments, R^{8a} and R^{8b} are each independently C₁₋₆ alkyl (e.g., C₁₋₃ alkyl). In some embodiments, R^{8a} is H; and R^{8b} is C₁₋₆ alkyl (e.g., C₁₋₃ alkyl).

In some embodiments, the L³-C(R^{8a}R^{8b})-L⁴-R⁹ moiety is:

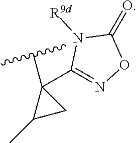

In some embodiments, R^{9d} is H or C₁₋₆ alkyl.

In some embodiments, the L³-C(R^{8a}R^{8b})-L⁴-R⁹ moiety is:

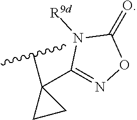

In certain of these embodiments, R^{9d} is H or C₁₋₆ alkyl. For example, R^{9d} can be H.

In some embodiments, R⁹ is

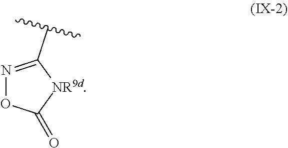

In some embodiments, R^{9d} is H or C₁₋₆ alkyl. In some embodiments, R^{9d} is H.

In some embodiments, R⁹ is

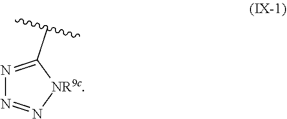

In some embodiments, R⁹ is

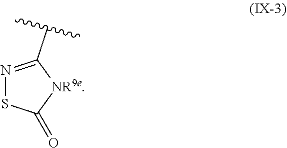

In some embodiments, R⁹ is

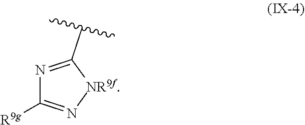

In some embodiments, R⁹ is C(═O)OH.

In some embodiments, R⁹ is C(═O)(OC₁₋₆ alkyl).

In some embodiments, R⁹ is C(═O)NR^{9a}R^{9b}. In some embodiments, R^{9a} is H. In some embodiments, R^{9b} is H. In some embodiments, R^{9b} is C₁₋₆ alkyl. In some embodiments, R^{9b} is selected from the group consisting of: C(═O)(C₁₋₆ alkyl), S(O)₀₋₂(C₁₋₆ alkyl), and cyano.

In some embodiments, the

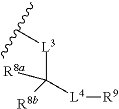

moiety is

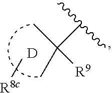

wherein Ring D is a C₃₋₆ cycloalkyl; and R^{8c} is selected from the group consisting of H and C₁₋₆ alkyl optionally substituted with from 1-3 independently selected R^{f}. Non-limiting examples include:

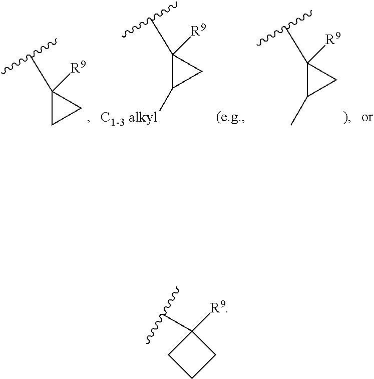

In some embodiments (when the

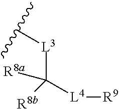

moiety is

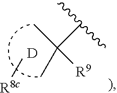

### R⁹ is

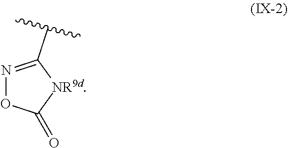

In some embodiments, R^{9d} is H or C₁₋₆ alkyl. In some embodiments, R^{9d} is H.

In some embodiments, Ring C is 3-12 membered heterocyclyl which is optionally substituted with from 1-3 independently selected R^{Ca}.

In some embodiments, Ring C is 4-8 membered heterocyclyl which is optionally substituted with from 1-3 independently selected R^{Ca}.

In some embodiments, Ring C is 5-6 membered heterocyclyl which is optionally substituted with from 1-3 independently selected R^{Ca}.

In some embodiments, Ring C is tetrahydropyranyl which is optionally substituted with from 1-3 independently R^{Ca}. In some embodiments, Ring C is selected from the group consisting of:

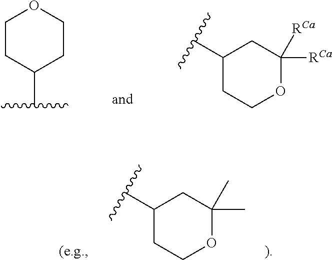

In some embodiments, Ring C is

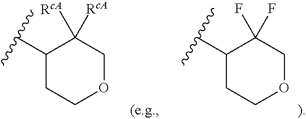

In certain embodiments, Ring C is morpholinyl which is optionally substituted with from 1-3 independently selected R^{Ca}. For example, Ring C can be

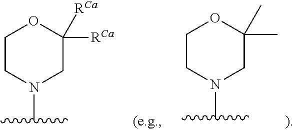

In some embodiments, Ring C is 5-6 membered heteroaryl which is optionally substituted with from 1-3 independently selected R^{Ca}.

In some embodiments, Ring C is C₃₋₁₀ cycloalkyl which is optionally substituted with from 1-3 R^{Ca}. In some embodiments, Ring C is C₃₋₈ cycloalkyl which is optionally substituted with from 1-3 independently selected R^{Ca}. In some embodiments, Ring C is

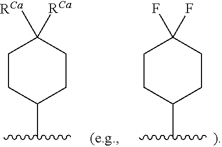

In some embodiments, each R^{Ca} is independently selected from the group consisting of halo, C₁₋₆alkyl, C₁₋₆ alkoxy, and NR^{c}R^{d}.

In some embodiments, each R^{Ca} is independently selected from the group consisting of: halo and C₁₋₆ alkyl.

In some embodiments, each R^{Ca} is independently C₁₋₆ alkyl. For example, each R^{Ca} can be methyl.

In some embodiments, each R^{Ca} is an independently selected halo. For example, each R^{Ca} can be —F.

In some embodiments, a pair of R^{Ca} on the same or different ring atoms, taken together with the ring atom(s) to which each is attached, forms a carbocyclic ring including from 3-6 ring atoms. In some embodiments, a pair of R^{Ca} on the same ring atom, taken together with the ring atom to which each is attached, forms a carbocyclic ring including from 3-5 ring atoms.

In some embodiments, the compound of Formula I is a compound of Formula IIA:

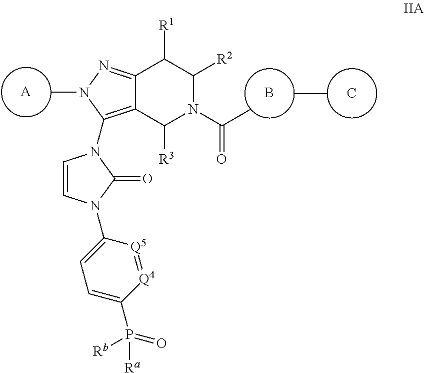

wherein Q⁴ and Q⁵ are independently selected from the group consisting of: N, CH, and CR^{QC}; or a pharmaceutically acceptable salt, deuterated analog, or solvate thereof.

In some embodiments of Formula IIA, each of Q⁴ and Q⁵ is CH.

In some embodiments of Formula IIA, Q⁴ is CR^{QC}; and Q⁵ is CH.

In some embodiments of Formula IIA, Q⁴ is CR^{QC}; and Q⁵ is CR^{QC}. In certain of these embodiments, Q⁴ is CR^{QC}; and Q⁵ is C-halo, such as CF.

In some embodiments of Formula IIA, Q⁴ is N; and Q⁵ is CR^{QC} or CH. In certain of these embodiments, Q⁵ is CH.

In some embodiments, the compound of Formula I is a compound of Formula IIB:

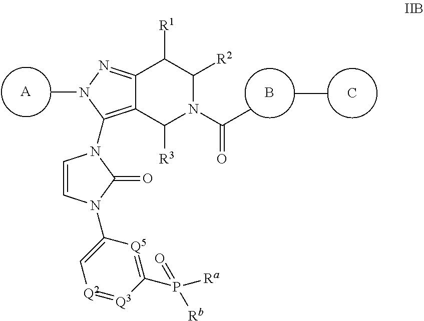

wherein Q², Q³, and Q⁵ are independently selected from the group consisting of: N, CH, and CR^{QC};
or a pharmaceutically acceptable salt, deuterated analog, or solvate thereof.

In some embodiments of Formula IIB, each of Q², Q³, and Q⁵ is CH.

In some embodiments of Formula IIB, Q⁵ is CR^{QC}; and each of Q² and Q³ is CH.

In some embodiments of Formula IIB, Q⁵ is CH; and Q² and Q³ are independently selected CR^{QC}.

In some embodiments, at least one of L³ and L⁴ is a bond. In some embodiments, both of L³ and L⁴ are bonds.

In some embodiments of Formula IIA or IIB, R^{8a} and R^{8b} taken together with the carbon atom to which each is attached forms a C₃₋₅ cycloalkyl ring which is optionally substituted with from 1-2 independently selected C₁₋₆ alkyl, wherein the C₁₋₆ alkyl is optionally substituted with from 1-3 independently selected R^{f}.

In some embodiments, R^{8a} and R^{8b} taken together with the carbon atom to which each is attached forms a C₃₋₄ cycloalkyl ring which is optionally substituted with from 1-2 independently selected C₁₋₆ alkyl.

In some embodiments, R^{8a} and R^{8b} taken together with the carbon atom to which each is attached forms:

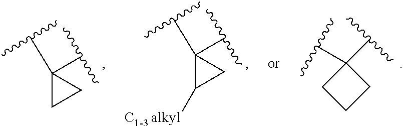

In some embodiments of Formula IIA or IIB, Ring C is 3-12 membered heterocyclyl which is optionally substituted with from 1-3 independently selected R^{Ca}. In some embodiments, Ring C is 5-6 membered heterocyclyl which is optionally substituted with from 1-3 independently selected R^{Ca}. In some embodiments, Ring C is tetrahydropyranyl which is optionally substituted with from 1-3 independently R^{Ca}. In some embodiments, Ring C can be selected from the group consisting of:

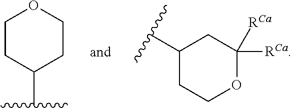

In certain embodiments, Ring C is morpholinyl which is optionally substituted with from 1-3 independently selected R^{Ca}. For example, Ring C can be

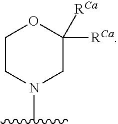

In some embodiments of Formula (IA) or (IB), Ring C is C₃₋₈ cycloalkyl which is optionally substituted with from 1-3 R^{Ca}. For example, Ring C can be

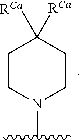

In some embodiments of Formula IIA or IIB, each R^{Ca} is independently C₁₋₆ alkyl. In some embodiments, each R^{Ca} is H. In some embodiments, each R^{Ca} is C₁₋₃ alkyl (e.g., methyl). In some embodiments of Formula IIA or IIB, each R^{Ca} is an independently selected halo (e.g., —F).

In some embodiments of Formula IIA or IIB, a pair of R^{Ca} on the same or different ring atoms, taken together with the ring atom(s) to which each is attached, forms a carbocyclic ring including from 3-6 ring atoms.

In some embodiments of Formula IIA or IIB, a pair of R^{Ca} on the same ring atom, taken together with the ring atom to which each is attached, forms a carbocyclic ring including from 3-5 ring atoms.

In some embodiments, the compound of Formula I is a compound of Formula IIC:

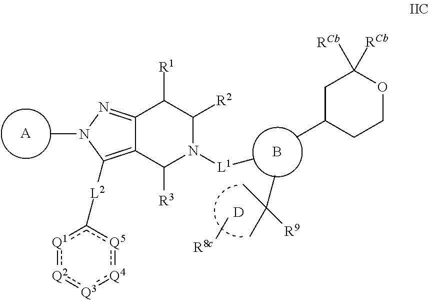

or a pharmaceutically acceptable salt, deuterated analog, or solvate thereof, wherein:
Ring D is a C₃₋₆ cycloalkyl;
R^{8c} is selected from the group consisting of H and C₁₋₆ alkyl optionally substituted with from 1-3 independently selected R^{f}; and
each R^{Cb} is independently selected from the group consisting of H and R^{Ca}.

In some embodiments of Formula IIC, Ring D is cyclopropyl.

In some embodiments of Formula IIC, Ring D is cyclobutyl.

In some embodiments of Formula IIC, R^{8c} is H.

In some embodiments of Formula IIC, R^{8c} is C₁₋₃ alkyl. For example, R^{8c} can be methyl.

In some embodiments of Formula IIC, each R^{Cb} is H.

In some embodiments of Formula IIC, each R^{Cb} is independently C₁₋₆ alkyl (e.g., C₁₋₃ alkyl (e.g., methyl)).

In some embodiments of Formula IIC, Q¹, Q², Q³, Q⁴, and Q⁵ are as defined according to (AA).

In some embodiments of Formula IIC, the

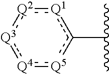

moiety is

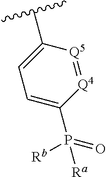

wherein Q⁴ and Q⁵ are independently selected from the group consisting of N, CH, and CR^{QC}.

In some embodiments, each of Q⁴ and Q⁵ is CH.

In some embodiments, Q⁴ is CR^{QC}; and Q⁵ is CH.

In some embodiments, Q⁴ is CR^{QC}; and Q⁵ is CR^{QC}. In some embodiments, Q⁴ is CR^{QC}; and Q⁵ is C-halo, such as CF.

In some embodiments, Q⁴ is N; and Q⁵ is CR^{QC} or CH. In some embodiments, Q⁵ is CH.

In some embodiments of Formula IIC, the

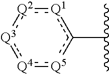

moiety is

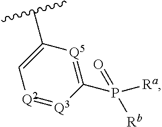

wherein Q², Q³, and Q⁵ are independently selected from the group consisting of N, CH, and CR^{QC}.

In some embodiments, each of Q², Q³, and Q⁵ is CH.

In some embodiments, Q⁵ is CR^{QC}; and each of Q² and Q³ is CH.

In some embodiments, Q⁵ is CH; and Q² and Q³ are independently selected CR^{QC}.

In some embodiments of Formula IIC, L¹ is C(═O).

In some embodiments of Formula IIA, IIB, or IIC, R^{a} and R^{b} are each independently selected from the group consisting of C₁₋₆ alkyl and C₃₋₆ cycloalkyl.

In some embodiments of Formula IIA, IIB, or IIC, R^{a} and R^{b} are each independently C₁₋₃ alkyl. In some embodiments, R^{a} and R^{b} are each methyl. In some embodiments, R^{a} and R^{b} are each be ethyl. In some embodiments, R^{a} and R^{b} are each be isopropyl.

In some embodiments of Formula IIA, IIB, or IIC, R^{a} is methyl; and R^{b} is tert-butyl.

In some embodiments of Formula IIA, IIB, or IIC, R^{a} and R^{b} are independently selected C₃₋₆ cycloalkyl. For example, R^{a} and R^{b} can both be cyclopropyl.

In some embodiments of Formula IIA, IIB, or IIC, each R^{QC} is selected from the group consisting of: halo; cyano; OH; NR^{c}R^{d}; C₁₋₆ alkyl optionally substituted with from 1-6 independently selected R^{f}; C₁₋₆ alkoxy optionally substituted with from 1-6 substituents each independently selected from the group consisting of: hydroxy, halo, and C₁₋₆ alkoxy; and 3-12 membered heterocyclyl optionally substituted with one or more substituents each independently selected from the group consisting of C₁₋₆ alkyl and C(═O)(C₁₋₆ alkyl).

In some embodiments, each R^{QC} is selected from the group consisting of: —F, OH, NH₂NHMe, NHEt, NHiPr, N(Me)₂, NHC(═O)(cyclopropyl), NHS(O)₂Me, methyl, CF₃, CHF₂, OMe, OCF₃, OCHF₂, and morpholinyl.

In some embodiments, R^{QC} is selected from the group consisting of: —F, OH, NH₂NHMe, NHEt, NHC(═O)(cyclopropyl), NHS(O)₂Me, methyl, OMe, OCF₃, and morpholinyl.

In some embodiments of Formula IIA, IIB, or IIC, each R^{QC} is selected from the group consisting of: halo, cyano, OH, and NR^{c}R^{d}.

In some embodiments, each R^{QC} is selected from the group consisting of: —F, OH, and NHMe.

In some embodiments of Formula IIA, IIB, or IIC, a pair of R^{QC} on adjacent carbon atoms, taken together with the atom to which each is attached, forms a ring including from 5-6 ring atoms, wherein from 1-2 ring atoms are heteroatoms each independently selected from the group consisting of O, N, and S, wherein said ring is optionally substituted with from 1-2 independently selected R^{h} groups. In some embodiments, a pair of R^{QC} on adjacent carbon atoms, taken together with the atom to which each is attached form:

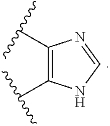 [SMILES: *c1nc[nH]c1*]

In some embodiments, a pair of R^{QC} on adjacent carbon atoms, taken together with the atom to which each is attached form:

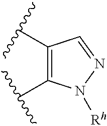

(e.g., R^{h} is methyl).

In some embodiments of Formula IIA, IIB, or IIC, L² is

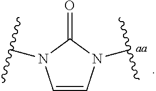 [SMILES: *n1ccn(*)c1=O]

In some embodiments of Formula IIA, IIB, or IIC, Ring A is phenyl or pyridyl, each of which is optionally substituted with from 2-4 substituents each independently selected from the group consisting of halo, C₁₋₆ alkyl, C₁₋₆ haloalkyl, and C₁₋₆ alkoxy.

In some embodiments of Formula IIA, IIB, or IIC, Ring A is phenyl, which is optionally substituted with from 2-4 substituents each independently selected from the group consisting of halo and C₁₋₆ alkyl.

In some embodiments, Ring A is

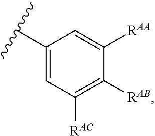

wherein R^{AA}, R^{AB}, and R^{AC} are independently halo or C₁₋₆ alkyl. For example each of R^{AA} and R^{AC} can be independently C₁₋₆ alkyl, such as C₁₋₃ alkyl (e.g., methyl); and/or R^{AB} can be halo (e.g., —F).

In some embodiments of Formula IIA, IIB, or IIC, R¹ and R² are H.

In some embodiments of Formula IIA, IIB, or IIC, R³ is H.

In some embodiments of Formula IIA, IIB, or IIC, R³ is C₁₋₆ alkyl.

In some embodiments of Formula IIA, IIB, or IIC, R¹ and R² are H; and R³ is C₁₋₆ alkyl. For example, R³ can be methyl.

In some embodiments of Formula IIA, IIB, or IIC, R⁴, R⁵, and R⁶ are each H or halo.

In some embodiments of Formula IIA, IIB, or IIC, R⁴, R⁵, and R⁶ are each H or —F. In certain embodiments of Formula IIA, IIB, or IIC, R⁴, R⁵, and R⁶ are each H. In some embodiments of Formula IIA, IIB, or IIC, R⁴ and R⁵ are H; and R⁶ is —F.

In some embodiments of Formula IIA, IIB, or IIC, R⁷ is H.

In some embodiments of Formula IIA, IIB, or IIC, R⁷ is —F.

In some embodiments of Formula IIA, IIB, or IIC, R⁹ is

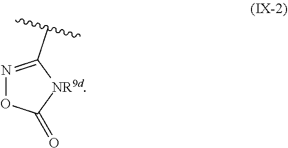

In some embodiments, R^{9d} is H.

In some embodiments, the compound of Formula I is a compound of Formula IID:

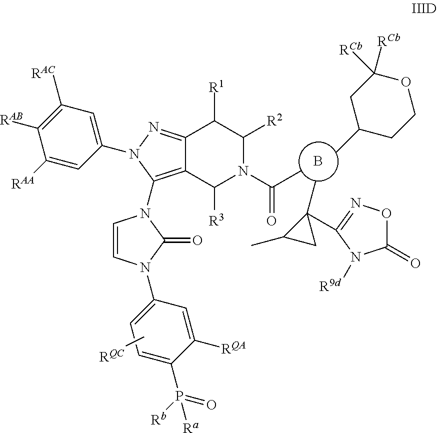

or a pharmaceutically acceptable salt, deuterated analog, or solvate thereof, wherein:
R^{AA}, R^{AB}, and R^{AC} are independently halo or C₁₋₆ alkyl;
R^{QC} is H or halo; and
each R^{Cb} is independently selected from the group consisting of H and R^{Ca}.

In some embodiments of Formula IID, R¹ and R² are H.

In some embodiments of Formula IID, R³ is C₁₋₃ alkyl. In some embodiments, R³ is methyl.

In some embodiments of Formula IID, R⁴, R⁵, R⁶, and R⁷ are H.

In some embodiments of Formula IID, R^{9d} is H.

In some embodiments of Formula IID, R^{AA} and R^{AC} are independently C₁₋₆ alkyl. In certain of these embodiments, R^{AA} and R^{AC} are methyl.

In some embodiments of Formula IID, R^{AB} is halo. In some embodiments, R^{AB} is —F.

In some embodiments of Formula IID, each R^{Cb} is H. In some embodiments of Formula IID, each R^{Cb} is C₁₋₃ alkyl. In some embodiments, each R^{Cb} is methyl.

In some embodiments of Formula IID, R^{a} and R^{b} are independently C₁₋₃ alkyl. In certain of these embodiments, R^{a} and R^{b} are methyl. In some embodiments, R^{a} and R^{b} are ethyl. In some embodiments, R^{a} and R^{b} are isopropyl.

In some embodiments of Formula IID, R^{a} is methyl; and R^{b} is tert-butyl.

In some embodiments of Formula IID, R^{a} and R^{b} are independently C₃₋₆ cycloalkyl. For example, R^{a} and R^{b} can both be cyclopropyl.

In some embodiments of Formula IID, P(═O)R^{a}R^{b} is

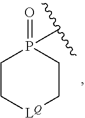

wherein L^{Q} is a bond, CH₂, O, S, NH, or N(C₁₋₆ alkyl). In some embodiments, P(═O)R^{a}R^{b} is

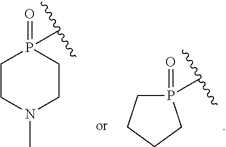

In some embodiments of Formula IID, R^{QC} is —F.

In some embodiments of Formula IID, R^{QC} is —OH.

In some embodiments of Formula IID, R^{QC} is NR^{c}R^{d}. In some embodiments, R^{QC} is NH(C₁₋₃ alkyl) (e.g., NHMe or NHEt). In some embodiments, R^{QC} is NH₂. In some embodiments, R^{QC} is N(C₁₋₃ alkyl)₂ (e.g., NMe₂). In some embodiments, R^{QC} is NHC(═O)(C₁₋₃ alkyl), NHC(═O)(C₃₋₆ cycloalkyl), or NHS(O)₂(C₁₋₃ alkyl).

In some embodiments of Formula IID, R^{QC} is 5-6 membered heterocyclyl optionally substituted with one or more substituents each independently selected from the group consisting of C₁₋₆ alkyl and C(═O)(C₁₋₆ alkyl).

In some embodiments of Formula IID, R^{QC} is C₁₋₆ alkoxy optionally substituted with from 1-6 independently selected halo. In some embodiments, R^{QC} is —OMe, —OCF₃, or —OCHF₂.

In some embodiments of Formula IID, R^{QC} is C₁₋₃ alkyl. In some embodiments, R^{QC} is methyl. In some embodiments of Formula IID, R^{QC} is C₁₋₃ alkyl substituted with from 1-3 independently selected halo. In some embodiments, R^{QC} is —CF₃ or —CHF₂.

In some embodiments of Formula IID, R^{QC} is H.

In some embodiments of Formula IID, R^{QC} is halo. In some embodiments, R^{QC} is —F. In some embodiments, R^{QC} is meta to P(═O)R^{a}R^{b}. In some embodiments, R^{QC} is meta to P(═O)R^{a}R^{b} and ortho to R^{QC}. In some embodiments, R^{QC} is —F which is ortho to R^{QC}. In some embodiments, R^{QC} is —F which is meta to P(═O)R^{a}R^{b} and ortho to R^{QC}.

In some embodiments of Formula IID, the compound is a compound of Formula (S, S, S)-IID:

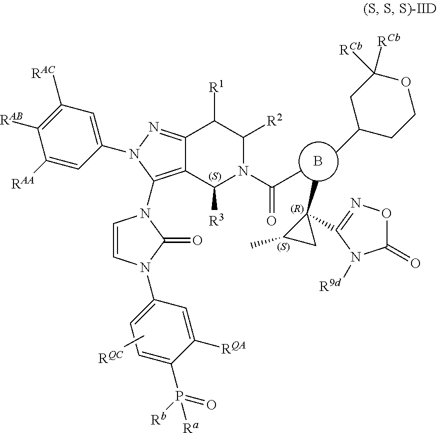

or a pharmaceutically acceptable salt thereof, wherein R³ is C₁₋₃ alkyl.

In some embodiments, the compound of Formula I is a compound of Formula IIE:

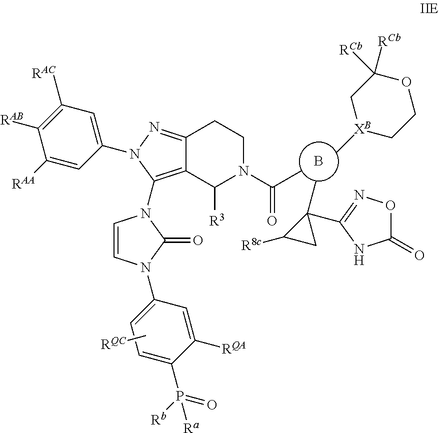

or a pharmaceutically acceptable salt, deuterated analog, or solvate thereof, wherein:
R^{AA}, R^{AB}, and R^{AC} are each independently halo or C₁₋₃ alkyl;
R³ is H or C₁₋₃ alkyl;
R^{QC} is H or halo;
R^{QD} is selected from the group consisting of: (a) H; (b) —NH₂; (c) —NH(C₁₋₃ alkyl); (d) halo; (e) C₁₋₃ alkoxy optionally substituted with from 1-3 independently selected halo; and (f) C₁₋₃ alkyl optionally substituted with from 1-3 independently selected halo;
R^{a} and R^{b} are each independently selected from the group consisting of: C₁₋₄ alkyl and C₃₋₆ cycloalkyl; or
R^{a} and R^{b} taken together with the phosphorous atom to which each is attached form a ring including from 5-8 ring atoms, wherein from 0-1 ring atom (in addition to the phosphorous attached to R^{a} and R^{b}) is a heteroatom independently selected from the group consisting of: O, S, and N;
R^{8c} is H or C₁₋₃ alkyl;
X^{B} is CH or N; and
each R^{Cb} is independently selected from the group consisting of H and C₁₋₃ alkyl.

In some embodiments, R^{QD} is selected from the group consisting of: (a) H; (b) —NH₂; (c) —NH(CD₃); (d) halo; (e) C₁₋₃ alkoxy optionally substituted with from 1-3 independently selected halo; and (f) C₁₋₃ alkyl optionally substituted with from 1-3 independently selected halo.

In some embodiments, Ring B of a compound of Formula IIC, IID, (S,S,S)-IID, IIE, or (S,S,S)-IIE is selected from the group consisting of:

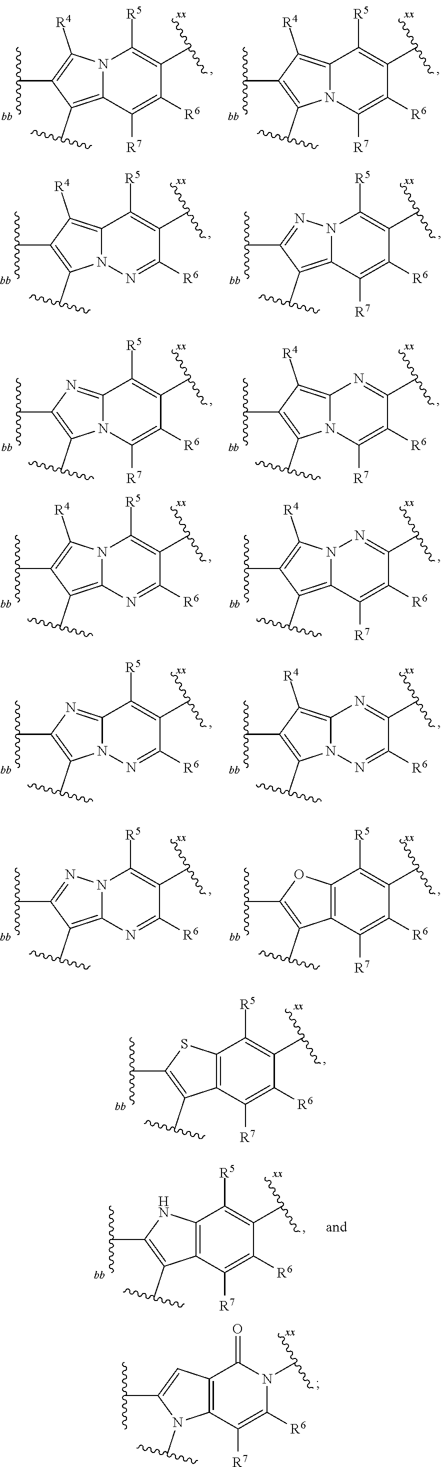

wherein R4, R5, R6, and R7 are each independently as defined herein; bb represents the point of attachment to the —C(O)—; and xx represents the point of attachment to the Ring C portion as described in Formula I.

In some embodiments, the compound of Formula I is a compound of Formula IIE:

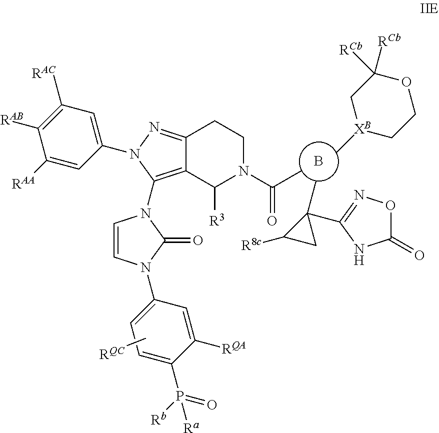

or a pharmaceutically acceptable salt, deuterated analog, or solvate thereof, wherein:
Ring B is selected from the group consisting of:

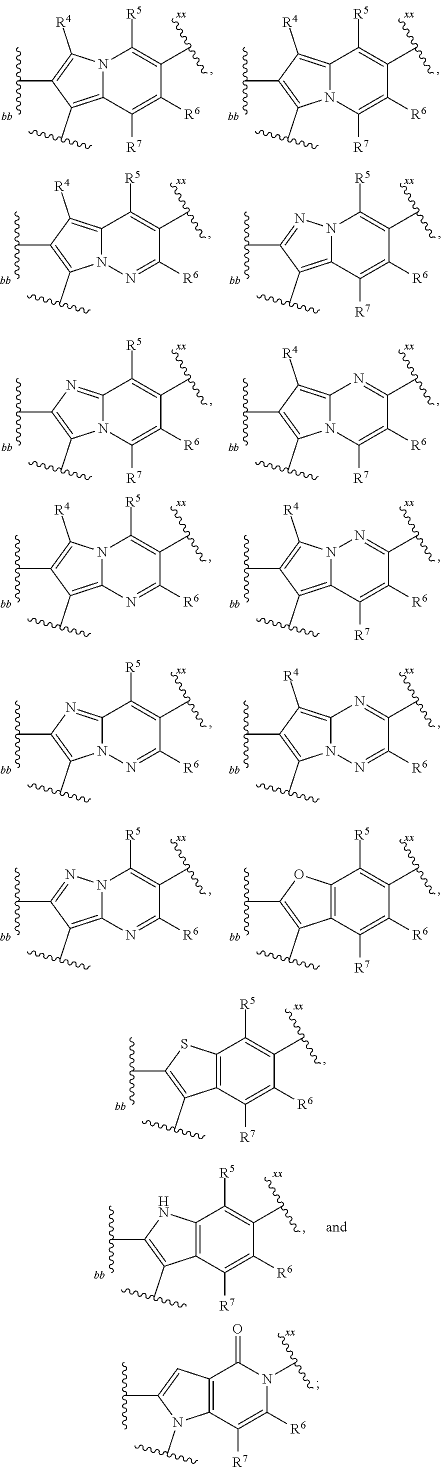

wherein bb represents the point of attachment to the —C(O)— and xx represents the point of attachment to X^{b};
R⁴, R⁵, R⁶, and R⁷ are independently selected from the group consisting of H, halo, and C₁₋₆ alkyl;
R^{AA}, R^{AB}, and R^{AC} are each independently hydrogen, halo, C₁₋₃ alkyl, or C₃₋₆ cycloalkyl;
R³ is H or C₁₋₃ alkyl;
R^{QC} is H or halo;
R^{QD} is selected from the group consisting of: (a) H; (b) —NH₂; (c) —NH(C₁₋₃ alkyl); (d) halo; (e) C₁₋₃ alkoxy optionally substituted with from 1-3 independently selected halo; and (f) C₁₋₃ alkyl optionally substituted with from 1-3 independently selected halo;
R^{a} and R^{b} are each independently selected from the group consisting of: C₁₋₄ alkyl and C₃₋₆ cycloalkyl; or
R^{a} and R^{b} taken together with the phosphorous atom to which each is attached form a ring including from 5-8 ring atoms, wherein from 0-1 ring atom (in addition to the phosphorous attached to R^{a} and R^{b}) is a heteroatom independently selected from the group consisting of: O, S, and N;
R^{8c} is H or C₁₋₃ alkyl;
X^{B} is CH or N; and
each R^{Cb} is independently selected from the group consisting of H and C₁₋₃ alkyl.

In some embodiments of Formula IIE, R^{AA} and R^{AC} are each independently C₁₋₃ alkyl. For example, R^{AA} and R^{AC} can each be methyl.

In some embodiments of Formula IIE, R^{AB} is halo. For example, R^{AB} can be F.

In some embodiments of Formula IIE, R^{AA} and R^{AC} are each independently C₁₋₃ alkyl; and R^{AB} is halo. For example, R^{AA} and R^{AC} can each be methyl; and R^{AB} can be F.

In some embodiments of Formula IIE, R³ is C₁₋₃ alkyl. For example, R³ can be methyl.

In some embodiments of Formula IIE, R^{QD} is H.

In some embodiments of Formula IIE, R^{QD} is selected from the group consisting of —NH₂ and —NH(C₁₋₃ alkyl). For example, R^{QD} can be —NH₂. As another non-limiting example, R^{QD} can be —NHMe. As a further non-limiting example, R^{QD} can be —NHEt.

In some embodiments of Formula IIE, R^{QD} is halo. For example, R^{QD} can be —F or —Cl.

In some embodiments of Formula IIE, R^{QD} is C₁₋₃ alkoxy optionally substituted with from 1-3 independently selected halo. For example, R^{QD} can be —OMe. As another non-limiting example, R^{QD} can be OCF₃. As a further non-limiting example, R^{QD} can be OCHF₂.

In some embodiments of Formula IIE, R^{QD} is C₁₋₃ alkyl optionally substituted with from 1-3 independently selected halo. For example, R^{QD} can be methyl. As another non-limiting example, R^{QD} can be CF₃. As another non-limiting example, R³ can be —CHF₂.

In some embodiments of Formula IIE, R^{a} and R^{b} are each an independently selected C₁₋₄ alkyl. For example, R^{a} and R^{b} can each be methyl. As another non-limiting example, R^{a} and R^{b} can each be ethyl. As a further non-limiting example, R^{a} and R^{b} can each be isopropyl. As a further non-limiting example, R^{a} can be methyl; and R^{b} can be tert-butyl.

In some embodiments of Formula IIE, R^{a} and R^{b} are each an independently selected C₃₋₆ cycloalkyl. For example, R^{a} and R^{b} can each be cyclopropyl.

In some embodiments of Formula IIE, R^{a} and R^{b} taken together with the phosphorous atom to which each is attached form a ring including from 5-8 ring atoms, wherein from 0-1 ring atom (in addition to the phosphorous attached to R^{a} and R^{b}) is a heteroatom independently selected from the group consisting of: O, S, and N. For example, R^{a} and R^{b} taken together with the phosphorous atom to which each is attached can form

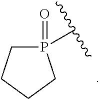 [SMILES: *P1(=O)CCCC1]

In some embodiments of Formula IIE, R^{QC} is H.

In some embodiments of Formula IIE, R^{QC} is halo. For example, R^{QC} can be —F. In some embodiments, R^{QC} is ortho to R^{QD}. In some embodiments, R^{QC} is para to R^{QD}. In some embodiments, R^{QC} is meta to R^{QD}.

In some embodiments of Formula IIE, R^{8c} is H. In some embodiments of Formula IIE, R^{8c} is C₁₋₃ alkyl. For example, R^{8c} can be methyl.

In some embodiments of Formula IIE, X^{B} is CH. In some embodiments of Formula IIE, XB is N.

In some embodiments of Formula IIE, each R^{Cb} is H. In some embodiments of Formula IE, each R^{Cb} is an independently selected C₁₋₃ alkyl. For example, each R^{Cb} can be methyl.

In some embodiments of Formula IIE, X^{B} is CH; and each R^{Cb} is H. In some embodiments of Formula IIE, X^{B} is CH; and each R^{Cb} is an independently selected C₁₋₃ alkyl (e.g., methyl). In some embodiments of Formula IIE, X^{B} is N; and each R^{Cb} is H. In some embodiments of Formula IIE, X^{B} is N; and each R^{Cb} is an independently selected C₁₋₃ alkyl (e.g., methyl).

In some embodiments of Formula IIE, the compound is a compound of Formula (S, S, S)-IIE:

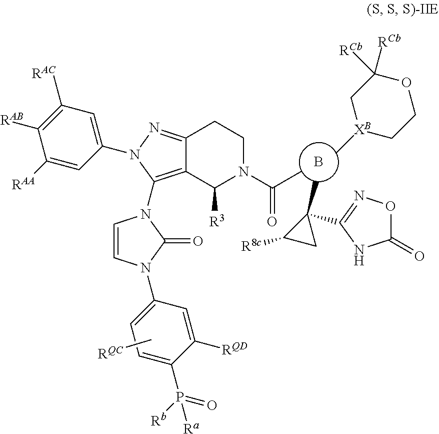

or a pharmaceutically acceptable salt thereof, wherein R³ is C₁₋₃ alkyl.

In some embodiments of Formula IIE:
R^{AA} and R^{A}C are methyl;
R^{AB} is —F;
R³ is methyl;
R^{QC} is H or —F;
R^{QD} is selected from the group consisting of: H; —NHMe; —NHEt; —NH₂; —OMe; —OCF₃; —OCHF₂; -Me; —CF₃; and —CHF₂;
R^{a} and R^{b} are independently selected from the group consisting of: methyl, ethyl, isopropyl, tert-butyl, and cyclopropyl;
R^{8c} is H or methyl;
X^{B} is CH; and
each R^{Cb} is independently H or methyl.

In certain of these embodiments, the compound is a compound of Formula (S, S, S)-IIE.

In some embodiments, provided is a compound of Formula III, or a pharmaceutically acceptable salt or solvate thereof, comprising any one or more of the features delineated below.

In some embodiments of Formula I or III, (aa) applies.

In some embodiments, Ring D is selected from the group consisting of: C₃₋₁₅ cycloalkyl; 3-12 membered heterocyclyl; 5-10 membered heteroaryl; and C₆₋₁₀ aryl, each of which is substituted with one R^{QB} and further optionally substituted with from 1-5 independently selected R^{QA}.

In some embodiments, Ring D is selected from the group consisting of: 5-10 membered heteroaryl; and C₆₋₁₀ aryl, each of which is substituted with one R^{QB} and further optionally substituted with from 1-5 independently selected R^{QA}. In some embodiments, Ring D is 5-6 membered heteroaryl or phenyl, each of which is substituted with one R^{QB} and further optionally substituted with from 1-5 independently selected R^{QA}.

In some embodiments, Ring D is:

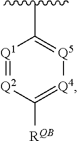

wherein Q¹, Q², Q⁴, and Q⁵ are each independently N, CH, or CR^{QA}. In some embodiments, Q¹, Q², Q⁴, and Q⁵ are independently CH or CR^{QA}. In some embodiments, each of Q¹, Q², Q⁴, and Q⁵ are CH. In some embodiments, from 1-2 (e.g., 1) of Q¹, Q², Q⁴, and Q⁵ can be CR^{QA}; and each remaining of Q¹, Q², Q⁴, and Q⁵ is CH. In some embodiments, one of Q¹, Q², Q⁴, and Q⁵ is N; and each remaining of Q¹, Q², Q⁴, and Q⁵ is independently CH or CR^{QA}. In some embodiments, each remaining of Q¹, Q², Q⁴, and Q⁵ are CH.

In some embodiments, Ring D is:

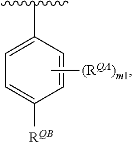

wherein m1 is 0, 1, or 2. For example, m1 can be 0. In some embodiments, m1 can be 1; and optionally R^{QA} is meta to R^{QB}.

In some embodiments, Ring D is:

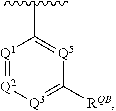

wherein Q¹, Q², Q³, and Q⁵ are each independently N, CH, or CR^{QA}. In some embodiments, Q¹, Q², Q³, and Q⁵ are independently CH or CR^{QA}. In some embodiments, each of Q¹, Q², Q³, and Q⁵ are CH. In some embodiments, from 1-2 (e.g., 1) of Q¹, Q², Q³, and Q⁵ are CR^{QA}; and each remaining of Q¹, Q², Q³, and Q⁵ are CH.

In some embodiments, Ring D is:

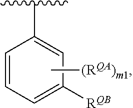

wherein m1 is 0, 1, or 2. In some embodiments, m1 is 0. In some embodiments, m1 is 1.

In some embodiments, R^{QB} is -L^{a}-S(O)₂-L^{b}-R^{e}. In some embodiments, L^{a} is —(CR^{h}R^{h})_{q1}—; and q1 is 1, 2, 3, or 4. For example, q1 can be 1. As another non-limiting example, q1 can be 2. In some embodiments, each occurrence of R^{h} is H. In some embodiments, one occurrence of R^{h} is C₁₋₆ alkyl, C₁₋₆ haloalkyl, C₃₋₆ cycloalkyl, or halo; and each remaining R^{h} is H. In some embodiments, one occurrence of R^{h} is C₁₋₃ alkyl (e.g., methyl); and each remaining R^{h} is H. In some embodiments, L^{b} is —(CR^{h}R^{h})_{q2}—. In some embodiments, q2 is 0. In some embodiments, R^{c} is C₁₋₆ alkyl, C₁₋₆ haloalkyl, or C₃₋₆ cycloalkyl, each of which is optionally substituted with from 1-3 substituents each independently selected from the group consisting of: halo, cyano, C₁₋₃ alkyl, C₁₋₃ haloalkyl, —OH, NH₂, NH(C₁₋₃ alkyl), N(C₁₋₃ alkyl)₂, C₁₋₃ alkoxy, and C₁₋₃ haloalkoxy. In some embodiments, R^{c} is C₁₋₆ alkyl or C₁₋₆ haloalkyl, each of which is optionally substituted with from 1-3 substituents each independently selected from the group consisting of: halo, cyano, C₁₋₃ alkyl, C₁₋₃ haloalkyl, —OH, NH₂, NH(C₁₋₃ alkyl), N(C₁₋₃ alkyl)₂, C₁₋₃ alkoxy, and C₁₋₃ haloalkoxy. In some embodiments, R^{e} is C₁₋₆ alkyl. For example, R^{c} can be C₁₋₃ alkyl, such as methyl or ethyl.

In some embodiments, R^{QB} is -L^{a}-S(O)₂-L^{b}-R^{e}; L^{a} is —(CR^{h}R^{h})_{q1}—; q1 is 1, 2, 3, or 4; and each occurrence of R^{h} is H. In some embodiments, q1 is 1. In some embodiments, q1 is 2. In some embodiments, L^{b} is —(CR^{h}R^{h})_{q2}—. In some embodiments, q2 is 0. In some embodiments, R^{e} is C₁₋₆ alkyl, C₁₋₆ haloalkyl, or C₃₋₆ cycloalkyl, each of which is optionally substituted with from 1-3 substituents each independently selected from the group consisting of: halo, cyano, C₁₋₃ alkyl, C₁₋₃ haloalkyl, —OH, NH₂, NH(C₁₋₃ alkyl), N(C₁₋₃ alkyl)₂, C₁₋₃ alkoxy, and C₁₋₃ haloalkoxy. In some embodiments, R^{c} is C₁₋₆ alkyl or C₁₋₆ haloalkyl, each of which is optionally substituted with from 1-3 substituents each independently selected from the group consisting of: halo, cyano, C₁₋₃ alkyl, C₁₋₃ haloalkyl, —OH, NH₂, NH(C₁₋₃ alkyl), N(C₁₋₃ alkyl)₂, C₁₋₃ alkoxy, and C₁₋₃ haloalkoxy. In some embodiments, R^{c} is C₁₋₆ alkyl. For example, R^{c} can be C₁₋₃ alkyl, such as methyl or ethyl.

In some embodiments, R^{QB} is -L^{a}-S(O)₂-L^{b}-R^{c}; L^{a} is —(CR^{h}R^{h})_{q1}—; q1 is 1, 2, 3, or 4; one occurrence of R^{h} is C₁₋₆ alkyl, C₁₋₆ haloalkyl, C₃₋₆ cycloalkyl, or halo; and each remaining R^{h} is H. In some embodiments, one occurrence of R^{h} is C₁₋₃ alkyl (e.g., methyl); and each remaining R^{h} is H. In some embodiments, q1 is 1. For example, R^{QB} can be

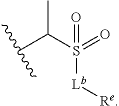

In some embodiments, q1 is 2. In some embodiments, L^{b} is —(CR^{h}R^{h})_{q2}—. In some embodiments, q2 is 0. In some embodiments, R^{c} is C₁₋₆ alkyl, C₁₋₆ haloalkyl, or C₃₋₆ cycloalkyl, each of which is optionally substituted with from 1-3 substituents each independently selected from the group consisting of: halo, cyano, C₁₋₃ alkyl, C₁₋₃ haloalkyl, —OH, NH₂, NH(C₁₋₃ alkyl), N(C₁₋₃ alkyl)₂, C₁₋₃ alkoxy, and C₁₋₃ haloalkoxy. In some embodiments, R^{c} is C₁₋₆ alkyl or C₁₋₆ haloalkyl, each of which is optionally substituted with from 1-3 substituents each independently selected from the group consisting of: halo, cyano, C₁₋₃ alkyl, C₁₋₃ haloalkyl, —OH, NH₂, NH(C₁₋₃ alkyl), N(C₁₋₃ alkyl)₂, C₁₋₃ alkoxy, and C₁₋₃ haloalkoxy. In some embodiments, R^{c} is C₁₋₆ alkyl. For example, R^{c} can be C₁₋₃ alkyl, such as methyl or ethyl.

In some embodiments, R^{QB} is —(CR^{h}R^{h})_{q1}—S(O)₂—(CR^{h}R^{h})_{q2}—R^{e}; q1 is 1 or 2; and R^{e} is C₁₋₆ alkyl, C₁₋₆ haloalkyl, or C₃₋₆ cycloalkyl, each of which is optionally substituted with from 1-3 substituents each independently selected from the group consisting of: halo, cyano, C₁₋₃ alkyl, C₁₋₃ haloalkyl, —OH, NH₂, NH(C₁₋₃ alkyl), N(C₁₋₃ alkyl)₂, C₁₋₃ alkoxy, and C₁₋₃ haloalkoxy. In some embodiments, q2 is 0. In some embodiments, q1 is 1. In some embodiments, q1 is 2. In some embodiments, each occurrence of R^{h} is H or C₁₋₃ alkyl. In some embodiments, R^{c} is C₁₋₆ alkyl or C₁₋₆ haloalkyl, each of which is optionally substituted with from 1-3 substituents each independently selected from the group consisting of: halo, cyano, C₁₋₃ alkyl, C₁₋₃ haloalkyl, —OH, NH₂, NH(C₁₋₃ alkyl), N(C₁₋₃ alkyl)₂, C₁₋₃ alkoxy, and C₁₋₃ haloalkoxy. For example, R^{c} can be C₁₋₆ alkyl, such as C₁₋₃ alkyl (e.g., methyl or ethyl).

In some embodiments, R^{QB} is —CH₂—S(O)₂(C₁₋₆ alkyl). For example, R^{QB} can be —CH₂—S(O)₂Me or —CH₂—S(O)₂Et.

In some embodiments, R^{QB} is —CH(C₁₋₆ alkyl)—S(O)₂(C₁₋₆ alkyl). For example, R^{QB} can be —CH(Me)S(O)₂(C₁₋₃ alkyl), such as —CH(Me)S(O)₂Me.

In some embodiments, R^{QB} is —CH₂CH₂—S(O)₂(C₁₋₆ alkyl). For example, R^{QB} can be —CH₂CH₂S(O)₂Me.

In some embodiments, R^{QB} is -L^{a}-S(O)₂-L^{b}-R^{c}, wherein L^{a} is —(CR^{h}R^{h})_{q1}—; and q1 is 0. In some embodiments, L^{b} is —NH— or —N(R)—. In some embodiments, L^{b} is —NH— or —N(C₁₋₃ alkyl)-. For example, L^{b} can be —NH—. In some embodiments, q2 is 0. In some embodiments, R^{e} is C₁₋₆ alkyl, C₁₋₆ haloalkyl, or C₃₋₆ cycloalkyl, each of which is optionally substituted with from 1-3 substituents each independently selected from the group consisting of: halo, cyano, C₁₋₃ alkyl, C₁₋₃ haloalkyl, —OH, NH₂, NH(C₁₋₃ alkyl), N(C₁₋₃ alkyl)₂, C₁₋₃ alkoxy, and C₁₋₃ haloalkoxy. In some embodiments, R^{c} is C₁₋₆ alkyl. For example, R^{e} can be C₁₋₃ alkyl, such as methyl or ethyl. In some embodiments, R^{c} is C₃₋₆ cycloalkyl. For example, R^{c} can be cyclopropyl.

In some embodiments, R^{QB} is -L^{a}-S(O)₂-L^{b}-R^{c}, wherein L^{a} is —(CR^{h}R^{h})_{q1}—; q1 is 0; and L^{b} is —(CR^{h}R^{h})_{q2}—. In some embodiments, q2 is 0. In some embodiments, R^{c} is C₁₋₆ alkyl, C₁₋₆ haloalkyl, or C₃₋₆ cycloalkyl, each of which is optionally substituted with from 1-3 substituents each independently selected from the group consisting of: halo, cyano, C₁₋₃ alkyl, C₁₋₃ haloalkyl, —OH, NH₂, NH(C₁₋₃ alkyl), N(C₁₋₃ alkyl)₂, C₁₋₃ alkoxy, and C₁₋₃ haloalkoxy, provided that R^{e} is other than unsubstituted C₁₋₃ alkyl. In some embodiments, R^{c} is C₃₋₆ cycloalkyl. For example, R^{c} can be cyclopropyl.

In some embodiments, wherein R^{QB} is —S(O)₂NH—R^{e}; and R^{c} is C₁₋₆ alkyl, C₁₋₆ haloalkyl, or C₃₋₆ cycloalkyl, each of which is optionally substituted with from 1-3 substituents each independently selected from the group consisting of: halo, cyano, C₁₋₃ alkyl, C₁₋₃ haloalkyl, —OH, NH₂, NH(C₁₋₃ alkyl), N(C₁₋₃ alkyl)₂, C₁₋₃ alkoxy, and C₁₋₃ haloalkoxy. In some embodiments, R^{c} is C₁₋₆ alkyl. For example, R^{c} can be methyl or ethyl, such as methyl.

In some embodiments, wherein R^{QB} is —S(O)₂—R^{e}; and R^{c} is C₁₋₆ alkyl, C₁₋₆ haloalkyl, or C₃₋₆ cycloalkyl, each of which is optionally substituted with from 1-3 substituents each independently selected from the group consisting of: halo, cyano, C₁₋₃ alkyl, C₁₋₃ haloalkyl, —OH, NH₂, NH(C₁₋₃ alkyl), N(C₁₋₃ alkyl)₂, C₁₋₃ alkoxy, and C₁₋₃ haloalkoxy, provided that R^{c} is other than unsubstituted C₁₋₃ alkyl. In some embodiments, R^{c} is C₃₋₆ cycloalkyl. For example, R^{c} can be cyclopropyl.

In some embodiments, R^{QB} is -L^{a}-S(O)₂-L^{b}-R^{c}, wherein L^{a} is —NH— or —N(R′)—. In some embodiments, L^{a} is —NH— or —N(C₁₋₃ alkyl)-. For example, L^{a} is —NH—. In some embodiments, L^{b} is —(CR^{h}R^{h})_{q2}—. In some embodiments, q2 is 0. In some embodiments, R^{c} is C₁₋₆ alkyl, C₁₋₆ haloalkyl, or C₃₋₆ cycloalkyl, each of which is optionally substituted with from 1-3 substituents each independently selected from the group consisting of: halo, cyano, C₁₋₃ alkyl, C₁₋₃ haloalkyl, —OH, NH₂, NH(C₁₋₃ alkyl), N(C₁₋₃ alkyl)₂, C₁₋₃ alkoxy, and C₁₋₃ haloalkoxy. In some embodiments, R^{c} is C₁₋₆ alkyl or C₁₋₆ haloalkyl, each of which is optionally substituted with from 1-3 substituents each independently selected from the group consisting of: halo, cyano, C₁₋₃ alkyl, C₁₋₃ haloalkyl, —OH, NH₂, NH(C₁₋₃ alkyl), N(C₁₋₃ alkyl)₂, C₁₋₃ alkoxy, and C₁₋₃ haloalkoxy. For example, R^{c} can be C₁₋₆ alkyl, such as C₁₋₃ alkyl (e.g., methyl or ethyl).

In some embodiments, R^{QB} is -L^{a}-S(O)₂-L^{b}-R^{e}, wherein L^{a} is —NH— or —N(R′)—; and L^{b} is —(CR^{h}R^{h})_{q2}—. In some embodiments, L^{a} is —NH— or —N(C₁₋₃ alkyl)-. In some embodiments, q2 is 0. In some embodiments, R^{c} is C₁₋₆ alkyl, C₁₋₆ haloalkyl, or C₃₋₆ cycloalkyl, each of which is optionally substituted with from 1-3 substituents each independently selected from the group consisting of: halo, cyano, C₁₋₃ alkyl, C₁₋₃ haloalkyl, —OH, NH₂, NH(C₁₋₃ alkyl), N(C₁₋₃ alkyl)₂, C₁₋₃ alkoxy, and C₁₋₃ haloalkoxy. In some embodiments, R^{c} is C₁₋₆ alkyl or C₁₋₆ haloalkyl, each of which is optionally substituted with from 1-3 substituents each independently selected from the group consisting of: halo, cyano, C₁₋₃ alkyl, C₁₋₃ haloalkyl, —OH, NH₂, NH(C₁₋₃ alkyl), N(C₁₋₃ alkyl)₂, C₁₋₃ alkoxy, and C₁₋₃ haloalkoxy. For example, R^{c} can be C₁₋₆ alkyl, such as C₁₋₃ alkyl (e.g., methyl or ethyl).

In some embodiments, R^{QB} is —NHS(O)₂—(CR^{h}R^{h})_{q2}—R^{c}; and R^{c} is C₁₋₆ alkyl, C₁₋₆ haloalkyl, or C₃₋₆ cycloalkyl, each of which is optionally substituted with from 1-3 substituents each independently selected from the group consisting of: halo, cyano, C₁₋₃ alkyl, C₁₋₃ haloalkyl, —OH, NH₂, NH(C₁₋₃ alkyl), N(C₁₋₃ alkyl)₂, C₁₋₃ alkoxy, and C₁₋₃ haloalkoxy. In some embodiments, q2 is 0. In some embodiments, R^{c} is C₁₋₆ alkyl. For example, R^{e} can be C₁₋₃ alkyl, such as methyl or ethyl.

In some embodiments, R^{QB} is -L^{a}-S(O)₂-L^{b}-R^{e}, wherein L^{b} is —NH— or —N(R′)—. For example, L^{b} can be —NH— or —N(C₁₋₃ alkyl)-. For example, L^{b} can be —NH—. In some embodiments, L^{a} is —(CR^{h}R^{h})_{q1}. In some of these embodiments, q1 is 0. In some embodiments, R^{e} is C₁₋₆ alkyl, C₁₋₆ haloalkyl, or C₃₋₆ cycloalkyl, each of which is optionally substituted with from 1-3 substituents each independently selected from the group consisting of: halo, cyano, C₁₋₃ alkyl, C₁₋₃ haloalkyl, —OH, NH₂, NH(C₁₋₃ alkyl), N(C₁₋₃ alkyl)₂, C₁₋₃ alkoxy, and C₁₋₃ haloalkoxy. In some embodiments, R^{c} is C₁₋₆ alkyl. For example, R^{c} can be C₁₋₃ alkyl, such as methyl or ethyl. In some embodiments, R^{c} is C₃₋₆ cycloalkyl. For example, R^{c} can be cyclopropyl.

In some embodiments, R^{QB} is -L^{a}-S(O)₂-L^{b}-R^{c}, wherein L^{b} is —(CR^{h}R^{h})_{q2}—. In some embodiments, q2 is 0. In some embodiments, R^{c} is C₁₋₆ alkyl, C₁₋₆ haloalkyl, or C₃₋₆ cycloalkyl, each of which is optionally substituted with from 1-3 substituents each independently selected from the group consisting of: halo, cyano, C₁₋₃ alkyl, C₁₋₃ haloalkyl, —OH, NH₂, NH(C₁₋₃ alkyl), N(C₁₋₃ alkyl)₂, C₁₋₃ alkoxy, and C₁₋₃ haloalkoxy. In some embodiments, R^{c} is C₁₋₆ alkyl. In some embodiments, R^{c} is C₃₋₆ cycloalkyl. In some embodiments, L^{a} is —NH— or N(C₁₋₃ alkyl), such as —NH—. In some embodiments, L^{a} is —(CR^{h}R^{h})_{q1}—. In some embodiments, q1 is 1 or 2. In some embodiments, q1 is 0.

In some embodiments, R^{QB} is -L^{a}-S(O)₂-L^{b}-R^{e}, wherein R^{e} is C₁₋₆ alkyl, C₁₋₆ haloalkyl, or C₃₋₆ cycloalkyl, each of which is optionally substituted with from 1-3 substituents each independently selected from the group consisting of: halo, cyano, C₁₋₃ alkyl, C₁₋₃ haloalkyl, —OH, NH₂, NH(C₁₋₃ alkyl), N(C₁₋₃ alkyl)₂, C₁₋₃ alkoxy, and C₁₋₃ haloalkoxy. In some embodiments, R^{c} is C₁₋₆ alkyl or C₁₋₆ haloalkyl, each of which is optionally substituted with from 1-3 substituents each independently selected from the group consisting of: halo, cyano, C₁₋₃ alkyl, C₁₋₃ haloalkyl, —OH, NH₂, NH(C₁₋₃ alkyl), N(C₁₋₃ alkyl)₂, C₁₋₃ alkoxy, and C₁₋₃ haloalkoxy. In some embodiments, R^{e} is C₁₋₆ alkyl. For example, R^{e} can be C₁₋₃ alkyl, such as methyl or ethyl.

In some embodiments, R^{QB} is 4-12 membered heterocyclyl or 7-10 membered bicyclic heteroaryl, each of which comprises an endocyclic S(O)₂ group, wherein the heterocyclyl or heteroaryl is optionally substituted with from 1-6 independently selected R⁹.

In some embodiments, R^{QB} is 4-10 membered heterocyclyl which comprises an endocyclic

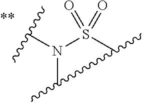

group, wherein ** represents the point of attachment to Ring D, and the heterocyclyl is optionally substituted with from 1-6 independently selected R⁹.

In some embodiments, R^{QB} is:

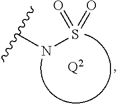

wherein Ring Q² is a 4-8 membered heterocyclyl including from 0-2 additional ring heteroatoms (in addition to the endocyclic N—S(O)₂ group) each independently selected from the group consisting of: N, O, and S(O)₀₋₂, wherein Ring Q² is optionally substituted with from 1-4 independently selected R⁹. In some embodiments, R^{QB} is:

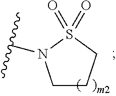

m2 is 0, 1, 2, or 3; and R^{QB} is optionally substituted with from 1-4 independently selected R^{g}. For example, R^{QB} can be:

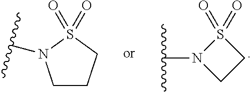 [SMILES: *N1CCCS1(=O)=O ; *N1CCS1(=O)=O]

As further non-limiting examples, R^{QB} can be selected from the group consisting of:

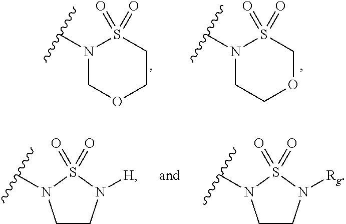

In some embodiments, one occurrence of R^{QA} is -halo. For example, one occurrence of R^{QA} can be —F.

In some embodiments of Formula I or III, (bb) applies.

In some embodiments, Ring D is 4-12 membered heterocyclyl or 7-10 membered bicyclic heteroaryl, wherein Ring D comprises an endocyclic S(O)₂ group; wherein Ring D is optionally substituted with from 1-4 R^{QA}.

In some embodiments, Ring D is 4-12 membered heterocyclyl or 7-10 membered bicyclic heteroaryl, wherein Ring D comprises an endocyclic —N(H)S(O)₂— group or —N(C₁₋₃ alkyl)S(O)₂— group; wherein Ring D is further optionally substituted with from 1-3 R^{QA}.

In some embodiments, Ring D is 7-10 membered bicyclic heteroaryl, wherein Ring D comprises an endocyclic —N(H)S(O)₂— group or —N(C₁₋₃ alkyl)S(O)₂— group; and Ring D is further optionally substituted with from 1-3 R^{QA}. For example, Ring D can be:

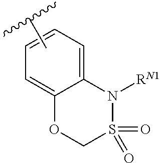

wherein R^{N1} is H or C₁₋₃ alkyl.

In some embodiments, R¹ is H. In some embodiments, R² is H. In some embodiments, R³ is C₁₋₆ alkyl. For example, R³ is C₁₋₃ alkyl such as methyl. In some embodiments, R³ is C₁₋₆ alkyl, and the carbon atom to which R³ is attached has (S)-configuration.

In some embodiments, R¹, R², and R³ are H.

In some embodiments, R¹ and R² are H; and R³ is C₁₋₆ alkyl. In some embodiments, R¹ and R² are H; and R³ is methyl. In some embodiments, the carbon atom to which R³ is attached has (S)-configuration.

In some embodiments, L² is:

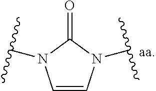 [SMILES: *n1ccn(*)c1=O]

In some embodiments, Ring A is C₆₋₁₀ aryl or 5-10 membered heteroaryl, each of which is optionally substituted with from 1-4 substituents each independently selected from the group consisting of halo, C₁₋₆ alkyl, C₁₋₆ haloalkyl, and C₁₋₆ alkoxy.

In some embodiments, Ring A is phenyl or pyridyl, each of which is optionally substituted with from 2-4 substituents each independently selected from the group consisting of halo, C₁₋₆ alkyl, C₁₋₆ haloalkyl, and C₁₋₆ alkoxy.

In some embodiments, Ring A is phenyl, which is optionally substituted with from 2-4 substituents each independently selected from the group consisting of halo and C₁₋₆ alkyl.

In some embodiments, Ring A is:

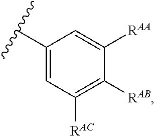

wherein R^{AA}, R^{AB}, and R^{AC} are independently halo or C₁₋₆ alkyl. In some embodiments, R^{AA} and R^{A}c are independently C₁₋₆ alkyl (e.g., C₁₋₃ alkyl, such as methyl). In some embodiments, R^{AB} is halo (e.g., —F).

In some embodiments, Ring A is pyridyl, which is optionally substituted with from 2-4 substituents each independently selected from the group consisting of halo and C₁₋₆ alkyl.

In some embodiments, L¹ is C(═O).

In some embodiments, R⁴, R⁵, and R⁶ are each H or halo. In some embodiments, R⁴, R⁵, and R⁶ are each H or —F. For example, R⁴, R⁵, and R⁶ can each be H. As another non-limiting example, R⁴ and R⁵ can be H; and R⁶ can be —F.

In some embodiments, R⁷ is H. In some embodiments, R⁷ is —F.

In some embodiments, at least one of L³ and L⁴ is a bond. In some embodiments, both of L³ and L⁴ are bonds. In some embodiments, L³ is a bond; and L⁴ is C₁₋₂ alkylene. In some embodiments, L⁴ is a bond; and L³ is C₁₋₂ alkylene.

In some embodiments, L³ and L⁴ are each independently C₁₋₂ alkylene.

In some embodiments, R^{8a} and R^{8b} taken together with the carbon atom to which each is attached forms a C₃₋₈ cycloalkyl ring which is optionally substituted with from 1-2 (e.g., 1) independently selected C₁₋₆ alkyl, wherein the C₁₋₆ alkyl is optionally substituted with from 1-3 independently selected R^{f}. In some embodiments, L³ is a bond; and L⁴ is a bond.

In some embodiments, R^{8a} and R^{8b} taken together with the carbon atom to which each is attached forms a C₃₋₅ (e.g., C₃ or C₄) cycloalkyl ring which is optionally substituted with from 1-2 (e.g., 1) independently selected C₁₋₆ alkyl, wherein the C₁₋₆ alkyl is optionally substituted with from 1-3 independently selected R^{f}. In some embodiments, L³ is a bond; and L⁴ is a bond.

In some embodiments, R^{8a} and R^{8b} taken together with the carbon atom to which each is attached forms a C₃₋₄ cycloalkyl ring which is optionally substituted with from 1-2 (e.g., 1) independently selected C₁₋₆ alkyl.

In some embodiments, R^{8a} and R^{8b} taken together with the carbon atom to which each is attached forms:

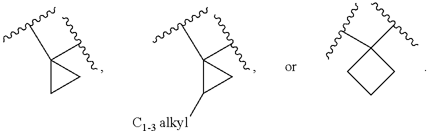

In some embodiments, R^{8a} and R^{8b} taken together with the carbon atom to which each is attached forms

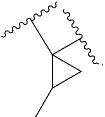 [SMILES: *C1(*)CC1C]

In some embodiments, R⁹ is:

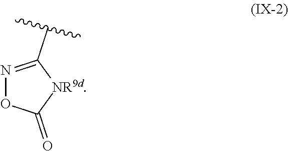

In some embodiments, R^{9d} is H or C₁₋₆ alkyl. For example, R^{9d} can be H.

In some embodiments, the L³-C(R^{8a}R^{8b})-L⁴-R⁹ moiety is:

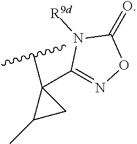

In some embodiments, the L³-C(R^{8a}R^{8b})-L⁴-R⁹ moiety is:

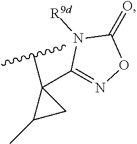

wherein each stereogenic center has (S)-configuration.

In some embodiments, R⁹ is (IX-2), wherein the L³-C(R^{8a}R^{8b})-L⁴-R⁹ moiety is:

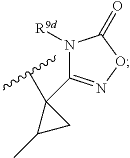

wherein R^{9d} is H or C₁₋₆ alkyl. For example, R^{9d} is H. In some embodiments, each stereogenic center of

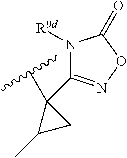

has (S)-configuration.

In some embodiments, Ring C is 3-12 membered heterocyclyl which is optionally substituted with from 1-3 independently selected R^{Ca}. In some embodiments, Ring C is 4-8 membered heterocyclyl which is optionally substituted with from 1-3 independently selected R^{Ca}. In some embodiments, Ring C is 5-6 membered heterocyclyl which is optionally substituted with from 1-3 independently selected R^{Ca} For example, Ring C can be tetrahydropyranyl which is optionally substituted with from 1-3 independently R^{Ca}.

In some embodiments, Ring C is selected from the group consisting of:

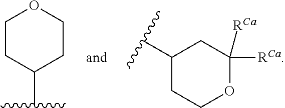

In some embodiments, Ring C is

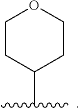 [SMILES: *C1CCOCC1]

In some embodiments, Ring C is

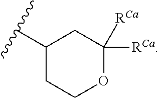

In some embodiments, R^{Ca} is independently C₁₋₆ alkyl.

In some embodiments, a pair of R^{Ca} on the same or different ring atoms, taken together with the ring atom(s) to which each is attached, forms a carbocyclic ring including from 3-6 ring atoms.

In some embodiments, a pair of R^{Ca} on the same ring atom, taken together with the ring atom to which each is attached, forms a carbocyclic ring including from 3-5 ring atoms.

In some embodiments, the compound of Formula I is a compound of Formula IIIA:

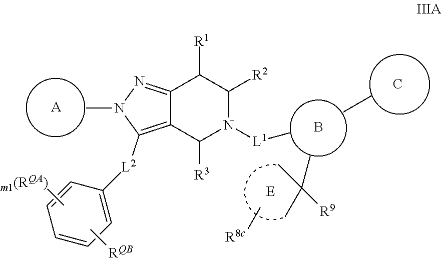

or a pharmaceutically acceptable salt, deuterated analog, or solvate thereof, wherein:
m1 is 0, 1, or 2;
R^{QB} is —(CR^{h}R^{h})_{q1}—S(O)₂—(CR^{h}R^{h})_{q2}—R^{e}, wherein R^{QB} is meta or para relative to L²;
q1 is 1 or 2;
Ring E is a C₃₋₆ cycloalkyl; and
R^{8c} is selected from the group consisting of H and C₁₋₆ alkyl optionally substituted with from 1-3 independently selected Rr.

In some embodiments of Formula IIIA, q2 is 0.

In some embodiments of Formula IIIA, q1 is 1.

In some embodiments of Formula IIIA, q1 is 2.

In some embodiments of Formula IIIA, each occurrence of R^{h} is H or C₁₋₃ alkyl. For example, each R^{h} can be H. As another non-limiting example, one occurrence of R^{h} can be C₁₋₃ alkyl (e.g., methyl); and each remaining R^{h} can be H.

In some embodiments of Formula IIIA, R^{c} is C₁₋₆ alkyl, C₁₋₆ haloalkyl, or C₃₋₆ cycloalkyl, each of which is optionally substituted with from 1-3 substituents each independently selected from the group consisting of: halo, cyano, C₁₋₃ alkyl, C₁₋₃ haloalkyl, —OH, NH₂, NH(C₁₋₃ alkyl), N(C₁₋₃ alkyl)₂, C₁₋₃ alkoxy, and C₁₋₃ haloalkoxy. In some embodiments, R^{e} is C₁₋₆ alkyl. For example, R^{c} can be C₁₋₃ alkyl, such as methyl or ethyl.

In some embodiments of Formula IIIA, R^{QB} is —CH₂—S(O)₂(C₁₋₆ alkyl). For example, R^{QB} can be —CH₂—S(O)₂(C₁₋₃ alkyl), such as —CH₂—S(O)₂Me or —CH₂—S(O)₂Et.

In some embodiments of Formula IIIA, R^{QB} is —CH(C₁₋₆ alkyl)—S(O)₂(C₁₋₆ alkyl). For example, R^{QB} can be —CH(C₁₋₃ alkyl)S(O)₂(C₁₋₃ alkyl), such as —CH(Me)S(O)₂Me.

In some embodiments of Formula IIIA, R^{QB} is —CH₂CH₂—S(O)₂(C₁₋₆ alkyl). For example, R^{QB} can be —CH₂CH₂—S(O)₂(C₁₋₃ alkyl), such as —CH₂CH₂—S(O)₂Me.

In some embodiments, the compound of Formula I is a compound of Formula IIIB:

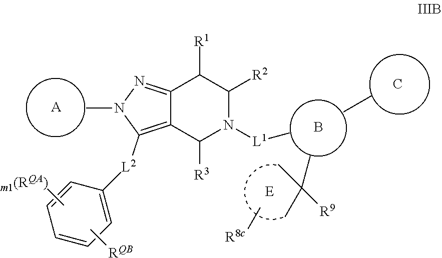

or a pharmaceutically acceptable salt, deuterated analog, or solvate thereof, wherein:
m1 is 0, 1, or 2;
R^{QB} is —S(O)₂N(R^{N2})—R^{e}, wherein R^{QB} is meta or para relative to L²;
R^{N2} is H or C₁₋₃ alkyl;
Ring E is a C₃₋₆ cycloalkyl; and
R^{8c} is selected from the group consisting of H and C₁₋₆ alkyl optionally substituted with from 1-3 independently selected R^{f}.

In some embodiments of Formula IIIB, R^{QB} is —S(O)₂N(H)—R^{c}.

In some embodiments of Formula IIIB, R^{c} is C₁₋₆ alkyl, C₁₋₆ haloalkyl, or C₃₋₆ cycloalkyl, each of which is optionally substituted with from 1-3 substituents each independently selected from the group consisting of: halo, cyano, C₁₋₃ alkyl, C₁₋₃ haloalkyl, —OH, NH₂, NH(C₁₋₃ alkyl), N(C₁₋₃ alkyl)₂, C₁₋₃ alkoxy, and C₁₋₃ haloalkoxy.

In some embodiments of Formula IIIB, R^{e} is C₁₋₆ alkyl. For example, R^{e} can be C₁₋₃ alkyl, such as methyl.

In some embodiments, the compound of Formula I is a compound of Formula IIIC:

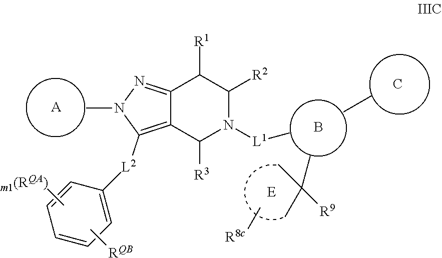

or a pharmaceutically acceptable salt, deuterated analog, or solvate thereof, wherein:
m1 is 0, 1, or 2;
R^{QB} is —N(R^{N2})S(O)₂—(CR^{h}R^{h})_{q2}—R^{c}, wherein R^{QB} is meta or para relative to L²;
R^{N2} is H or C₁₋₃ alkyl;
Ring E is a C₃₋₆ cycloalkyl; and
R^{8c} is selected from the group consisting of H and C₁₋₆ alkyl optionally substituted with from 1-3 independently selected R^{f}.

In some embodiments of Formula IIIC, R^{N2} is H.

In some embodiments of Formula IIIC, q2 is 0.

In some embodiments of Formula IIIC, R^{e} is C₁₋₆ alkyl, C₁₋₆ haloalkyl, or C₃₋₆ cycloalkyl, each of which is optionally substituted with from 1-3 substituents each independently selected from the group consisting of: halo, cyano, C₁₋₃ alkyl, C₁₋₃ haloalkyl, —OH, NH₂, NH(C₁₋₃ alkyl), N(C₁₋₃ alkyl)₂, C₁₋₃ alkoxy, and C₁₋₃ haloalkoxy.

In some embodiments of Formula IIIC, R^{c} is C₁₋₆ alkyl. For example, R^{c} can be C₁₋₃ alkyl, such as methyl or ethyl.

In some embodiments of Formula IIIC, R^{QB} is —N(H)S(O)₂(C₁₋₆ alkyl). For example, R^{QB} can be —N(H)S(O)₂(C₁₋₃ alkyl), such as —N(H)S(O)₂Me.

In some embodiments, the compound of Formula I is a compound of Formula IIID:

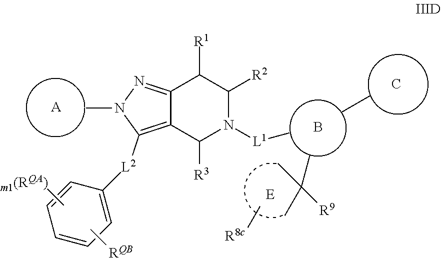

or a pharmaceutically acceptable salt, deuterated analog, or solvate thereof, wherein:
m1 is 0, 1, or 2;
R^{QB} is:

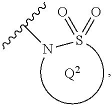

wherein Ring Q² is a 4-8 membered heterocyclyl including from 0-2 additional ring heteroatoms (in addition to the endocyclic N—S(O)₂ group) each independently selected from the group consisting of: N, O, and S(O)₀₋₂, wherein Ring Q² is optionally substituted with from 1-4 independently selected R^{g};
Ring E is a C₃₋₆ cycloalkyl; and
R^{8c} is selected from the group consisting of H and C₁₋₆ alkyl optionally substituted with from 1-3 independently selected R^{f}.

In some embodiments of Formula IIID, R^{QB} is:

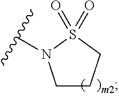

m2 is 0, 1, 2, or 3; and R^{QB} is optionally substituted with from 1-4 independently selected R^{g}.

In some embodiments of Formula IIID, R^{QB} is:

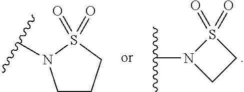 [SMILES: *N1CCCS1(=O)=O ; *N1CCS1(=O)=O]

In some embodiments of Formula IIIA, IIIB, IIIC, or HID, R^{QB} is meta to L².

In some embodiments of Formula IIIA, IIIB, IIIC, or IID, R^{QB} is para to L².

In some embodiments of Formula IIIA, IIIB, IIIC, or IID, m1 is 0.

In some embodiments of Formula IIIA, IIIB, IIIC, or IID, m1 is 1 or 2.

In some embodiments of Formula IIIA, IIIB, IIIC, or IIID, one occurrence of R^{QA} is halo. For example, one occurrence of R^{QA} can be —F.

In some embodiments of Formula IIIA, IIIB, IIIC, or IIID, Ring E is cyclopropyl.

In some embodiments of Formula IIIA, IIIB, IIIC, or IIID, Ring E is cyclobutyl.

In some embodiments of Formula IIIA, IIIB, IIIC, or IIID, R^{8c} is H.

In some embodiments of Formula IIIA, IIIB, IIIC, or IIID, R^{8c} is C₁₋₃ alkyl. For example, R^{8c} can be methyl.

In some embodiments of Formula IIIA, IIIB, IIIC, or IIID, R⁹ is:

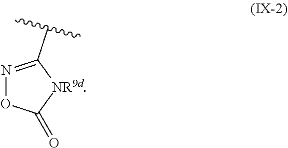

In some embodiments, R^{9d} is H.

In some embodiments of Formula IIIA, IIIB, IIIC, or IIID, the

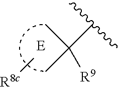

moiety is:

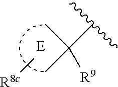

In some embodiments of Formula IIIA, IIIB, IIIC, or IIID, the

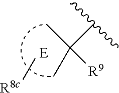

moiety is:

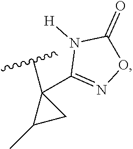 [SMILES: *C1(c2noc(=O)[nH]2)CC1C]

wherein each stereogenic center has (S)-configuration.

In some embodiments of Formula IIIA, IIIB, IIIC, or HID, Ring C is 3-12 membered heterocyclyl which is optionally substituted with from 1-3 independently selected R^{Ca}.

In some embodiments of Formula IIIA, IIIB, IIIC, or IID, Ring C is 5-6 membered heterocyclyl which is optionally substituted with from 1-3 independently selected R^{Ca}.

In some embodiments of Formula IIIA, IIIB, IIIC, or IID, Ring C is tetrahydropyranyl which is optionally substituted with from 1-3 independently R^{Ca}.

In some embodiments of Formula IIIA, IIIB, IIIC, or IID, Ring C is selected from the group consisting of:

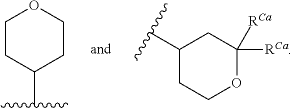

In some embodiments of Formula IIIA, IIIB, IIIC, or IIID, each R^{Ca} is independently C₁₋₆ alkyl.

In some embodiments of Formula IIIA, IIIB, IIIC, or IIID, a pair of R^{Ca} on the same or different ring atoms, taken together with the ring atom(s) to which each is attached, forms a carbocyclic ring including from 3-6 ring atoms.

In some embodiments of Formula IIIA, IIIB, IIIC, or IIID, R⁴, R⁵, R⁶, and R⁷ are each H.

In some embodiments of Formula IIIA, IIIB, IIIC, or IIID, L¹ is C(═O).

In some embodiments of Formula IIIA, IIIB, IIIC, or IIID, R¹ and R² are H.

In some embodiments of Formula IIIA, IIIB, IIIC, or IIID, R³ is C₁₋₃ alkyl.

In some embodiments of Formula IIIA, IIIB, IIIC, or IIID, R³ is methyl.

In some embodiments of Formula IIIA, IIIB, IIIC, or IIID, R¹ and R² are H; and R³ is C₁₋₃ alkyl (e.g., methyl).

In some embodiments of Formula IIIA, IIIB, IIIC, or IIID, Ring A is phenyl or pyridyl, each of which is optionally substituted with from 2-4 substituents each independently selected from the group consisting of halo, C₁₋₆ alkyl, C₁₋₆ haloalkyl, and C₁₋₆ alkoxy.

In some embodiments of Formula IIIA, IIIB, IIIC, or IIID, Ring A is phenyl, which is optionally substituted with from 2-4 substituents each independently selected from the group consisting of halo and C₁₋₆ alkyl.

In some embodiments of Formula IIIA, IIIB, IIIC, or HID, Ring A is:

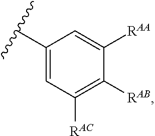

wherein R^{AA}, R^{AB}, and R^{AC} are each independently hydrogen, halo, C₁₋₃ alkyl, or C₃₋₆ cycloalkyl.

In some embodiments of Formula IIIA, IIIB, IIIC, or IID, Ring A is:

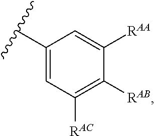

wherein R^{AA}, R^{AB}, and R^{A}C are independently halo or C₁₋₆ alkyl. In some embodiments, R^{AA} and R^{A}C are independently C₁₋₆ alkyl. For example, R^{AA} and R^{AC} can be independently selected C₁₋₃ alkyl, such as methyl. In some embodiments, R^{AB} is halo, such as —F.

In some embodiments of Formula IIIA, IIIB, IIIC, or IID, L² is:

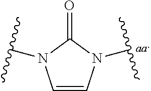 [SMILES: *n1ccn(*)c1=O]

In some embodiments, provided is a compound of Formula IV, or a pharmaceutically acceptable salt or solvate thereof, comprising any one or more of the features delineated below.

In some embodiments, Q is selected from the group consisting of: C₃₋₁₅ cycloalkyl; 3-12 membered heterocyclyl; 5-10 membered heteroaryl; and C₆₋₁₀ aryl, each of which is optionally substituted with from 1-6 independently selected R^{Q}. In some embodiments, Q is selected from the group consisting of: 5-10 membered heteroaryl and C₆₋₁₀ aryl, each of which is optionally substituted with from 1-6 independently selected R^{Q}. In some embodiments, Q is 5-10 membered heteroaryl optionally substituted with from 1-6 independently selected R^{Q}. In some embodiments, Q is 9-10 membered heteroaryl optionally substituted with from 1-6 independently selected R^{Q}. In some embodiments, Q is 9 membered heteroaryl optionally substituted with from 1-6 independently selected R^{Q}. In some embodiments, Q is 9 membered heteroaryl optionally substituted with from 1-2 independently selected R^{Q}.

In some embodiments, Q is

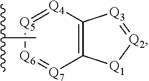

wherein: Q¹ is NH or NR^{Q}; Q² and Q³ are each independently N, CH, or CR^{Q}; one of Q⁴, Q⁵, Q⁶, and Q⁷ is a carbon atom bonded to L²; and the other three of Q⁴, Q⁵, Q⁶, and Q⁷ are each independently N, CH, or CR^{Q}. In some embodiments, Q¹ is NH or NR^{Q}; Q² and Q³ are each independently N, CH, or CR^{Q}; one of Q⁴, Q⁵, Q⁶, and Q⁷ is a carbon atom bonded to L²; and the other three of Q⁴, Q⁵, Q⁶, and Q⁷ are each independently CH or CR^{Q}. In some embodiments, Q¹ is NH or NR^{Q}; Q² and Q³ are each independently N, CH, or CR^{Q}; one of Q⁴, Q⁵, Q⁶, and Q⁷ is a carbon atom bonded to L²; and the other three of Q⁴, Q⁵, Q⁶, and Q⁷ are each independently CH or CR^{Q}.

In some embodiments, Q¹ is NH. In some embodiments, Q¹ is NR^{Q}.

In some embodiments, Q² is N and Q³ is N, CH or CR^{Q}.

In some embodiments, one of Q⁴, Q⁵, Q⁶, and Q⁷ is a carbon atom bonded to L²; two of Q⁴, Q⁵, Q⁶, and Q⁷ are each CH; and one of Q⁴, Q⁵, Q⁶, and Q⁷ is CH or CR^{Q} (e.g., CR^{Q}).

In some embodiments, one R^{Q} is present. In some embodiments, two R^{Q} are present. In some embodiments, three R^{Q} are present. In some embodiments, at least one R^{Q} is attached to the 6-membered ring. In some embodiments, two R^{Q} are present, and at least one of the two R^{Q} is attached to the 6-membered ring.

In some embodiments, Q is

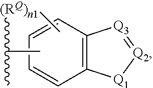

wherein: Q¹ is NH or NR^{Q}; Q² and Q³ are each independently N, CH, or CR^{Q}; and n1 is 0 or 1.

In some embodiments, Q¹ is NH. In some embodiments, Q¹ is NR^{Q}.

In some embodiments, Q² is N and Q³ is N, CH or CR^{Q}.

In some embodiments, one R^{Q} is present. In some embodiments, two R^{Q} are present. In some embodiments, three R^{Q} are present. In some embodiments, at least one R^{Q} is attached to the 6-membered ring. In some embodiments, two R^{Q} are present, and at least one of the two R^{Q} is attached to the 6-membered ring.

In some embodiments, Q is

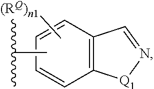

wherein: Q¹ is NH or NR^{Q}; and n1 is 0 or 1.

In some embodiments, Q¹ is NH. In some embodiments, Q¹ is NR^{Q}.

In some embodiments, one R^{Q} is present. In some embodiments, two R^{Q} are present. In some embodiments, two R^{Q} are present, and at least one of the two R^{Q} is attached to the 6-membered ring.

In some embodiments, Q is

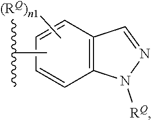

wherein n1 is 0 or 1.

In some embodiments, Q is

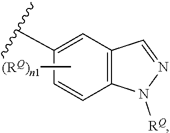

wherein n1 is 0 or 1.

In some embodiments, n1 is 0. In some embodiments, n1 is 1.

In some embodiments, Q is

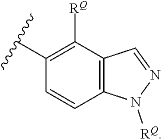

In some embodiments, Q is

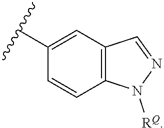

In some embodiments, when at least one R^{Q} (e.g., an R^{Q} attached to a ring N) is present, one R^{Q} is selected from the group consisting of:
(a) halo;
(b) cyano;
(c) OH;
(d) —NR^{c}R^{d};
(e) —C(═O)NR^{c}R^{d} or —S(O)₂NR^{c}R^{d};
(f) —S(═O)₀₋₂R^{e};
(g) C₁₋₆ alkyl optionally substituted with from 1-6 independently selected R^{f};
(h) C₁₋₆ alkoxy optionally substituted with from 1-6 independently selected R^{f};
(i) 3-12 membered heterocyclyl optionally substituted with from 1-6 independently selected R^{g};
(j) C₆₋₁₀ aryl optionally substituted with from 1-6 independently selected R^{g};
(k) 5-10 membered heteroaryl optionally substituted with from 1-6 independently selected R^{g};
(l) C₃₋₈ cycloalkyl optionally substituted with from 1-6 independently selected R^{g};
(m) P(═O)R^{a}R^{b}; and
(n) —(CR^{h}R^{h})_{q1}—S(O)₂-L^{c}-R^{c}, wherein q1 is 1, 2, or 3;
and each remaining R^{Q}, when present, is independently selected from: halo, cyano, OH, C₁₋₆ alkyl, and C₁₋₆ alkoxy.

In some embodiments, when at least one R^{Q} is present, one R^{Q} (e.g., an R^{Q} attached to a ring N) is selected from the group consisting of:
(a) halo;
(b) cyano;
(c) OH;
(d) —NR^{c}R^{d};
(e) —C(═O)NR^{c}R^{d} or —S(O)₂NR^{c}R^{d};
(f) —S(═O)₀₋₂R^{e};
(g) C₁₋₆ alkyl optionally substituted with from 1-6 independently selected R^{f}; and
(h) C₁₋₆ alkoxy optionally substituted with from 1-6 independently selected R^{f};
and each remaining R^{Q}, when present, is an independently selected halo.

In some embodiments, when at least one R^{Q} is present, one R^{Q} (e.g., an R^{Q} attached to a ring N) is an independently selected C₁₋₆ alkyl optionally substituted with from 1-6 independently selected R^{f}; and each remaining R^{Q}, when present, is independently selected halo. In some embodiments, when at least one R^{Q} is present, one R^{Q} (e.g., an R^{Q} attached to a ring N) is an independently selected C₁₋₃ alkyl optionally substituted with from 1-6 independently selected R^{f}; and each remaining R^{Q}, when present, is fluoro. In some embodiments, when at least one R^{Q} is present, one R^{Q} (e.g., an R^{Q} attached to a ring N) is an independently selected unsubstituted C₁₋₃ alkyl; and each remaining R^{Q}, when present, is fluoro. In some embodiments, when at least one R^{Q} is present, one R^{Q} (e.g., an R^{Q} attached to a ring N) is methyl; and each remaining R^{Q}, when present, is fluoro.

In some embodiments, two R^{Q} are present; one R^{Q} (e.g., an R^{Q} attached to a ring N) is an independently selected C₁₋₆ alkyl optionally substituted with from 1-6 independently selected R^{f}; and the other R^{Q} is halo. In some embodiments, two R^{Q} are present; one R^{Q} (e.g., an R^{Q} attached to a ring N) is an independently selected C₁₋₃ alkyl optionally substituted with from 1-6 independently selected R^{f}; and the other R^{Q} is fluoro. In some embodiments, two R^{Q} are present; one R^{Q} (e.g., an R^{Q} attached to a ring N) is an independently selected unsubstituted C₁₋₃ alkyl; and the other R^{Q}, is fluoro. In some embodiments, two R^{Q} are present; one R^{Q} (e.g., an R^{Q} attached to a ring N) is methyl; and the other R^{Q} is fluoro.

In some embodiments, one R^{Q} is present (e.g., an R^{Q} attached to a ring N) and is an independently selected C₁₋₆ alkyl optionally substituted with from 1-6 independently selected Rr. In some embodiments, one R^{Q} is present (e.g., an R^{Q} attached to a ring N) and is an independently selected C₁₋₃ alkyl optionally substituted with from 1-6 independently selected R^{f}. In some embodiments, one R^{Q} is present (e.g., an R^{Q} attached to a ring N) and is an independently selected unsubstituted C₁₋₃ alkyl. In some embodiments, one R^{Q} is present (e.g., an R^{Q} attached to a ring N) and is methyl.

In some embodiments, Q is

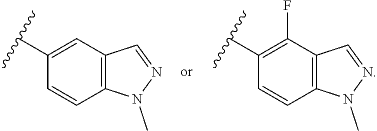 [SMILES: *c1ccc2c(cnn2C)c1 ; *c1ccc2c(cnn2C)c1F]

In some embodiments, Q is C₆₋₁₀ aryl optionally substituted with from 1-6 independently selected R^{Q}. In some embodiments, Q is phenyl optionally substituted with from 1-6 independently selected R^{Q}. In some embodiments, Q is phenyl optionally substituted with from 1-2 independently selected R^{Q}. In some embodiments, Q is phenyl optionally substituted with 1-2 independently selected R^{Q}.

In some embodiments, Q is:

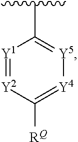

wherein Y¹, Y², Y⁴, and Y⁵ are each independently N, CH, or CR^{Q}. In some embodiments, Y¹, Y², Y⁴, and Y⁵ are each independently CH or CR^{Q}. In some embodiments, one or two of Y¹, Y², Y⁴, and Y⁵ are each independently CH or CR^{Q}; and the remaining Y¹, Y², Y⁴, and Y⁵ are CH.

In some embodiments, Q is

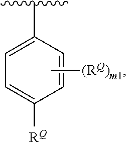

wherein m1 is 0, 1, or 2.

In some embodiments, the R^{Q} para to L² is selected from the group consisting of:
(e) —C(═O)NR^{c}R^{d} or —S(O)₂NR^{c}R^{d};
(f) —S(═O)₀₋₂R^{c};
(m) P(═O)R^{a}R^{b}; and
(n) —(CR^{h}R^{h})_{q1}—S(O)₂-L°—R^{c}.

In some embodiments, the R^{Q} para to L² is —P(═O)R^{a}R^{b}.

In some embodiments, R^{a} and R^{b} are independently selected C₁₋₆ alkyl optionally substituted with from 1-6 substituents each independently selected from the group consisting of C₁₋₆ alkoxy, C₃₋₆ cycloalkyl, and halo. In some embodiments, R^{a} and R^{b} are independently selected C₁₋₆ alkyl optionally substituted with from 1-2 substituents each independently selected from the group consisting of C₁₋₆ alkoxy, C₃₋₆ cycloalkyl, and halo. In some embodiments, R^{a} and R^{b} are independently selected C₁₋₃ alkyl optionally substituted with from 1-2 independently selected halo. In some embodiments, R^{a} and R^{b} are independently selected unsubstituted C₁₋₃ alkyl. In some embodiments, R^{a} and R^{b} are each ethyl.

In some embodiments, m1 is 0. In some embodiments, m1 is 1. In some embodiments, m1 is 2.

In some embodiments, Q is

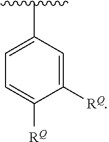

In some embodiments, the R^{Q} that is meta to L² is —NR^{c}R^{d}. In some embodiments, each R^{c} and R^{d} are independently selected from the group consisting of: H, C₁₋₆ alkyl, C(═O)(C₁₋₆ alkyl), wherein the C₁₋₆ alkyl and C(═O)(C_{1I6} alkyl) are each optionally substituted with from 1-6 substituents independently selected from the group consisting of: —OH, halo, and C₁₋₆ alkoxy. In some embodiments, each R^{c} and R^{d} are independently selected from the group consisting of: H, C₁₋₆ alkyl, C(═O)(C₁-6 alkyl), wherein the C₁₋₆ alkyl and C(═O)(C_{1I6} alkyl) are each optionally substituted with from 1-6 substituents independently selected from the group consisting of: —OH, halo, and C₁₋₆ alkoxy. In some embodiments, each R_{c} and R^{d} are independently selected from H and C₁₋₆ alkyl, wherein the C₁₋₆ alkyl is optionally substituted with from 1-6 substituents independently selected from the group consisting of: —OH, halo, and C₁₋₆ alkoxy. In some embodiments, each R^{c} and R^{d} are independently selected from H and C₁₋₃ alkyl. In some embodiments, one of R^{c} and R^{d} is H, and the other of R^{c} and R^{d} is C₁₋₃ alkyl. In some embodiments, one of R^{c} and R^{d} is H, and the other of R^{c} and R^{d} is methyl.

In some embodiments, Q is

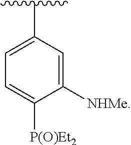

In some embodiments, R¹ is H. In some embodiments, R² is H. In some embodiments, R³ is C₁₋₆ alkyl. For example, R³ can be C₁₋₃ alkyl such as methyl. In some embodiments, R³ is C₁₋₆ alkyl, and the carbon atom to which R³ is attached has (S)-configuration.

In some embodiments, R¹, R², and R³ are H.

In some embodiments, R¹ and R² are H; and R³ is C₁₋₆ alkyl. In some embodiments, R¹ and R² are H; and R³ is methyl. In some embodiments, the carbon atom to which R³ is attached has (S)-configuration.

In some embodiments, L² is:

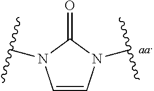 [SMILES: *n1ccn(*)c1=O]

In some embodiments, Ring A is C₆₋₁₀ aryl or 5-10 membered heteroaryl, each of which is optionally substituted with from 1-4 substituents each independently selected from the group consisting of halo, C₁₋₆ alkyl, C₁₋₆ haloalkyl, and C₁₋₆ alkoxy.

In some embodiments, Ring A is phenyl or pyridyl, each of which is optionally substituted with from 2-4 substituents each independently selected from the group consisting of halo, C₁₋₆ alkyl, C₁₋₆ haloalkyl, and C₁₋₆ alkoxy.

In some embodiments, Ring A is phenyl, which is optionally substituted with from 2-4 substituents each independently selected from the group consisting of halo and C₁₋₆ alkyl.

In some embodiments, Ring A is:

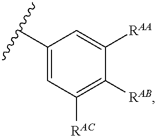

wherein R^{AA}, R^{AB}, and R^{AC} are each independently hydrogen, halo, C₁₋₃ alkyl, or C₃₋₆ cycloalkyl.

In some embodiments, Ring A is:

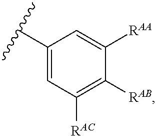

wherein R^{AA}, R^{AB}, and R^{AC} are independently halo or C₁₋₆ alkyl. In some embodiments, R^{AA} and R^{A}C are independently C₁₋₆ alkyl (e.g., C₁₋₃ alkyl, such as methyl). In some embodiments, R^{AB} is halo (e.g., —F).

In some embodiments, Ring A is pyridyl, which is optionally substituted with from 2-4 substituents each independently selected from the group consisting of halo and C₁₋₆ alkyl.

In some embodiments, L¹ is C(═O).

In some embodiments, R⁴, R⁵, and R⁶ are each H or halo. In some embodiments, R⁴, R⁵, and R⁶ are each H or —F. For example, R⁴, R⁵, and R⁶ can each be H. As another non-limiting example, R⁴ and R⁵ can be H; and R⁶ can be —F.

In some embodiments, at least one of L³ and L⁴ is a bond. In some embodiments, both of L³ and L⁴ are bonds. In some embodiments, L³ is a bond; and L⁴ is C₁₋₂ alkylene. In some embodiments, L⁴ is a bond; and L³ is C₁₋₂ alkylene.

In some embodiments, L³ and L⁴ are each independently C₁₋₂ alkylene.

In some embodiments, R^{8a} and R^{8b} taken together with the carbon atom to which each is attached forms a C₃₋₈ cycloalkyl ring which is optionally substituted with from 1-2 (e.g., 1) independently selected C₁₋₆ alkyl, wherein the C₁₋₆ alkyl is optionally substituted with from 1-3 independently selected R^{f}. In some embodiments, L³ is a bond; and L⁴ is a bond.

In some embodiments, R^{8a} and R^{8b} taken together with the carbon atom to which each is attached forms a C₃₋₅ (e.g., C₃ or C₄) cycloalkyl ring which is optionally substituted with from 1-2 (e.g., 1) independently selected C₁₋₆ alkyl, wherein the C₁₋₆ alkyl is optionally substituted with from 1-3 independently selected R^{f}. In some embodiments, L³ is a bond; and L⁴ is a bond.

In some embodiments, R^{8a} and R^{8b} taken together with the carbon atom to which each is attached forms a C₃₋₄ cycloalkyl ring which is optionally substituted with from 1-2 (e.g., 1) independently selected C₁₋₆ alkyl.

In some embodiments, R^{8a} and R^{8b} taken together with the carbon atom to which each is attached forms:

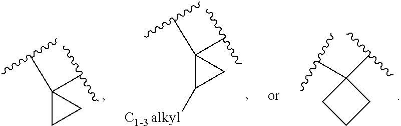

In some embodiments, R⁸ and R^{8b} taken together with the carbon atom to which each is attached forms:

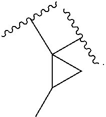 [SMILES: *C1(*)CC1C]

In some embodiments, R⁹ is:

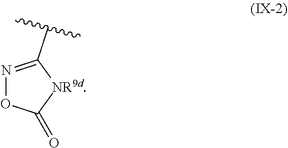

In some embodiments, R^{9d} is H or C₁₋₆ alkyl. For example, R^{9d} can be H.

In some embodiments, the L³-C(R^{8a}R^{8b})-L⁴-R⁹ moiety is:

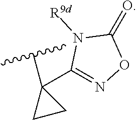

In some embodiments, the L³-C(R^{8a}R^{8b})-L⁴-R⁹ moiety is:

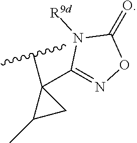

In some embodiments, the L³-C(R^{8a}R^{8b})-L⁴-R⁹ moiety is:

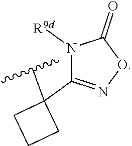

In some embodiments, the L³-C(R^{8a}R^{8b})-L⁴-R⁹ moiety is:

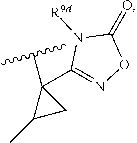

wherein each stereogenic center has (S)-configuration.

In some embodiments, R⁹ is (IX-2), wherein the L³-C(R^{8a}R^{8b})-L⁴-R⁹ moiety is:

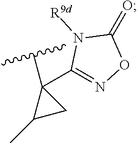

wherein R^{9d} is H or C₁₋₆ alkyl. For example, R^{9d} can be H. In some embodiments, each stereogenic center of

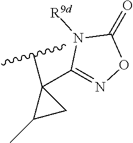

has (S)-configuration.

In some embodiments, R⁹ is C(═O)OH.

In some embodiments, Ring C is 3-12 membered heterocyclyl which is optionally substituted with from 1-3 independently selected R^{Ca}. In some embodiments, Ring C is 4-8 membered heterocyclyl which is optionally substituted with from 1-3 independently selected R^{Ca}. In some embodiments, Ring C is 5-6 membered heterocyclyl which is optionally substituted with from 1-3 independently selected R^{Ca}. For example, Ring C can be tetrahydropyranyl which is optionally substituted with from 1-3 independently R^{Ca}.

In some embodiments, Ring C is selected from the group consisting of:

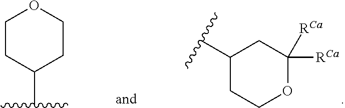

In some embodiments, Ring C is

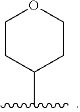 [SMILES: *C1CCOCC1]

In some embodiments, Ring C is

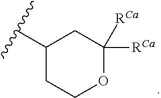

In some embodiments, R^{Ca} is independently C₁₋₆ alkyl.

In some embodiments, a pair of R^{Ca} on the same or different ring atoms, taken together with the ring atom(s) to which each is attached, forms a carbocyclic ring including from 3-6 ring atoms.

In some embodiments, a pair of R^{Ca} on the same ring atom, taken together with the ring atom to which each is attached, forms a carbocyclic ring including from 3-5 ring atoms.

In some embodiments, the compound of Formula I is a compound of Formula IVA:

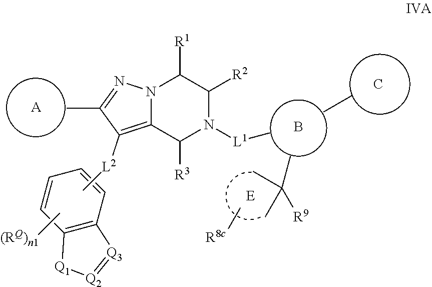

or a pharmaceutically acceptable salt, deuterated analog, or solvate thereof, wherein:
Q¹ is NH or NR^{Q};
Q² and Q³ are each independently N, CH, or CR^{Q};
n1 is 0 or 1;
Ring E is a C₃₋₆ cycloalkyl; and
R^{8c} is selected from the group consisting of H and C₁₋₆ alkyl optionally substituted with from 1-3 independently selected R^{f}.

In some embodiments of Formula IVA, Q¹ is NR^{Q}.

In some embodiments of Formula IVA, the R^{Q} attached to N is selected from the group consisting of:
(a) halo;
(b) cyano;
(c) OH;
(d) —NR^{c}R^{d};
(e) —C(═O)NR^{c}R^{d} or —S(O)₂NR^{c}R^{d};
(f) —S(═O)₀₋₂R^{e};
(g) C₁₋₆ alkyl optionally substituted with from 1-6 independently selected R^{f}; and
(h) C₁₋₆ alkoxy optionally substituted with from 1-6 independently selected R^{f}.

As a non-limiting example, the R^{Q} attached to N can be unsubstituted C₁₋₃ alkyl.

In some embodiments of Formula IVA, Q² and Q³ are each independently N or CH. As a non-limiting example, Q² is N and Q³ can be CH.

In some embodiments of Formula IVA, n1 is 0. In some embodiments of Formula IVA, n1 is 1.

In some embodiments of Formula IVA, the R^{Q} attached to the 6-membered ring is selected from halo, cyano, OH, C₁₋₆ alkyl, and C₁₋₆ alkoxy. In some embodiments, the R^{Q} attached to the 6-membered ring is halo.

In some embodiments of Formula IVA, L² attaches para to Q¹.

In some embodiments of Formula IVA, the R^{Q} attached to the 6-membered ring is attached at the position ortho to Q³.

In some embodiments, the compound of Formula I is a compound of Formula IVB:

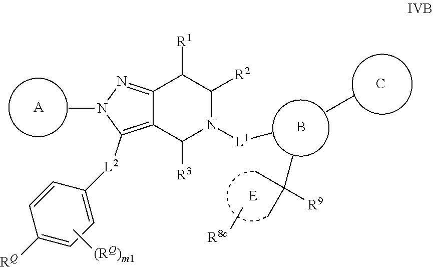

or a pharmaceutically acceptable salt, deuterated analog, or solvate thereof, wherein:
m1 is 0, 1, or 2;
the R^{Q} para to L² is —P(═O)R^{a}R^{b};
Ring E is a C₃₋₆ cycloalkyl; and
R^{8c} is selected from the group consisting of H and C₁₋₆ alkyl optionally substituted with from 1-3 independently selected Rr.

In some embodiments of Formula IVB, R^{a} and R^{b} are independently selected C₁₋₆ alkyl optionally substituted with from 1-2 substituents each independently selected from the group consisting of C₁₋₆ alkoxy, C₃₋₆ cycloalkyl, and halo. In some embodiments, R^{a} and R^{b} are independently selected unsubstituted C₁₋₃ alkyl.

In some embodiments of Formula IVB, m1 is 1 and the R^{Q} that is not para to L² is meta to L². In some embodiments, the R^{Q} that is meta to L² is —NR^{c}R^{d}.

In some embodiments of Formula IVB, each R^{c} and R^{d} are independently selected from the group consisting of: H, C₁₋₆ alkyl, C(═O)(C₁₋₆ alkyl), wherein the C₁₋₆ alkyl and C(═O)(C₁₋₆ alkyl) are each optionally substituted with from 1-6 substituents independently selected from the group consisting of: —OH, halo, and C₁₋₆ alkoxy. In some embodiments, one of R^{c} and R^{d} is H, and the other of R^{c} and R^{d} is methyl.

In some embodiments of Formula IVA or IVB, Ring E is cyclopropyl.

In some embodiments of Formula IVA or IVB, Ring E is cyclobutyl.

In some embodiments of Formula IVA or IVB, R^{8c} is H.

In some embodiments of Formula IVA or IVB, R^{8c} is C₁₋₃ alkyl. For example, R^{8c} can be methyl.

In some embodiments of Formula IVA or IVB, R⁹ is:

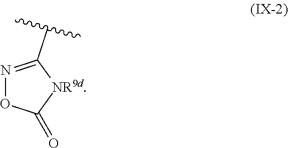

In some embodiments, R^{9d} is H.

In some embodiments of Formula IVA or IVB, the

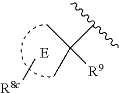

moiety is:

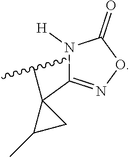 [SMILES: *C1(c2noc(=O)[nH]2)CC1C]

In some embodiments of Formula IVA or IVB, the

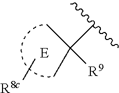

moiety is:

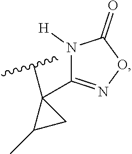 [SMILES: *C1(c2noc(=O)[nH]2)CC1C]

wherein each stereogenic center in has (S)-configuration.

In some embodiments of Formula IVA or IVB, the

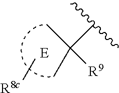

moiety is:

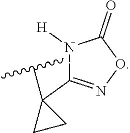 [SMILES: *C1(c2noc(=O)[nH]2)CC1]

In some embodiments of Formula IVA or IVB,

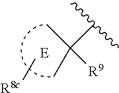

moiety is:

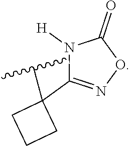 [SMILES: *C1(c2noc(=O)[nH]2)CCC1]

In some embodiments of Formula IVA or IVB, R⁹ is C(═O)OH.

In some embodiments of Formula IVA or IVB, Ring C is 3-12 membered heterocyclyl which is optionally substituted with from 1-3 independently selected R^{Ca}.

In some embodiments of Formula IVA or IVB, Ring C is 5-6 membered heterocyclyl which is optionally substituted with from 1-3 independently selected R^{Ca}.

In some embodiments of Formula IVA or IVB, Ring C is tetrahydropyranyl which is optionally substituted with from 1-3 independently R^{Ca}.

In some embodiments of Formula IVA or IVB, Ring C is selected from the group consisting of:

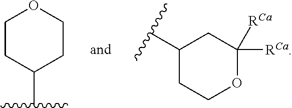

In some embodiments of Formula IVA or IVB, each R^{Ca} is independently C₁₋₆ alkyl.

In some embodiments of Formula IVA or IVB, a pair of R^{Ca} on the same or different ring atoms, taken together with the ring atom(s) to which each is attached, forms a carbocyclic ring including from 3-6 ring atoms.

In some embodiments of Formula IVA or IVB, R⁴, R⁵, R⁶, and R⁷ are each H.

In some embodiments of Formula IVA or IVB, L¹ is C(═O).

In some embodiments of Formula IVA or IVB, R¹ and R² are H.

In some embodiments of Formula IVA or IVB, R³ is C₁₋₃ alkyl.

In some embodiments of Formula IVA or IVB, R³ is methyl.

In some embodiments of Formula IVA or IVB, R¹ and R² are H; and R³ is C₁₋₃ alkyl (e.g., methyl).

In some embodiments of Formula IVA or IVB, Ring A is phenyl or pyridyl, each of which is optionally substituted with from 2-4 substituents each independently selected from the group consisting of halo, C₁₋₆ alkyl, C₁₋₆ haloalkyl, and C₁₋₆ alkoxy.

In some embodiments of Formula IVA or IVB, Ring A is phenyl, which is optionally substituted with from 2-4 substituents each independently selected from the group consisting of halo and C₁₋₆ alkyl.

In some embodiments of Formula IVA or IVB, Ring A is:

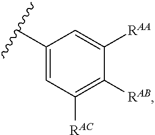

wherein R^{AA}, R^{AB} and R^{AC} are independently halo or C₁₋₆ alkyl. In some embodiments, R^{AA} and R^{A}C are each independently C₁₋₆ alkyl. In some embodiments, R^{AA} and R^{A}C are independently selected from C₁₋₃ alkyl, such as methyl. In some embodiments, R^{AB} is halo, such as —F.

In some embodiments of Formula IVA or IVB, L² is:

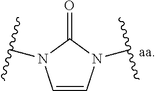 [SMILES: *n1ccn(*)c1=O]

In some embodiments, provided is a compound selected from Table 1, or a pharmaceutically acceptable salt, deuterated analog, or solvate thereof.

TABLE 1

| Ex. | Structure |
|---|---|
| 1 | 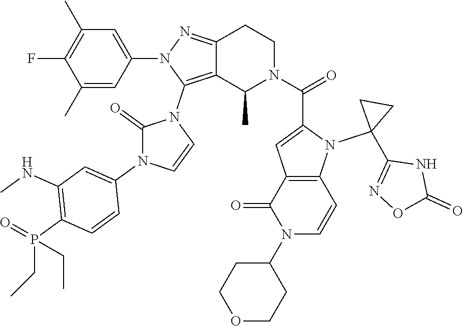 [SMILES: CCP(=O)(CC)c1ccc(-n2ccn(-c3c4c(nn3-c3cc(C)c(F)c(C)c3)CCN(C(=O)c3cc5c(=O)n(C6CCOCC6)ccc5n3C3(c5noc(=O)[nH]5)CC3)[C@H]4C)c2=O)cc1NC] |
| 3 | 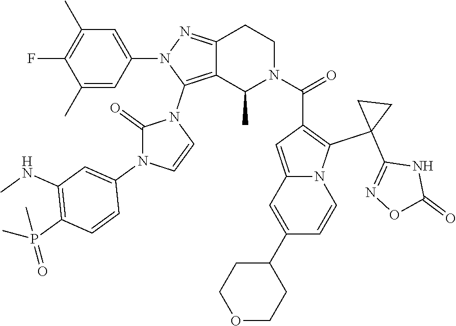 [SMILES: CNc1cc(-n2ccn(-c3c4c(nn3-c3cc(C)c(F)c(C)c3)CCN(C(=O)c3cc5cc(C6CCOCC6)ccn5c3C3(c5noc(=O)[nH]5)CC3)[C@H]4C)c2=O)ccc1P(C)(C)=O] |
| 4 | 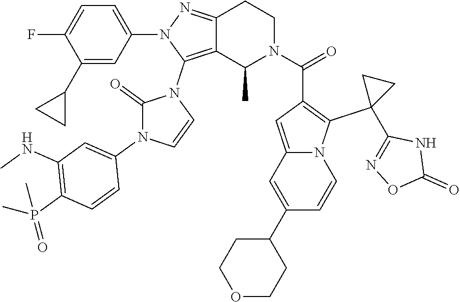 [SMILES: CNc1cc(-n2ccn(-c3c4c(nn3-c3ccc(F)c(C5CC5)c3)CCN(C(=O)c3cc5cc(C6CCOCC6)ccn5c3C3(c5noc(=O)[nH]5)CC3)[C@H]4C)c2=O)ccc1P(C)(C)=O] |
| 5 | 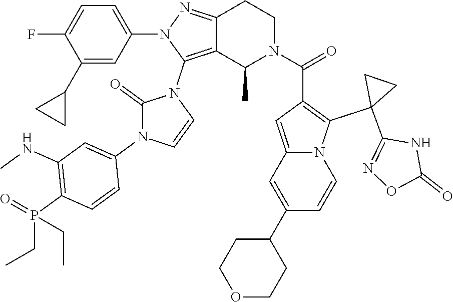 [SMILES: CCP(=O)(CC)c1ccc(-n2ccn(-c3c4c(nn3-c3ccc(F)c(C5CC5)c3)CCN(C(=O)c3cc5cc(C6CCOCC6)ccn5c3C3(c5noc(=O)[nH]5)CC3)[C@H]4C)c2=O)cc1NC] |
| 6 | 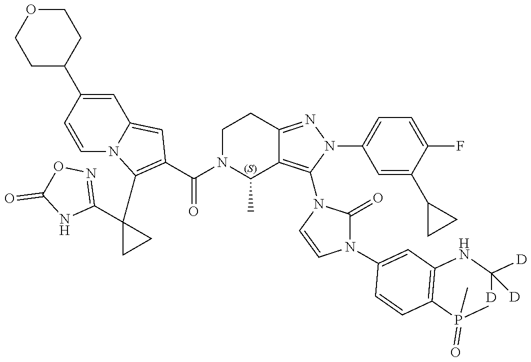 |
| 7 | 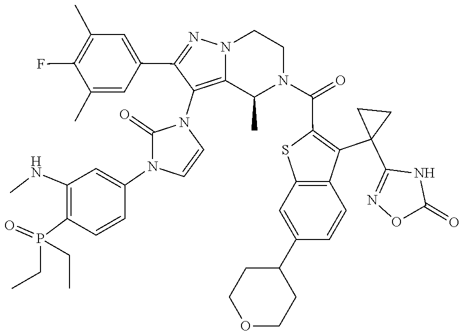 [SMILES: CCP(=O)(CC)c1ccc(-n2ccn(-c3c(-c4cc(C)c(F)c(C)c4)nn4c3[C@H](C)N(C(=O)c3sc5cc(C6CCOCC6)ccc5c3C3(c5noc(=O)[nH]5)CC3)CC4)c2=O)cc1NC] |
| 8 | 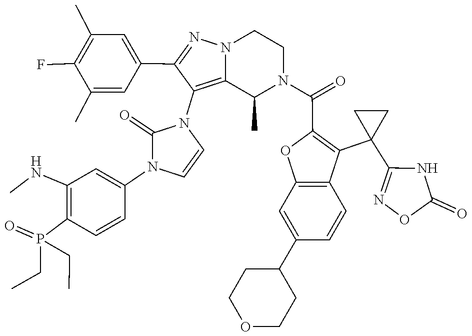 [SMILES: CCP(=O)(CC)c1ccc(-n2ccn(-c3c(-c4cc(C)c(F)c(C)c4)nn4c3[C@H](C)N(C(=O)c3oc5cc(C6CCOCC6)ccc5c3C3(c5noc(=O)[nH]5)CC3)CC4)c2=O)cc1NC] |
| 9 | 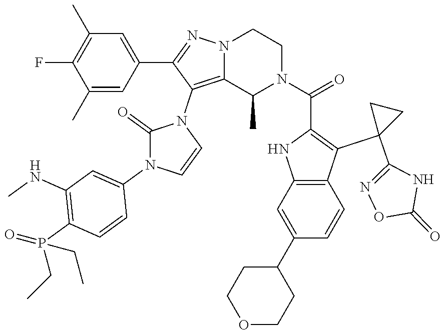 [SMILES: CCP(=O)(CC)c1ccc(-n2ccn(-c3c(-c4cc(C)c(F)c(C)c4)nn4c3[C@H](C)N(C(=O)c3[nH]c5cc(C6CCOCC6)ccc5c3C3(c5noc(=O)[nH]5)CC3)CC4)c2=O)cc1NC] |
| 10 | 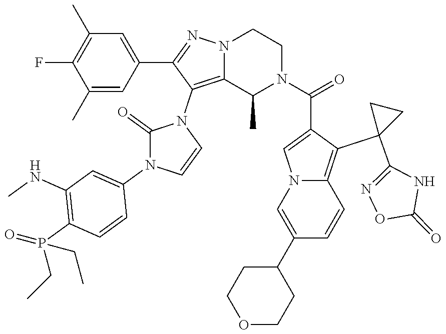 [SMILES: CCP(=O)(CC)c1ccc(-n2ccn(-c3c(-c4cc(C)c(F)c(C)c4)nn4c3[C@H](C)N(C(=O)c3cn5cc(C6CCOCC6)ccc5c3C3(c5noc(=O)[nH]5)CC3)CC4)c2=O)cc1NC] |
| 11 | 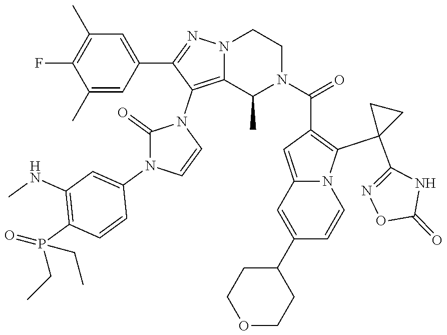 [SMILES: CCP(=O)(CC)c1ccc(-n2ccn(-c3c(-c4cc(C)c(F)c(C)c4)nn4c3[C@H](C)N(C(=O)c3cc5cc(C6CCOCC6)ccn5c3C3(c5noc(=O)[nH]5)CC3)CC4)c2=O)cc1NC] |
| 12 | 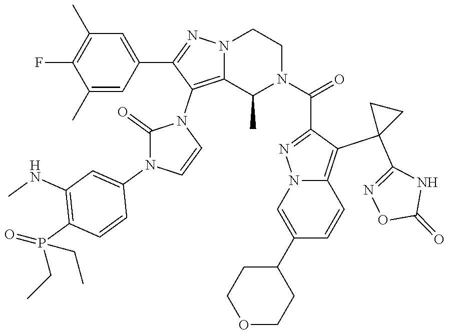 [SMILES: CCP(=O)(CC)c1ccc(-n2ccn(-c3c(-c4cc(C)c(F)c(C)c4)nn4c3[C@H](C)N(C(=O)c3nn5cc(C6CCOCC6)ccc5c3C3(c5noc(=O)[nH]5)CC3)CC4)c2=O)cc1NC] |
| 13 | 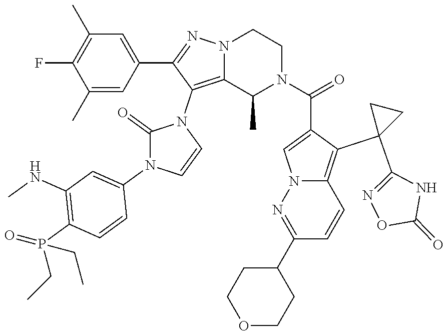 [SMILES: CCP(=O)(CC)c1ccc(-n2ccn(-c3c(-c4cc(C)c(F)c(C)c4)nn4c3[C@H](C)N(C(=O)c3cn5nc(C6CCOCC6)ccc5c3C3(c5noc(=O)[nH]5)CC3)CC4)c2=O)cc1NC] |
| 14 | 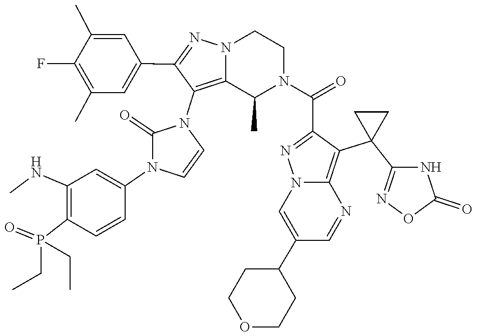 [SMILES: CCP(=O)(CC)c1ccc(-n2ccn(-c3c(-c4cc(C)c(F)c(C)c4)nn4c3[C@H](C)N(C(=O)c3nn5cc(C6CCOCC6)cnc5c3C3(c5noc(=O)[nH]5)CC3)CC4)c2=O)cc1NC] |
| 15 | 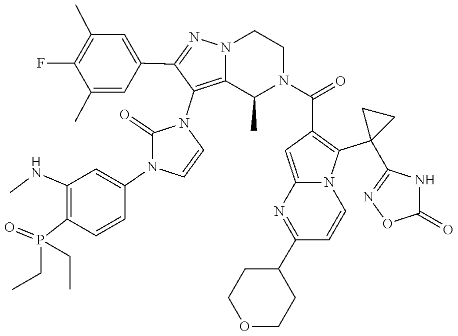 [SMILES: CCP(=O)(CC)c1ccc(-n2ccn(-c3c(-c4cc(C)c(F)c(C)c4)nn4c3[C@H](C)N(C(=O)c3cc5nc(C6CCOCC6)ccn5c3C3(c5noc(=O)[nH]5)CC3)CC4)c2=O)cc1NC] |
| 16 | 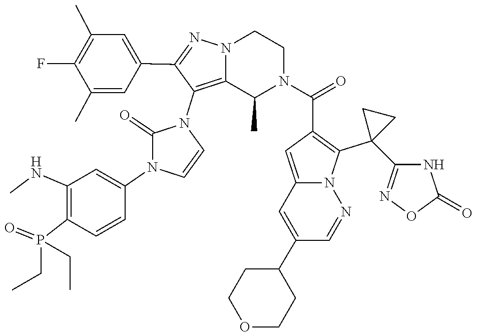 [SMILES: CCP(=O)(CC)c1ccc(-n2ccn(-c3c(-c4cc(C)c(F)c(C)c4)nn4c3[C@H](C)N(C(=O)c3cc5cc(C6CCOCC6)cnn5c3C3(c5noc(=O)[nH]5)CC3)CC4)c2=O)cc1NC] |
| 17 | 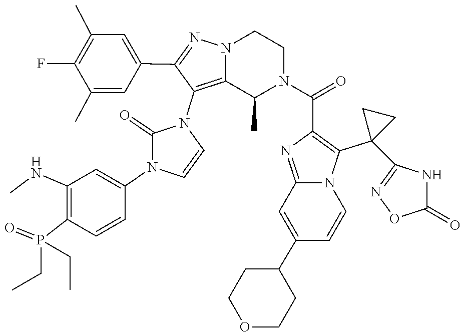 [SMILES: CCP(=O)(CC)c1ccc(-n2ccn(-c3c(-c4cc(C)c(F)c(C)c4)nn4c3[C@H](C)N(C(=O)c3nc5cc(C6CCOCC6)ccn5c3C3(c5noc(=O)[nH]5)CC3)CC4)c2=O)cc1NC] |
| 18 | 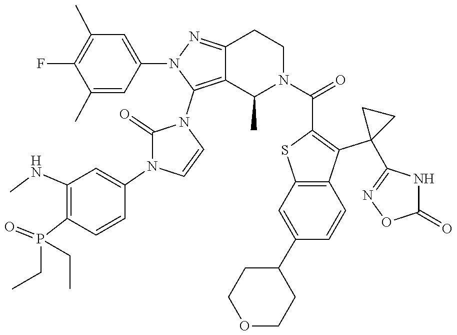 [SMILES: CCP(=O)(CC)c1ccc(-n2ccn(-c3c4c(nn3-c3cc(C)c(F)c(C)c3)CCN(C(=O)c3sc5cc(C6CCOCC6)ccc5c3C3(c5noc(=O)[nH]5)CC3)[C@H]4C)c2=O)cc1NC] |
| 19 | 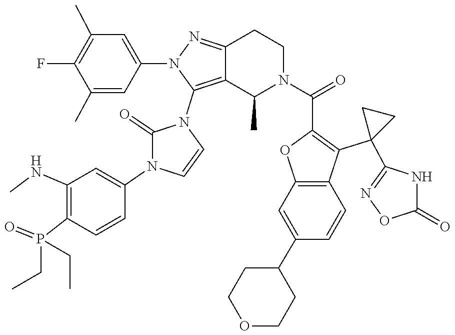 [SMILES: CCP(=O)(CC)c1ccc(-n2ccn(-c3c4c(nn3-c3cc(C)c(F)c(C)c3)CCN(C(=O)c3oc5cc(C6CCOCC6)ccc5c3C3(c5noc(=O)[nH]5)CC3)[C@H]4C)c2=O)cc1NC] |
| 20 | 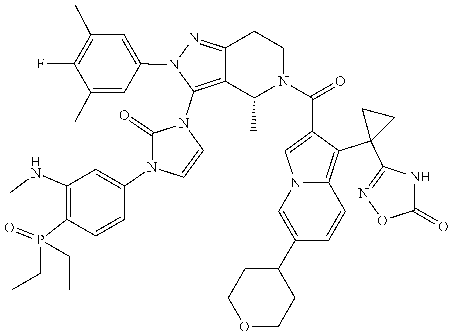 [SMILES: CCP(=O)(CC)c1ccc(-n2ccn(-c3c4c(nn3-c3cc(C)c(F)c(C)c3)CCN(C(=O)c3cn5cc(C6CCOCC6)ccc5c3C3(c5noc(=O)[nH]5)CC3)[C@@H]4C)c2=O)cc1NC] |
| 21 | 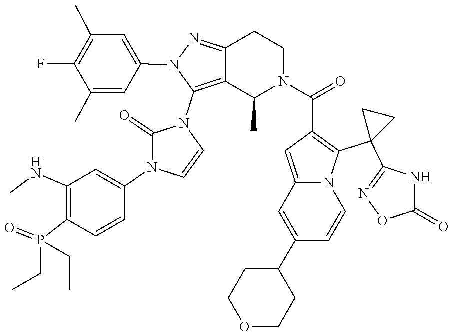 [SMILES: CCP(=O)(CC)c1ccc(-n2ccn(-c3c4c(nn3-c3cc(C)c(F)c(C)c3)CCN(C(=O)c3cc5cc(C6CCOCC6)ccn5c3C3(c5noc(=O)[nH]5)CC3)[C@H]4C)c2=O)cc1NC] |
| 22 | 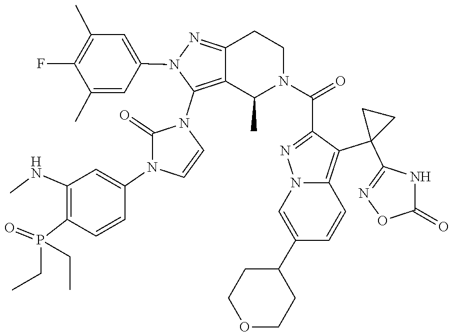 [SMILES: CCP(=O)(CC)c1ccc(-n2ccn(-c3c4c(nn3-c3cc(C)c(F)c(C)c3)CCN(C(=O)c3nn5cc(C6CCOCC6)ccc5c3C3(c5noc(=O)[nH]5)CC3)[C@H]4C)c2=O)cc1NC] |
| 23 | 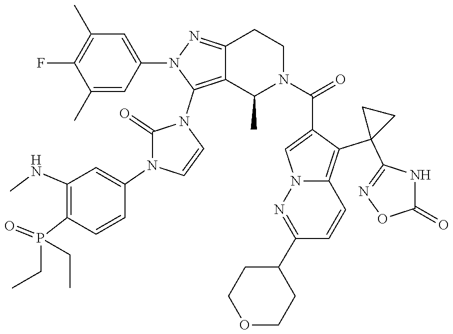 [SMILES: CCP(=O)(CC)c1ccc(-n2ccn(-c3c4c(nn3-c3cc(C)c(F)c(C)c3)CCN(C(=O)c3cn5nc(C6CCOCC6)ccc5c3C3(c5noc(=O)[nH]5)CC3)[C@H]4C)c2=O)cc1NC] |
| 24 | 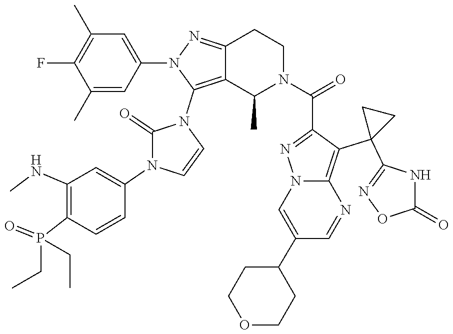 [SMILES: CCP(=O)(CC)c1ccc(-n2ccn(-c3c4c(nn3-c3cc(C)c(F)c(C)c3)CCN(C(=O)c3nn5cc(C6CCOCC6)cnc5c3C3(c5noc(=O)[nH]5)CC3)[C@H]4C)c2=O)cc1NC] |
| 25 | 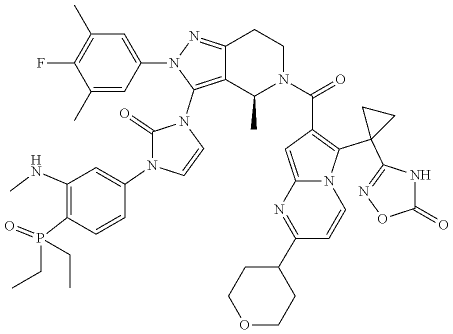 [SMILES: CCP(=O)(CC)c1ccc(-n2ccn(-c3c4c(nn3-c3cc(C)c(F)c(C)c3)CCN(C(=O)c3cc5nc(C6CCOCC6)ccn5c3C3(c5noc(=O)[nH]5)CC3)[C@H]4C)c2=O)cc1NC] |
| 26 | 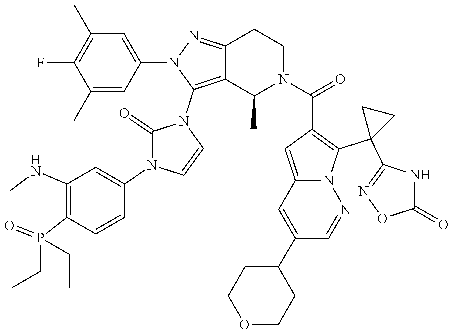 [SMILES: CCP(=O)(CC)c1ccc(-n2ccn(-c3c4c(nn3-c3cc(C)c(F)c(C)c3)CCN(C(=O)c3cc5cc(C6CCOCC6)cnn5c3C3(c5noc(=O)[nH]5)CC3)[C@H]4C)c2=O)cc1NC] |
| 27 | 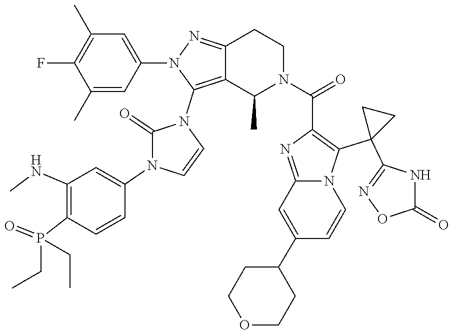 [SMILES: CCP(=O)(CC)c1ccc(-n2ccn(-c3c4c(nn3-c3cc(C)c(F)c(C)c3)CCN(C(=O)c3nc5cc(C6CCOCC6)ccn5c3C3(c5noc(=O)[nH]5)CC3)[C@H]4C)c2=O)cc1NC] |
| 28 | 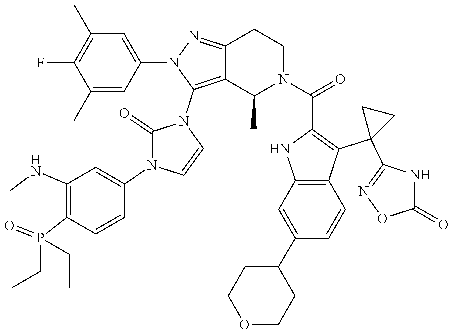 [SMILES: CCP(=O)(CC)c1ccc(-n2ccn(-c3c4c(nn3-c3cc(C)c(F)c(C)c3)CCN(C(=O)c3[nH]c5cc(C6CCOCC6)ccc5c3C3(c5noc(=O)[nH]5)CC3)[C@H]4C)c2=O)cc1NC] |
| 29 | 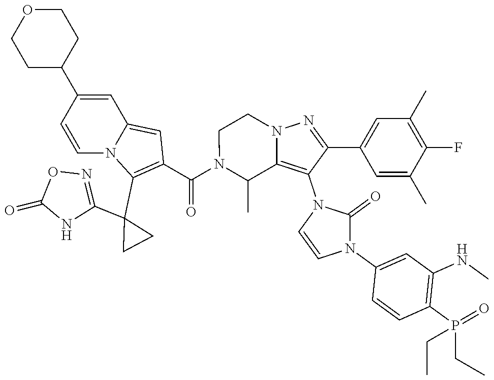 [SMILES: CCP(=O)(CC)c1ccc(-n2ccn(-c3c(-c4cc(C)c(F)c(C)c4)nn4c3C(C)N(C(=O)c3cc5cc(C6CCOCC6)ccn5c3C3(c5noc(=O)[nH]5)CC3)CC4)c2=O)cc1NC] |
| 30 | 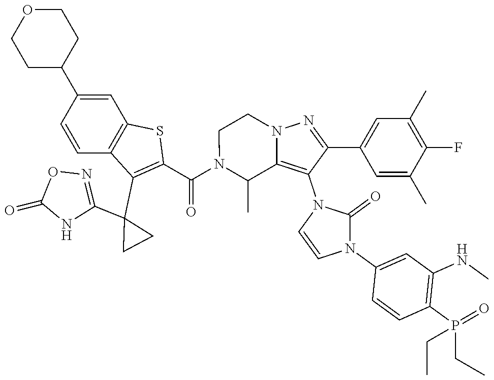 [SMILES: CCP(=O)(CC)c1ccc(-n2ccn(-c3c(-c4cc(C)c(F)c(C)c4)nn4c3C(C)N(C(=O)c3sc5cc(C6CCOCC6)ccc5c3C3(c5noc(=O)[nH]5)CC3)CC4)c2=O)cc1NC] |

The compounds of Formula I include pharmaceutically acceptable salts thereof. In addition, the compounds of Formula I also include other salts of such compounds which are not necessarily pharmaceutically acceptable salts, and which may be useful as intermediates for preparing and/or purifying compounds of Formula I and/or for separating enantiomers of compounds of Formula I. Non-limiting examples of pharmaceutically acceptable salts of compounds of Formula I include trifluoroacetic acid salts.

It will further be appreciated that the compounds of Formula I or their salts may be isolated in the form of solvates, and accordingly that any such solvate is included within the scope of the present disclosure. For example, compounds of Formula I and salts thereof can exist in unsolvated as well as solvated forms with pharmaceutically acceptable solvents such as water, ethanol, and the like.

### Treatment Methods and Uses

In some embodiments, this disclosure features methods for treating a patient (e.g., a human) having a disease, disorder, or condition in which modulation of GLP-1R (e.g., repressed or impaired and/or elevated or unwanted GLP-1R) is beneficial for the treatment of the underlying pathology and/or symptoms and/or progression of the disease, disorder, or condition. In some embodiments, the methods described herein can include or further include treating one or more conditions associated, co-morbid or sequela with any one or more of the conditions described herein.

Provided herein is a method for treating a GLP-1 associated disease, disorder, or condition, the method comprising administering to a patient in need thereof an effective amount of a compound of Formula I, or a pharmaceutically acceptable salt, deuterated analog, or solvate thereof (e.g., a compound of any one of Formula II, III, or IV, or a pharmaceutically acceptable salt, deuterated analog, or solvate thereof), or a pharmaceutical composition as disclosed herein.

In some embodiments, the disease, disorder, or condition includes, but is not limited to type 1 diabetes mellitus, type 2 diabetes mellitus, early onset type 2 diabetes mellitus, idiopathic type 1 diabetes mellitus (Type 1b), youth-onset atypical diabetes (YOAD), maturity onset diabetes of the young (MODY), latent autoimmune diabetes in adults (LADA), obesity (including hypothalamic obesity and monogenic obesity), weight gain from use of other agents, idiopathic intracranial hypertension, Wolfram syndrome, gout, excessive sugar craving, hypertriglyceridemia, dyslipidemia, malnutrition-related diabetes, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, traumatic brain injury, peripheral vascular disease, endothelial dysfunction, impaired vascular compliance, vascular restenosis, thrombosis, hypertension, pulmonary hypertension, restenosis after angioplasty, intermittent claudication, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, macular degeneration, cataract, glomerulosclerosis, arthritis, osteoporosis, treatment of addiction, cocaine dependence, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), ulcerative colitis, inflammatory bowel disease, colitis, irritable bowel syndrome, Crohn's disease, short bowel syndrome, Parkinson's, Alzheimer's disease, impaired cognition, schizophrenia, and Polycystic Ovary Syndrome (PCOS).

In some embodiments, the disease, disorder, or condition includes, but is not limited to type 2 diabetes mellitus, early onset type 2 diabetes mellitus, obesity, idiopathic intracranial hypertension, Wolfram syndrome, weight gain from use of other agents, gout, excessive sugar craving, hypertriglyceridemia, dyslipidemia, gestational diabetes, kidney disease (e.g., acute kidney disorder, tubular dysfunction, proinflammatory changes to the proximal tubules), adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), short bowel syndrome, Parkinson's disease, Polycystic Ovary Syndrome (PCOS), or any combination thereof.

In some embodiments, the disease, disorder, or condition includes, but is not limited to type 2 diabetes mellitus, early onset type 2 diabetes mellitus, obesity, idiopathic intracranial hypertension, Wolfram syndrome, weight gain from use of other agents, gout, excessive sugar craving, hypertriglyceridemia, dyslipidemia, gestational diabetes, adipocyte dysfunction, visceral adipose deposition, myocardial infarction, peripheral arterial disease, stroke, transient ischemic attacks, hyperglycemia, post-prandial lipemia, metabolic acidosis, ketosis, hyperinsulinemia, impaired glucose metabolism, insulin resistance, hepatic insulin resistance, chronic renal failure, syndrome X, angina pectoris, diabetic nephropathy, impaired glucose tolerance, diabetic neuropathy, diabetic retinopathy, skin and connective tissue disorders, foot ulcerations, or any combination thereof.

In some embodiments, the compounds and pharmaceutical compositions and methods for treating a patient described herein induce one or more of a reduction of blood glucose levels (e.g., reduce blood glucose levels), a reduction of blood hemoglobin A1c (HbA1c) levels, a promotion of insulin synthesis, a stimulation of insulin secretion, an increase in the mass of β-cells, a modulation of gastric acid secretion, a modulation of gastric emptying, a decrease in the body mass index (BMI), and/or a decrease in glucagon production (e.g., level). In some embodiments, the compounds and pharmaceutical compositions and methods for treating a patient described herein can reduce blood glucose levels, reduce blood hemoglobin A1c (HbA1c) levels, promote insulin synthesis, stimulate insulin secretion, increase the mass of β-cells, modulate gastric acid secretion, modulate gastric emptying, decrease the body mass index (BMI), decrease glucagon production (e.g., level), or any combination thereof. In certain embodiments, the compounds and pharmaceutical compositions and methods for treating a patient described herein stabilize serum glucose and serum insulin levels (e.g., serum glucose and serum insulin concentrations). Also provided herein are methods for modulating glucose or insulin levels in a patient in need of such modulating, the method comprising administering to the patient an effective amount of a compound of Formula I, or a pharmaceutically acceptable salt, deuterated analog, or solvate thereof (e.g., a compound of any one of Formula II, III, or IV, or a pharmaceutically acceptable salt, deuterated analog, or solvate thereof), or a pharmaceutical composition as disclosed herein.

In some embodiments, provided herein is a method for reducing the risk (e.g., by about at least 20%, at least 30%, at least 40%, at least 50%, at least 60%, at least 70%, or at least 80%) of major adverse cardiovascular events (MACE) in a patient in need thereof, the method comprising administering to the patient an effective amount of a compound of Formula I, or a pharmaceutically acceptable salt, deuterated analog, or solvate thereof (e.g., a compound of any one of Formula II, III, or IV, or a pharmaceutically acceptable salt, deuterated analog, or solvate thereof), or a pharmaceutical composition as disclosed herein. In certain of these embodiments, the patient is an adult that has been diagnosed with type 2 diabetes (T2D). In certain embodiments, the patient is an adult that has been diagnosed with a heart disease. In certain embodiments, the patient is an adult that has been diagnosed with type 2 diabetes (T2D) and a heart disease. In certain embodiments, the patient is an adult that has type 2 diabetes (T2D). In certain embodiments, the patient is an adult that has a heart disease. In certain embodiments, the patient has type 2 diabetes (T2D) and a heart disease.

### Indications

### Obesity

In some embodiments, the condition, disease or disorder is obesity and conditions, diseases or disorders that are associated with or related to obesity. Non-limiting examples of obesity and obesity related conditions include symptomatic obesity, simple obesity, childhood obesity, morbid obesity, and abdominal obesity (central obesity characterized by abdominal adiposity). Non-limiting examples of symptomatic obesity include endocrine obesity (e.g., Cushing syndrome, hypothyroidism, insulinoma, obese type II diabetes, pseudohypoparathyroidism, hypogonadism), hypothalamic obesity, hereditary obesity (e.g., Prader-Willi syndrome, Laurence-Moon-Biedl syndrome), and drug-induced obesity (e.g., steroid, phenothiazine, insulin, sulfonylurea agent, or β-blocker-induced obesity).

In some embodiments, the condition, disease or disorder is associated with obesity. Examples of such conditions, diseases or disorders include, without limitation, glucose tolerance disorders, diabetes (e.g., type 2 diabetes, obese diabetes), lipid metabolism abnormality, hyperlipidemia, hypertension, cardiac failure, hyperuricemia, gout, fatty liver (including non-alcoholic steatohepatitis (NASH)), coronary heart disease (e.g., myocardial infarction, angina pectoris), cerebral infarction (e.g., brain thrombosis, transient cerebral ischemic attack), bone or articular disease (e.g., knee osteoarthritis, hip osteoarthritis, spondylitis deformans, lumbago), sleep apnea syndrome, obesity hypoventilation syndrome (Pickwickian syndrome), menstrual disorder (e.g., abnormal menstrual cycle, abnormality of menstrual flow and cycle, amenorrhea, abnormal catamenial symptom), visceral obesity syndrome, urine incontinence, and metabolic syndrome. In some embodiments, the chemical compound and pharmaceutical compositions described herein can be used to treat patients exhibiting symptoms of both obesity and insulin deficiency.

### Diabetes

In some embodiments, the condition, disease or disorder is diabetes. Non-limiting examples of diabetes include type 1 diabetes mellitus, type 2 diabetes mellitus (e.g., diet-treated type 2-diabetes, sulfonylurea-treated type 2-diabetes, a far-advanced stage type 2-diabetes, long-term insulin-treated type 2-diabetes), diabetes mellitus (e.g., non-insulin-dependent diabetes mellitus, insulin-dependent diabetes mellitus), gestational diabetes, obese diabetes, autoimmune diabetes, and borderline type diabetes. In some embodiments, the condition, disease or disorder is type 2 diabetes mellitus (e.g., diet-treated type 2-diabetes, sulfonylurea-treated type 2-diabetes, a far-advanced stage type 2-diabetes, long-term insulin-treated type 2-diabetes).

Provided herein is a method of treating a diabetes mellitus in a patient, the method comprising (a) determining that the patient has type 2 diabetes mellitus, and (b) administering to the patient a therapeutically effective amount of a compound of Formula I, or a pharmaceutically acceptable salt, deuterated analog, or solvate thereof (e.g., a compound of Formula II, III, or IV, or a pharmaceutically acceptable salt, deuterated analog, or solvate thereof, or a pharmaceutically acceptable salt, deuterated analog, or solvate thereof) or a pharmaceutical composition as disclosed herein.

Provided herein is a method for treating type 2 diabetes mellitus in a patient, the method comprising administering to a patient identified or diagnosed as having type 2 diabetes mellitus a therapeutically effective amount of a compound of Formula I, or a pharmaceutically acceptable salt, deuterated analog, or solvate thereof (e.g., a compound of any one of a compound of any one of Formula II, III, or IV, or a pharmaceutically acceptable salt, deuterated analog, or solvate thereof, or a pharmaceutically acceptable salt, deuterated analog, or solvate thereof), or a pharmaceutical composition as disclosed herein.

Also provided herein is a method of treating type 2 diabetes mellitus in a patient in need thereof, the method comprising administering to the patient a therapeutically effective amount of a compound of Formula I, or a pharmaceutically acceptable salt, deuterated analog, or solvate thereof (e.g., a compound of any one of a compound of any one of Formula II, III, or IV, or a pharmaceutically acceptable salt, deuterated analog, or solvate thereof, or a pharmaceutically acceptable salt, deuterated analog, or solvate thereof), or a pharmaceutical composition as disclosed herein.

In some embodiments, the compounds and pharmaceutical compositions and methods for treating a patient with a condition, disease, or disorder (e.g., type 2 diabetes mellitus) described herein reduce fasting plasma glucose levels. In some embodiments, the compounds and pharmaceutical compositions and methods for treating a patient with a condition, disease, or disorder (e.g., type 2 diabetes mellitus) described herein reduce non-fasting plasma glucose levels. In some embodiments, the compounds and pharmaceutical compositions and methods for treating a patient with a condition, disease, or disorder (e.g., type 2 diabetes mellitus) described herein reduce HbA1c levels. In some embodiments, the compounds and pharmaceutical compositions and methods for treating a patient with a condition, disease, or disorder (e.g., type 2 diabetes mellitus) described herein reduce glucagon levels. In some embodiments, the compounds and pharmaceutical compositions and methods for treating a patient with a condition, disease, or disorder (e.g., type 2 diabetes mellitus) described herein increase insulin levels. In some embodiments, the compounds and pharmaceutical compositions and methods for treating a patient with a condition, disease, or disorder (e.g., type 2 diabetes mellitus) described herein reduce BMI.

In some embodiments, a reduction in fasting plasma glucose levels of about 5% to about 95% indicates treatment of type 2 diabetes mellitus. In some embodiments, a reduction in fasting plasma glucose levels of about 15% to about 80% indicates treatment of type 2 diabetes mellitus. In some embodiments, a reduction in fasting plasma glucose levels of about 25% to about 60% indicates treatment of type 2 diabetes mellitus. In some embodiments, a reduction in fasting plasma glucose levels to about or below 126 mg/dL, about or below 110 mg/dL, or about or below 90 mg/dL indicates treatment of the type 2 diabetes mellitus.

In some embodiments, a reduction in non-fasting plasma glucose levels of about 5% to about 95% indicates treatment of type 2 diabetes mellitus. In some embodiments, a reduction in non-fasting plasma glucose levels of about 15% to about 80% indicates treatment of type 2 diabetes mellitus. In some embodiments, a reduction in non-fasting plasma glucose levels of about 25% to about 60% indicates treatment of type 2 diabetes mellitus. In some embodiments, a reduction in non-fasting plasma glucose levels to about or below 200 mg/dL, about or below 150 mg/dL, or about or below 130 mg/dL indicates treatment of type 2 diabetes mellitus.

In some embodiments, a reduction in HbA1c levels of about 5% to about 95% indicates treatment of type 2 diabetes mellitus. In some embodiments, a reduction in HbA1c levels of about 15% to about 80% indicates treatment of type 2 diabetes mellitus. In some embodiments, a reduction in HbA1c levels of about 25% to about 60% indicates treatment of type 2 diabetes mellitus. In some embodiments, reduction in HbA1c levels to about or below 6.5%, about or below 6.0%, or about or below 5.0% indicates treatment of type 2 diabetes mellitus.

In some embodiments, a reduction in glucagon levels of about 5% to about 95% indicates treatment of type 2 diabetes mellitus. In some embodiments, a reduction in glucagon levels of about 15% to about 80% indicates treatment of type 2 diabetes mellitus. In some embodiments, a reduction in glucagon levels of about 25% to about 60% indicates treatment of type 2 diabetes mellitus. In some embodiments, an increase in insulin levels of about 5% to about 95% indicates treatment of type 2 diabetes mellitus. In some embodiments, an increase in insulin levels of about 15% to about 80% indicates treatment of type 2 diabetes mellitus. In some embodiments, an increase in insulin levels of about 25% to about 60% indicates treatment of type 2 diabetes mellitus.

In some embodiments, a reduction in BMI of about 5% to about 95% indicates treatment of type 2 diabetes mellitus. In some embodiments, a reduction in BMI of about 15% to about 80% indicates treatment of the type 2 diabetes mellitus. In some embodiments, a reduction in BMI of about 25% to about 60% indicates treatment of type 2 diabetes mellitus. In some embodiments, a reduction in BMI of about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% indicates treatment of type 2 diabetes mellitus. In some embodiments, a reduction in BMI to about or below 40, about or below 30, or about or below 20 indicates treatment of type 2 diabetes mellitus.

In some embodiments, the condition, disease or disorder is associated with diabetes (e.g., a complication of diabetes). Non-limiting examples of disorders associated with diabetes include obesity, obesity-related disorders, metabolic syndrome, neuropathy, nephropathy (e.g., diabetic nephropathy), retinopathy, diabetic cardiomyopathy, cataract, macroangiopathy, osteopenia, hyperosmolar diabetic coma, infectious disease (e.g., respiratory infection, urinary tract infection, gastrointestinal infection, dermal soft tissue infections, inferior limb infection), diabetic gangrene, xerostomia, hypacusis, cerebrovascular disorder, diabetic cachexia, delayed wound healing, diabetic dyslipidemia peripheral blood circulation disorder, cardiovascular risk factors. (e.g., coronary artery disease, peripheral artery disease, cerebrovascular disease, hypertension, and risk factors related to unmanaged cholesterol and/or lipid levels, and/or inflammation), NASH, bone fracture, and cognitive dysfunction

Other non-limiting examples of disorders related to diabetes include pre-diabetes, hyperlipidemia (e.g., hypertriglyceridemia, hypercholesterolemia, high LDL-cholesterolemia, low HDL-cholesterolemia, postprandial hyperlipemia), metabolic syndrome (e.g., metabolic disorder where activation of GLP-1R is beneficial, metabolic syndrome X), hypertension, impaired glucose tolerance (IGT), insulin resistance, and sarcopenia.

In some embodiments, the condition, disease or disorder is diabetes and obesity (diabesity). In some embodiments, the compounds described herein are also useful in improving the therapeutic effectiveness of metformin.

### Disorders of Metabolically Important Tissues

In some embodiments, the condition, disease or disorder is a disorder of a metabolically important tissue. Non-limiting examples of metabolically important tissues include liver, fat, pancreas, kidney, and gut.

In some embodiments, the condition, disease or disorder is a fatty liver disease. Fatty liver diseases include, but are not limited to, non-alcoholic fatty acid liver disease (NAFLD), steatohepatitis, non-alcoholic steatohepatitis (NASH), fatty liver disease resulting from hepatitis, fatty liver disease resulting from obesity, fatty liver disease resulting from diabetes, fatty liver disease resulting from insulin resistance, fatty liver disease resulting from hypertriglyceridemia, Abetalipoproteinemia, hyperlipoproteinemia, glycogen storage diseases, Weber-Christian disease, Wolman disease, acute fatty liver of pregnancy, and lipodystrophy.

Non-alcoholic fatty liver disease (NAFLD) represents a spectrum of disease occurring in the absence of alcohol abuse and is typically characterized by the presence of steatosis (fat in the liver). NAFLD is believed to be linked to a variety of conditions, e.g., metabolic syndrome (including obesity, diabetes and hypertriglyceridemia) and insulin resistance. It can cause liver disease in adults and children and may ultimately lead to cirrhosis (Skelly et al., J Hepatol 2001; 35: 195-9; Chitturi et al., Hepatology 2002; 35(2):373-9). The severity of NAFLD ranges from the relatively benign isolated predominantly macrovesicular steatosis (i.e., nonalcoholic fatty liver or NAFL) to non-alcoholic steatohepatitis (NASH) (Angulo et al., J Gastroenterol Hepatol 2002; 17 Suppl:S186-90).

In some embodiments, the patient is a pediatric patient. The term “pediatric patient” as used herein refers to a patient under the age of 21 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W.B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994. In some embodiments, a pediatric patient is from birth through the first 28 days of life, from 29 days of age to less than two years of age, from two years of age to less than 12 years of age, or 12 years of age through 21 years of age (up to, but not including, the twenty-second birthday). In some embodiments, a pediatric patient is from birth through the first 28 days of life, from 29 days of age to less than 1 year of age, from one month of age to less than four months of age, from three months of age to less than seven months of age, from six months of age to less than 1 year of age, from 1 year of age to less than 2 years of age, from 2 years of age to less than 3 years of age, from 2 years of age to less than seven years of age, from 3 years of age to less than 5 years of age, from 5 years of age to less than 10 years of age, from 6 years of age to less than 13 years of age, from 10 years of age to less than 15 years of age, or from 15 years of age to less than 22 years of age. In some embodiments, the patient is an adult patient.

Other non-limiting examples of disorders in metabolically important tissues include joint disorders (e.g., osteoarthritis, secondary osteoarthritis), steatosis (e.g. in the liver); fibrosis (e.g., in the liver); cirrhosis (e.g., in the liver); gall stones; gallbladder disorders; gastroesophageal reflux; sleep apnea; hepatitis; fatty liver; bone disorder characterized by altered bone metabolism, such as osteoporosis, including post-menopausal osteoporosis, poor bone strength, osteopenia, Paget's disease, osteolytic metastasis in cancer patients, osteodistrophy in liver disease and the altered bone metabolism caused by renal failure or hemodialysis, bone fracture, bone surgery, aging, pregnancy, protection against bone fractures, and malnutrition polycystic ovary syndrome; renal disease (e.g., chronic renal failure, glomerulonephritis, glomerulosclerosis, nephrotic syndrome, hypertensive nephrosclerosis, end-stage renal disease); muscular dystrophy, angina pectoris, acute or chronic diarrhea, testicular dysfunction, respiratory dysfunction, frailty, sexual dysfunction (e.g., erectile dysfunction), and geriatric syndrome. In some embodiments, the compounds and pharmaceutical compositions described herein can be used for treating surgical trauma by improving recovery after surgery and/or by preventing the catabolic reaction caused by surgical trauma.

### Cardiovascular and Vascular Diseases

In some embodiments, the condition, disease or disorder is a cardiovascular disease. Non-limiting examples of cardiovascular disease include congestive heart failure, atherosclerosis, arteriosclerosis, coronary heart disease, coronary artery disease, congestive heart failure, coronary heart disease, hypertension, cardiac failure, cerebrovascular disorder (e.g., cerebral infarction), vascular dysfunction, myocardial infarction, elevated blood pressure (e.g., 130/85 mm Hg or higher), and prothrombotic state (exemplified by high fibrinogen or plasminogen activator inhibitor in the blood).

In some embodiments, the condition, disease or disorder is related to a vascular disease. Non-limiting examples of vascular diseases include peripheral vascular disease, macrovascular complications (e.g., stroke), vascular dysfunction, peripheral artery disease, abdominal aortic aneurysm, carotid artery disease, cerebrovascular disorder (e.g., cerebral infarction), pulmonary embolism, chronic venous insufficiency, critical limb ischemia, retinopathy, nephropathy, and neuropathy.

### Neurological Diseases

In some embodiments, the condition, disease or disorder is a neurological disorder (e.g., neurodegenerative disorder) or a psychiatric disorder. Non-limiting examples of neurological disorders include idiopathic intracranial hypertension (IIH), brain insulin resistance, mild cognitive impairment (MCI), Alzheimer's disease (AD), Parkinson's disease (PD), anxiety, dementia (e.g., senile dementia), traumatic brain injury, Huntington's chores, tardive dyskinesia, hyperkinesia, mania, Morbus Parkinson, steel-Richard syndrome, Down's syndrome, myasthenia gravis, nerve trauma, brain trauma, vascular amyloidosis, cerebral hemorrhage I with amyloidosis, brain inflammation, Friedrich's ataxia, acute confusion disorder, amyotrophic lateral sclerosis (ALS), glaucoma, and apoptosis-mediated degenerative diseases of the central nervous system (e.g., Creutzfeld-Jakob Disease, bovine spongiform encephalopathy (mad cow disease), and chronic wasting syndrome). See, e.g., US20060275288A1.

In some embodiments, the condition, disease or disorder is idiopathic intracranial hypertension. Idiopathic intracranial hypertension is characterized by increased intracranial pressure and papilloedema. See, e.g., Virdee et al. Ophthalmol Ther. 2020; 9(4):767-781. In some embodiments, the compounds and pharmaceutical compositions and methods described herein reduce cerebrospinal fluid secretion in a patient with idiopathic intracranial hypertension. In some embodiments, the compounds and pharmaceutical compositions and methods described herein reduce intracranial pressure in a patient with idiopathic intracranial hypertension. In some embodiments, the compounds and pharmaceutical compositions and methods described herein reduce one or more symptoms in a patient with idiopathic intracranial hypertension. Symptoms of idiopathic intracranial hypertension can include severe headaches and visual impairment. In some embodiments, the patient with idiopathic intracranial hypertension is female. In some embodiments, the patient with idiopathic intracranial hypertension is about 20 to about 30 years old. In some embodiments, the patient with idiopathic intracranial hypertension is obese.

In some embodiments, the condition, disease or disorder is Wolfram syndrome. Wolfram syndrome is caused by biallelic mutations of the Wolframin E R transmembrane glycoprotein (Wfs1) gene. See, e.g., Seppa et al. Sci Rep 9, 15742 (2019). Wolfram syndrome can first appear as diabetes mellitus, followed by optic nerve atrophy, deafness, and symptoms of neurodegeneration. Patients with Wolfram syndrome can have symptoms of ataxia, sleep apnea, dysphagia, hearing loss, and loss of taste due to brainstem atrophy. In some embodiments, the compounds and pharmaceutical compositions and methods described herein reduce neuroinflammation in a patient with Wolfram syndrome. In some embodiments, the neuroinflammation is reduced in the inferior olive in the patient. In some embodiments, the compounds and pharmaceutical compositions and methods described herein reduce retinal ganglion cell death in a patient with Wolfram syndrome. In some embodiments, the compounds and pharmaceutical compositions and methods described herein reduce axonal degeneration in a patient with Wolfram syndrome. In some embodiments, the compounds and pharmaceutical compositions and methods described herein reduce one or more symptoms (e.g., any of the symptoms described herein) in a patient with Wolfram syndrome.

Non-limiting examples of psychiatric disorders include drug dependence/addiction (narcotics and amphetamines and attention deficit/hyperactivity disorder (ADHD). The compounds and pharmaceutical compositions described herein can be useful in improving behavioral response to addictive drugs, decreasing drug dependence, prevention drug abuse relapse, and relieving anxiety caused by the absence of a given addictive substance. See, e.g., US20120021979A1.

In some embodiments, the compounds and pharmaceutical compositions described herein are useful in improving learning and memory by enhancing neuronal plasticity and facilitation of cellular differentiation, and also in preserving dopamine neurons and motor function in Morbus Parkinson.

### Insulin-Related

In some embodiments, the condition, disease or disorder is impaired fasting glucose (IFG), impaired fasting glycemia (IFG), hyperglycemia, insulin resistance (impaired glucose homeostasis), hyperinsulinemia, elevated blood levels of fatty acids or glycerol, a hypoglycemic condition, insulin resistant syndrome, paresthesia caused by hyperinsulinemia, hyperlipidemia, hypercholesteremia, impaired wound healing, leptin resistance, glucose intolerance, increased fasting glucose, dyslipidemia (e.g., hyperlipidemia, atherogenic dyslipidemia characterized by high triglycerides and low HDL cholesterol), glucagonoma, hyperuricacidemia, hypoglycemia (e.g., nighttime hypoglycemia), and concomitant comatose endpoint associated with insulin.

In some embodiments, the compounds and pharmaceutical compositions described herein can reduce or slow down the progression of borderline type, impaired fasting glucose or impaired fasting glycemia into diabetes.

### Autoimmune Disorders

In some embodiments, the condition, disease or disorder is an autoimmune disorder. Non-limiting examples of autoimmune disorders include multiple sclerosis, experimental autoimmune encephalomyelitis, autoimmune disorder is associated with immune rejection, graft versus host disease, uveitis, optic neuropathies, optic neuritis, transverse myelitis, inflammatory bowel disease, rheumatoid arthritis, ankylosing spondylitis, systemic lupus erythematosus, myasthenia gravis, and Graves' disease. See, e.g., US20120148586A1.

### Stomach and Intestine-Related Disorders

In some embodiments, the condition, disease or disorder is a stomach or intestine related disorder. Non-limiting examples of these disorders include ulcers of any etiology (e.g. peptic ulcers, Zollinger-Ellison syndrome, drug-induced ulcers, ulcers related to infections or other pathogens), digestion disorders, malabsorption, short bowel syndrome, cul-de-sac syndrome, inflammatory bowel diseases (Crohn's disease and ulcerative colitis), celiac sprue, hypogammaglobulinemic sprue, chemotherapy and/or radiation therapy-induced mucositis and diarrhea, gastrointestinal inflammation, short bowel syndrome, colitis ulcerosa, gastric mucosal injury (e.g., gastric mucosal injury caused by aspirin), small intestinal mucosal injury, and cachexia (e.g., cancerous cachexia, tuberculous cachexia, cachexia associated with blood disease, cachexia associated with endocrine disease, cachexia associated with infectious disease, and cachexia caused by acquired immunodeficiency syndrome).

### Body Weight

In some embodiments, the compounds and pharmaceutical compositions described herein can be used to reduce body weight (e.g., excess body weight), prevent body weight gain, induce weight loss, decrease body fat, or reduce food intake in a patient (e.g., a patient in need thereof). In some embodiments, the weight increase in a patient may be attributed to excessive ingestion of food or unbalanced diets, or may be weight increase derived from a concomitant drug (e.g., insulin sensitizers having a PPARγ agonist-like action, such as troglitazone, rosiglitazone, englitazone, ciglitazone, pioglitazone and the like). In some embodiments, the weight increase may be weight increase before reaching obesity, or may be weight increase in an obese patient. In some embodiments, the weight increase may also be medication-induced weight gain or weight gain subsequent to cessation of smoking.

In some embodiments, the weight gain is induced by the use of steroids or antipsychotics.

In some embodiments, the condition, disease or disorder is an eating disorder, such as hyperphagia, binge eating, bulimia, compulsive eating, or syndromic obesity such as Prader-Willi and Bardet-Biedl syndromes.

### Inflammatory Diseases

In some embodiments, the condition, disease or disorder is an inflammatory disorder. Non-limiting examples of inflammatory disorders include chronic rheumatoid arthritis, spondylitis deformans, arthritis deformans, lumbago, gout, post-operational or post-traumatic inflammation, bloating, neuralgia, laryngopharyngitis, cystitis, pneumonia, pancreatitis, enteritis, inflammatory bowel disease (including inflammatory large bowel disease), inflammation in metabolically important tissues including liver, fat, pancreas, kidney and gut, and a proinflammatory state (e.g., elevated levels of proinflammatory cytokines or markers of inflammation-like C-reactive protein in the blood).

### Cancer

In some embodiments, the condition, disease or disorder is cancer. Suitable examples of cancer include breast cancer (e.g., invasive ductal breast cancer, noninvasive ductal breast cancer, inflammatory breast cancer), prostate cancer (e.g., hormone-dependent prostate cancer, hormone-independent prostate cancer), pancreatic cancer (e.g., ductal pancreatic cancer), gastric cancer (e.g., papillary adenocarcinoma, mucous adenocarcinoma, adenosquamous carcinoma), lung cancer (e.g., non-small cell lung cancer, small-cell lung cancer, malignant mesothelioma), colon cancer (e.g., gastrointestinal stromal tumor), rectal cancer (e.g., gastrointestinal stromal tumor), colorectal cancer (e.g., familial colorectal cancer, hereditary non-polyposis colorectal cancer, gastrointestinal stromal tumor), small intestinal cancer (e.g., non-Hodgkin's lymphoma, gastrointestinal stromal tumor), esophageal cancer, duodenal cancer, tongue cancer, pharyngeal cancer (e.g., nasopharyngeal cancer, oropharynx cancer, hypopharyngeal cancer), salivary gland cancer, brain tumor (e.g., pineal astrocytoma, pilocytic astrocytoma, diffuse astrocytoma, anaplastic astrocytoma), neurilemmoma, liver cancer (e.g., primary liver cancer, extrahepatic bile duct cancer), renal cancer (e.g., renal cell cancer, transitional cell cancer of the renal pelvis and ureter), bile duct cancer, endometrial cancer, uterine cervical cancer, ovarian cancer (e.g., epithelial ovarian cancer, extragonadal germ cell tumor, ovarian germ cell tumor, ovarian tumor of low malignant potential), bladder cancer, urethral cancer, skin cancer (e.g., intraocular (ocular) melanoma, Merkel cell carcinoma), hemangioma, malignant lymphoma, malignant melanoma, thyroid cancer (e.g., medullary thyroid cancer), parathyroid cancer, nasal cavity cancer, sinus cancer, bone tumor (e.g., osteosarcoma, Ewing tumor, uterine sarcoma, soft tissue sarcoma), angiofibroma, sarcoma of the retina, penis cancer, testicular tumor, pediatric solid tumor (e.g., Wilms' tumor, childhood kidney tumor), Kaposi's sarcoma, Kaposi's sarcoma caused by AIDS, tumor of maxillary sinus, fibrous histiocytoma, leiomyosarcoma, rhabdomyosarcoma, and leukemia (e.g., acute myeloid leukemia, acute lymphoblastic leukemia).

### Hypothalamic-Pituitary Disorders

In some embodiments, the condition, disease or disorder is related to the hypothalamic-pituitary-gonadal axis. For example, the condition, disease or disorder is related to the hypothalamus-pituitary-ovary axis. In another example, the condition, disease or disorder is related to the hypothalamus-pituitary-testis axis. Hypothalamic-pituitary-gonadal axis diseases include, but are not limited to, hypogonadism, polycystic ovary syndrome, hypothyroidism, hypopituitarism, sexual dysfunction, and Cushing's disease.

In some embodiments, the condition, disease or disorder associated with diabetes is related to the hypothalamic-pituitary-gonadal axis.

### Pulmonary Disease

In some embodiments, the condition, disease or disorder is related to a pulmonary disease. Pulmonary diseases include, but are not limited to, asthma, idiopathic pulmonary fibrosis, pulmonary hypertension, obstructive sleep apnoea-hypopnoea syndrome, and chronic obstructive pulmonary disease (COPD) (e.g., emphysema, chronic bronchitis, and refractory (non-reversible) asthma).

In some embodiments, the condition, disease or disorder associated with diabetes is a pulmonary disease.

### Combination Therapies

In some embodiments, this disclosure contemplates both monotherapy regimens as well as combination therapy regimens.

In some embodiments, the methods described herein can further include administering one or more additional therapies (e.g., one or more additional therapeutic agents and/or one or more therapeutic regimens) in combination with administration of the compounds described herein.

In some embodiments, the methods described herein include administering a compound described herein in combination with one or more of a diet therapy (e.g., dietary monitoring, diet therapy for diabetes), an exercise therapy (e.g., physical activity), blood sugar monitoring, gastric electrical stimulation (e.g., TANTALUS®), and diet modifications.

In some embodiments, the compounds of X, or a pharmaceutically acceptable salt, deuterated analog, or solvate thereof as described herein can be administered in combination with one or more additional therapeutic agents.

Representative additional therapeutic agents include, but are not limited to, anti-obesity agents, therapeutic agents for diabetes, therapeutic agents for diabetic complications, therapeutic agents for hyperlipidemia, antihypertensive agents, diuretics, chemotherapeutics, immunotherapeutics, anti-inflammatory drugs, antithrombotic agents, anti-oxidants, therapeutic agents for osteoporosis, vitamins, antidementia drugs, erectile dysfunction drugs, therapeutic drugs for urinary frequency or urinary incontinence, therapeutic agents for NAFLD, therapeutic agents for NASH, therapeutic agents for dysuria, and anti-emetic agents.

In some embodiments, the one or more additional therapeutic agents include those useful, for example, as anti-obesity agents. Non-limiting examples include monoamine uptake inhibitors (e.g., tramadol, phentermine, sibutramine, mazindol, fluoxetine, tesofensine), serotonin 2C receptor agonists (e.g., lorcaserin), serotonin 6 receptor antagonists, histamine H3 receptor modulator, GABA modulator (e.g., topiramate), including GABA receptor agonists (e.g., gabapentin, pregabalin), neuropeptide Y antagonists (e.g., velneperit), peptide YY or an analogue thereof, cannabinoid receptor antagonists (e.g., rimonabant, taranabant), ghrelin antagonists, ghrelin receptor antagonists, ghrelin acylation enzyme inhibitors, opioid receptor antagonists (e.g., GSK-1521498, naltrexone), orexin receptor antagonists, melanocortin 4 receptor agonists, 11-hydroxysteroid dehydrogenase inhibitors (e.g., AZD-4017, BVT-3498, INCB-13739), pancreatic lipase inhibitors (e.g., orlistat, cetilistat), β3 agonists (e.g., N-5984), diacylglycerol acyltransferase 1 (DGAT1) inhibitors, acetylCoA carboxylase (ACC) inhibitors (e.g., compounds described in WO 2020/234726, WO 2020/044266, and U.S. Pat. No. 8,859,577), stearoyl-CoA desaturated enzyme inhibitors, microsomal triglyceride transfer protein inhibitors (e.g., R-256918), sodium-glucose cotransporter 2 (SGLT-2) inhibitors (e.g., JNJ-28431754, dapagliflozin, AVE2268, TS-033, YM543, TA-7284, ASP1941, remogliflozin, empagliflozin, canagliflozin, ipragliflozin, tofogliflozin, sergliflozin etabonate, remogliflozin etabonate, or ertugliflozin), SGLT-1 inhibitors, MCR-4 agonists, monoamine reuptake inhibitors, melanocytestimulating hormone analogs, 5HT2c agonists, galanin antagonists, anorectic agents (such as a bombesin agonist), thyromimetic agents, dehydroepiandrosterone or analogs thereof, human agouti-related protein (AGRP) inhibitors, neuromedin U agonists, NFK inhibitors (e.g., HE-3286), PPAR agonists (e.g., GFT-505, DRF-11605, gemfibrozil, fenofibrate, balaglitazone, ciglitazone, darglitazone, englitazone, isaglitazone, pioglitazone, rosiglitazone, CLX-0940, GW-1536, GW-1 929, GW-2433, KRP-297, L-796449, LR-90, MK-0767, and SB-21 9994), phosphotyrosine phosphatase inhibitors (e.g., sodium vanadate, trodusquemin), GPR119 agonists (e.g., PSN-821, MBX-2982, APD597, compounds described in WO 2010/140092, WO 2010/128425, WO 2010/128414, WO 2010/106457), glucokinase activators (e.g., piragliatin, AZD-1656, AZD6370, TTP-355, TTP-399, TTP547, ARRY403, MK-0599, TAK-329, AZD5658 or GKM-001 compounds described in WO 2010/103437, WO 2010/103438, WO 2010/013161, WO 2007/122482, WO 2006/112549, WO 2007/028135, WO 2008/047821, WO 2008/050821, WO 2008/136428 and WO 2008/156757), leptin, leptin derivatives (e.g., metreleptin), leptin resistance improving drugs, CNTF (ciliary neurotrophic factor), BDNF (brain-derived neurotrophic factor), cholecystokinin agonists, amylin preparations (e.g., pramlintide, AC-2307), neuropeptide Y agonists (e.g., PYY3-36, derivatives of PYY3-36, obineptide, TM-30339, TM-30335), oxyntomodulin (OXM) preparations, appetite suppressants (e.g. ephedrine), FGF21 preparations (e.g., animal FGF21 preparations extracted from the pancreas of bovine or swine; human FGF21 preparations genetically synthesized using Escherichia coli or yeast; fragments or derivatives of FGF21), anorexigenic agents (e.g., P-57), human proislet peptide (HIP), melanocortin receptor 4 agonist (e.g., setmelanotide), melanin concentrating hormone receptor 1 antagonist, serotonergic agents (e.g. sibutramine, lorcaserin), farnesoid X receptor (FXR) agonist (e.g., obeticholic acid, tropifexor, cilofexor, LY2562175, Met409, TERN-101, EDP305, compounds described in WO 2020/234726 and WO 2020/044266), phentermine, zonisamide, norepinephrine/dopamine reuptake inhibitor (e.g., bupropion), GDF-15 analog, methionine aminopeptidase 2 (MetAP2) inhibitor (e.g., beloranib or ZGN-1061), diethylpropion, phendimetrazine, benzphetamine, fibroblast growth factor receptor (FGFR) modulator, biotin, a MAS receptor modulator, glucagon receptor agonist, CCKa agonists (e.g., compounds described in WO 2005/116034 and U.S. Publication No. 2005/0287100), and AMP-activated protein kinase (AMPK) activator.

In some embodiments, the one or more additional therapeutic agents include those useful, for example, as anti-diabetic agents. Non-limiting examples include insulin and insulin preparations (e.g., animal insulin preparations extracted from the pancreas of bovine or swine; human insulin preparations genetically synthesized using Escherichia coli or yeast; zinc insulin; protamine zinc insulin; fragment or derivative of insulin (e.g., INS—1), oral insulin preparation, synthetic human insulin), insulin sensitizers (e.g., pioglitazone or a salt thereof), biguanides (e.g., metformin, buformin or a salt thereof (e.g., hydrochloride, fumarate, succinate)), glucagon analogs (e.g., any of glucagon analogs described, e.g., in WO 2010/011439), agents which antagonize the actions of or reduce secretion of glucagon, sulfonylurea agents (e.g., chlorpropamide, tolazamide, glimepiride, tolbutamide, glibenclamide, gliclazide, acetohexamide, glyclopyramide, glybuzole, glyburide, glipizide), thiazolidinedione agents (e.g. rosiglitazone, lobeglitazone, troglitazone, balaglitazone, rivoglitazone, lobeglitazone or pioglitazone), glitazars (e.g., aleglitazar, chiglitazar, saroglitazar, muraglitazar, tesaglitazar), SGLT2 inhibitors (e.g., JNJ-28431754, dapagliflozin, AVE2268, TS-033, YM543, TA-7284, ASP1941, THR1474, TS-071, ISIS388626, LX4211, remogliflozin, empagliflozin, canagliflozin, ipragliflozin, tofogliflozin, sergliflozin etabonate, remogliflozin etabonate, ertugliflozin, compounds described in WO 2010/023594), GPR40 agonists (e.g., a FFAR1/FFA1 agonist, e.g. fasiglifam), α-glucosidase inhibitors (e.g., adiposin, camiglibose, pradimicin-Q, salbostatin, voglibose, acarbose, miglitol, emiglitate), insulin secretagogues, such as prandial glucose regulators (sometimes called “short-acting secretagogues”), e.g., meglitinides (e.g. repaglinide and nateglinide), cholinesterase inhibitors (e.g., donepezil, galantamine, rivastigmine, tacrine), NMDA receptor antagonists, dual GLP-1/GIP receptor agonists (e.g., LBT-2000, ZPD1-70), GLP-1R agonists (e.g., exenatide, liraglutide, albiglutide, dulaglutide, abiglutide, taspoglutide, lixisenatide, semaglutide, AVE-0010, S4P and Boc5), and dipeptidyl peptidase IV (DPP-4) inhibitors (e.g., vildagliptin, dutogliptin, gemigliptin, alogliptin, saxagliptin, sitagliptin, linagliptin, berberine, adogliptin, anagliptin (SK-0403), teneligliptin, omarigliptin, B11356, GRC8200, MP-513, PF-00734200, PHX1149, ALS2-0426, TA-6666, TS-021, KRP-104, trelagliptin).

In some embodiments, the one or more additional therapeutic agents include those useful, for example, for treating NAFL and NASH. Non-limiting examples include FXR agonists (e.g., obeticholic acid), PF-05221304, PPAR a/6 agonists (e.g., elafibranor), a synthetic fatty acid-bile conjugate (e.g., aramchol), an anti-lysyl oxidase homologue 2 (LOXL2) monoclonal antibody (e.g., simtuzumab), a caspase inhibitor (e.g., emricasan), a MAPK5 inhibitor (e.g., GS-4997), a galectin 3 inhibitor (e.g., GR-MD-02), a fibroblast growth factor 21 (FGF21) (e.g., BMS-986036), a niacin analogue (e.g., ARJ 3037MO), a leukotriene D4 (LTD4) receptor antagonist (e.g., tipelukast), an acetyl-CoA carboxylase (ACC) inhibitor (e.g., NDI 010976 and compounds described in WO 2009/144554, WO 2003/072197, WO 2009/144555, and WO 2008/065508), a ketohexokinase (KHK) inhibitor (e.g., compounds described in WO 2020/234726), an apoptosis signal-regulating kinase 1 (ASK1) inhibitor, an ileal bile acid transporter (IBAT) inhibitor, a dual antagonist of chemokine receptor 2 (CCR2) and CCR5 (e.g., cenicriviroc), diacylglyceryl acyltransferase 2 (DGAT2) inhibitor (e.g., compounds described in WO 2020/234726 and U.S. Publication No. 20180051012), a CB1 receptor antagonist, an anti-CB1R antibody, glycyrrhizin, schisandra extract, ascorbic acid, glutathione, silymarin, lipoic acid, and d-alpha-tocopherol, ascorbic acid, glutathione, vitamin B-complex, glitazones/thiazolidinediones (e.g., troglitazone, rosiglitazone, pioglitazone, balaglitazone, rivoglitazone, lobeglitazone), metformin, cysteamine, sulfonylureas, alpha-glucosidase inhibitors, meglitinides, vitamin E, tetrahydrolipstatin, milk thistle protein, anti-virals, and anti-oxidants.

In some embodiments, the one or more additional therapeutic agents include those useful, for example, for treating diabetic complications. Non-limiting examples include aldose reductase inhibitors (e.g., tolrestat, epalrestat, zopolrestat, fidarestat, CT-112, ranirestat, lidorestat), neurotrophic factor and increasing agents thereof (e.g., NGF, NT-3, BDNF, neurotrophic production/secretion promoting agents described in WO01/14372 (e.g., 4-(4-chlorophenyl)-2-(2-methyl-1-imidazolyl)-5-[3-(2-methylphenoxyl)propyl]oxazole), compounds described in WO2004/039365), PKC inhibitors (e.g., ruboxistaurin mesylate), AGE inhibitors (e.g., ALT946, N-phenacylthiazolium bromide (ALT766), EXO-226, pyridorin, pyridoxamine), serotonin and noradrenalin reuptake inhibitors (e.g., duloxetine), sodium channel inhibitors (e.g., lacosamide), active oxygen scavengers (e.g., thioctic acid), cerebral vasodilators (e.g., tiapuride, mexiletine), somatostatin receptor agonists (e.g., BIM23190), and apoptosis signal regulating kinase-1 (ASK-1) inhibitors.

In some embodiments, the one or more additional therapeutic agents include those useful, for example, for treating hyperlipidemia. Non-limiting examples include HMG-COA reductase inhibitors (e.g., pravastatin, simvastatin, lovastatin, atorvastatin, fluvastatin, rosuvastatin, pitavastatin or a salt thereof (e.g., sodium salt, calcium salt)), squalene synthase inhibitors (e.g., compounds described in WO97/10224, e.g., N-[[(3R,5S)-1-(3-acetoxy-2,2-dimethylpropyl)-7-chloro-5-(2,3-dimethoxyphenyl)-2-oxo-1,2,3,5-tetrahydro-4, 1-benzoxazepin-3-yl]acetyl]piperidin-4-acetic acid), fibrate compounds (e.g., bezafibrate, clofibrate, simfibrate, clinofibrate), anion exchange resin (e.g., colestyramine), nicotinic acid drugs (e.g., nicomol, niceritrol, niaspan), phytosterols (e.g., soysterol, gamma oryzanol (γ-oryzanol)), cholesterol absorption inhibitors (e.g., zechia), CETP inhibitors (e.g., dalcetrapib, anacetrapib) and ω-3 fatty acid preparations (e.g., ω-3-fatty acid ethyl esters 90).

In some embodiments, the one or more additional therapeutic agents include those useful, for example, as anti-hypertensive agents. Non-limiting examples include angiotensin converting enzyme inhibitors (e.g., captopril, zofenopril, fbsinopril, enalapril, ceranopril, cilazopril, delapril, pentopril, quinapril, ramipril, lisinopril), angiotensin II antagonists (e.g., candesartan cilexetil, candesartan, losartan, losartan potassium, eprosartan, valsartan, telmisartan, irbesartan, tasosartan, olmesartan, olmesartan medoxomil, azilsartan, azilsartan medoxomil), calcium antagonists (e.g., manidipine, nifedipine, amlodipine, efonidipine, nicardipine, cilnidipine) and β-blockers (e.g., metoprolol, atenolol, propranolol, carvedilol, pindolol). Further non-limiting examples of antihypertensive agents include: diuretics (e.g., chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, torsemide, furosemide, musolimine, bumetanide, triamtrenene, amiloride, spironolactone), alpha adrenergic blockers, beta adrenergic blockers, calcium channel blockers (e.g., diltiazem, verapamil, nifedipine and amlodipine), vasodilators (e.g., hydralazine), renin inhibitors, AT-1 receptor antagonists (e.g., losartan, irbesartan, valsartan), ET receptor antagonists (e.g., sitaxsentan, atrsentan, compounds disclosed in U.S. Pat. Nos. 5,612,359 and 6,043,265), dual ET/AII antagonist (e.g., compounds disclosed in WO 2000/01389), neutral endopeptidase (NEP) inhibitors, If channel blocker ivabradinand, vasopepsidase inhibitors (dual NEP-ACE inhibitors) (e.g., gemopatrilat and nitrates).

In some embodiments, the one or more additional therapeutic agents include those useful, for example, as diuretics. Non-limiting examples include xanthine derivatives (e.g., theobromine sodium salicylate, theobromine calcium salicylate), thiazide preparations (e.g., ethiazide, cyclopenthiazide, trichloromethiazide, hydrochlorothiazide, hydroflumethiazide, benzylhydrochlorothiazide, penfluthiazide, polythiazide, methyclothiazide), antialdosterone preparations (e.g., spironolactone, triamterene), carbonic anhydrase inhibitors (e.g., acetazolamide) and chlorobenzenesulfonamide agents (e.g., chlortalidone, mefruside, indapamide).

In some embodiments, the one or more additional therapeutic agents include those useful, for example, as immunotherapeutic agents. Non-limiting examples include microbial or bacterial compounds (e.g., muramyl dipeptide derivative, picibanil), polysaccharides having immunoenhancing activity (e.g., lentinan, sizofiran, krestin), cytokines obtained by genetic engineering approaches (e.g., interferon, interleukin (IL) such as IL-1, IL-2, IL-12), and colony-stimulating factors (e.g., granulocyte colony-stimulating factor, erythropoietin).

In some embodiments, the one or more additional therapeutic agents include those useful, for example, as anti-thrombotic agents. Non-limiting examples include heparins (e.g., heparin sodium, heparin calcium, enoxaparin sodium, dalteparin sodium) warfarin (e.g., warfarin potassium); anti-thrombin drugs (e.g., aragatroban, dabigatran, boroarginine derivatives, boropeptides, heparins, hirudin, and melagatran), FXa inhibitors (e.g., rivaroxaban, apixaban, edoxaban, YM150, compounds described in WO02/06234, WO 2004/048363, WO 2005/030740, WO 2005/058823, and WO 2005/113504) thrombolytic agents (e.g., anistreplase, streptokinase, tenecteplase (TNK), lanoteplase (nPA), urokinase, tisokinase, alteplase, nateplase, monteplase, pamiteplase, factor VIla inhibitors, PAT-1 inhibitors, alpha2-antiplasmin inhibitors, and anisoylated plasminogen streptokinase activator complex), and platelet aggregation inhibitors (e.g., ticlopidine hydrochloride, clopidogrel, prasugrel, E5555, SHC530348, cilostazol, ethyl icosapentate, beraprost sodium, and sarpogrelate hydrochloride).

In some embodiments, the one or more additional therapeutic agents include those useful, for example, for treating osteoporosis. Non-limiting examples include alfacalcidol, calcitriol, elcatonin, calcitonin salmon, estriol, ipriflavone, pamidronate disodium, alendronate sodium hydrate, incadronate disodium, and risedronate disodium. Suitable examples of vitamins include vitamin B1 and vitamin B12. Suitable examples of erectile dysfunction drugs include apomorphine and sildenafil citrate. Suitable examples of therapeutic agents for urinary frequency or urinary incontinence include flavorxate hydrochloride, oxybutynin hydrochloride and propiverine hydrochloride. Suitable examples of therapeutic agents for dysuria include acetylcholine esterase inhibitors (e.g., distigmine). Suitable examples of anti-inflammatory agents include nonsteroidal anti-inflammatory drugs such as aspirin, acetaminophen, indomethacin.

Other exemplary additional therapeutic agents include agents that modulate hepatic glucose balance (e.g., fructose 1,6-bisphosphatase inhibitors, glycogen phosphorylase inhibitors, glycogen synthase kinase inhibitors, glucokinase activators), agents designed to treat the complications of prolonged hyperglycemia, such as aldose reductase inhibitors (e.g. epalrestat and ranirestat), agents used to treat complications related to micro-angiopathies, anti-dyslipidemia agents, such as HMG-CoA reductase inhibitors (statins, e.g. rosuvastatin pravastatin, pitavastatin, lovastatin, atorvastatin, simvastatin, fluvastatin, itavastatin, ZD-4522), HMG-CoA synthase inhibitors, cholesterol-lowering agents, bile acid sequestrants (e.g., cholestyramine, questran, colestipol, and colesevelam), cholesterol absorption inhibitors (e.g. plant sterols such as phytosterols), cholesteryl ester transfer protein (CETP) inhibitors, inhibitors of the ileal bile acid transport system (IBAT inhibitors), diacylglyceryl acyltransferase 1 (DGAT1) inhibitors (e.g., AZD7687, LCQ908, compounds described in WO 2009/016462, WO 2010/086820), monoacylglycerol O-acyltransferase inhibitors, α-amylase inhibitors (e.g., tendamistat, trestatin, AL-3688), α-glucoside hydrolase inhibitors, SIRT-1 activators, c-Jun N-terminal kinase (JNK) inhibitors, a VPAC2 receptor agonist, TGR5 receptor modulators (e.g., compounds described in), GPBAR1 receptor modulators, GPR120 modulators, high affinity nicotinic acid receptor (HM74A) activators, carnitine palmitoyl transferase enzyme inhibitors, mineralocorticoid receptor inhibitors, inhibitors of TORC2, fatty acid synthetase inhibitors, serine palmitoyl transferase inhibitors, GPR81 modulators, GPR39 modulators, GPR43 modulators, GPR41 modulators, GPR105 modulators, Kv1.3 modulators, retinol binding protein 4 modulators, somatostain receptor modulators, PDHK2 modulators, PDHK4 modulators, MAP4K4 inhibitors, IL1 family modulators (e.g., IL1 beta modulators), ACAT inhibitors, MTP inhibitors (e.g., diriotapide, mitratapide, and implitapide), lipooxygenase inhibitors, PCSK9 modulators (e.g., alirocumab and evolocumab), RXRalpha modulators, cysteamine, cystamine, an RNA antisense construct to inhibit protein tyrosine phosphatase PTPRU, vitamin B complex, pentraxin proteins, a protein tyrosine phosphatase-1 B (PTP-1 B) inhibitor (e.g., trodusquemine, hyrtiosal extract, and compounds described by Zhang et al. Drug Discovery Today. 2007, 12(9-10): 373-381), ezitimbe, betaine, pentoxifylline, alpha delta-9 desaturase, BCKDK inhibitors, branched-chain alpha keto acid dehydrogenase kinase (BCBK) inhibitors, PNPLA3 inhibitors, FGF1 9 analogs, SCD1 inhibitors, bile acid binding resins, nicotinic acid (niacin) and analogues thereof, anti-oxidants (e.g., probucol), omega-3 fatty acids, antihypertensive agents, including adrenergic receptor antagonists, such as beta blockers (e.g. atenolol), alpha blockers (e.g. doxazosin), and mixed alpha/beta blockers (e.g. labetalol), adrenergic receptor agonists, including alpha-2 agonists (e.g. clonidine), angiotensin converting enzyme (ACE) inhibitors (e.g. lisinopril), calcium channel blockers, such as dihydropyridines (e.g. nifedipine), phenylalkylamines (e.g. verapamil), and benzothiazepines (e.g. diltiazem), angiotensin II receptor antagonists (e.g. candesartan), aldosterone receptor antagonists (e.g. eplerenone, spironolactone), centrally acting adrenergic drugs, such as central alpha agonists (e.g. clonidine), diuretic agents (e.g. furosemide, torsemide, bemetanide, ethacrynic acid, thiazide-type diuretics (e.g., chlorothiazide, hydrochlorothiazide, benzthiazide, hydroflumethiazide, bendroflumethiazide, methychlorthiazide, polythiazide, trichlormethiazide, indapamide), phthalimidine-type diuretics (e.g., chlorthalidone, metolazone), quinazoline-type diuretics (e.g., quinethazone), potassium-sparing diuretics (e.g., triamterene and amiloride), thyroid receptor agonists (e.g., compounds described in WO 2020/117987), haemostasis modulators, including antithrombotics (e.g., activators of fibrinolysis), thrombin antagonists, factor VIIa inhibitors, anticoagulants (e.g., vitamin K antagonists such as warfarin), heparin and low molecular weight analogues thereof, factor Xa inhibitors, and direct thrombin inhibitors (e.g. argatroban), antiplatelet agents (e.g., cyclooxygenase inhibitors (e.g. aspirin), non-steroidal anti-inflammatory drugs (NSAIDS), thromboxane-A2-receptor antagonists (e.g., ifetroban), thromboxane-A2-synthetase inhibitors, PDE inhibitors (e.g., Pletal, dipyridamole)), antagonists of purinergic receptors (e.g., P2Y1 and P2Y12), adenosine diphosphate (ADP) receptor inhibitors (e.g. clopidogrel), phosphodiesterase inhibitors (e.g. cilostazol), glycoprotein IIB/IIA inhibitors (e.g. tirofiban, eptifibatide, and abcixima), adenosine reuptake inhibitors (e.g. dipyridamole), noradrenergic agents (e.g. phentermine), serotonergic agents (e.g. sibutramine, lorcaserin), diacyl glycerolacyltransferase (DGAT) inhibitors, feeding behavior modifying agents, pyruvate dehydrogenase kinase (PDK) modulators, serotonin receptor modulators, monoamine transmission-modulating agents, such as selective serotonin reuptake inhibitors (SSRI) (e.g. fluoxetine), noradrenaline reuptake inhibitors (NARI), noradrenaline-serotonin reuptake inhibitors (SNRI), and monoamine oxidase inhibitors (MAOI) (e.g. toloxatone and amiflamine), compounds described in WO 2007/013694, WO 2007/018314, WO 2008/093639 and WO 2008/099794, GPR40 agonists (e.g., fasiglifam or a hydrate thereof, compounds described in WO 2004/041266, WO 2004/106276, WO 2005/063729, WO 2005/063725, WO 2005/087710, WO 2005/095338, WO 2007/013689 and WO 2008/001931), SGLT1 inhibitors, adiponectin or agonist thereof, IKK inhibitors (e.g., AS-2868), somatostatin receptor agonists, ACC2 inhibitors, cachexia-ameliorating agents, such as a cyclooxygenase inhibitors (e.g., indomethacin), progesterone derivatives (e.g., megestrol acetate), glucocorticoids (e.g., dexamethasone), metoclopramide agents, tetrahydrocannabinol agents, agents for improving fat metabolism (e.g., eicosapentaenoic acid), growth hormones, IGF-1, antibodies against a cachexia-inducing factor TNF-α, LIF, IL-6, and oncostatin M, metabolism-modifying proteins or peptides such as glucokinase (GK), glucokinase regulatory protein (GKRP), uncoupling proteins 2 and 3 (UCP2 and UCP3), peroxisome proliferator-activated receptor α (PPARα), MC4r agonists, insulin receptor agonist, PDE 5 inhibitors, glycation inhibitors (e.g., ALT-711), nerve regeneration-promoting drugs (e.g., Y-128, VX853, prosaptide), antidepressants (e.g., desipramine, amitriptyline, imipramine), antiepileptic drugs (e.g., lamotrigine, trileptal, keppra, zonegran, pregabalin, harkoseride, carbamazepine), antiarrhythmic drugs (e.g., K⁺ channel openers, mexiletine, propafenone, metoprolol, atenolol, carvadiol, propranolol, sotalol, dofetilide, amiodarone, azimilide, ibutilide, ditiazem, and verapamil), acetylcholine receptor ligands (e.g., ABT-594), endothelin receptor antagonists (e.g., ABT-627), narcotic analgesics (e.g., morphine), α2 receptor agonists (e.g., clonidine), local analgesics (e.g., capsaicin), antianxiety drugs (e.g., benzothiazepine), phosphodiesterase inhibitors (e.g., sildenafil), dopamine receptor agonists (e.g., apomorphine), cytotoxic antibodies (e.g., T-cell receptor and IL-2 receptor-specific antibodies), B cell depleting therapies (e.g., anti-CD20 antibody (e.g., rituxan), i-BLyS antibody), drugs affecting T cell migration (e.g., anti-integrin alpha 4/beta 1 antibody (e.g., tysabri), drugs that act on immunophilins (e.g., cyclosporine, tacrolimus, sirolimus, rapamicin), interferons (e.g., IFN-β), immunomodulators (e.g., glatiramer), TNF-binding proteins (e.g., circulating receptors), immunosupressants (e.g., mycophenolate), metaglidasen, AMG-131, balaglitazone, MBX-2044, rivoglitazone, aleglitazar, chiglitazar, saroglitazar, muraglitazar, tesaglitazar, lobeglitazone, PLX-204, PN-2034, GFT-505, THR-0921, exenatide, exendin-4, memantine, midazolam, ketoconazole, ethyl icosapentate, clonidine, azosemide, isosorbide, ethacrynic acid, piretanide, bumetanide, etoposide, piroxicam, NO donating agents (e.g., organonitrates), and NO promoting agents (e.g., phosphodiesterase inhibitors).

In some embodiments, the one or more additional therapeutic agents include those useful, for example, as anti-emetic agents. As used herein, an “anti-emetic” agent refers to any agent that counteracts (e.g., reduces or removes) nausea or emesis (vomiting). It is to be understood that when referring to a therapeutically effective amount of an anti-emetic agent, the amount administered is an amount needed to counteract (e.g., reduce or remove) nausea or emesis (vomiting). While not wishing to be bound by theory, it is believed that administering one or more anti-emetic agents in combination with the formula (I) compounds described herein may allow higher dosages of the formula (I) compounds to be administered, e.g., because the patient may be able to have a normal food intake and thereby respond faster to the treatment.

Non-limiting examples of anti-emetic agents include 5HT3-receptor antagonists (serotonin receptor antagonists), neuroleptics/anti-psychotics, antihistamines, anticholinergic agents, steroids (e.g., corticosteroids), NK1-receptor antagonists (e.g., Neurokinin 1 substance P receptor antagonists), antidopaminergic agents/dopamine receptor antagonists, benzodiazepines, cannabinoids.

For example, the antiemetic agent can be selected from the group consisting of; neuroleptics, antihistamines, anti-cholinergic agents, steroids, 5HT-3-receptor antagonists, NK1-receptor antagonists, anti-dopaminergic agents/dopamine receptor antagonists, benzodiazepines and non-psychoactive cannabinoids.

In some embodiments, the anti-emetic agent is a 5HT3-receptor antagonist (serotonin receptor antagonist). Non-limiting examples of 5HT3-receptor antagonists (serotonin receptor antagonists) include: Granisetron (Kytril), Dolasetron, Ondansetron (Zofran), Tropisetron, Ramosetron, Palonosetron, Alosetron, azasetron, Bemesetron, Zatisetron, Batanopirde, MDL-73147EF; Metoclopramide, N-3389 (endo-3,9-dimethyl-3,9-diazabicyclo[3,3,1]non-7-yl-1H-indazole-3-carboxamide dihydrochloride), Y-25130 hydrochloride, MDL 72222, tropanyl-3,5-dimethylbenzoate, 3-(4-allylpiperazin-1-yl)-2-quinoxalinecarbonitrile maleate, Zacopride hydrochloride, and Mirtazepine. Other non-limiting examples of 5HT3-receptor antagonists (serotonin receptor antagonists) include: cilansetron, clozapine, cyproheptadine, dazopride, hydroxyzine, lerisetron, metoclopramide, mianserin, olanzapine, palonosetron (+netupitant), quetiapine, qamosetron, ramosteron, ricasetron, risperidone, ziprasidone, and zatosetron.

In certain embodiments, the 5HT-3-receptor antagonist is granisetron, dolasetron, ondansetron hydrochloride, tropisetron, ramosetron, palonosetron, alosetron, bemesetron, zatisetron, batanopirde, MDL-73147EF, metoclopramide, N-3389, Y—25130 hydrochloride, MDL 72222, tropanyl-3,5-dimethylbenzoate 3-(4-allyl-piperazin-1-yl)-2-quinoxalinecarbonitrile maleate, zacopride hydrochloride and Mirtazepine.

In certain embodiments, the 5HT-3-receptor antagonist is granisetron, dolasetron, ondansetron hydrochloride, tropisetron, ramosetron, palonosetron, alosetron, bemesetron, and zatisetron.

In certain embodiments, the 5HT-3-receptor antagonist is granisetron, dolasetron and ondansetron.

In certain embodiments, the 5HT-3-receptor antagonist is granisetron.

In certain embodiments, the 5HT-3-receptor antagonist is ondansetron.

In some embodiments, the anti-emetic agent is an antihistamine. Non-limiting examples of antihistamines include: piperazine derivatives (e.g., cyclizine, meclizine, and cinnarizine); promethazine; dimenhydrinate (Dramamine, Gravol); diphenhydramine; hydroxyzine; buclizine; and meclizine hydrochloride (Bonine, Antivert), doxylamine, and mirtazapine.

In some embodiments, the anti-emetic agent is an anticholinergic agent (inhibitors of the acetylcholine receptors). Non-limiting examples of anticholinergic agents include: atropine, Scopolamine, Glycopyrron, Hyoscine, Artane (trihexy-5 trihexyphenidyl hydrochloride), Cogentin (benztropine mesylate), Akineton (biperiden hydrochloride), Disipal (Norflex orphenadrine citrate), diphenhydramine, hydroxyzine, hyoscyamine, and Kemadrin (procyclidine hydrochloride).

In some embodiments, the anti-emetic agent is a steroid (e.g., a corticosteroid). Non-limiting examples of steroids include: betamethasone, dexamethasone, methylprednisolone, Prednisone®, and trimethobenzamide (Tigan).

In some embodiments, the anti-emetic agent is an NK1-receptor antagonists (e.g., Neurokinin 1 substance P receptor antagonists). Non-limiting examples of NK1-receptor antagonists include: aprepitant, casopitant, ezlopitant, fosaprepitant, maropitant, netupitant, rolapitant, and vestipitant.

Other non-limiting examples of NK1-receptor antagonists include: MPC-4505, GW597599, MPC-4505, GR205171, L-759274, SR 140333, CP-96,345, BIIF 1149, NKP 608C, NKP 608A, CGP 60829, SR 140333 (Nolpitantium besilate/chloride), LY 303870 (Lanepitant), MDL-105172A, MDL-103896, MEN-11149, MEN-11467, DNK 333A, YM-49244, YM-44778, ZM-274773, MEN-10930, S-19752, Neuronorm, YM-35375, DA-5018, MK-869, L-754030, CJ-11974, L-758298, DNK-33A, 6b-1, CJ-11974 j. Benserazide and carbidopa k. TAK-637 [(aR,9R)-7-[3,5-bis(trifluoromethyl)benzyl]-8,9,10,11-tetrahydro-9-methyl-5-(4-methylphenyl)-7H-[1,4]diazocino[2,1-g][1,7]naphthyridine-6,13-dione], PD 154075, ([(2-benzofuran)-CH2OCO]—(R)-alpha-MeTrp-(S)-NHCH(CH3) Ph), FK888, and (D-Pro4, D-Trp7,9,10, Phe11)SP4-11.

In some embodiments, the anti-emetic agent is an anti-dopaminergic agents/dopamine receptor antagonist (e.g., dopamine receptor antagonist, e.g., D2 or D3 antagonists). Non-limiting examples include phenothiazines (e.g., promethazine, chlorpromazine, prochlorperazine, perphenazine, hydroxyzine, thiethylperazine, metopimazine,); benzamides (e.g., metoclopramide, domperidone), butyrophenones (e.g., haloperidol, droperidol); alizapride, bromopride, clebopride, domperidone, itopride, metoclopramide, trimethobenzamide, and amisulpride.

In some embodiments, the anti-emetic agent is a non-psychoactive cannabinoids (e.g., cannabidiol (CBD), cannabidiol dimethylheptyl (CBD-DMH), tetra-hydro-cannabinol (THC), cannabinoid agonists such as WIN 55-212 (a CB1 and CB2 receptor agonist), dronabinol (Marinol®), and nabilone (Cesamet)).

Other exemplary anti-emetic agents include: c-9280 (Merck); benzodiazepines (diazepam, midazolam, lorazepam); neuroleptics/anti-psychotics (e.g., dixyrazine, haloperidol, and prochlorperazine (Compazine®)); cerium oxalate; propofol; sodium citrate; dextrose; fructose (Nauzene); orthophosphoric acid; fructose; glucose (Emetrol); bismuth subsalicylate (Pepto Bismol); ephedrine; vitamin B6; peppermint, lavender, and lemon essential oils; and ginger.

In some embodiments, the additional therapeutic agent or regimen is administered to the patient prior to contacting with or administering the compounds and pharmaceutical compositions (e.g., about one hour prior, or about 6 hours prior, or about 12 hours prior, or about 24 hours prior, or about 48 hours prior, or about 1 week prior, or about 1 month prior).

In some embodiments, the additional therapeutic agent or regimen is administered to the patient at about the same time as contacting with or administering the compounds and pharmaceutical compositions. By way of example, the additional therapeutic agent or regimen and the compounds and pharmaceutical compositions are provided to the patient simultaneously in the same dosage form. As another example, the additional therapeutic agent or regimen and the compounds and pharmaceutical compositions are provided to the patient concurrently in separate dosage forms.

### Pharmaceutical Compositions and Modes of Administration

When employed as pharmaceuticals, the compounds of Formula I, including pharmaceutically acceptable salts or solvates thereof can be administered in the form of a pharmaceutical compositions. These compositions can be prepared in a manner well known in the pharmaceutical art, and can be administered by a variety of routes, depending upon whether local or systemic treatment is desired and upon the area to be treated. Administration can be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Oral administration can include a dosage form formulated for once-daily or twice-daily (BID) administration. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or can be, for example, by a continuous perfusion pump. Pharmaceutical compositions and formulations for topical administration can include transdermal patches, ointments, lotions, creams, gels, drops, suppositories, sprays, liquids and powders. Conventional pharmaceutical carriers, aqueous, powder or oily bases, thickeners and the like may be necessary or desirable.

Also provided herein are pharmaceutical compositions which contain, as the active ingredient, a compound of Formula I, or a pharmaceutically acceptable salt, deuterated analog, or solvate thereof, in combination with one or more pharmaceutically acceptable excipients (carriers). For example, a pharmaceutical composition prepared using a compound of Formula I, or a pharmaceutically acceptable salt, deuterated analog, or solvate thereof. In some embodiments, the composition is suitable for topical administration. In making the compositions provided herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders. In some embodiments, the composition is formulated for oral administration. In some embodiments, the composition is a solid oral formulation. In some embodiments, the composition is formulated as a tablet or capsule.

Further provided herein are pharmaceutical compositions containing a compound of Formula I, or a pharmaceutically acceptable salt, deuterated analog, or solvate thereof with a pharmaceutically acceptable excipient. Pharmaceutical compositions containing a compound of Formula I, or a pharmaceutically acceptable salt, deuterated analog, or solvate thereof as the active ingredient can be prepared by intimately mixing the compound of Formula I, or a pharmaceutically acceptable salt, deuterated analog, or solvate thereof with a pharmaceutical carrier according to conventional pharmaceutical compounding techniques. The carrier can take a wide variety of forms depending upon the desired route of administration (e.g., oral, parenteral). In some embodiments, the composition is a solid oral composition.

Suitable pharmaceutically acceptable carriers are well known in the art. Descriptions of some of these pharmaceutically acceptable carriers can be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

In some embodiments, the compound or pharmaceutical composition can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a chemical entity as described herein in the range of 0.005% to 100% with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, U K. 2012).

In some embodiments, the compounds and pharmaceutical compositions described herein or a pharmaceutical composition thereof can be administered to patient in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal (e.g., intranasal), nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In some embodiments, a route of administration is oral or parenteral (e.g., intratumoral).

In some embodiments, a compound of Formula I, or a pharmaceutically acceptable salt, deuterated analog, or solvate thereof (e.g., a compound of any one of Formula II, III, or IV, or a pharmaceutically acceptable salt, deuterated analog, or solvate thereof) as described herein or pharmaceutical compositions thereof can be formulated for parenteral administration, e.g., formulated for injection via the intraarterial, intrasternal, intracranial, intravenous, intramuscular, sub-cutaneous, or intraperitoneal routes. For example, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure. In some embodiments, devices are used for parenteral administration. For example, such devices may include needle injectors, microneedle injectors, needle-free injectors, and infusion techniques.

In some embodiments, the pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In some embodiments, the form must be sterile and must be fluid to the extent that it may be easily injected. In some embodiments, the form should be stable under the conditions of manufacture and storage and must be preserved against the contaminating action of microorganisms, such as bacteria and fungi.

In some embodiments, the carrier also can be a solvent or dispersion medium containing, for example, water, ethanol, polyol (for example, glycerol, propylene glycol, and liquid polyethylene glycol, and the like), suitable mixtures thereof, and vegetable oils. In some embodiments, the proper fluidity can be maintained, for example, by the use of a coating, such as lecithin, by the maintenance of the required particle size in the case of dispersion, and by the use of surfactants. In some embodiments, the prevention of the action of microorganisms can be brought about by various antibacterial and antifungal agents, for example, parabens, chlorobutanol, phenol, sorbic acid, thimerosal, and the like. In some embodiments, isotonic agents, for example, sugars or sodium chloride are included. In some embodiments, prolonged absorption of the injectable compositions can be brought about by the use in the compositions of agents delaying absorption, for example, aluminum monostearate and gelatin.

In some embodiments, sterile injectable solutions are prepared by incorporating a compound of Formula I, or a pharmaceutically acceptable salt, deuterated analog, or solvate thereof (e.g., a compound of any one of Formula II, III, or IV, or a pharmaceutically acceptable salt, deuterated analog, or solvate thereof) in the required amount in the appropriate solvent with various of the other ingredients enumerated above, as required, followed by filtered sterilization. In some embodiments, dispersions are prepared by incorporating the various sterilized active ingredients into a sterile vehicle which contains the basic dispersion medium and the required other ingredients from those enumerated above. In some embodiments, sterile powders are used for the preparation of sterile injectable solutions. In some embodiments, the methods of preparation are vacuum-drying and freeze-drying techniques, which yield a powder of the active ingredient, plus any additional desired ingredient from a previously sterile-filtered solution thereof.

In some embodiments, pharmacologically acceptable excipients usable in a rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol, Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In some embodiments, suppositories can be prepared by mixing a compound of Formula I, or a pharmaceutically acceptable salt, deuterated analog, or solvate thereof (e.g., a compound of any one of Formula II, III, or IV, or a pharmaceutically acceptable salt, deuterated analog, or solvate thereof) or pharmaceutical compositions as described herein with suitable non-irritating excipients or carriers such as cocoa butter, polyethylene glycol or a suppository wax which are solid at ambient temperature but liquid at body temperature and therefore melt in the rectum and release the active compound. In some embodiments, compositions for rectal administration are in the form of an enema.

In some embodiments, a compound of Formula I, or a pharmaceutically acceptable salt, deuterated analog, or solvate thereof (e.g., a compound of any one of Formula II, III, or IV, or a pharmaceutically acceptable salt, deuterated analog, or solvate thereof) as described herein or a pharmaceutical composition thereof is formulated for local delivery to the digestive or GI tract by way of oral administration (e.g., solid or liquid dosage forms.).

In some embodiments, solid dosage forms for oral administration include capsules, tablets, pills, powders, and granules. In some embodiments, a compound of Formula I, or a pharmaceutically acceptable salt, deuterated analog, or solvate thereof (e.g., a compound of any one of Formula II, III, or IV, or a pharmaceutically acceptable salt, deuterated analog, or solvate thereof) is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For example, in the case of capsules, tablets and pills, the dosage form may also comprise buffering agents. In some embodiments, solid compositions of a similar type may also be employed as fillers in soft and hard-filled gelatin capsules using such excipients as lactose or milk sugar as well as high molecular weight polyethylene glycols and the like.

In some embodiments, the pharmaceutical compositions will take the form of a unit dosage form such as a pill or tablet and thus the composition may contain, along with a compound of Formula I, or a pharmaceutically acceptable salt, deuterated analog, or solvate thereof (e.g., a compound of any one of Formula II, III, or IV, or a pharmaceutically acceptable salt, deuterated analog, or solvate thereof) as provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In some embodiments, another solid dosage form, a powder, marume, solution or suspension (e.g., in propylene carbonate, vegetable oils, PEG's, poloxamer 124 or triglycerides) is encapsulated in a capsule (gelatin or cellulose base capsule). In some embodiments, unit dosage forms in which one or more compounds and pharmaceutical compositions as provided herein or additional active agents are physically separated are also contemplated; e.g., capsules with granules (or tablets in a capsule) of each drug; two-layer tablets; two-compartment gel caps, etc. In some embodiments, enteric coated or delayed release oral dosage forms are also contemplated.

In some embodiments, other physiologically acceptable compounds may include wetting agents, emulsifying agents, dispersing agents or preservatives that are particularly useful for preventing the growth or action of microorganisms. For example, various preservatives are well known and include, for example, phenol and ascorbic acid.

In some embodiments, the excipients are sterile and generally free of undesirable matter. For example, these compositions can be sterilized by conventional, well-known sterilization techniques. In some embodiments, for various oral dosage form excipients such as tablets and capsules, sterility is not required. For example, the United States Pharmacopeia/National Formulary (USP/NF) standard can be sufficient.

In some embodiments, a compound of Formula I, or a pharmaceutically acceptable salt, deuterated analog, or solvate thereof (e.g., a compound of any one of Formula II, III, or IV, or a pharmaceutically acceptable salt, deuterated analog, or solvate thereof) as described herein or a pharmaceutical composition thereof is formulated for ocular administration. In some embodiments, ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, EDTA, SofZia (boric acid, propylene glycol, sorbitol, and zinc chloride; Alcon Laboratories, Inc.), Purite (stabilized oxychloro complex; Allergan, Inc.)).

In some embodiments, a compound of Formula I, or a pharmaceutically acceptable salt, deuterated analog, or solvate thereof (e.g., a compound of any one of Formula II, III, or IV, or a pharmaceutically acceptable salt, deuterated analog, or solvate thereof) as described herein or a pharmaceutical composition thereof is formulated for topical administration to the skin or mucosa (e.g., dermally or transdermally). In some embodiments, topical compositions can include ointments and creams. In some embodiments, ointments are semisolid preparations that are typically based on petrolatum or other petroleum derivatives. In some embodiments, creams containing the selected active agent are typically viscous liquid or semisolid emulsions, often either oil-in-water or water-in-oil. For example, cream bases are typically water-washable, and contain an oil phase, an emulsifier and an aqueous phase. For example, the oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. In some embodiments, the emulsifier in a cream formulation is generally a nonionic, anionic, cationic or amphoteric surfactant. In some embodiments, as with other carriers or vehicles, an ointment base should be inert, stable, nonirritating and non-sensitizing.

In any of the foregoing embodiments, pharmaceutical compositions as described herein can include one or more one or more of the following: lipids, interbilayer crosslinked multilamellar vesicles, biodegradable poly(D,L-lactic-co-glycolic acid) [PLGA]-based or poly anhydride-based nanoparticles or microparticles, and nanoporous particle-supported lipid bilayers.

In some embodiments, the dosage for a compound of Formula I, or a pharmaceutically acceptable salt, deuterated analog, or solvate thereof (e.g., a compound of any one of Formula II, III, or IV, or a pharmaceutically acceptable salt, deuterated analog, or solvate thereof), is determined based on a multiple factors including, but not limited to, type, age, weight, sex, medical condition of the patient, severity of the medical condition of the patient, route of administration, and activity of the compound or pharmaceutically acceptable salt, deuterated analog, or solvate thereof. In some embodiments, proper dosage for a particular situation can be determined by one skilled in the medical arts. In some embodiments, the total daily dosage may be divided and administered in portions throughout the day or by means providing continuous delivery.

In some embodiments, a compound of Formula I, or a pharmaceutically acceptable salt, deuterated analog, or solvate thereof (e.g., a compound of any one of Formula II, III, or IV, or a pharmaceutically acceptable salt, deuterated analog, or solvate thereof), is administered at a dose from about 0.01 to about 1000 mg. For example, from about 0.1 to about 30 mg, about 10 to about 80 mg, about 0.5 to about 15 mg, about 50 mg to about 200 mg, about 100 mg to about 300 mg, about 200 to about 400 mg, about 300 mg to about 500 mg, about 400 mg to about 600 mg, about 500 mg to about 800 mg, about 600 mg to about 900 mg, or about 700 mg to about 1000 mg. In some embodiments, the dose is a therapeutically effective amount.

In some embodiments, a compound of Formula I, or a pharmaceutically acceptable salt, deuterated analog, or solvate thereof (e.g., a compound of any one of Formula II, III, or IV, or a pharmaceutically acceptable salt, deuterated analog, or solvate thereof) as described herein is administered at a dosage of from about 0.0002 mg/Kg to about 100 mg/Kg (e.g., from about 0.0002 mg/Kg to about 50 mg/Kg; from about 0.0002 mg/Kg to about 25 mg/Kg; from about 0.0002 mg/Kg to about 10 mg/Kg; from about 0.0002 mg/Kg to about 5 mg/Kg; from about 0.0002 mg/Kg to about 1 mg/Kg; from about 0.0002 mg/Kg to about 0.5 mg/Kg; from about 0.0002 mg/Kg to about 0.1 mg/Kg; from about 0.001 mg/Kg to about 50 mg/Kg; from about 0.001 mg/Kg to about 25 mg/Kg; from about 0.001 mg/Kg to about 10 mg/Kg; from about 0.001 mg/Kg to about 5 mg/Kg; from about 0.001 mg/Kg to about 1 mg/Kg; from about 0.001 mg/Kg to about 0.5 mg/Kg; from about 0.001 mg/Kg to about 0.1 mg/Kg; from about 0.01 mg/Kg to about 50 mg/Kg; from about 0.01 mg/Kg to about 25 mg/Kg; from about 0.01 mg/Kg to about 10 mg/Kg; from about 0.01 mg/Kg to about 5 mg/Kg; from about 0.01 mg/Kg to about 1 mg/Kg; from about 0.01 mg/Kg to about 0.5 mg/Kg; from about 0.01 mg/Kg to about 0.1 mg/Kg; from about 0.1 mg/Kg to about 50 mg/Kg; from about 0.1 mg/Kg to about 25 mg/Kg; from about 0.1 mg/Kg to about 10 mg/Kg; from about 0.1 mg/Kg to about 5 mg/Kg; from about 0.1 mg/Kg to about 1 mg/Kg; from about 0.1 mg/Kg to about 0.5 mg/Kg). In some embodiments, a compound of Formula I, or a pharmaceutically acceptable salt, deuterated analog, or solvate thereof (e.g., a compound of any one of Formula II, III, or IV, or a pharmaceutically acceptable salt, deuterated analog, or solvate thereof) as described herein is administered as a dosage of about 100 mg/Kg.

In some embodiments, the foregoing dosages of a compound of Formula I, or a pharmaceutically acceptable salt, deuterated analog, or solvate thereof (e.g., a compound of any one of Formula II, III, or IV, or a pharmaceutically acceptable salt, deuterated analog, or solvate thereof), can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a compound of Formula I, or a pharmaceutically acceptable salt, deuterated analog, or solvate thereof (e.g., a compound of any one of Formula II, III, or IV, or a pharmaceutically acceptable salt, deuterated analog, or solvate thereof) as described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In some embodiments, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In some embodiments, a compound of Formula I, or a pharmaceutically acceptable salt, deuterated analog, or solvate thereof (e.g., a compound of any one of Formula II, III, or IV, or a pharmaceutically acceptable salt, deuterated analog, or solvate thereof) is administered to a patient for a period of time followed by a separate period of time where administration of the compound of Formula I, or a pharmaceutically acceptable salt, deuterated analog, or solvate thereof (e.g., a compound of any one of Formula II, III, or IV, or a pharmaceutically acceptable salt, deuterated analog, or solvate thereof) is stopped. In some embodiments, a compound of Formula I, or a pharmaceutically acceptable salt, deuterated analog, or solvate thereof (e.g., a compound of any one of Formula II, III, or IV, or a pharmaceutically acceptable salt, deuterated analog, or solvate thereof) is administered for a first period and a second period following the first period, with administration stopped during the second period, followed by a third period where administration of the compound of Formula I, or a pharmaceutically acceptable salt, deuterated analog, or solvate thereof (e.g., a compound of any one of Formula II, III, or IV, or a pharmaceutically acceptable salt, deuterated analog, or solvate thereof) is started and then a fourth period following the third period where administration is stopped. For example, the period of administration of a compound of Formula I, or a pharmaceutically acceptable salt, deuterated analog, or solvate thereof (e.g., a compound of any one of Formula II, III, or IV, or a pharmaceutically acceptable salt, deuterated analog, or solvate thereof) followed by a period where administration is stopped is repeated for a determined or undetermined period of time. In some embodiments, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In some embodiments, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In some embodiments, a compound of Formula I, or a pharmaceutically acceptable salt, deuterated analog, or solvate thereof (e.g., a compound of any one of Formula II, III, or IV, or a pharmaceutically acceptable salt, deuterated analog, or solvate thereof), is orally administered to the patient one or more times per day (e.g., one time per day, two times per day, three times per day, four times per day per day or a single daily dose).

In some embodiments, a compound of Formula I, or a pharmaceutically acceptable salt, deuterated analog, or solvate thereof (e.g., a compound of any one of Formula II, III, or IV, or a pharmaceutically acceptable salt, deuterated analog, or solvate thereof), is administered by parenteral administration to the patient one or more times per day (e.g., 1 to 4 times per day, two times per day, three times per day, four times per day or a single daily dose).

In some embodiments, a compound of Formula I, or a pharmaceutically acceptable salt, deuterated analog, or solvate thereof (e.g., a compound of any one of Formula II, III, or IV, or a pharmaceutically acceptable salt, deuterated analog, or solvate thereof), is administered by parenteral administration to the patient weekly.

### Patient Selection

In some embodiments, the methods described herein further include the step of identifying a patient (e.g., a subject) in need of such treatment (e.g., by way of blood assay, body mass index, or other conventional method known in the art).

In some embodiments, the methods described herein further include the step of identifying a patient (e.g., patient) that has a disease, disorder, or condition as provided here (e.g., a GLP-1 associated disease, disorder, or condition).

In some embodiments, the methods described herein further include the step of identifying a patient (e.g., patient) that has type 2 diabetes mellitus. In some embodiments, determining if the patient has type 2 diabetes mellitus includes performing an assay to determine the level of hemoglobin Alc (HbA1c), fasting plasma glucose, non-fasting plasma glucose, or any combination thereof. In some embodiments, the level of HbA1c is about 6.5% to about 24.0%. In some embodiments, the level of HbA1c is greater than or about 6.5%. In some embodiments, the level of HbA1c is greater than or about 8.0%. In some embodiments, the level of HbA1c is greater than or about 10.0%. In some embodiments, the level of HbA1c is greater than or about 12.0%. In some embodiments, the level of HbA1c is greater than or about 14.0%. In some embodiments, the level of HbA1c is greater than or about 16.0%. In some embodiments, the level of HbA1c is greater than or about 18.0%. In some embodiments, the level of HbA1c is greater than or about 20.0%. In some embodiments, the level of HbA1c is greater than or about 22.0%. In some embodiments, the level of HbA1c is greater than or about 24.0%.

In some embodiments, the level of fasting plasma glucose is greater than or about 120 mg/dL to greater than or about 750 mg/dL. In some embodiments, the level of fasting plasma glucose is greater than or about 200 mg/dL to greater than or about 500 mg/dL. In some embodiments, the level of fasting plasma glucose is greater than or about 300 mg/dL to greater than or about 700 mg/dL.

In some embodiments, the level of non-fasting plasma glucose is greater than or about 190 mg/dL to greater than or about 750 mg/dL. In some embodiments, the level of non-fasting plasma glucose is greater than or about 250 mg/dL to greater than or about 450 mg/dL. In some embodiments, the level of non-fasting plasma glucose is greater than or about 400 mg/dL to greater than or about 700 mg/dL.

In some embodiments, determining if the patient has type 2 diabetes mellitus further includes determining the patient's BMI. In some embodiments, the BMI of the patient is greater than or about 22 kg/m² to greater than or about 100 kg/m². In some embodiments, the BMI of the patient is greater than or about 30 kg/m² to greater than or about 90 kg/m². In some embodiments, the BMI of the patient is greater than or about 40 kg/m² to greater than or about 80 kg/m². In some embodiments, the BMI of the patient is greater than or about 50 kg/m² to greater than or about 70 kg/m².

In some embodiments, additional factors (e.g. risk factors) used for determining if the patient has type 2 diabetes mellitus further includes age and ethnicity of the patient. In some embodiments, the patient's age is greater than or about 10 years. In some embodiments, the patient's age is greater than or about 15 years. In some embodiments, the patient's age is greater than or about 20 years. In some embodiments, the patient's age is greater than or about 25 years. In some embodiments, the patient's age is greater than or about 30 years. In some embodiments, the patient's age is greater than or about 35 years. In some embodiments, the patient's age is greater than or about 40 years. In some embodiments, the patient's age is greater than or about 42 years. In some embodiments, the patient's age is greater than or about 44 years. In some embodiments, the patient's age is greater than or about 46 years. In some embodiments, the patient's age is greater than or about 48 years. In some embodiments, the patient's age is greater than or about 50 years. In some embodiments, the patient's age is greater than or about 52 years. In some embodiments, the patient's age is greater than or about 54 years. In some embodiments, the patient's age is greater than or about 56 years. In some embodiments, the patient's age is greater than or about 58 years. In some embodiments, the patient's age is greater than or about 60 years. In some embodiments, the patient's age is greater than or about 62 years. In some embodiments, the patient's age is greater than or about 64 years. In some embodiments, the patient's age is greater than or about 66 years. In some embodiments, the patient's age is greater than or about 68 years. In some embodiments, the patient's age is greater than or about 70 years. In some embodiments, the patient's age is greater than or about 72 years. In some embodiments, the patient's age is greater than or about 74 years. In some embodiments, the patient's age is greater than or about 76 years. In some embodiments, the patient's age is greater than or about 78 years. In some embodiments, the patient's age is greater than or about 80 years. In some embodiments, the patient's age is greater than or about 85 years. In some embodiments, the patient's age is greater than or about 90 years. In some embodiments, the patient's age is greater than or about 95 years. In some embodiments, the ethnicity of the patient may be African American, American Indian or Alaska Native, Asian American, Hispanics or Latinos, or Native Hawaiian or Pacific Islander.

### Synthesis of the Compounds

The compounds may be prepared using the methods disclosed herein and routine modifications thereof, which will be apparent given the disclosure herein and methods well known in the art. Conventional and well-known synthetic methods may be used in addition to the teachings herein. The synthesis of typical compounds described herein may be accomplished as described in the following examples. If available, reagents may be purchased commercially, e.g., from Sigma Aldrich or other chemical suppliers.

### General Synthesis

The compounds of this disclosure can be prepared from readily available starting materials using, for example, the following general methods, and procedures. It will be appreciated that where certain process conditions (i.e., reaction temperatures, times, mole ratios of reactants, solvents, pressures, etc.) are given, other process conditions can also be used unless otherwise stated. Optimum reaction conditions may vary with the reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting certain functional groups are well known in the art. For example, numerous protecting groups are described in T. W. Greene and G. M. Wuts (1999) Protecting Groups in Organic Synthesis, 3rd Edition, Wiley, New York, and references cited therein.

Furthermore, the compounds of this disclosure may contain one or more chiral centers. Accordingly, if desired, such compounds can be prepared or isolated as pure stereoisomers, i.e., as individual enantiomers or diastereomers, or as stereoisomer-enriched mixtures. All such stereoisomers (and enriched mixtures) are included within the scope of this disclosure, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents, and the like.

The starting materials for the following reactions are generally known compounds or can be prepared by known procedures or obvious modifications thereof. For example, many of the starting materials are available from commercial suppliers such as Aldrich Chemical Co. (Milwaukee, Wisconsin, USA), Bachem (Torrance C A USA), EMKA-Chemie Gmbh & Co. KG (Eching Germany), or Millipore Sigma (Burlington MA USA). Others may be prepared by procedures, or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989), Organic Reactions, Volumes 1-40 (John Wiley, and Sons, 1991), March's Advanced Organic Chemistry, (John Wiley, and Sons, 5^{th} Edition, 2001), and Larock's Comprehensive Organic Transformations (VCH Publishers Inc., 1989).

Scheme I illustrates a general method which can be employed for the synthesis of compounds described herein, where each of Ring A, Ring B, Ring C, Q, X, Y, L¹, L², R¹, R², and R³ are independently as defined herein, and LG is a leaving group, such as alkoxy, hydroxy, halo (e.g., Cl, Br, or I), and the like.

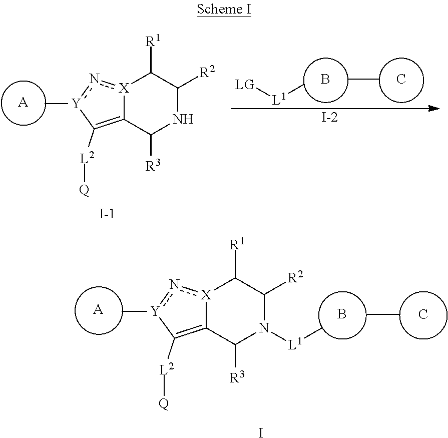

Referring to Scheme I, coupling of compounds I-1 with I-2 under standard coupling conditions, such as amide bond forming conditions when L¹ is —C(═O)—, e.g., in the presence of a carboxyl activation agent such as HATU (e.g., in a polar aprotic solvent such as DMF), provides compounds of Formula I. Upon reaction completion, compounds of Formula I can be recovered by conventional techniques such as neutralization, extraction, precipitation, chromatography, filtration, and the like.

Scheme II illustrates a general method which can be employed for the synthesis of compound I-2, where Ring B and Ring C are independently as defined herein, L3 is a bond, and R^{8a} and R^{8b} are taken together with the carbon atom to which each is attached to form an optionally substituted C₃₋₁₅ cycloalkyl (e.g., cyclopropyl), which is denoted in Scheme II as Ring D′, the dashed curve Lx represents a C₂₋₄ alkylene which may be optionally substituted as defined herein, (e.g., optionally substituted with a C₁₋₆ alkyl, which is optionally substituted with from 1-3 R^{f}), R⁵⁰ is an alkyl group, which may be optionally substituted, and LG is a leaving group, such as alkoxy, hydroxy, halo (e.g., Cl, Br, or I), and the like.

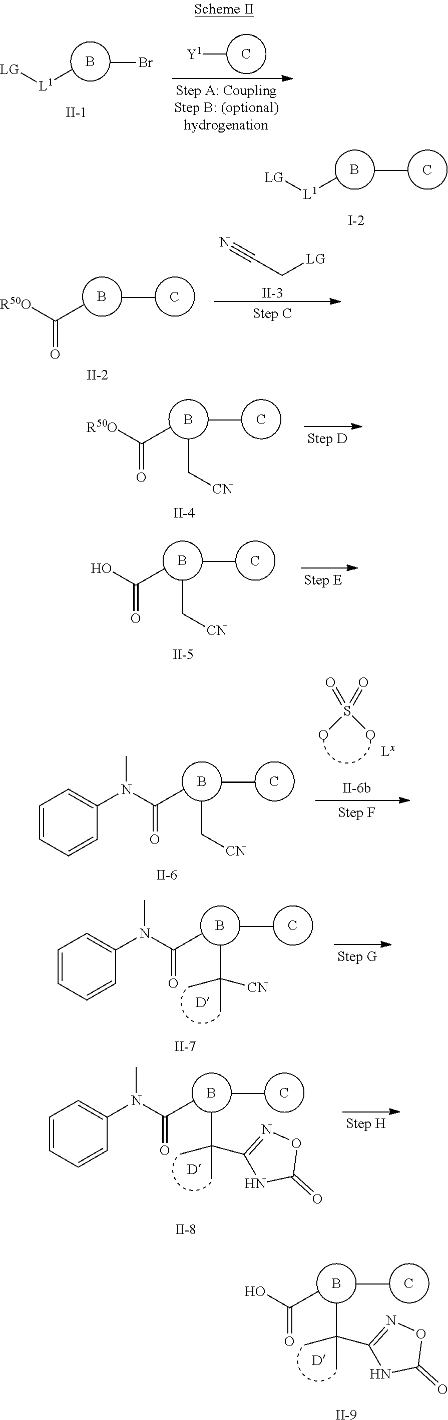

Referring to Scheme II, compound II-1 is subjected to a metal-catalyzed cross-coupling reaction (e.g., Suzuki coupling using Pd(dppf)ClCH₂Cl₂ in the presence of potassium carbonate) with an appropriate coupling partner such as a reagent with Formula (Ring C)—Y¹ (wherein Ring C is as defined for Formula I; and Y¹ is B(OH)₂ or boronate ester such as BPin) to provide compound I-2. Optionally when Ring C is a saturated heterocyclyl, II-1 can be coupled with a reagent of Formula (Ring C)—Y¹ wherein Ring C is a partially saturated heterocyclyl, followed by optional hydrogenation (e.g., with palladium on carbon) to afford 1-2. As a non-limiting example, when Ring C is tetrahydropyran-4-yl, II-1 can be coupled with (Ring-C)—Y¹, e.g., 2-(3,6-dihydro-2H-pyran-4-yl)-4,4,5,5-tetramethyl-1,3,2-dioxaborolane of formula:

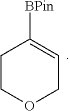

Certain compounds of Formula I-2 can be prepared from compound 11-2 as shown in Scheme II. For example, reaction of 11-2 with 11-3 (e.g., in the presence of NaH in DMF) provides compound 11-4, whereupon hydrolysis of the ester group under standard conditions (e.g., with LiOH in H₂O/THF) affords compound II-5. Amide coupling of II-5 with NHMePh under standard conditions (e.g., HATU and Hunig's base) provides II-6. The reaction of 11-6 with a reagent II-6b under basic conditions (e.g., KHMDS, DMPU) then provides II-7. As a non-limiting example, II-6b can be

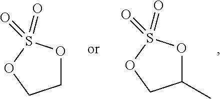

wherein the Ring D′ in the corresponding product is

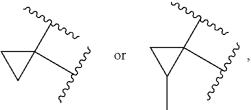

respectively. Treatment of II-7 with a hydroxylamine source (e.g., NH₂OH·HCl in EtOH under reflux) followed by reaction with a phosgene equivalent (e.g., CDI (e.g., in the presence of DBU and DMSO at 80° C.)) provides compound 11-8. Hydrolysis of the amide group in compound 11-8 (e.g., with KOH in CH₃OCH₂CH₂OH at e.g., 125° C.) provides compound II-9. Compound II-9 can be used in Scheme I as compound I-2.

In certain embodiments, the procedure outlined in Scheme II for the installation of the

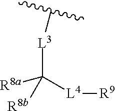

moiety on Ring B can be performed according to Scheme IIA shown below, where each of Ring A, Ring B, Ring C, Ring D′, Q, X, Y, L¹, L², R¹, R², and R³ are independently as defined herein.

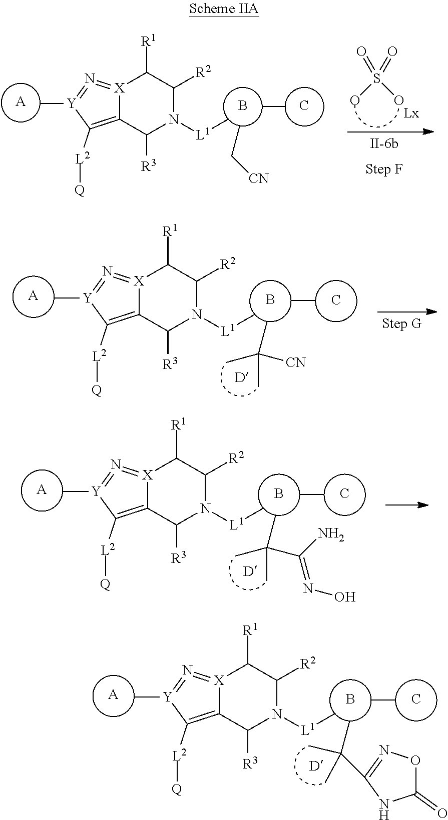

Scheme III illustrates a general method which can be employed for the synthesis of exemplary compounds of Formula I-1 (e.g., compound III-7), which can be employed in Scheme I for providing compounds of Formula I, where each of Ring A, Q, X, Y, L¹, L², R¹, R², and R³ are independently as defined herein, and X is a leaving group, such as halo (e.g., Cl, Br, or I), and PG is a protecting group.

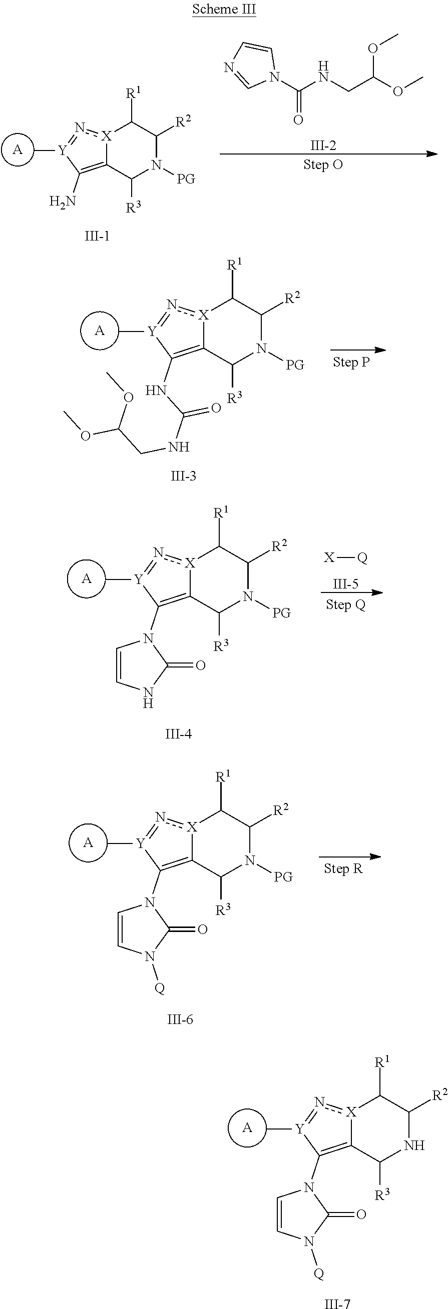

In Scheme III, reaction between III-1 and III-2 (e.g., in the presence of a polar aprotic solvent such as DMA) provides compound 11I-3, whereupon treatment under acid (e.g., MeSO₃H, THF, 60° C.) followed by reprotection of the secondary amino group (e.g., with Boc₂O) provides compound III-4, wherein PG is a nitrogen protecting group such as a carbamate protecting group like Boc. Compound III-4 is then coupled with compound III-5, wherein X is a halo (e.g., —Br or —I) or pseudohalo (e.g., -OTf), e.g., under typical Ullman coupling conditions known in the art, to provide compound III-6. As a non-limiting example, the coupling can be carried out in the presence of CuI, K₂CO₃, and

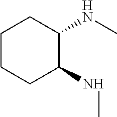

in NMP (e.g., at 130° C.). Removal of the nitrogen protecting group (i.e., PG) on III-6 (e.g., under acidic conditions such as HCl in dioxanes) then provides compound III-7.

Appropriate starting materials and reagents for the steps outlined in any of Scheme I, II, or III can be purchased or prepared by methods known to one of skill in the art. Upon each reaction completion, each of the intermediate or final compounds can be recovered, and optionally purified, by conventional techniques such as neutralization, extraction, precipitation, chromatography, filtration, and the like. Other modifications to arrive at compounds of this disclosure are within the skill of the art.

In certain embodiments, when control of stereochemistry is desired, proper control of reaction conditions and selection of substituents for the reagents can at least partially dictate or preserve the formation of the various stereoisomers. In some embodiments, the various substituents of the various compounds of Scheme I, II, or III (e.g., Ring A, Ring B, Ring C, Q, X, Y, L¹, L², R¹, R², and R³) are each independently as defined herein. However, derivatization of any of the various compounds of Scheme I, II, or III prior to a reaction step, and/or further derivatization of the resulting reaction product, provides various compounds of Formula I.

It will be apparent to a person of ordinary skill in the art that in the synthetic sequences delineated above in Scheme I, II, or III, reactive groups such as NH₂, NH, and OH can be protected with appropriate protecting groups, followed by a deprotection steps at appropriate stages of the synthesis. It is also within the purview of a person of ordinary skill in the art to conceive variations of the foregoing methods to synthesize other Formula I compounds. For example, by exchanging III-2 with other electrophilic reagents, compounds with other L³-C(R^{8a}R^{8b})-L⁴-R⁹ groups can be obtained. As another example, by replacing III-1 with other appropriate reagents, compounds with other L² moieties can be obtained.

The compounds provided in Table 1 can be prepared according to the methods shown in Schemes I, II, and III.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing form the spirit and scope of the disclosure.

Abbreviations (as used herein):

| | |
|---|---|
| aq. | aqueous |
| BPO | benzoyl peroxide |
| CCl4 | carbon tetrachloride |
| CDI | 1,1′-carbonyldiimidazole |
| CH₂Cl₂ | dichloromethane |
| CH₃CN | acetonitrile |
| CuBr | cuprous bromide |
| DABCO | 1,4-diaza[2.2.2]bicyclooctane |
| DCM or CH₂Cl₂ | dichloromethane |
| DBU | 1,8-diazabicyclo[5.4.0]undec-7-ene |
| DIPEA | N,N-Diisopropylethylamine |
| DMF | N,N-dimethyl-formamide |
| DMPU | 1,3-dimethyl-3,4,5,6-tetrahydro-2(1H)-pyrimidinone |
| DMSO | dimethyl sulfoxide |
| EtOAc | ethyl acetate |
| EtOH | ethanol |
| FeSO₄•7H₂O | ferrous sulfate |
| h | hour |
| HATU | 2-(7-Azabenzotriazol-1-yl)-N,N,N′,N′-tetramethyluronium |
| | hexafluorophosphate |
| HCl | hydrochloric acid |
| HMDSLi | lithium bis(trimethylsilyl)amide |
| H₂O | water |
| H₂O₂ | hydrogen peroxide |
| K₂CO₃ | potassium carbonate |
| KF | potassium fluoride |
| LiOH | lithium hydroxide |
| MeOH | methanol |
| NaBH₄ | sodium borohydride |
| NaH | sodium hydride |
| NaI | sodium iodide |
| Na₂SO₄ | sodium sulfate |
| NBS | bromosuccinimide |
| NH₂OH•HCl | hydroxylamine hydrochloride |
| NiCl₂ | nickel chloride |
| Pd₂(dba)₃ | tris(dibenzylideneacetone)dipalladium |
| Pd(dppf)Cl₂ | [1,1′-bis(diphenylphosphino)-ferrocene]palladium(II) chloride |
| PE | Petrol ether |
| prep. HPLC | Preparative High Performance Liquid Chromatography |
| prep. TLC | Preparative Thin layer chromatography |
| sat. | saturated |
| TBAF | tetrabutylammonium fluoride |
| THF | tetrahydrofuran |
| TMSCN | trimethylsilyl cyanide |

General information: All evaporations or concentrations were carried out in vacuo with a rotary evaporator. Analytical samples were dried in vacuo (1-5 mmHg) at rt. Thin layer chromatography (TLC) was performed on silica gel plates, spots were visualized by UV light (214 and 254 nm). Purification by column and flash chromatography was carried out using silica gel (100-200 mesh). Solvent systems were reported as mixtures by volume. NMR spectra were recorded on a Bruker 400 or Varian (400 MHz) spectrometer. ¹H chemical shifts are reported in 6 values in ppm with the deuterated solvent as the internal standard. Data are reported as follows: chemical shift, multiplicity (s=singlet, d=doublet, t=triplet, q=quartet, br=broad, m=multiplet), coupling constant (Hz), integration. LCMS spectra were obtained on SHIMADZU LC20-MS2020 or Agilent 1260 series 6125B mass spectrometer or Agilent 1200 series, 6110 or 6120 mass spectrometer with electrospray ionization and excepted as otherwise indicated.

### Example 1

### 3-(1-{2-[(4S)-3-{3-[4-(diethylphosphoryl)-3-(methylamino)phenyl]-2-oxo-2,3-dihydro-1H-imidazol-1-yl}-2-(4-fluoro-3,5-dimethylphenyl)-4-methyl-2H,4H,5H,6H,7H-pyrazolo[4,3-c]pyridine-5-carbonyl]-6-(oxan-4-yl)-1H-indol-3-yl}cyclopropyl)-2,5-dihydro-1,2,4-oxadiazol-5-one (Compound 28)

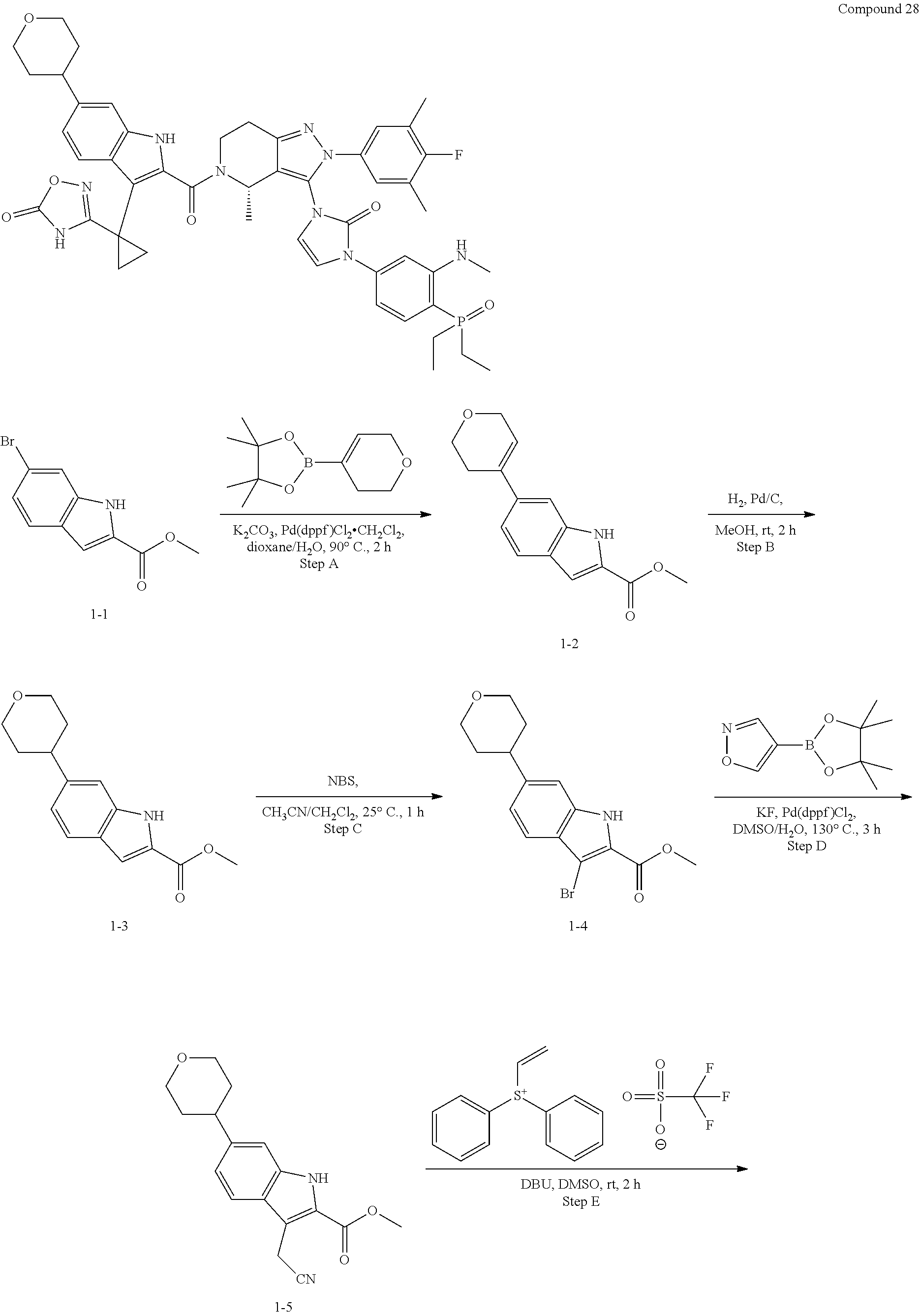

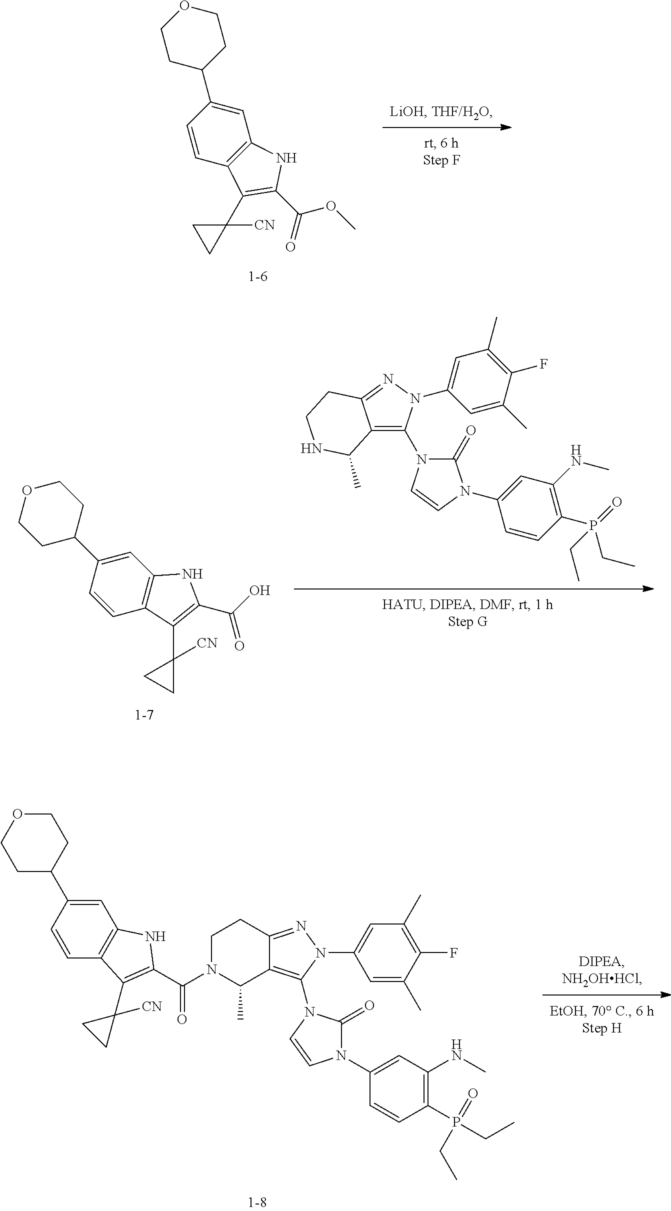 [SMILES: COC(=O)c1[nH]c2cc(C3CCOCC3)ccc2c1C1(C#N)CC1 ; N#CC1(c2c(C(=O)O)[nH]c3cc(C4CCOCC4)ccc23)CC1 ; CCP(=O)(CC)c1ccc(-n2ccn(-c3c4c(nn3-c3cc(C)c(F)c(C)c3)CCN[C@H]4C)c2=O)cc1NC ; CCP(=O)(CC)c1ccc(-n2ccn(-c3c4c(nn3-c3cc(C)c(F)c(C)c3)CCN(C(=O)c3[nH]c5cc(C6CCOCC6)ccc5c3C3(C#N)CC3)[C@H]4C)c2=O)cc1NC]

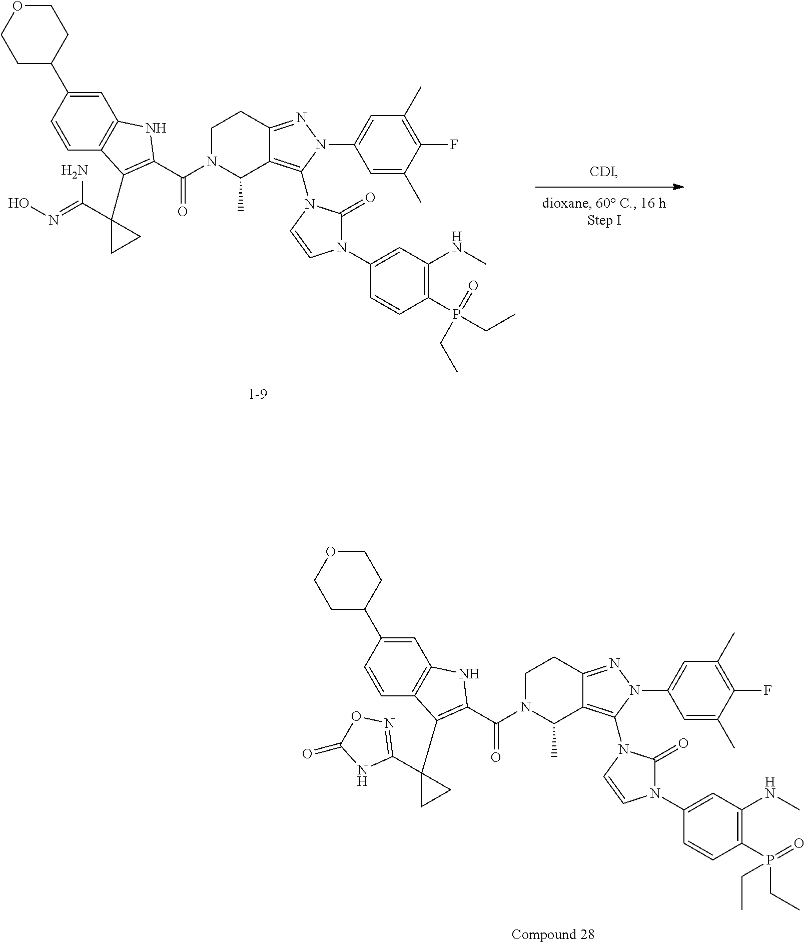 [SMILES: CCP(=O)(CC)c1ccc(-n2ccn(-c3c4c(nn3-c3cc(C)c(F)c(C)c3)CCN(C(=O)c3[nH]c5cc(C6CCOCC6)ccc5c3C3(/C(N)=N/O)CC3)[C@H]4C)c2=O)cc1NC ; CCP(=O)(CC)c1ccc(-n2ccn(-c3c4c(nn3-c3cc(C)c(F)c(C)c3)CCN(C(=O)c3[nH]c5cc(C6CCOCC6)ccc5c3C3(c5noc(=O)[nH]5)CC3)[C@H]4C)c2=O)cc1NC]

### Step A methyl 6-(3,6-dihydro-2H-pyran-4-yl)-1H-indole-2-carboxylate

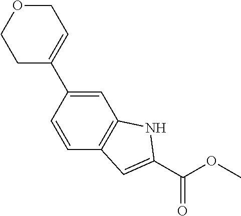 [SMILES: COC(=O)c1cc2ccc(C3=CCOCC3)cc2[nH]1]

To the mixture of methyl 6-bromo-1H-indole-2-carboxylate (5 g, 19.69 mmol), 2-(3,6-dihydro-2H-pyran-4-yl)-4,4,5,5-tetramethyl-1,3,2-dioxaborolane (4.96 g, 23.62 mmol) and K₂CO₃ (8.15 g, 59.06 mmol) in dioxane (80 mL) and H₂O (8 mL), was added Pd(dppf)Cl₂·CH₂Cl₂ (0.8 g, 0.98 mmol). After being charged with nitrogen three times. The resulting mixture was stirred at 90° C. for 3 h under nitrogen. After cooling, the mixture was filtered and concentrated under vacuum to dryness. The residue was purified by flash silica gel chromatography (Silica Flash Column, Eluent of 0-25% EtOAc/PE gradient) to give methyl 6-(3,6-dihydro-2H-pyran-4-yl)-1H-indole-2-carboxylate (3.58 g, 70.8% yield). LC-MS: m/z: 258.4 (M+H)⁺.

### Step B methyl 6-(tetrahydro-2H-pyran-4-yl)-1H-indole-2-carboxylate

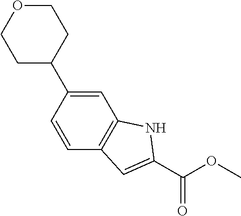 [SMILES: COC(=O)c1cc2ccc(C3CCOCC3)cc2[nH]1]

The mixture of methyl 6-(3,6-dihydro-2H-pyran-4-yl)-1H-indole-2-carboxylate (3.48 g, 13.54 mmol) and 5% Pd/C (348 mg) in MeOH (35 mL) was degassed and charged with H₂ three times. The reaction mixture was stirred at room temperature under H₂ gas balloon for 2 h. The mixture was filtered and concentrated to give methyl 6-(tetrahydro-2H-pyran-4-yl)-1H-indole-2-carboxylate (3.43 g, 98.0% yield). LC-MS: m/z: 260.1 (M+H)⁺.

### Step C methyl 3-bromo-6-(tetrahydro-2H-pyran-4-yl)-1H-indole-2-carboxylate

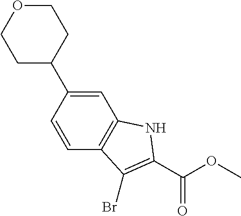 [SMILES: COC(=O)c1[nH]c2cc(C3CCOCC3)ccc2c1Br]

To the solution of methyl 6-(tetrahydro-2H-pyran-4-yl)-1H-indole-2-carboxylate (3.33 g, 12.86 mmol) in CH₃CN (30 mL) and CH₂Cl₂ (20 mL), was added NBS (1.6 g, 9.0 mmol). The resulting mixture was stirred at room temperature for 1 h. The reaction mixture was diluted with H₂O (300 mL), extracted with CH₂Cl₂ (50 mL×3). The organic layers were washed with brine, dried over anhydrous Na₂SO₄, filtered and concentrated to give crude methyl 3-bromo-6-(tetrahydro-2H-pyran-4-yl)-1H-indole-2-carboxylate (3.9 g, 90.7% yield). LC-MS: m/z: 338.1 (M+H)⁺.

### Step D methyl 3-(cyanomethyl)-6-(tetrahydro-2H-pyran-4-yl)-1H-indole-2-carboxylate

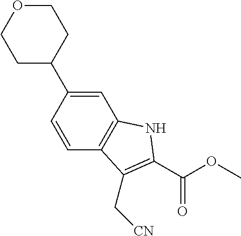 [SMILES: COC(=O)c1[nH]c2cc(C3CCOCC3)ccc2c1CC#N]

To a solution of methyl 3-bromo-6-(tetrahydro-2H-pyran-4-yl)-1H-indole-2-carboxylate (300 mg, 0.89 mmol) in DMSO (6 mL) and H₂O (1 mL) was added 4-(4,4,5,5-tetramethyl-1,3,2-dioxaborolan-2-yl)isoxazole (346 mg, 1.77 mmol), KF (154 mg, 2.66 mmol) and Pd(dppf)Cl₂ (65 mg, 0.09 mmol). After being charged with nitrogen three times. The resulting mixture was stirred at 130° C. for 3 h under nitrogen. After cooling, the mixture was concentrated and purified by flash silica gel chromatography (Silica Flash Column, Eluent of 0-20% EtOAc/PE gradient) to afford methyl 3-(cyanomethyl)-6-(tetrahydro-2H-pyran-4-yl)-1H-indole-2-carboxylate (290 mg, 65.1% yield). LC-MS: m/z 299.3 (M+H)⁺.

### Step E methyl 3-(1-cyanocyclopropyl)-6-(tetrahydro-2H-pyran-4-yl)-1H-indole-2-carboxylate

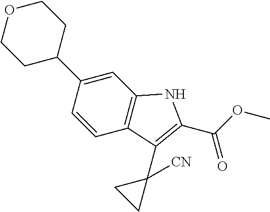 [SMILES: COC(=O)c1[nH]c2cc(C3CCOCC3)ccc2c1C1(C#N)CC1]

The mixture of methyl 3-(cyanomethyl)-6-(tetrahydro-2H-pyran-4-yl)-1H-indole-2-carboxylate (100 mg, 0.34 mmol), ethenyldiphenylsulfanium trifluoromethanesulfonate (146 mg, 0.40 mmol) and DBU (153 mg, 1.01 mmol) in DMSO (1 mL) was stirred at room temperature for 2 h under nitrogen. The mixture was diluted with H₂O (30 mL), extracted with EtOAc (10 mL×3). The organic layers were washed with brine, dried over anhydrous Na₂SO₄, filtered and concentrated. The residue was purified by prep. TLC (20% EtOAc/PE) to afford the methyl 3-(1-cyanocyclopropyl)-6-(tetrahydro-2H-pyran-4-yl)-1H-indole-2-carboxylate (38 mg, 35.0% yield). LC-MS: m/z 325.2 (M+H)⁺.

### Step F 3-(1-cyanocyclopropyl)-6-(tetrahydro-2H-pyran-4-yl)-1H-indole-2-carboxylic acid

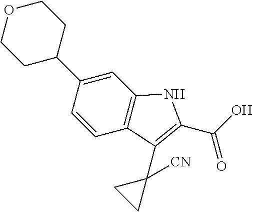 [SMILES: N#CC1(c2c(C(=O)O)[nH]c3cc(C4CCOCC4)ccc23)CC1]

The mixture of methyl 3-(cyanocyclopropyl)-6-(3,4,5,6-tetrahydro-2H-pyran-4-yl)benzo[b]thiophene-2-carboxylate (38 mg, 0.12 mmol) and LiOH (9.8 mg, 0.23 mmol) in THF (1 mL) and H₂O (0.2 mL) was stirred at room temperature for 6 h. The mixture was diluted with water (30 mL), acidified with 1 M aq. HCl to pH=5.0, extracted with extracted with EtOAc (10 mL×3). The combined organic layers were washed with brine (10 mL×2), dried over anhydrous Na₂SO₄, filtered and concentrated to give 3-(1-cyanocyclopropyl)-6-(tetrahydro-2H-pyran-4-yl)-1H-indole-2-carboxylic acid (37 mg, crude). LC-MS: m/z 311.3 (M+H)⁺.

### Step G (S)-1-(2-(3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-6-(tetrahydro-2H-pyran-4-yl)-1H-indol-3-yl)cyclopropane-1-carbonitrile

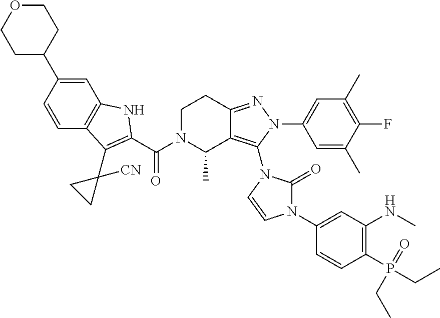 [SMILES: CCP(=O)(CC)c1ccc(-n2ccn(-c3c4c(nn3-c3cc(C)c(F)c(C)c3)CCN(C(=O)c3[nH]c5cc(C6CCOCC6)ccc5c3C3(C#N)CC3)[C@H]4C)c2=O)cc1NC]

To a solution of 3-(1-cyanocyclopropyl)-6-(tetrahydro-2H-pyran-4-yl)-1H-indole-2-carboxylic acid (37 mg, 0.12 mmol) in DMF (2 mL) were added (S)-1-(4-(diethylphosphoryl)-3-(methylamino)phenyl)-3-(2-(4-fluoro-3,5-dimethylphenyl)-4-methyl-4,5,6,7-tetrahydro-2H-pyrazolo[4,3-c]pyridin-3-yl)-1,3-dihydro-2H-imidazol-2-one (65 mg, 0.12 mmol) (HCl salt, preparation was described in WO2021155841A1), HATU (54 mg, 0.14 mmol) and DIPEA (46 mg, 0.36 mmol). The reaction mixture was stirred at room temperature for 1 h. Then the mixture was diluted with brine (30 mL), extracted with EtOAc (10 mL×3). The combined organic layers were dried over anhydrous Na₂SO₄, filtered and concentrated. The residue was purified by flash silica gel chromatography (12 g SepaFlash® Silica Flash Column, Eluent of 0-10% MeOH/CH₂Cl₂ gradient @50 mL/min) to afford (S)-1-(2-(3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-6-(tetrahydro-2H-pyran-4-yl)-1H-indol-3-yl)cyclopropane-1-carbonitrile (66 mg, 65.3% yield). LC-MS: m/z 843.4 (M+H)⁺.

### Step H (S,Z)-1-(2-(3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-6-(tetrahydro-2H-pyran-4-yl)-1H-indol-3-yl)-N′-hydroxycyclopropane-1-carboximidamide

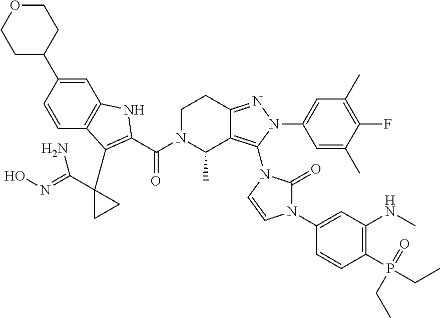 [SMILES: CCP(=O)(CC)c1ccc(-n2ccn(-c3c4c(nn3-c3cc(C)c(F)c(C)c3)CCN(C(=O)c3[nH]c5cc(C6CCOCC6)ccc5c3C3(/C(N)=N/O)CC3)[C@H]4C)c2=O)cc1NC]

To a solution of (S)-1-(2-(3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-6-(tetrahydro-2H-pyran-4-yl)-1H-indol-3-yl)cyclopropane-1-carbonitrile (66 mg, 0.078 mmol) in EtOH (1 mL) was added DIPEA (30 mg, 0.234 mmol) and NH₂OH·HCl (16.3 mg, 0.234 mmol). The resulting mixture was stirred at 70° C. for 6 h. After cooling, the mixture was diluted with brine (30 ml), extracted with EtOAc (10 mL×3). The combined organic layers were dried over anhydrous Na₂SO₄, filtered and concentrated. The residue was purified by flash silica gel chromatography (12 g SepaFlash® Silica Flash Column, Eluent of 0-5% MeOH/CH₂Cl₂ gradient @50 mL/min) to afford the title compound (S,Z)-1-(2-(3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-6-(tetrahydro-2H-pyran-4-yl)-1H-indol-3-yl)-N′-hydroxycyclopropane-1-carboximidamide (36 mg, 57.6% yield). LC-MS: m/z 876.2 (M+H)⁺.

### Step I 3-(1-{2-[(4S)-3-{3-[4-(diethylphosphoryl)-3-(methylamino)phenyl]-2-oxo-2,3-dihydro-1H-imidazol-1-yl}-2-(4-fluoro-3,5-dimethylphenyl)-4-methyl-2H,4H,5H,6H,7H-pyrazolo[4,3-c]pyridine-5-carbonyl]-6-(oxan-4-yl)-1H-indol-3-yl}cyclopropyl)-2,5-dihydro-1,2,4-oxadiazol-5-one (Compound 28)

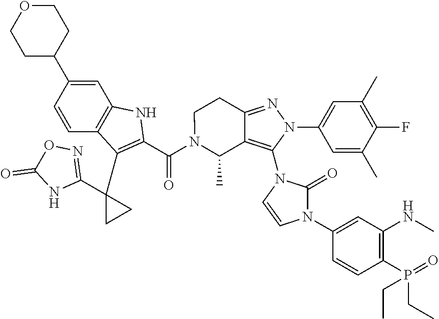 [SMILES: CCP(=O)(CC)c1ccc(-n2ccn(-c3c4c(nn3-c3cc(C)c(F)c(C)c3)CCN(C(=O)c3[nH]c5cc(C6CCOCC6)ccc5c3C3(c5noc(=O)[nH]5)CC3)[C@H]4C)c2=O)cc1NC]

To a solution of (S,Z)-1-(2-(3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-6-(tetrahydro-2H-pyran-4-yl)-1H-indol-3-yl)-N′-hydroxycyclopropane-1-carboximidamide (36 mg, 0.041 mmol) in dioxane (1 mL) was added CDI (20 mg, 0.123 mmol). The resulting mixture was stirred at 60° C. for 16 h. After cooling, the reaction mixture was concentrated. The residue was purified by prep. HPLC (Column: Pursuit XRs C18 19*250 mm*10 μm; Mobile Phase A: Water (0.1% formic acid), Mobile Phase B: CH₃CN; Flow rate: 20 mL/min; Gradient: 57% B to 67% B, retention time: 9.2-10.3 min) to give 3-(1-{2-[(4S)-3-{3-[4-(diethylphosphoryl)-3-(methylamino)phenyl]-2-oxo-2,3-dihydro-1H-imidazol-1-yl}-2-(4-fluoro-3,5-dimethylphenyl)-4-methyl-2H,4H,5H,6H,7H-pyrazolo[4,3-c]pyridine-5-carbonyl]-6-(oxan-4-yl)-1H-indol-3-yl}cyclopropyl)-2,5-dihydro-1,2,4-oxadiazol-5-one (12.0 mg, 32.3% yield). LC-MS: m/z 902.9 (M+H)⁺. ¹H NMR (400 MHz, DMSO-d6): δ 11.88 (br s, 1H), 11.47 (s, 1H), 7.67-7.78 (m, 1H), 7.52 (d, J=8.0 Hz, 1H), 7.33-7.44 (m, 1H), 7.20-7.32 (m, 2H), 7.08-7.19 (m, 2H), 6.97-7.07 (m, 2H), 6.81-6.96 (m, 2H), 5.56-5.70 (m, 1H), 3.84-4.02 (m, 3H), 3.39-3.51 (m, 3H), 2.61-2.91 (m, 6H), 2.20 (s, 6H), 1.86-1.98 (m, 4H), 1.64-1.78 (m, 4H), 1.43-1.58 (m, 2H), 1.17-1.36 (m, 5H), 0.93-1.05 (m, 6H). ¹⁹F NMR (377 MHz, DMSO-d6): δ −122.07.

### Example 2

### 3-(1-{6-[(4S)-3-{3-[4-(diethylphosphoryl)-3-(methylamino)phenyl]-2-oxo-2,3-dihydro-1H-imidazol-1-yl}-2-(4-fluoro-3,5-dimethylphenyl)-4-methyl-2H,4H,5H,6H,7H-pyrazolo[4,3-c]pyridine-5-carbonyl]-3-(oxan-4-yl)pyrrolo[1,2-b]pyridazin-7-yl}cyclopropyl)-4,5-dihydro-1,2,4-oxadiazol-5-one (Compound 26)

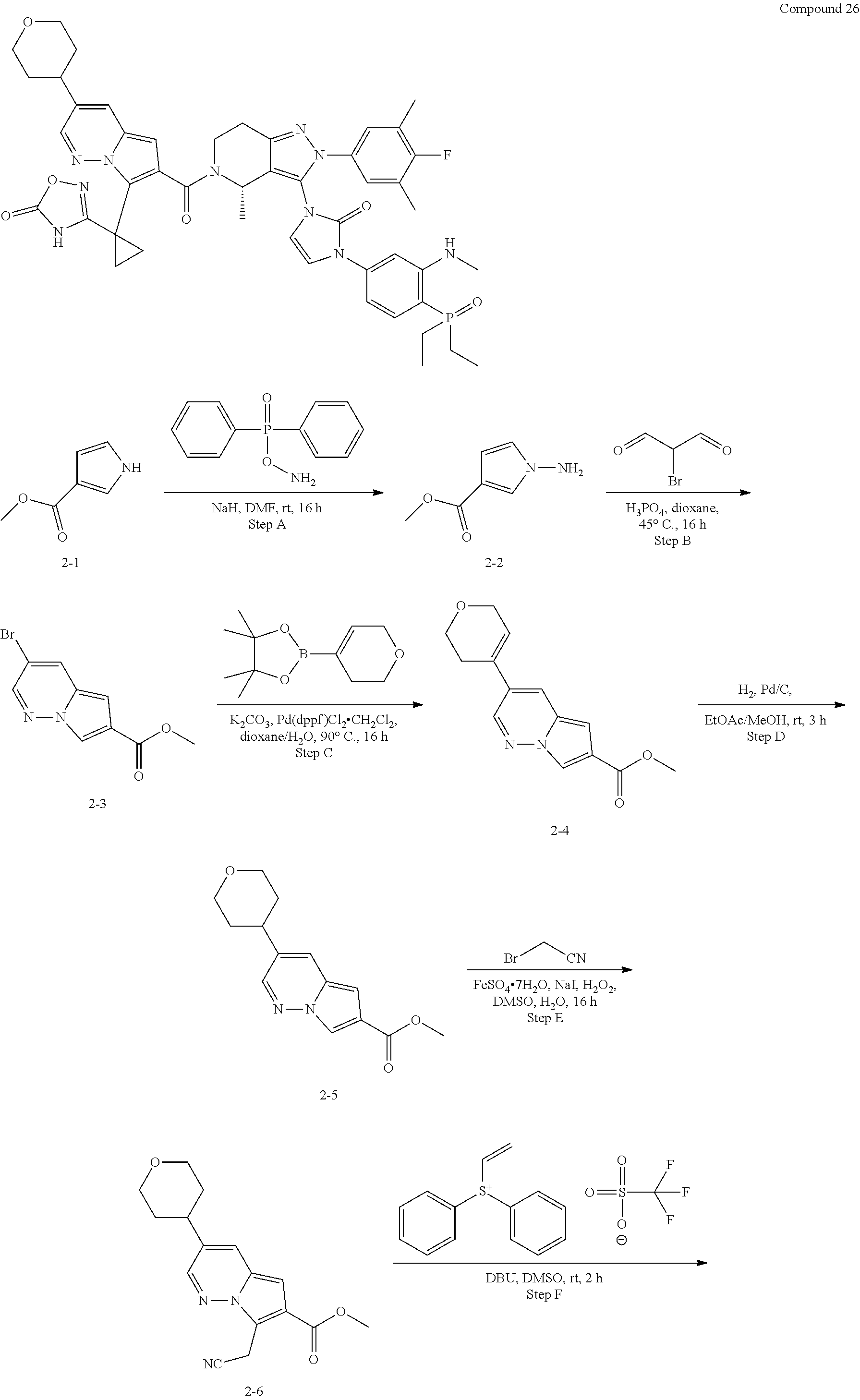

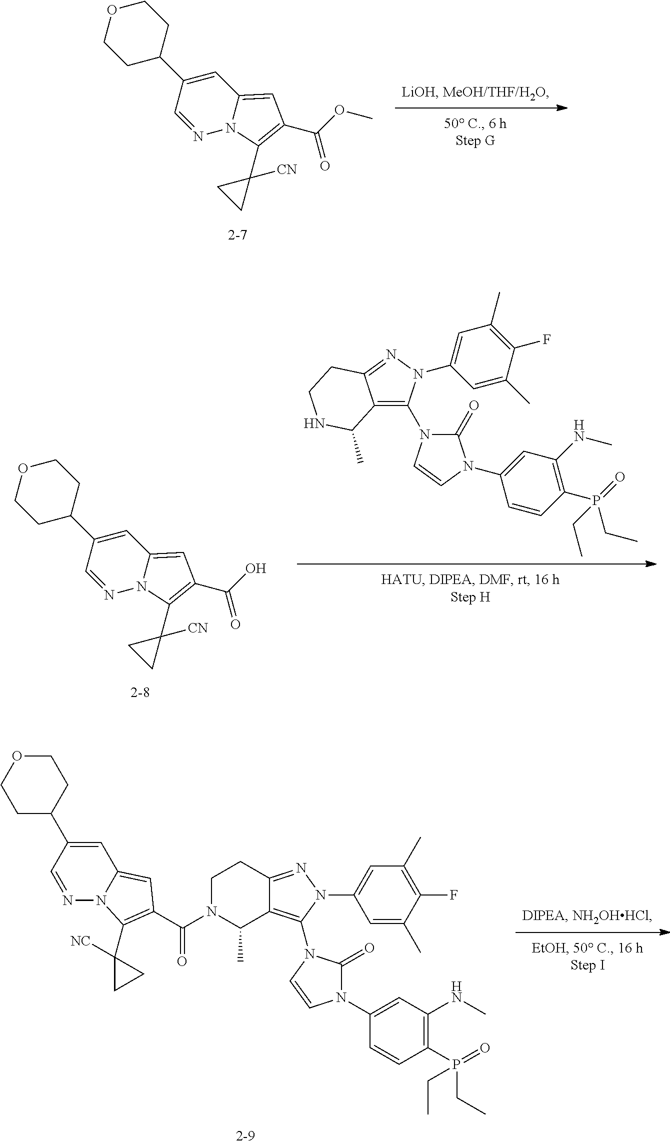 [SMILES: COC(=O)c1cc2cc(C3CCOCC3)cnn2c1C1(C#N)CC1 ; N#CC1(c2c(C(=O)O)cc3cc(C4CCOCC4)cnn23)CC1 ; CCP(=O)(CC)c1ccc(-n2ccn(-c3c4c(nn3-c3cc(C)c(F)c(C)c3)CCN[C@H]4C)c2=O)cc1NC ; CCP(=O)(CC)c1ccc(-n2ccn(-c3c4c(nn3-c3cc(C)c(F)c(C)c3)CCN(C(=O)c3cc5cc(C6CCOCC6)cnn5c3C3(C#N)CC3)[C@H]4C)c2=O)cc1NC]

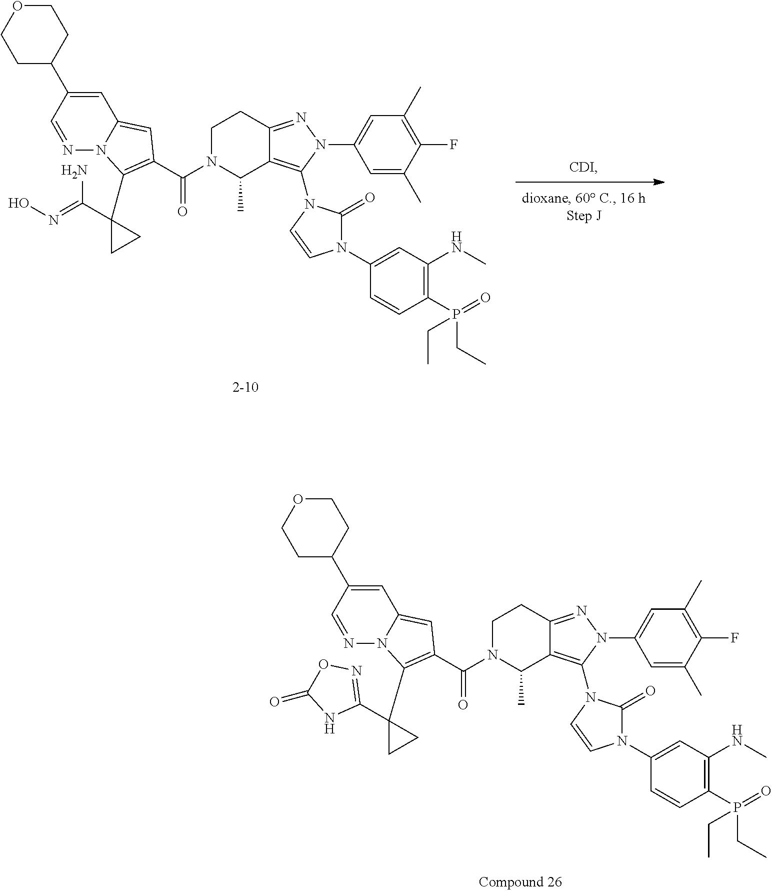 [SMILES: CCP(=O)(CC)c1ccc(-n2ccn(-c3c4c(nn3-c3cc(C)c(F)c(C)c3)CCN(C(=O)c3cc5cc(C6CCOCC6)cnn5c3C3(/C(N)=N/O)CC3)[C@H]4C)c2=O)cc1NC ; CCP(=O)(CC)c1ccc(-n2ccn(-c3c4c(nn3-c3cc(C)c(F)c(C)c3)CCN(C(=O)c3cc5cc(C6CCOCC6)cnn5c3C3(c5noc(=O)[nH]5)CC3)[C@H]4C)c2=O)cc1NC]

### Step A methyl 1-amino-1H-pyrrole-3-carboxylate

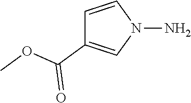

To a solution of methyl 1H-pyrrole-3-carboxylate (10.0 g, 80.0 mmol) in DMF (300 mL) was added NaH (60% wt, dispersion in paraffin liquid) (2.50 g, 104 mmol) at 0° C. with stirring. After being stirred at 0° C. for 20 min. Then (aminooxy)diphenylphosphine oxide (28.0 g, 120.0 mmol) was added at 0° C. The resulting mixture was stirred at room temperature for 16 h. The mixture was poured into ice water (300 mL) and extracted with EtOAc (300 mL×2). The combined organic layer was washed with brine, dried over anhydrous Na₂SO₄, filtered and concentrated. The residue was purified by flash silica gel chromatography (220 g SepaFlash® Silica Flash Column, Eluent of 0-10% MeOH/CH₂Cl₂ gradient @100 mL/min) to afford methyl 1-amino-1H-pyrrole-3-carboxylate (8.8 g, 78.6% yield). LC-MS: m/z 141.0 (M+H)⁺.

### Step B methyl 3-bromopyrrolo[1,2-b]pyridazine-6-carboxylate

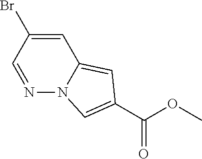

To the mixture of methyl 1-amino-1H-pyrrole-3-carboxylate (8.8 g, 62.85 mmol), 2-bromomalonaldehyde (18.98 g, 125.71 mmol) in dioxane (50 mL) was added H₃PO₄ (8.5 mL). The resulting mixture was stirred at 45° C. for 16 h. After cooling, the reaction mixture was basified by sat. aq. NaHCO₃, extracted with DCM (100 mL×3). The combined organic layers were washed with brine (50 mL), dried over anhydrous Na₂SO₄, filtered and concentrated. The residue was purified by flash silica gel chromatography (120 g SepaFlash® Silica Flash Column, Eluent of 0-10% EtOAc/PE gradient @40 mL/min) to give methyl 3-bromopyrrolo[1,2-b]pyridazine-6-carboxylate (601 mg, 3.8% yield). LC-MS: m/z 255.0 (M+H)⁺.

### Step C methyl 3-(3,6-dihydro-2H-pyran-4-yl)pyrrolo[1,2-b]pyridazine-6-carboxylate

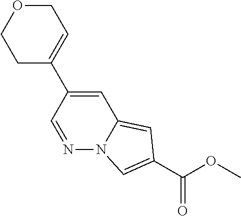 [SMILES: COC(=O)c1cc2cc(C3=CCOCC3)cnn2c1]

To the mixture of methyl 3-bromopyrrolo[1,2-b]pyridazine-6-carboxylate (600 mg, 2.36 mmol) and 2-(3,6-dihydro-2H-pyran-4-yl)-4,4,5,5-tetramethyl-1,3,2-dioxaborolane (992 mg, 4.72 mmol) in dioxane (5 mL) and H₂O (1 mL) was added Pd(dppf)Cl₂ (173 mg, 0.236 mmol) and K₂CO₃ (978 mg, 7.09 mmol). The reaction mixture was charged with nitrogen three time and then stirred at 90° C. for 16 h. After cooling, the mixture was poured into water (50 mL) and extracted with EtOAc (50 mL×2). The combined organic layer was washed with brine, dried over anhydrous Na₂SO₄, filtered and concentrated. The residue was purified by flash silica gel chromatography (4 g SepaFlash® Silica Flash Column, Eluent of 0˜25% EtOAc/PE gradient @50 mL/min) to obtain methyl 3-(3,6-dihydro-2H-pyran-4-yl)pyrrolo[1,2-b]pyridazine-6-carboxylate (427 mg, 70.1% yield). LC-MS: m/z 259.1 (M+H)⁺.

### Step D methyl 3-(tetrahydro-2H-pyran-4-yl)pyrrolo[1,2-b]pyridazine-6-carboxylate

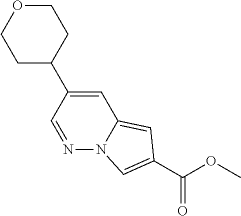 [SMILES: COC(=O)c1cc2cc(C3CCOCC3)cnn2c1]

The mixture of methyl 3-(3,6-dihydro-2H-pyran-4-yl)pyrrolo[1,2-b]pyridazine-6-carboxylate (470 mg, 1.82 mmol) and 10% Pd/C (388 mg) in EtOAc/MeOH (5 mL/5 mL) were stirred at room temperature under H₂ balloon for 3 h. The mixture was filtered and the residue was concentrated to obtain methyl 3-(tetrahydro-2H-pyran-4-yl)pyrrolo[1,2-b]pyridazine-6-carboxylate (395 mg, 83.4% yield). LC-MS: m/z 261.1 (M+H)⁺.

### Step E methyl 7-(cyanomethyl)-3-(tetrahydro-2H-pyran-4-yl)pyrrolo[1,2-b]pyridazine-6-carboxylate

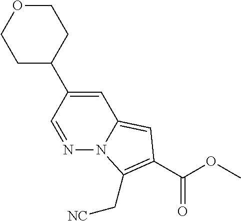 [SMILES: COC(=O)c1cc2cc(C3CCOCC3)cnn2c1CC#N]

To the ice water cooled mixture of methyl 3-(tetrahydro-2H-pyran-4-yl)pyrrolo[1,2-b]pyridazine-6-carboxylate (350 mg, 1.35 mmol), 2-bromoacetonitrile (178 mg, 1.48 mmol), FeSO₄·7H₂O (187 mg, 0.67 mmol) and NaI (202 mg, 1.35 mmol) in DMSO (7 mL) and H₂O (1 mL), was added 30% H₂O₂ (0.4 g) with stirring. After addition, the mixture was stirred at room temperature for 16 h. The mixture was diluted with water (20 mL), extracted with ethyl acetate (30 mL×3). The combined organic layers were dried over anhydrous Na₂SO₄, filtered and concentrated. The residue was purified by flash silica gel chromatography (4 g SepaFlash® Silica Flash Column, Eluent of 0-25% EtOAc/PE gradient @50 mL/min) to obtain methyl 7-(cyanomethyl)-3-(tetrahydro-2H-pyran-4-yl)pyrrolo[1,2-b]pyridazine-6-carboxylate (100 mg, 24.9% yield). LC-MS: m/z 300.0 (M+H)⁺.

### Step F methyl 7-(1-cyanocyclopropyl)-3-(tetrahydro-2H-pyran-4-yl)pyrrolo[1,2-b]pyridazine-6-carboxylate

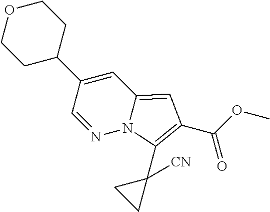 [SMILES: COC(=O)c1cc2cc(C3CCOCC3)cnn2c1C1(C#N)CC1]

To the mixture of methyl 7-(cyanomethyl)-3-(tetrahydro-2H-pyran-4-yl)pyrrolo[1,2-b]pyridazine-6-carboxylate (100 mg, 0.33 mmol) and ethenyldiphenylsulfanium trifluoromethanesulfonate (145 mg, 0.40 mmol) in DMSO (3 mL) was added DBU (154 mg, 1.00 mmol). The resulting mixture was stirred at room temperature under nitrogen for 2 h. The mixture was poured into water (50 mL) and extracted with EtOAc (50 mL×2). The combined organic layers were dried over anhydrous Na₂SO₄, filtered and concentrated. The residue was purified by flash silica gel chromatography (4 g SepaFlash® Silica Flash Column, Eluent of 0-25% EtOAc/PE gradient @50 mL/min) to obtain methyl 7-(1-cyanocyclopropyl)-3-(tetrahydro-2H-pyran-4-yl)pyrrolo[1,2-b]pyridazine-6-carboxylate (95 mg, 87.4% yield). LC-MS: m/z 326.5 (M+H)⁺.

### Step G 7-(1-cyanocyclopropyl)-3-(tetrahydro-2H-pyran-4-yl)pyrrolo[1,2-b]pyridazine-6-carboxylic acid

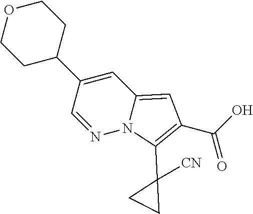 [SMILES: N#CC1(c2c(C(=O)O)cc3cc(C4CCOCC4)cnn23)CC1]

The mixture of methyl 7-(1-cyanocyclopropyl)-3-(tetrahydro-2H-pyran-4-yl)pyrrolo[1,2-b]pyridazine-6-carboxylate (95 mg, 0.292 mmol) and LiOH (123 mg, 2.923 mmol) in THF/MeOH/H₂O (5 mL/1 mL/1 mL) was stirred at 50° C. for 6 h. After cooling, the mixture was diluted with water (50 mL), extracted with EtOAc (25 mL×2). The aqueous phase was acidified by 1 M aq. HCl to pH=4, extracted with CH₂Cl₂ (30 mL×3). The combined organic layers were dried over anhydrous Na₂SO₄ and then concentrated to obtain 7-(1-cyanocyclopropyl)-3-(tetrahydro-2H-pyran-4-yl)pyrrolo[1,2-b]pyridazine-6-carboxylic acid (80 mg, 88.0% yield). LC-MS: m/z 312.1 (M+H)⁺.

### Step H (S)-1-(6-(3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-3-(tetrahydro-2H-pyran-4-yl)pyrrolo[1,2-b]pyridazin-7-yl)cyclopropane-1-carbonitrile

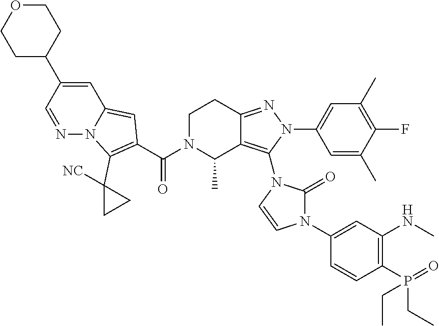 [SMILES: CCP(=O)(CC)c1ccc(-n2ccn(-c3c4c(nn3-c3cc(C)c(F)c(C)c3)CCN(C(=O)c3cc5cc(C6CCOCC6)cnn5c3C3(C#N)CC3)[C@H]4C)c2=O)cc1NC]

To the mixture of 7-(1-cyanocyclopropyl)-3-(tetrahydro-2H-pyran-4-yl)pyrrolo[1,2-b]pyridazine-6-carboxylic acid (80 mg, 0.258 mmol) and (S)-1-(4-(diethylphosphoryl)-3-(methylamino)phenyl)-3-(2-(4-fluoro-3,5-dimethylphenyl)-4-methyl-4,5,6,7-tetrahydro-2H-pyrazolo[4,3-c]pyridin-3-yl)-1,3-dihydro-2H-imidazol-2-one (167 mg, 0.284 mmol) (HCl salt, preparation was described in WO2021155841A1) in DMF (3 mL), was added with DIPEA (133 mg, 1.032 mmol) and HATU (147 mg, 0.387 mmol). The resulting mixture was stirred at room temperature for 16 h. The mixture was poured into water (50 mL) and extracted with EtOAc (50 mL×2). The combined organic layer was washed with brine, dried over Na₂SO₄, filtered and the residue was concentrated. The residue was purified by prep. TLC (5% MeOH/CH₂Cl₂) to obtain (S)-1-(6-(3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-3-(tetrahydro-2H-pyran-4-yl)pyrrolo[1,2-b]pyridazin-7-yl)cyclopropane-1-carbonitrile (305 mg, crude). LC-MS: m/z 844.2 (M+H)⁺.

### Step I (S,Z)-1-(6-(3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-3-(tetrahydro-2H-pyran-4-yl)pyrrolo[1,2-b]pyridazin-7-yl)-N′-hydroxycyclopropane-1-carboximidamide

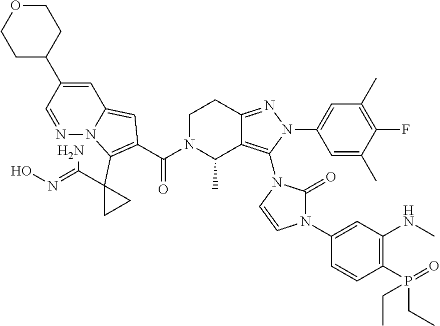 [SMILES: CCP(=O)(CC)c1ccc(-n2ccn(-c3c4c(nn3-c3cc(C)c(F)c(C)c3)CCN(C(=O)c3cc5cc(C6CCOCC6)cnn5c3C3(/C(N)=N/O)CC3)[C@H]4C)c2=O)cc1NC]

To the mixture of (S)-1-(6-(3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-3-(tetrahydro-2H-pyran-4-yl)pyrrolo[1,2-b]pyridazin-7-yl)cyclopropane-1-carbonitrile (300 mg, 0.356 mmol) and NH₂OH·HCl (246 mg, 3.559 mmol) in EtOH (10 mL), was added DIPEA (505 mg, 3.915 mmol). The resulting mixture was stirred at 50° C. for 16 h. After cooling, the reaction mixture was concentrated, the residue was diluted with water (20 mL), extracted with CH₂Cl₂ (20 mL×3). The combined organic layers were dried over anhydrous Na₂SO₄, filtered and concentrated to obtain (S,Z)-1-(6-(3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-3-(tetrahydro-2H-pyran-4-yl)pyrrolo[1,2-b]pyridazin-7-yl)-N′-hydroxycyclopropane-1-carboximidamide (200 mg, crude). LC-MS: m/z 877.7 (M+H)⁺.

### Step J 3-(1-{6-[(4S)-3-{3-[4-(diethylphosphoryl)-3-(methylamino)phenyl]-2-oxo-2,3-dihydro-1H-imidazol-1-yl}-2-(4-fluoro-3,5-dimethylphenyl)-4-methyl-2H,4H,5H,6H,7H-pyrazolo[4,3-c]pyridine-5-carbonyl]-3-(oxan-4-yl)pyrrolo[1,2-b]pyridazin-7-yl}cyclopropyl)-4,5-dihydro-1,2,4-oxadiazol-5-one (Compound 26)

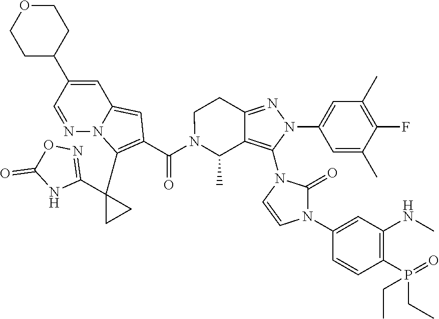 [SMILES: CCP(=O)(CC)c1ccc(-n2ccn(-c3c4c(nn3-c3cc(C)c(F)c(C)c3)CCN(C(=O)c3cc5cc(C6CCOCC6)cnn5c3C3(c5noc(=O)[nH]5)CC3)[C@H]4C)c2=O)cc1NC]

The mixture of (S,Z)-1-(6-(3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-3-(tetrahydro-2H-pyran-4-yl)pyrrolo[1,2-b]pyridazin-7-yl)-N′-hydroxycyclopropane-1-carboximidamide (200 mg, 0.228 mmol) and CDI (148 mg, 0.913 mmol) in THF (2 mL) was stirred at 50° C. for 2 h and then stirred at 60° C. for 16 h. After cooling, the mixture was filtered and purified by prep. HPLC (Column: Sunfire C18 19*250 mm*10 μm; Mobile Phase A: Water (0.1% formic acid), Mobile Phase B: CH₃CN; Flow rate: 20 mL/min; Gradient: 57% B to 67% B, retention time: 8.8-9.5 min) to obtain 3-(1-{6-[(4S)-3-{3-[4-(diethylphosphoryl)-3-(methylamino)phenyl]-2-oxo-2,3-dihydro-1H-imidazol-1-yl}-2-(4-fluoro-3,5-dimethylphenyl)-4-methyl-2H,4H,5H,6H,7H-pyrazolo[4,3-c]pyridine-5-carbonyl]-3-(oxan-4-yl)pyrrolo[1,2-b]pyridazin-7-yl}cyclopropyl)-4,5-dihydro-1,2,4-oxadiazol-5-one (79.3 mg, 38.5% yield). LC-MS: m/z 903.7 (M+H)⁺. ¹H NMR (400 MHz, DMSO-d6): δ 8.33 (s, 1H), 7.68-7.79 (m, 2H), 7.21-7.31 (m, 2H), 7.07-7.13 (m, 2H), 6.77-6.93 (m, 2H), 6.55 (s, 1H), 5.66-5.69 (m, 1H), 3.98-4.09 (m, 3H), 3.26-3.51 (m, 3H), 2.65-2.87 (m, 6H), 2.21 (s, 6H), 1.87-1.97 (m, 4H), 1.67-1.82 (m, 4H), 1.56 (s, 2H), 1.42-1.46 (m, 1H), 1.42 (d, J=1.2 Hz, 4H), 0.99-1.17 (m, 6H). ¹⁹F NMR (377 MHz, DMSO-d6); δ −122.14.

### Example 3

### 3-(1-{2-[(4S)-3-{3-[4-(diethylphosphoryl)-3-(methylamino)phenyl]-2-oxo-2,3-dihydro-1H-imidazol-1-yl}-2-(4-fluoro-3,5-dimethylphenyl)-4-methyl-2H,4H,5H,6H,7H-pyrazolo[4,3-c]pyridine-5-carbonyl]-7-(oxan-4-yl)indolizin-3-yl}cyclopropyl)-4,5-dihydro-1,2,4-oxadiazol-5-one (Compound 21)

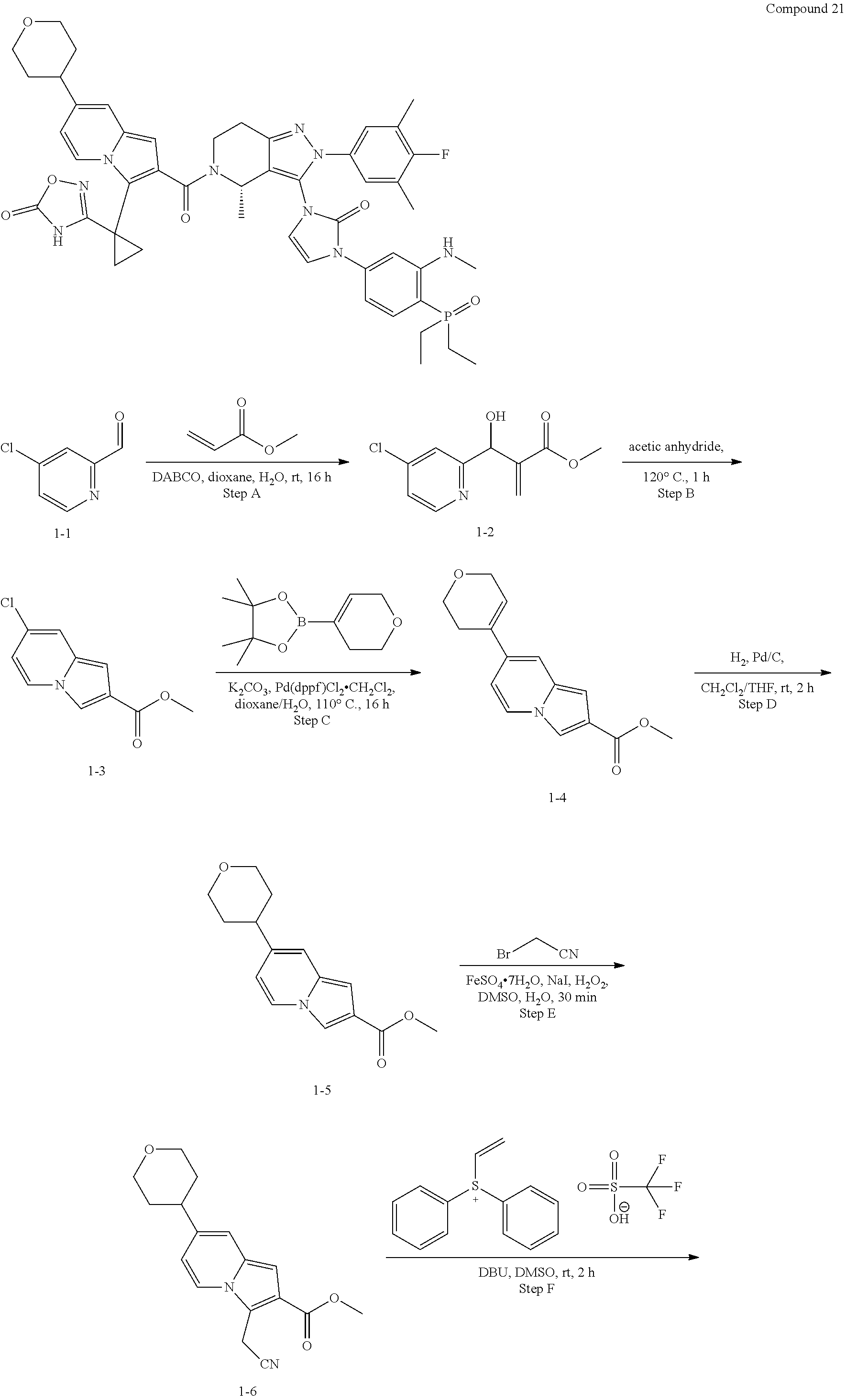

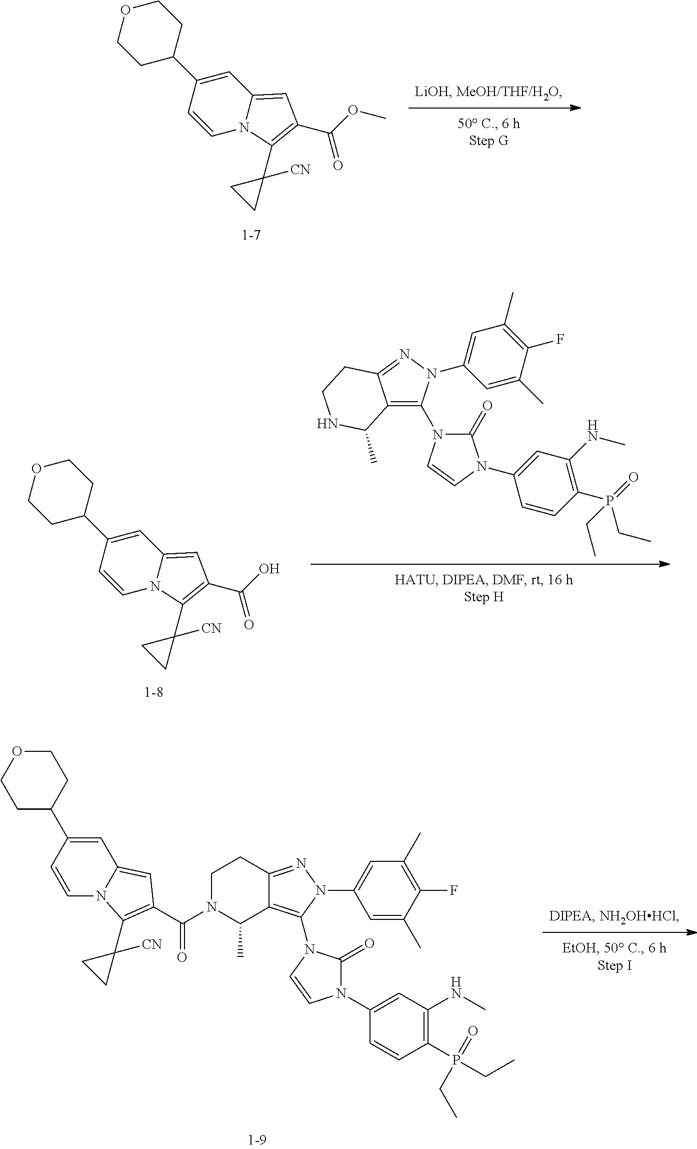 [SMILES: COC(=O)c1cc2cc(C3CCOCC3)ccn2c1C1(C#N)CC1 ; N#CC1(c2c(C(=O)O)cc3cc(C4CCOCC4)ccn23)CC1 ; CCP(=O)(CC)c1ccc(-n2ccn(-c3c4c(nn3-c3cc(C)c(F)c(C)c3)CCN[C@H]4C)c2=O)cc1NC ; CCP(=O)(CC)c1ccc(-n2ccn(-c3c4c(nn3-c3cc(C)c(F)c(C)c3)CCN(C(=O)c3cc5cc(C6CCOCC6)ccn5c3C3(C#N)CC3)[C@H]4C)c2=O)cc1NC]

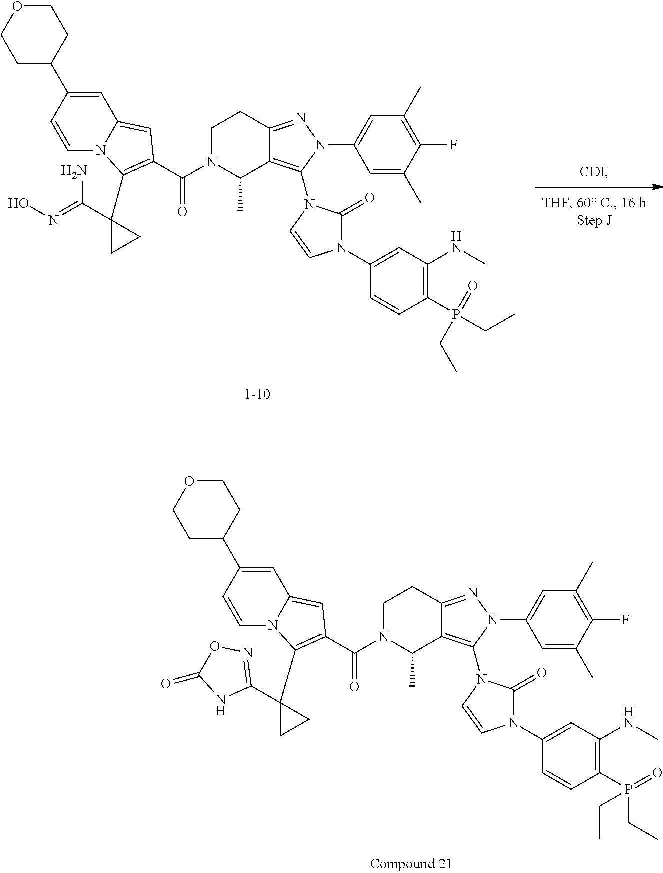 [SMILES: CCP(=O)(CC)c1ccc(-n2ccn(-c3c4c(nn3-c3cc(C)c(F)c(C)c3)CCN(C(=O)c3cc5cc(C6CCOCC6)ccn5c3C3(/C(N)=N/O)CC3)[C@H]4C)c2=O)cc1NC ; CCP(=O)(CC)c1ccc(-n2ccn(-c3c4c(nn3-c3cc(C)c(F)c(C)c3)CCN(C(=O)c3cc5cc(C6CCOCC6)ccn5c3C3(c5noc(=O)[nH]5)CC3)[C@H]4C)c2=O)cc1NC]

### Step A methyl 2-((4-chloropyridin-2-yl)(hydroxy)methyl)acrylate

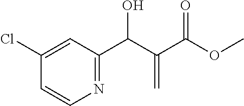

To the mixture of 4-chloropicolinaldehyde (10.0 g, 70.65 mmol), methyl acrylate (18.3 g, 211.93 mmol) in dioxane (100 mL) and H₂O (100 mL), was added DABCO (7.91 g, 70.65 mmol). The reaction mixture was stirred at room temperature for 16 h. Then the reaction was diluted with H₂O (200 mL), extracted with EtOAc (300 mL×3). The combined organic layers were washed with brine (200 mL×2), dried over anhydrous Na₂SO₄, filtered and concentrated. The residue was purified by flash silica gel chromatography (120 g SepaFlash® Silica Flash Column, Eluent of 0-25% EtOAc/PE gradient @80 mL/min) to afford methyl 2-[(4-chloropyridin-2-yl)(hydroxy)methyl]prop-2-enoate (13.9 g, 86.4% yield). LC-MS: m/z 228.1 (M+H)⁺.

### Step B methyl 7-chloroindolizine-2-carboxylate

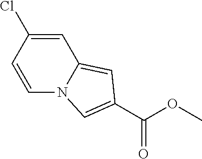

The mixture of methyl 2-((4-chloropyridin-2-yl)(hydroxy)methyl)acrylate (5 g, 21.97 mmol) in acetic anhydride (100 mL) was stirred at 100° C. for 1 h and then 130° C. for 2 h. After cooling, the reaction mixture was quenched with sat. aq. NaHCO₃ solution (30 mL), extracted with EtOAc (30 mL×3). The combined organic layers were washed with brine (30 mL×2), dried over anhydrous Na₂SO₄, filtered and concentrated. The residue was purified by flash silica gel chromatography (120 g SepaFlash® Silica Flash Column, Eluent of 0-20% EtOAc/PE gradient @80 mL/min) to give methyl 7-chloroindolizine-2-carboxylate (4.3 g, 93.4% yield). LC-MS: m/z 210.2 (M+H)⁺.

### Step C methyl 7-(3,6-dihydro-2H-pyran-4-yl)indolizine-2-carboxylate

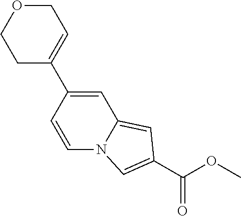 [SMILES: COC(=O)c1cc2cc(C3=CCOCC3)ccn2c1]

To the mixture of methyl 7-chloroindolizine-2-carboxylate (4.3 g, 20.51 mmol), 2-(3,6-dihydro-2H-pyran-4-yl)-4,4,5,5-tetramethyl-1,3,2-dioxaborolane (6.46 g, 30.77 mmol) and K₂CO₃ (8.49 g, 61.54 mmol) in dioxane (40 mL) and water (4 mL), was added Pd(dppf)Cl₂·CH₂Cl₂ (1.67 g, 2.05 mmol). After being charged with nitrogen three times, the reaction mixture was stirred at 110° C. for 16 h. After cooling, the reaction mixture was filtered, the filtrate was concentrated. The residue was purified flash silica gel chromatography (120 g SepaFlash® Silica Flash Column, Eluent of 0-75% EtOAc/PE gradient @50 mL/min) to get methyl 7-(3,6-dihydro-2H-pyran-4-yl)indolizine-2-carboxylate (2.2 g, 41.7% yield). LC-MS: m/z 258.2 (M+H)⁺.

### Step D methyl 7-(tetrahydro-2H-pyran-4-yl)indolizine-2-carboxylate

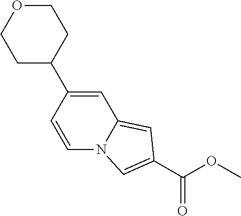 [SMILES: COC(=O)c1cc2cc(C3CCOCC3)ccn2c1]

The mixture of methyl 7-(3,6-dihydro-2H-pyran-4-yl)indolizine-2-carboxylate (2.2 g, 8.55 mmol) and 10% Pd/C (2.2 g) in DCM (10 mL) and THF (30 mL) was stirred at room temperature under H₂ balloon for 2 h. The mixture was filtered and the residue was concentrated to get methyl 7-(tetrahydro-2H-pyran-4-yl)indolizine-2-carboxylate (2.2 g, 99.2% yield). LC-MS: m/z 260.1 (M+H)⁺.

### Step E methyl 3-(cyanomethyl)-7-(3,4,5,6-tetrahydro-2H-pyran-4-yl)indolizine-2-carboxylate

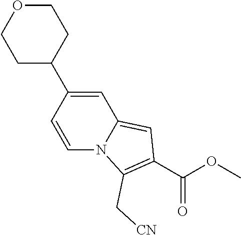 [SMILES: COC(=O)c1cc2cc(C3CCOCC3)ccn2c1CC#N]

To the ice water cooled mixture of methyl 7-(3,4,5,6-tetrahydro-2H-pyran-4-yl)indolizine-2-carboxylate (360 mg, 1.39 mmol), bromoacetonitrile (249.8 mg, 2.08 mmol), NaI (208.2 mg, 1.39 mmol) and FeSO₄.7H₂O (193.0 mg, 0.69 mmol) in DMSO (5.5 mL) and water (1.1 mL), was added 30% H₂O₂ (0.4 g) with stirring. After addition, the reaction mixture was stirred at 0° C. for another 30 min. The reaction mixture was diluted with EtOAc (50 mL) and water (50 mL). The organic layer was separated, washed with brine (50 mL), dried with anhydrous Na₂SO₄ and concentrated. The residue was purified by prep. HPLC (Column: Sunfire C18 19*250 mm*10 μm; Mobile Phase A: Water (0.1% formic acid), Mobile Phase B: CH₃CN; Flow rate: 20 mL/min; Gradient: 50% B to 60% B, retention time: 9.8-10.5 min) to afford methyl 3-(cyanomethyl)-7-(3,4,5,6-tetrahydro-2H-pyran-4-yl)indolizine-2-carboxylate (136 mg, 32.7% yield). LC-MS: m/z 299.1 (M+H)⁺.

### Step F methyl 3-(cyanocyclopropyl)-7-(3,4,5,6-tetrahydro-2H-pyran-4-yl)indolizine-2-carboxylate

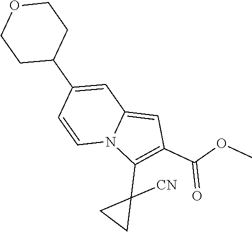 [SMILES: COC(=O)c1cc2cc(C3CCOCC3)ccn2c1C1(C#N)CC1]

To a solution of methyl 3-(cyanomethyl)-7-(3,4,5,6-tetrahydro-2H-pyran-4-yl)indolizine-2-carboxylate (270 mg, 0.91 mmol) in DMSO (10.0 mL) were added DBU (0.41 mL, 2.72 mmol) and ethenyldiphenylsulfanium trifluoromethanesulfonate (393.6 mg, 1.09 mmol). The reaction mixture was stirred at room temperature for 2 h. Then the reaction mixture was diluted with EtOAc (50 mL) and water (20 mL). The organic layer was separated, washed with brine (50 mL), dried with anhydrous Na₂SO₄ and concentrated. The residue was purified by flash silica gel chromatography (12 g SepaFlash® Silica Flash Column, Eluent of 0-50% EtOAc/PE gradient @50 mL/min) to give methyl 3-(cyanocyclopropyl)-7-(3,4,5,6-tetrahydro-2H-pyran-4-yl)indolizine-2-carboxylate (172 mg, 58.3% yield). LC-MS: m/z 325.2 (M+H)⁺.

### Step G 3-(cyanocyclopropyl)-7-(3,4,5,6-tetrahydro-2H-pyran-4-yl)indolizine-2-carboxylic acid

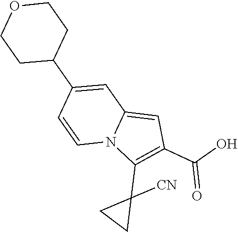 [SMILES: N#CC1(c2c(C(=O)O)cc3cc(C4CCOCC4)ccn23)CC1]

To a solution of methyl 3-(cyanocyclopropyl)-7-(3,4,5,6-tetrahydro-2H-pyran-4-yl)indolizine-2-carboxylate (275 mg, 0.85 mmol) in THF (8.0 mL), MeOH (1.5 mL) and H₂O (1.5 mL), was added LiOH·H₂O (356 mg, 8.48 mmol). The reaction mixture was stirred at 50° C. for 6 h. After cooling, the reaction mixture was diluted with EtOAc (10 mL) and water (20 mL). The organic layer was separated, the aqueous layer was acidified with 1M aq. HCl to pH=5, then extracted with DCM/MeOH (V/V=10:1, 50 mL) twice. The organic layer was combined, dried with anhydrous Na₂SO₄, filtered and concentrated to provide 3-(cyanocyclopropyl)-7-(3,4,5,6-tetrahydro-2H-pyran-4-yl)indolizine-2-carboxylic acid as a white solid (215 mg, 81.9% yield). LC-MS: m/z 311.1 (M+H)⁺.

### Step H 1-(2-{[(4S)-3-(1-[4-(diethylphosphoryl)-3-(methylamino)phenyl]-2-oxoimidazol-3-yl)-2-(4-fluoro-3,5-dimethylphenyl)-4-methyl-4,5,6,7-tetrahydropyrazolo[4,3-c]pyridin-5-yl]carbonyl}-7-(3,4,5,6-tetrahydro-2H-pyran-4-yl)indolizin-3-yl)cyclopropane-1-carbonitrile

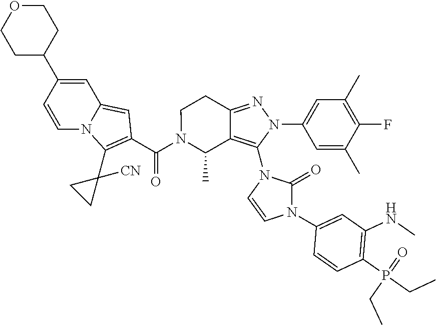 [SMILES: CCP(=O)(CC)c1ccc(-n2ccn(-c3c4c(nn3-c3cc(C)c(F)c(C)c3)CCN(C(=O)c3cc5cc(C6CCOCC6)ccn5c3C3(C#N)CC3)[C@H]4C)c2=O)cc1NC]

To a solution of 3-(cyanocyclopropyl)-7-(3,4,5,6-tetrahydro-2H-pyran-4-yl)indolizine-2-carboxylic acid (225 mg, 0.73 mmol) and (S)-1-(4-(diethylphosphoryl)-3-(methylamino)phenyl)-3-(2-(4-fluoro-3,5-dimethylphenyl)-4-methyl-4,5,6,7-tetrahydro-2H-pyrazolo[4,3-c]pyridin-3-yl)-1,3-dihydro-2H-imidazol-2-one (467.5 mg, 0.80 mmol) (HCl salt, preparation was described in WO2021155841A1) in DMF (10 mL) was added DIPEA (0.24 mL, 1.45 mmol) and HATU (413.5 mg, 1.09 mmol). The reaction mixture was stirred at room temperature for 16 h. The reaction mixture was diluted with water (50 mL), extracted with EtOAc (50 mL×3). The combined organic layers were washed with brine, dried with anhydrous Na₂SO₄, filtered and concentrated to provide the crude 1-(2-{[(4S)-3-(1-[4-(diethylphosphoryl)-3-(methylamino)phenyl]-2-oxoimidazol-3-yl)-2-(4-fluoro-3,5-dimethylphenyl)-4-methyl-4,5,6,7-tetrahydropyrazolo[4,3-c]pyridin-5-yl]carbonyl}-7-(3,4,5,6-tetrahydro-2H-pyran-4-yl)indolizin-3-yl)cyclopropane-1-carbonitrile (0.69 g). LC-MS: m/z 843.4 (M+H)⁺.

### Step I 1-[4-(diethylphosphoryl)-3-(methylamino)phenyl]-3-[(4S)-2-(4-fluoro-3,5-dimethylphenyl)-5-[(3-{[(Z)-amino(hydroxyimino)methyl]cyclopropyl}-7-(3,4,5,6-tetrahydro-2H-pyran-4-yl)indolizin-2-yl)carbonyl]-4-methyl-4,5,6,7-tetrahydropyrazolo[4,3-c]pyridin-3-yl]-2,3-dihydro-1H-imidazol-2-one

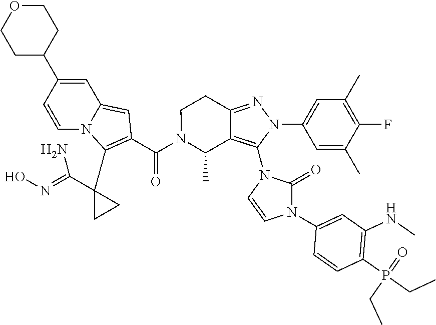 [SMILES: CCP(=O)(CC)c1ccc(-n2ccn(-c3c4c(nn3-c3cc(C)c(F)c(C)c3)CCN(C(=O)c3cc5cc(C6CCOCC6)ccn5c3C3(/C(N)=N/O)CC3)[C@H]4C)c2=O)cc1NC]

To a solution of 1-(2-{[(4S)-3-(1-[4-(diethylphosphoryl)-3-(methylamino)phenyl]-2-oxoimidazol-3-yl)-2-(4-fluoro-3,5-dimethylphenyl)-4-methyl-4,5,6,7-tetrahydropyrazolo[4,3-c]pyridin-5-yl]carbonyl}-7-(3,4,5,6-tetrahydro-2H-pyran-4-yl)indolizin-3-yl)cyclopropane-1-carbonitrile (1230 mg, 1.46 mmol) in EtOH (80 mL), was added NH₂OH·HCl (500 mg, 7.25 mmol) and DIPEA (2.381 mL, 14.59 mmol). The reaction mixture was stirred at 50° C. for 16 h. After cooling, the reaction mixture was concentrated. The residue was diluted with EtOAc (100 mL) and water (50 mL). The organic layer was separated, washed with brine, dried over anhydrous Na₂SO₄ and concentrated to afford crude 1-[4-(diethylphosphoryl)-3-(methylamino)phenyl]-3-[(4S)-2-(4-fluoro-3,5-dimethylphenyl)-5-[(3-{[(Z)-amino(hydroxyimino)methyl]cyclopropyl}-7-(3,4,5,6-tetrahydro-2H-pyran-4-yl)indolizin-2-yl)carbonyl]-4-methyl-4,5,6,7-tetrahydropyrazolo[4,3-c]pyridin-3-yl]-2,3-dihydro-1H-imidazol-2-one (1080 mg). LC-MS: m/z 876.5 (M+H)⁺.

### Step J 3-(1-{2-[(4S)-3-{3-[4-(diethylphosphoryl)-3-(methylamino)phenyl]-2-oxo-2,3-dihydro-1H-imidazol-1-yl}-2-(4-fluoro-3,5-dimethylphenyl)-4-methyl-2H,4H,5H,6H,7H-pyrazolo[4,3-c]pyridine-5-carbonyl]-7-(oxan-4-yl)indolizin-3-yl}cyclopropyl)-4,5-dihydro-1,2,4-oxadiazol-5-one (Compound 21)

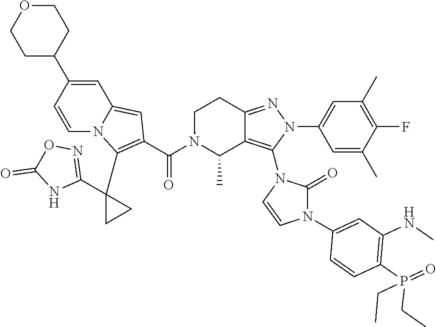 [SMILES: CCP(=O)(CC)c1ccc(-n2ccn(-c3c4c(nn3-c3cc(C)c(F)c(C)c3)CCN(C(=O)c3cc5cc(C6CCOCC6)ccn5c3C3(c5noc(=O)[nH]5)CC3)[C@H]4C)c2=O)cc1NC]

To the solution of 1-[4-(diethylphosphoryl)-3-(methylamino)phenyl]-3-[(4S)-2-(4-fluoro-3,5-dimethylphenyl)-5-[(3-{[(Z)-amino(hydroxyimino)methyl]cyclopropyl}-7-(3,4,5,6-tetrahydro-2H-pyran-4-yl)indolizin-2-yl)carbonyl]-4-methyl-4,5,6,7-tetrahydropyrazolo[4,3-c]pyridin-3-yl]-2,3-dihydro-1H-imidazol-2-one (540 mg, 0.62 mmol) in THF (8.0 mL) was added CDI (99.96 mg, 0.62 mmol). The reaction mixture was stirred at 50° C. for 2 h and then 60° C. for 16 h. After cooling, the reaction mixture was concentrated, the residue was purified by prep. HPLC (Column: Sunfire C18 19*250 mm*10 μm; Mobile Phase A: Water (0.1% formic acid), Mobile Phase B: CH₃CN; Flow rate: 20 mL/min; Gradient: 60% B to 70% B, retention time: 8.5 min) to provide 3-(1-{2-[(4S)-3-{3-[4-(diethylphosphoryl)-3-(methylamino)phenyl]-2-oxo-2,3-dihydro-1H-imidazol-1-yl}-2-(4-fluoro-3,5-dimethylphenyl)-4-methyl-2H,4H,5H,6H,7H-pyrazolo[4,3-c]pyridine-5-carbonyl]-7-(oxan-4-yl)indolizin-3-yl}cyclopropyl)-4,5-dihydro-1,2,4-oxadiazol-5-one (144.3 mg, 12.9% yield). LC-MS: m/z 902.4 (M+H)⁺. ¹H NMR (400 MHz, DMSO-d6): δ 8.08-8.12 (m, 1H), 7.63 (s, 1H), 7.22-7.34 (m, 3H), 7.11-7.19 (m, 2H), 6.81-6.92 (m, 2H), 6.68 (d, J=6.8 Hz, 1H), 6.39 (s, 1H), 5.60-5.73 (m, 1H), 4.78-5.08 (m, 1H), 4.04-4.14 (m, 1H), 3.94-3.96 (m, 2H), 3.44-3.47 (m, 4H), 2.66-2.90 (m, 6H), 2.18 (s, 6H), 1.88-1.93 (m, 4H), 1.60-1.76 (m, 6H), 1.24-1.53 (m, 5H), 0.96-1.04 (m, 6H). ¹⁹F NMR (377 MHz, DMSO-d6): δ −122.13.

### Example 4

### 3-(1-{2-[(4S)-3-{3-[4-(diethylphosphoryl)-3-(methylamino)phenyl]-2-oxo-2,3-dihydro-1H-imidazol-1-yl}-2-(4-fluoro-3,5-dimethylphenyl)-4-methyl-2H,4H,5H,6H,7H-pyrazolo[4,3-c]pyridine-5-carbonyl]-6-(oxan-4-yl)indolizin-1-yl}cyclopropyl)-4,5-dihydro-1,2,4-oxadiazol-5-one (Compound 20)

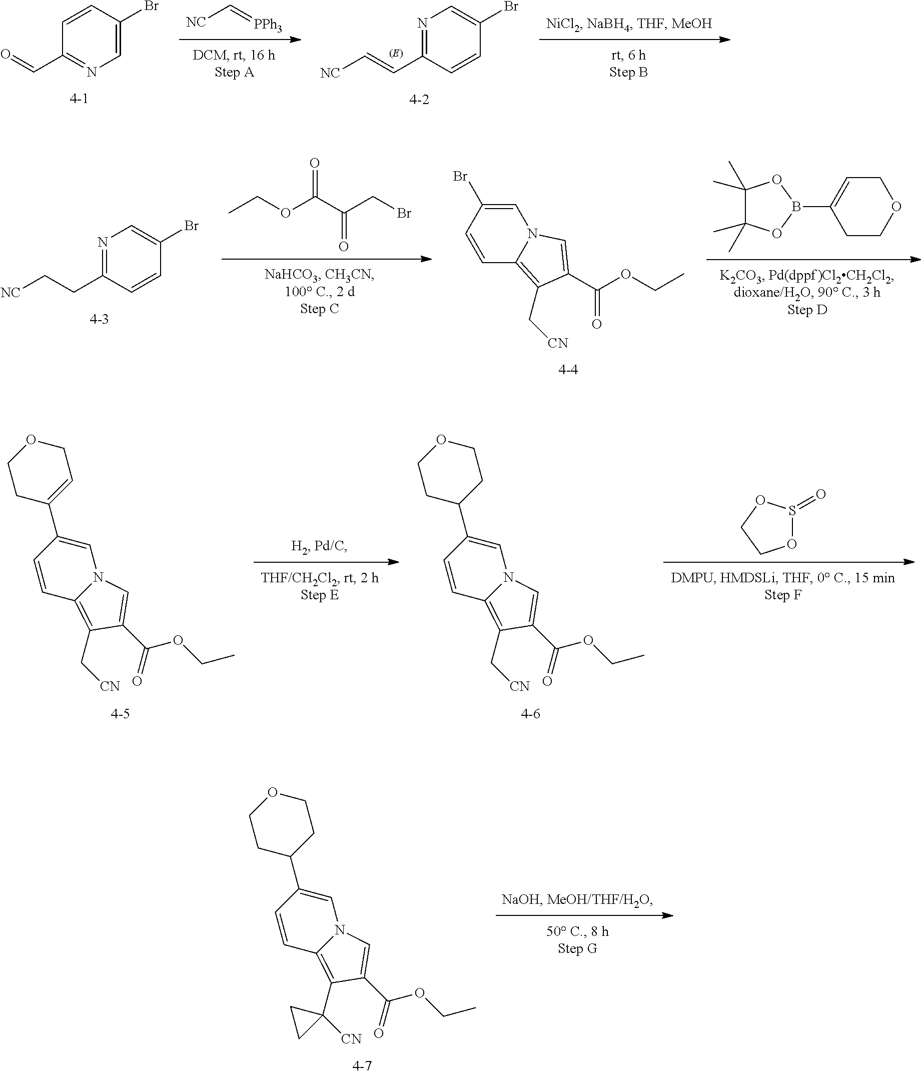

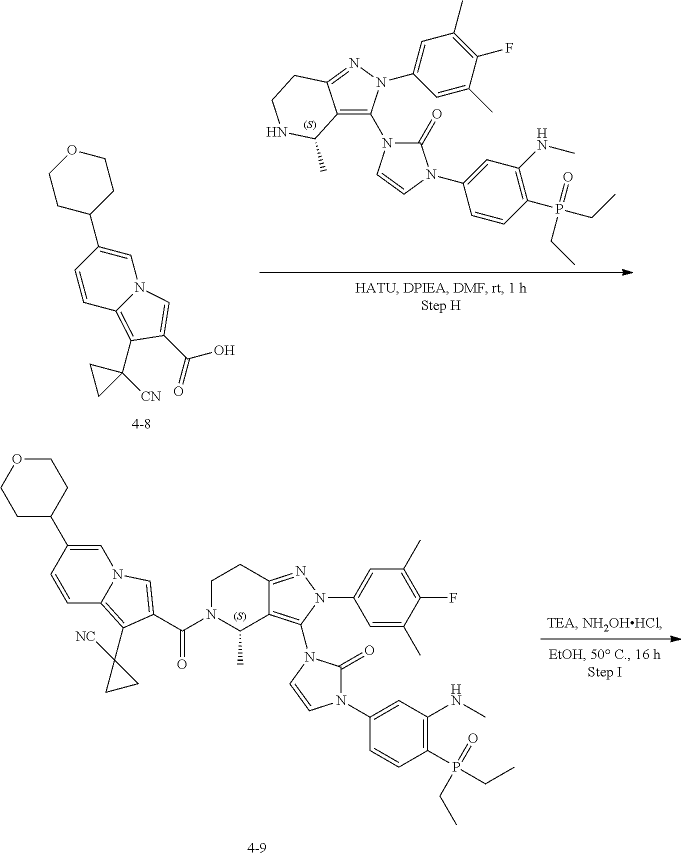

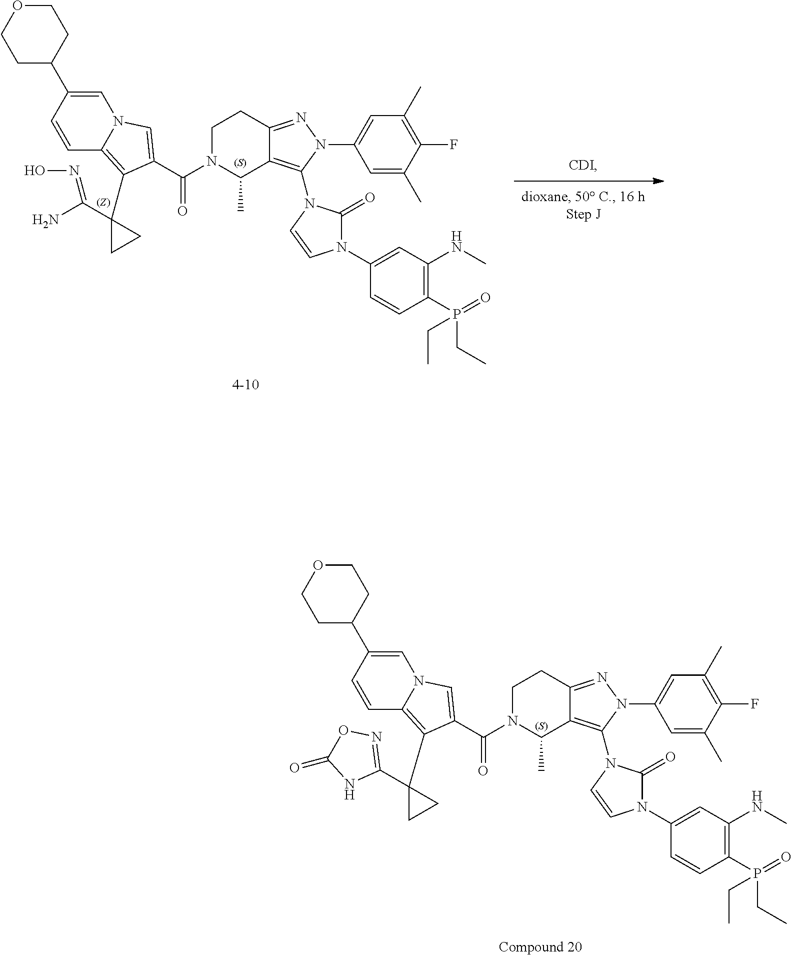

### Step A (E)-3-(5-bromopyridin-2-yl)acrylonitrile

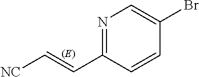 [SMILES: N#C/C=C/c1ccc(Br)cn1]

The mixture of 5-bromopicolinaldehyde (10.0 g, 54.05 mmol) and 2-(triphenyl-15-phosphaneylidene)acetonitrile (16.3 g, 54.15 mmol) in DCM (50 mL) was stirred at room temperature for 2 days. The reaction mixture was filtered, the filtrate was concentrated. The residue was purified by flash silica gel chromatography (120 g SepaFlash® Silica Flash Column, Eluent of 0-8% EtOAc/PE gradient @50 mL/min) to give (E)-3-(5-bromopyridin-2-yl)acrylonitrile (7.0 g, 62.0% yield). LC-MS: m/z 209.0 (M+H)⁺.

### Step B 3-(5-bromopyridin-2-yl)propanenitrile

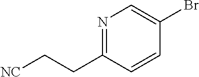 [SMILES: N#CCCc1ccc(Br)cn1]

To a mixture of (E)-3-(5-bromopyridin-2-yl)acrylonitrile (7.0 g, 33.65 mmol) in THF (60 mL) and MeOH (20 mL) was added NaBH₄ (2.30 g, 60.57 mmol) and NiCl₂ (438 mg, 3.36 mmol) at 0° C. The reaction was stirred at room temperature for 6 h. The reaction mixture was quenched with H₂O (300 mL), extracted with ethyl acetate (200 mL×3). The organic layer was combined and washed with brine (100 mL×2), dried over Na₂SO₄, filtered and concentrated. The residue was purified by flash silica gel chromatography (120 g SepaFlash® Silica Flash Column, Eluent of 0-70% EtOAc/PE gradient @80 mL/min) to give 3-(5-bromopyridin-2-yl)propanenitrile (6.0 g, 85.0% yield). LC-MS: m/z 211.0 (M+H)⁺.

### Step C ethyl 6-bromo-1-(cyanomethyl)indolizine-2-carboxylate

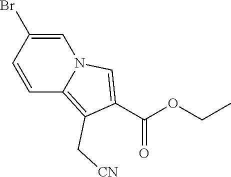 [SMILES: CCOC(=O)c1cn2cc(Br)ccc2c1CC#N]

To the mixture of 3-(5-bromopyridin-2-yl)propanenitrile (4.5 g, 21.43 mmol) and NaHCO₃ (5.4 g, 64.28 mmol) in CH₃CN (8.0 mL) was added ethyl 3-bromo-2-oxopropanoate (12.5 g, 64.10 mmol). The resulting mixture was stirred at 100° C. for 2 days. After cooling, the reaction mixture was filtered, the filtrate was concentrated. The residue was purified by flash silica gel chromatography (120 g SepaFlash® Silica Flash Column, Eluent of 0-25% EtOAc/PE gradient @80 mL/min) to give ethyl 6-bromo-1-(cyanomethyl)indolizine-2-carboxylate (350 mg, 11.4% yield). LC-MS: m/z 307.3 (M+H)⁺.

### Step D ethyl 1-(cyanomethyl)-6-(3,6-dihydro-2H-pyran-4-yl)indolizine-2-carboxylate

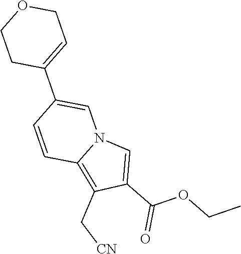 [SMILES: CCOC(=O)c1cn2cc(C3=CCOCC3)ccc2c1CC#N]

To the mixture of ethyl 6-bromo-1-(cyanomethyl)indolizine-2-carboxylate (350 mg, 1.14 mmol), 2-(3,6-dihydro-2H-pyran-4-yl)-4,4,5,5-tetramethyl-1,3,2-dioxaborolane (360 mg, 1.71 mmol) and K₂CO₃ (439 mg, 3.18 mmol) in dioxane (6.0 mL) and H₂O (0.6 mL) was added Pd(dppf)Cl₂CH₂Cl₂ (86.35 mg, 0.106 mmol). After being charged with nitrogen three times, the mixture was stirred at 80° C. for 3 h under nitrogen. After cooling, the mixture was filtered and concentrated in vacuum. The residue was purified by flash silica gel chromatography (4 g SepaFlash® Silica Flash Column, Eluent of 0-25% EtOAc/PE gradient @40 mL/min) to give ethyl 1-(cyanomethyl)-6-(3,6-dihydro-2H-pyran-4-yl)indolizine-2-carboxylate (325 mg, 91.8% yield). LC-MS: m/z 311.2 (M+H)⁺.

### Step E ethyl 1-(cyanomethyl)-6-(tetrahydro-2H-pyran-4-yl)indolizine-2-carboxylate

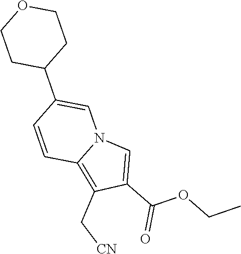 [SMILES: CCOC(=O)c1cn2cc(C3CCOCC3)ccc2c1CC#N]

To the solution of ethyl 1-(cyanomethyl)-6-(3,6-dihydro-2H-pyran-4-yl)indolizine-2-carboxylate (325 mg, 1.05 mmol) in THF (3 mL) and DCM (1 mL) was added 5% Pd/C (487.5 mg) at room temperature. The reaction mixture was stirred at room temperature under H₂ balloon for 2 h. The reaction mixture was filtered, the filtrate was concentrated to give ethyl 1-(cyanomethyl)-6-(tetrahydro-2H-pyran-4-yl)indolizine-2-carboxylate (300 mg, 91.7% yield). LC-MS: m/z 313.2 (M+H)⁺.

### Step F ethyl 1-(1-cyanocyclopropyl)-6-(tetrahydro-2H-pyran-4-yl)indolizine-2-carboxylate

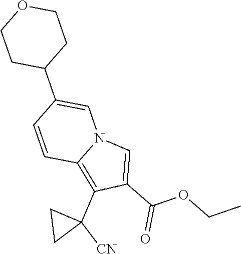 [SMILES: CCOC(=O)c1cn2cc(C3CCOCC3)ccc2c1C1(C#N)CC1]

To the mixture of ethyl 1-(cyanomethyl)-6-(3,4,5,6-tetrahydro-2H-pyran-4-yl)indolizine-2-carboxylate (300 mg, 0.96 mmol), 1,3,2-dioxathiolane 2-oxide (124.6 mg, 1.15 mmol) in THF (6 mL) was added HMDSLi (2.88 mL, 2.88 mmol, 1 M in THF) and DMPU (245.9 mg, 1.92 mmol) at 0° C. under nitrogen. The reaction mixture was stirred at 0° C. for 15 min. The reaction was quenched with sat. aq. NH₄Cl (30 mL), extracted with ethyl acetate (30 mL×3). The combined organic layers were washed with brine (30 mL×2), dried over Na₂SO₄, filtered and concentrated. The residue was purified by flash silica gel chromatography (4 g SepaFlash® Silica Flash Column, Eluent of 0-25% EtOAc/PE gradient @50 mL/min) to get ethyl 1-(1-cyanocyclopropyl)-6-(tetrahydro-2H-pyran-4-yl)indolizine-2-carboxylate (130 mg, 40.0% yield). LC-MS: m/z 339.2 (M+H)⁺.

### Step G 1-(1-cyanocyclopropyl)-6-(tetrahydro-2H-pyran-4-yl)indolizine-2-carboxylic acid

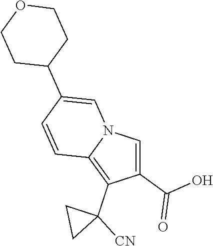 [SMILES: N#CC1(c2c(C(=O)O)cn3cc(C4CCOCC4)ccc23)CC1]

To the mixture of ethyl 1-(1-cyanocyclopropyl)-6-(tetrahydro-2H-pyran-4-yl)indolizine-2-carboxylate (130 mg, 0.38 mmol) in MeOH (2 mL), THF (3 mL) and H₂O (1 mL) was added NaOH (61.5 mg, 1.54 mmol) at room temperature. The reaction mixture was stirred at 50° C. for 8 h. After cooling, the mixture was diluted with water (20 mL), acidified with 1M aq. HCl to pH=5, extracted with ethyl acetate (30 mL×3). The combined organic layers were dried over anhydrous Na₂SO₄, filtered and concentrated. The residue was purified by flash silica gel chromatography (4 g SepaFlash® Silica Flash Column, Eluent of 0-4% MeOH/CH₂Cl₂ gradient @50 mL/min) to give 1-(1-cyanocyclopropyl)-6-(tetrahydro-2H-pyran-4-yl)indolizine-2-carboxylic acid (70 mg, 58.8% yield). LC-MS: m/z 311.5 (M+H)⁺.

### Step H (S)-1-(2-(3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-6-(tetrahydro-2H-pyran-4-yl)indolizin-1-yl)cyclopropane-1-carbonitrile

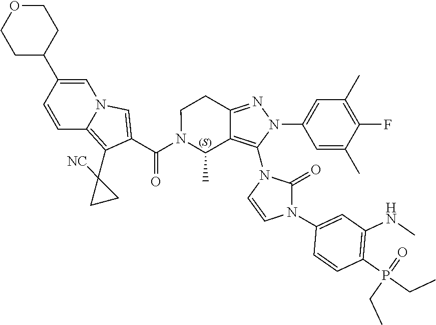

To the solution of 1-(1-cyanocyclopropyl)-6-(tetrahydro-2H-pyran-4-yl)indolizine-2-carboxylic acid (70 mg, 0.23 mmol) and (S)-1-(4-(diethylphosphoryl)-3-(methylamino)phenyl)-3-(2-(4-fluoro-3,5-dimethylphenyl)-4-methyl-4,5,6,7-tetrahydro-2H-pyrazolo[4,3-c]pyridin-3-yl)-1,3-dihydro-2H-imidazol-2-one (132.5 mg, 0.23 mmol) (HCl salt, preparation was described in WO2021155841A1) in DMF (2 mL), was added DIPEA (0.5 mL) and HATU (103.0 mg, 0.27 mmol). The reaction mixture was stirred at room temperature for 1 h. The reaction mixture was quenched with H₂O (25 mL), extracted with ethyl acetate (20 mL×3). The combined organic layers were washed with brine (20 mL×2), dried over anhydrous Na₂SO₄, filtered and concentrated. The residue was purified by flash silica gel chromatography (12 g SepaFlash® Silica Flash Column, Eluent of 0-5% MeOH/CH₂Cl₂ gradient @50 mL/min) to give (S)-1-(2-(3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-6-(tetrahydro-2H-pyran-4-yl)indolizin-1-yl)cyclopropane-1-carbonitrile (160 mg, 84.2% yield). LC-MS: m/z 843.9 (M+H)⁺.

### Step I (S,Z)-1-(2-(3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-6-(tetrahydro-2H-pyran-4-yl)indolizin-1-yl)-N′-hydroxycyclopropane-1-carboximidamide

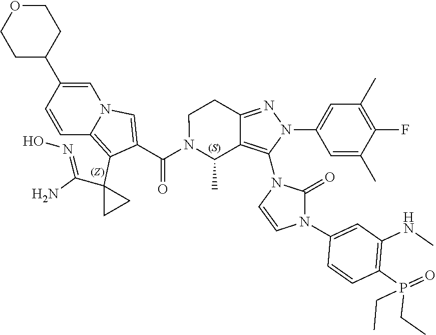

The mixture of (S)-1-(2-(3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-6-(tetrahydro-2H-pyran-4-yl)indolizin-1-yl)cyclopropane-1-carbonitrile (50 mg, 0.059 mmol), NH₂OH·HCl (12 mg, 0.17 mmol) and TEA (0.3 mL) in EtOH (2.0 mL) was stirred at 50° C. for 16 h. After cooling, the reaction was quenched with H₂O (20 mL), extracted with ethyl acetate (20 mL×3). The combined organic layers were washed with brine (20 mL×2), dried over anhydrous Na₂SO₄, filtered and concentrated to give (S,Z)-1-(2-(3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-6-(tetrahydro-2H-pyran-4-yl)indolizin-1-yl)-N′-hydroxycyclopropane-1-carboximidamide (50 mg, crude). LC-MS: m/z 876.4 (M+H)⁺.

### Step J 3-(1-{2-[(4S)-3-{3-[4-(diethylphosphoryl)-3-(methylamino)phenyl]-2-oxo-2,3-dihydro-1H-imidazol-1-yl}-2-(4-fluoro-3,5-dimethylphenyl)-4-methyl-2H,4H,5H,6H,7H-pyrazolo[4,3-c]pyridine-5-carbonyl]-6-(oxan-4-yl)indolizin-1-yl}cyclopropyl)-4,5-dihydro-1,2,4-oxadiazol-5-one (Compound 20)

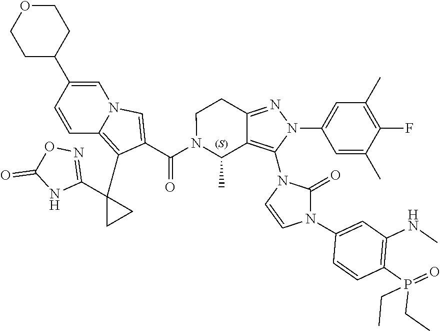

The mixture of (S,Z)-1-(2-(3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-6-(tetrahydro-2H-pyran-4-yl)indolizin-1-yl)-N′-hydroxycyclopropane-1-carboximidamide (50 mg, crude) and CDI (28 mg, 0.17 mmol) in dioxane (2.0 mL) was stirred at 50° C. for 16 h. After cooling, the reaction mixture concentrated. The residue was purified by prep. HPLC (Column: Sunfire C18 19*250 mm*10 μm; Mobile Phase A: Water (0.05% TFA), Mobile Phase B: CH₃CN; Flow rate: 20 mL/min; Gradient: 62% B to 72% B, retention time: 7.5-8.4 min) to give 3-(1-{2-[(4S)-3-{3-[4-(diethylphosphoryl)-3-(methylamino)phenyl]-2-oxo-2,3-dihydro-1H-imidazol-1-yl}-2-(4-fluoro-3,5-dimethylphenyl)-4-methyl-2H,4H,5H,6H,7H-pyrazolo[4,3-c]pyridine-5-carbonyl]-6-(oxan-4-yl)indolizin-1-yl}cyclopropyl)-4,5-dihydro-1,2,4-oxadiazol-5-one (12.5 mg, yield: 24.4% yield). LC-MS: m/z 902.2 (M+H)⁺. ¹H NMR (400 MHz, DMSO-d6): δ 11.75 (br s, 1H), 8.10 (s, 1H), 7.68 (s, 1H), 7.47 (d, J=9.6 Hz, 1H), 7.20-7.33 (m, 2H), 7.10-7.17 (m, 2H), 6.78-6.93 (m, 4H), 3.96-4.00 (m, 2H), 3.41-3.53 (m, 3H), 2.66-2.87 (m, 6H), 2.25 (s, 6H), 1.85-2.01 (m, 4H), 1.41-1.81 (m, 7H), 1.26-1.37 (m, 5H), 1.13-1.21 (m, 1H), 0.95-1.01 (m. 6H). ¹⁹F NMR:(377 MHz, DMSO-d6): δ −122.33.

### Example 5

### 3-(1-{2-[(4S)-3-{3-[4-(diethylphosphoryl)-3-(methylamino)phenyl]-2-oxo-2,3-dihydro-1H-imidazol-1-yl}-2-(4-fluoro-3,5-dimethylphenyl)-4-methyl-2H,4H,5H,6H,7H-pyrazolo[4,3-c]pyridine-5-carbonyl]-6-(oxan-4-yl)-1-benzofuran-3-yl}cyclopropyl)-4,5-dihydro-1,2,4-oxadiazol-5-one (Compound 19)

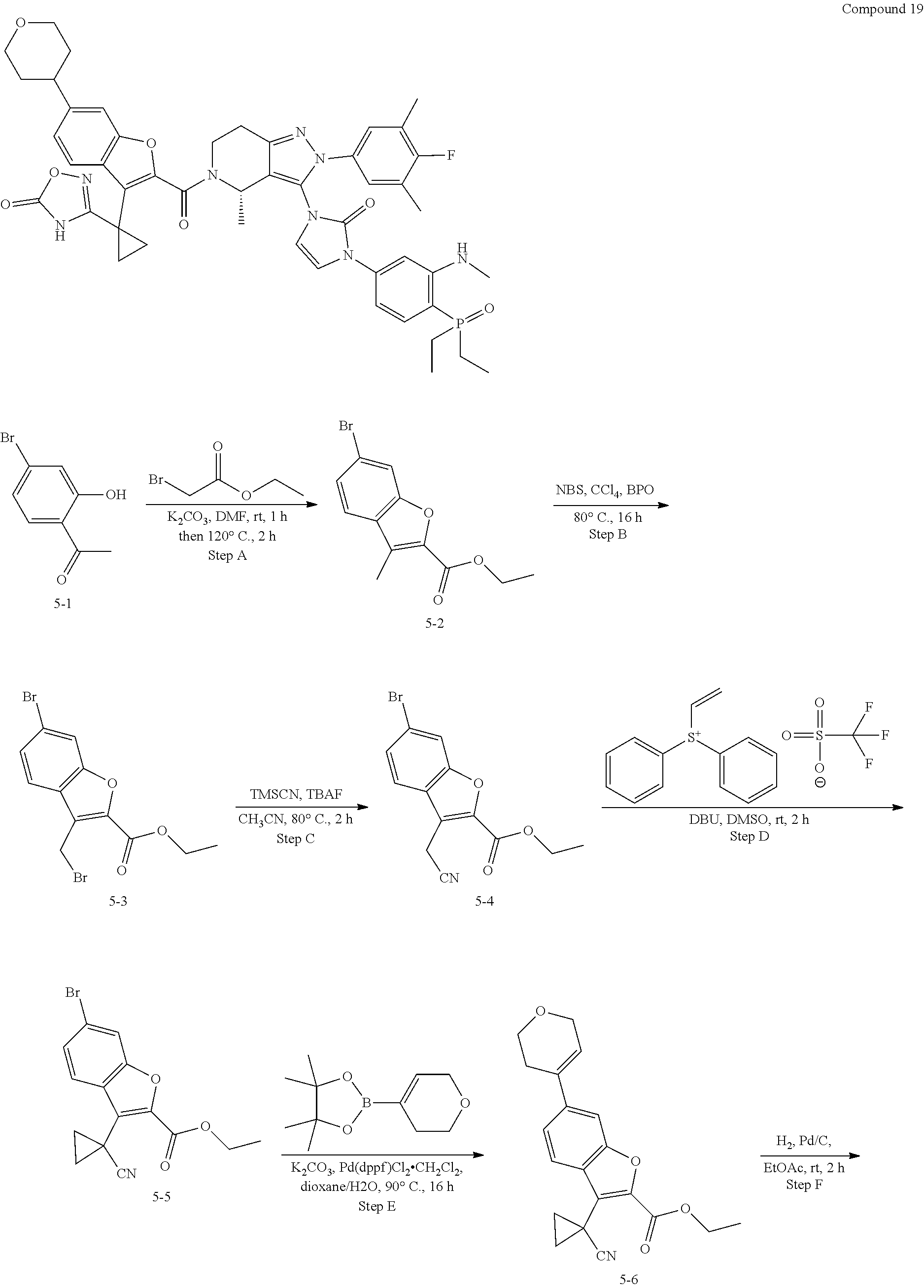

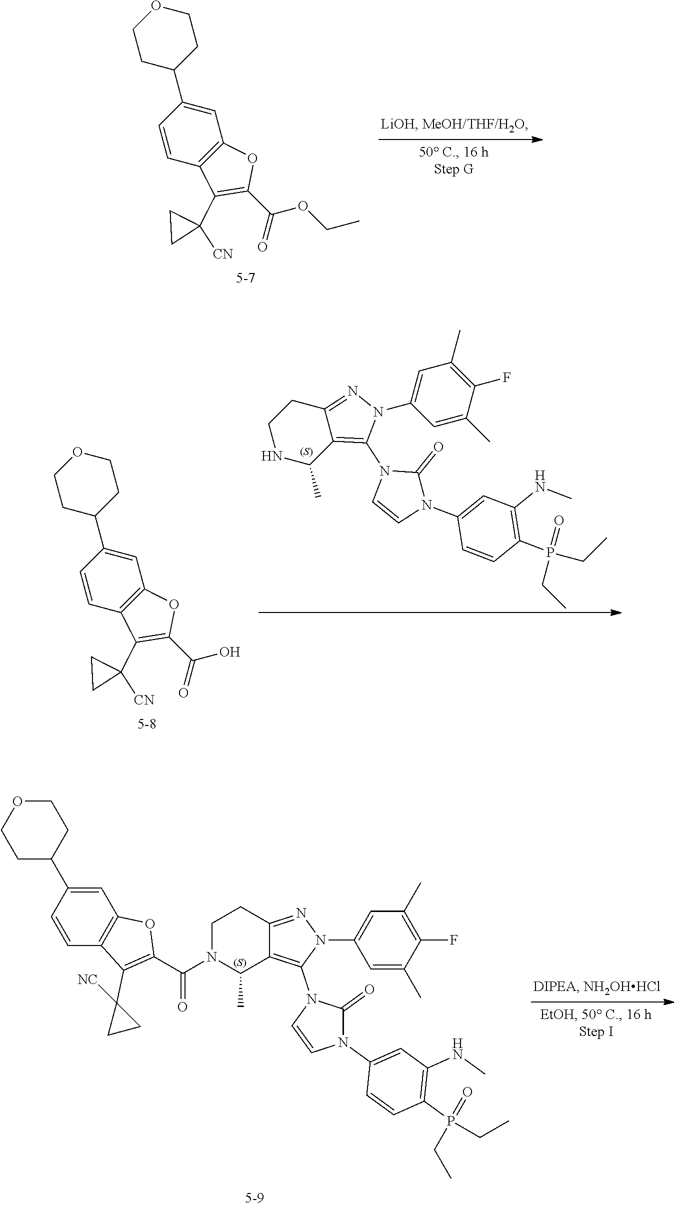

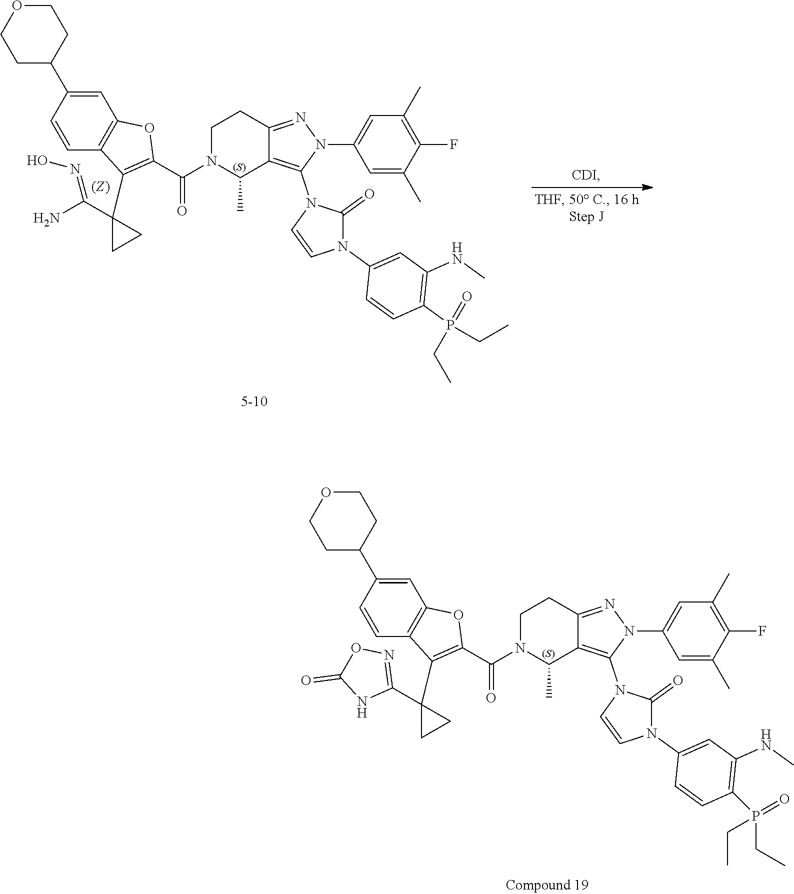

### Step A ethyl 6-bromo-3-methylbenzofuran-2-carboxylate

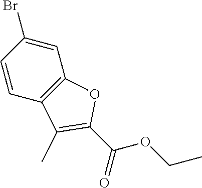 [SMILES: CCOC(=O)c1oc2cc(Br)ccc2c1C]

To the solution of 1-(4-bromo-2-hydroxyphenyl)ethan-1-one (10.0 g, 46.5 mmol) in DMF (50 mL) was added ethyl 2-bromoacetate (9.3 g, 55.8 mmol) and K₂CO₃ (19.3 g, 139.5 mmol). The reaction mixture was stirred at room temperature for 1 h and then heated at 120° C. and continued to stir for 2 h. After cooling, the reaction mixture was poured into water (200 mL), extracted with ethyl acetate (200 mL). The organic layer was washed with brine (200 mL×2), dried over anhydrous Na₂SO₄, filtered and concentrated. The residue was purified by flash silica gel chromatography (120 g SepaFlash® Silica Flash Column, Eluent of 0-10% EtOAc/PE gradient @100 mL/min) to give ethyl 6-bromo-3-methylbenzofuran-2-carboxylate (6.0 g, 45.6% yield). LC-MS: m/z 283.0 (M+H)⁺.

### Step B ethyl 6-bromo-3-(bromomethyl)benzofuran-2-carboxylate

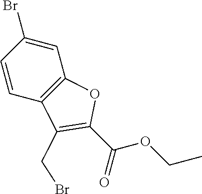 [SMILES: CCOC(=O)c1oc2cc(Br)ccc2c1CBr]

To the solution of ethyl 6-bromo-3-methylbenzofuran-2-carboxylate (6.0 g, 21.2 mmol) in CCl₄ (60 mL) was added BPO (136 mg, 0.42 mmol) and NBS (3.8 g, 21.2 mmol). The resulting reaction mixture was stirred at 80° C. for 16 h under nitrogen. After cooling and concentration, the residue was diluted with water (200 mL), extracted with ethyl acetate (200 mL). The organic layer was washed with brine (200 mL×2), dried over anhydrous Na₂SO₄, filtered and concentrated. The residue was purified by flash silica gel chromatography (120 g SepaFlash® Silica Flash Column, Eluent of 0-10% EtOAc/PE gradient @80 mL/min) to give ethyl 6-bromo-3-(bromomethyl)benzofuran-2-carboxylate (5.7 g, 74.3% yield). LC-MS: m/z 362.6 (M+H)⁺.

### Step C ethyl 6-bromo-3-(cyanomethyl)benzofuran-2-carboxylate

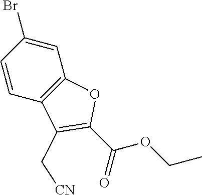 [SMILES: CCOC(=O)c1oc2cc(Br)ccc2c1CC#N]

The mixture of ethyl 6-bromo-3-(bromomethyl)benzofuran-2-carboxylate (2.30 g, 6.35 mmol), TMSCN (2.52 g, 25.41 mmol) and TBAF (9.53 mL, 9.53 mmol, 1M THF solution) in CH₃CN (10 mL) was stirred at 80° C. for 2 h. After cooling, the reaction mixture was poured into water (50 mL), extracted with EtOAc (50 mL×2). The combined organic layer was washed with brine, dried over anhydrous Na₂SO₄, filtered and concentrated. The residue was purified by flash silica gel chromatography (40 g SepaFlash® Silica Flash Column, Eluent of 0-50% EtOAc/PE gradient @50 mL/min) to afford ethyl 6-bromo-3-(cyanomethyl)benzofuran-2-carboxylate (730 mg, 37.3% yield). LC-MS: m/z 308.0 (M+H)⁺.

### Step D ethyl 6-bromo-3-(1-cyanocyclopropyl)benzofuran-2-carboxylate

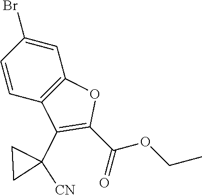 [SMILES: CCOC(=O)c1oc2cc(Br)ccc2c1C1(C#N)CC1]

The mixture of ethyl 6-bromo-3-(cyanomethyl)benzofuran-2-carboxylate (940 mg, 3.05 mmol), ethenyldiphenylsulfanium trifluoromethanesulfonate (1.33 g, 3.66 mmol) and DBU (1.37 mL, 9.15 mmol) in DMSO (5 mL) was stirred at room temperature for 2 h. The mixture was poured into water (20 mL), extracted with EtOAc (20 mL×2). The combined organic layer was washed with brine, dried over anhydrous Na₂SO₄, filtered and concentrated. The residue was purified by prep. TLC (PE/EtOAc=3/1) to afford ethyl 6-bromo-3-(1-cyanocyclopropyl)benzofuran-2-carboxylate (535 mg, 52.5% yield). LC-MS: m/z 334.0 (M+H)⁺.

### Step E ethyl 3-(1-cyanocyclopropyl)-6-(3,6-dihydro-2H-pyran-4-yl)benzofuran-2-carboxylate

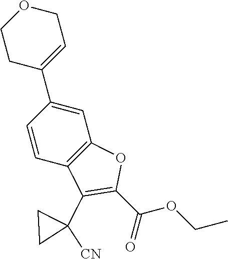 [SMILES: CCOC(=O)c1oc2cc(C3=CCOCC3)ccc2c1C1(C#N)CC1]

To the mixture of ethyl 6-bromo-3-(1-cyanocyclopropyl)benzofuran-2-carboxylate (350 mg, 1.05 mmol), 2-(3,6-dihydro-2H-pyran-4-yl)-4,4,5,5-tetramethyl-1,3,2-dioxaborolane (330 mg, 1.57 mmol) and K₂CO₃ (434 mg, 3.14 mmol) in dioxane/H₂O (5 mL/0.5 mL), was added Pd(dppf)Cl₂·CH₂Cl₂ (77 mg, 0.11 mmol). After being charged with nitrogen three times, the reaction mixture was stirred at 90° C. for 16 h. After cooling, the mixture was concentrated and purified by flash silica gel chromatography (12 g SepaFlash® Silica Flash Column, Eluent of 0-10% EtOAc/PE gradient @50 mL/min) to afford ethyl 3-(1-cyanocyclopropyl)-6-(3,6-dihydro-2H-pyran-4-yl)benzofuran-2-carboxylate (290 mg, 82.1% yield). LC-MS: m/z 338.2 (M+H)⁺.

### Step F ethyl 3-(1-cyanocyclopropyl)-6-(tetrahydro-2H-pyran-4-yl)benzofuran-2-carboxylate

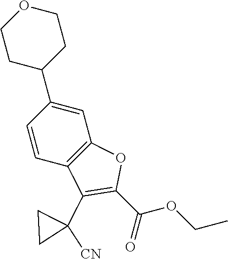 [SMILES: CCOC(=O)c1oc2cc(C3CCOCC3)ccc2c1C1(C#N)CC1]

The mixture of ethyl 3-(1-cyanocyclopropyl)-6-(3,6-dihydro-2H-pyran-4-yl)benzofuran-2-carboxylate (290 mg, 0.86 mmol) and 10% Pd/C (60 mg) in EtOAc (20 mL) was stirred at room temperature under H₂ balloon for 16 h. The reaction mixture was filtered, the filtrate was concentrated to obtain ethyl 3-(1-cyanocyclopropyl)-6-(tetrahydro-2H-pyran-4-yl)benzofuran-2-carboxylate (291 mg, 99% yield). LC-MS: m/z 340.2 (M+H)⁺.

### Step G 3-(1-cyanocyclopropyl)-6-(tetrahydro-2H-pyran-4-yl)benzofuran-2-carboxylic acid

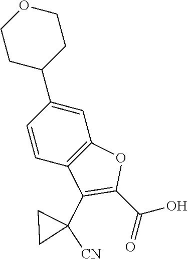 [SMILES: N#CC1(c2c(C(=O)O)oc3cc(C4CCOCC4)ccc23)CC1]

To the solution of ethyl 3-(1-cyanocyclopropyl)-6-(tetrahydro-2H-pyran-4-yl)benzofuran-2-carboxylate (291 mg, 0.86 mmol) in THF/EtOH/H₂O (5 mL/1 mL/1 mL) was added LiOH·H₂O (288 mg, 6.86 mmol). The resulting mixture was stirred at 50° C. for 16 h. After cooling, the mixture was diluted with water (50 mL), extracted with EtOAc (25 mL×2). The aqueous phase was acidified by 2 M aq. HCl to pH=2, extracted with EtOAc (30 mL×3). The combined organic layer was washed with brine, dried over anhydrous Na₂SO₄, filtered and concentrated to afford 3-(1-cyanocyclopropyl)-6-(tetrahydro-2H-pyran-4-yl)benzofuran-2-carboxylic acid (170 mg, 63.7% yield). LC-MS: m/z 312.2 (M+H)⁺.

### Step H (S)-1-(2-(3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-6-(tetrahydro-2H-pyran-4-yl)benzofuran-3-yl)cyclopropane-1-carbonitrile

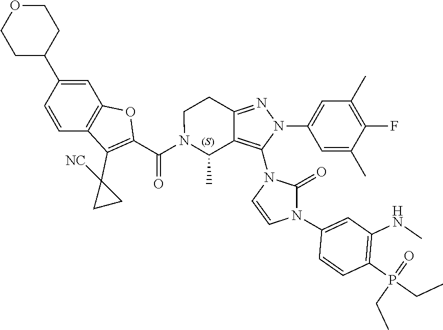

To the mixture of 3-(1-cyanocyclopropyl)-6-(tetrahydro-2H-pyran-4-yl)benzofuran-2-carboxylic acid (170 mg, 0.54 mmol), (S)-1-(4-(diethylphosphoryl)-3-(methylamino)phenyl)-3-(2-(4-fluoro-3,5-dimethylphenyl)-4-methyl-4,5,6,7-tetrahydro-2H-pyrazolo[4,3-c]pyridin-3-yl)-1,3-dihydro-2H-imidazol-2-one (320 mg, 0.54 mmol) (HCl salt, preparation was described in WO2021155841A1) in DMF (4 mL), was added HATU (311.4 mg, 0.82 mmol) and DIPEA (212 mg, 1.64 mmol). The resulting mixture was stirred at room temperature for 16 h. The mixture was poured into water, extracted with CH₂Cl₂ (50 mL×2). The combined organic layers were washed with brine (20 mL×2), dried over anhydrous Na₂SO₄, filtered and concentrated. The residue was purified by flash silica gel chromatography (12 g SepaFlash® Silica Flash Column, Eluent of 0-5% MeOH/CH₂Cl₂ gradient @50 mL/min) to obtain (S)-1-(2-(3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-6-(tetrahydro-2H-pyran-4-yl)benzofuran-3-yl)cyclopropane-1-carbonitrile (260 mg, 56.4% yield). LC-MS: m/z 844.4 (M+H)⁺.

### Step I (S,Z)-1-(2-(3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-6-(tetrahydro-2H-pyran-4-yl)benzofuran-3-yl)-N′-hydroxycyclopropane-1-carboximidamide

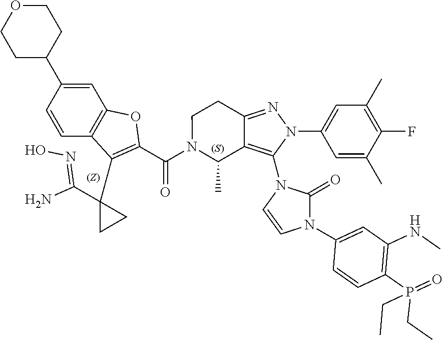

The mixture of (S)-1-(2-(3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-6-(tetrahydro-2H-pyran-4-yl)benzofuran-3-yl)cyclopropane-1-carbonitrile (130 mg, 0.154 mmol), NH₂OH·HCl (107 mg, 5.40 mmol) and DIPEA (0.32 mL, 1.84 mmol) in EtOH (6 mL) was stirred at 50° C. for 16 h. After cooling, the reaction was quenched with H₂O (20 mL), extracted with ethyl acetate (20 mL×3). The combined organic layers were washed with brine (20 mL×2), dried over anhydrous Na₂SO₄, filtered and concentrated to give (S,Z)-1-(2-(3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-6-(tetrahydro-2H-pyran-4-yl)benzofuran-3-yl)-N′-hydroxycyclopropane-1-carboximidamide (134 mg, crude). LC-MS: m/z 877.4 (M+H)⁺.

### Step J 3-(1-{2-[(4S)-3-{3-[4-(diethylphosphoryl)-3-(methylamino)phenyl]-2-oxo-2,3-dihydro-1H-imidazol-1-yl}-2-(4-fluoro-3,5-dimethylphenyl)-4-methyl-2H,4H,5H,6H,7H-pyrazolo[4,3-c]pyridine-5-carbonyl]-6-(oxan-4-yl)-1-benzofuran-3-yl}cyclopropyl)-4,5-dihydro-1,2,4-oxadiazol-5-one (Compound 19)

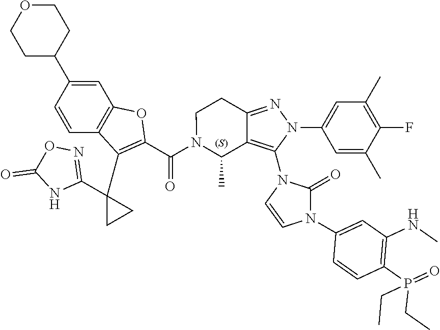

The mixture of (S,Z)-1-(2-(3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-6-(tetrahydro-2H-pyran-4-yl)benzofuran-3-yl)-N′-hydroxycyclopropane-1-carboximidamide (134 mg, 0.15 mmol) and CDI (98 mg, 0.61 mmol) in THF (2 mL) was stirred at 50° C. for 16 h. After cooling, the reaction mixture concentrated. The residue was purified by prep. HPLC (Column: Sunfire C18 19*250 mm*10 μm; Mobile Phase A: Water (0.1% formic acid), Mobile Phase B: CH₃CN; Flow rate: 20 mL/min; Gradient: 63% B to 68% B, retention time: 7.5-8.5 min) to afford 3-(1-{2-[(4S)-3-{3-[4-(diethylphosphoryl)-3-(methylamino)phenyl]-2-oxo-2,3-dihydro-1H-imidazol-1-yl}-2-(4-fluoro-3,5-dimethylphenyl)-4-methyl-2H,4H,5H,6H,7H-pyrazolo[4,3-c]pyridine-5-carbonyl]-6-(oxan-4-yl)-1-benzofuran-3-yl}cyclopropyl)-4,5-dihydro-1,2,4-oxadiazol-5-one (26.2 mg, 19.0% yield). LC-MS: m/z 903.3 (M+H)⁺. ¹H NMR (400 MHz, DMSO-d6): δ 8.19 (s, 1H), 7.74 (s, 1H), 7.60 (d, J=8.4 Hz, 1H), 7.51 (s, 1H), 7.21-7.35 (m, 3H), 7.15-7.16 (m, 2H), 6.81-7.00 (m, 3H), 5.61 (brs, 1H), 3.95-4.02 (m, 2H), 3.42-3.56 (m, 4H), 2.91-2.96 (m, 1H), 2.80-2.86 (m, 2H), 2.75 (s, 3H), 2.21 (d, J=1.2 Hz, 6H), 1.89-1.97 (m, 4H), 1.72-1.81 (m, 4H), 1.44-1.53 (m, 2H), 1.34 (d, J=6.8 Hz, 3H), 1.15-1.23 (m, 2H), 0.99-1.07 (m, 6H). ¹⁹F NMR (377 MHz, DMSO-d6): δ −122.11.

### Example 6

### 3-(1-{2-[(4S)-3-{3-[4-(diethylphosphoryl)-3-(methylamino)phenyl]-2-oxo-2,3-dihydro-1H-imidazol-1-yl}-2-(4-fluoro-3,5-dimethylphenyl)-4-methyl-2H,4H,5H,6H,7H-pyrazolo[4,3-c]pyridine-5-carbonyl]-6-(oxan-4-yl)-1-benzothiophen-3-yl}cyclopropyl)-4,5-dihydro-1,2,4-oxadiazol-5-one (Compound 18)

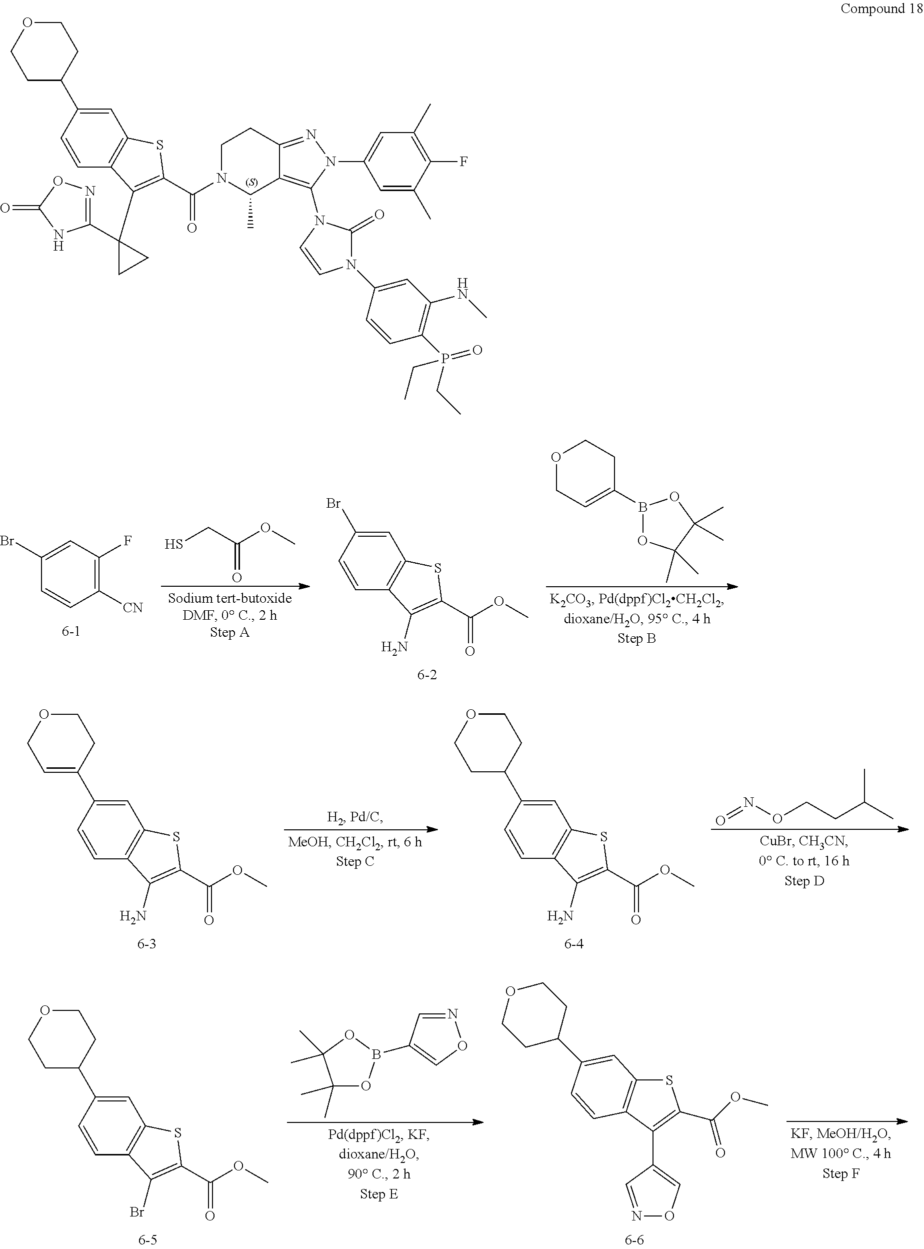

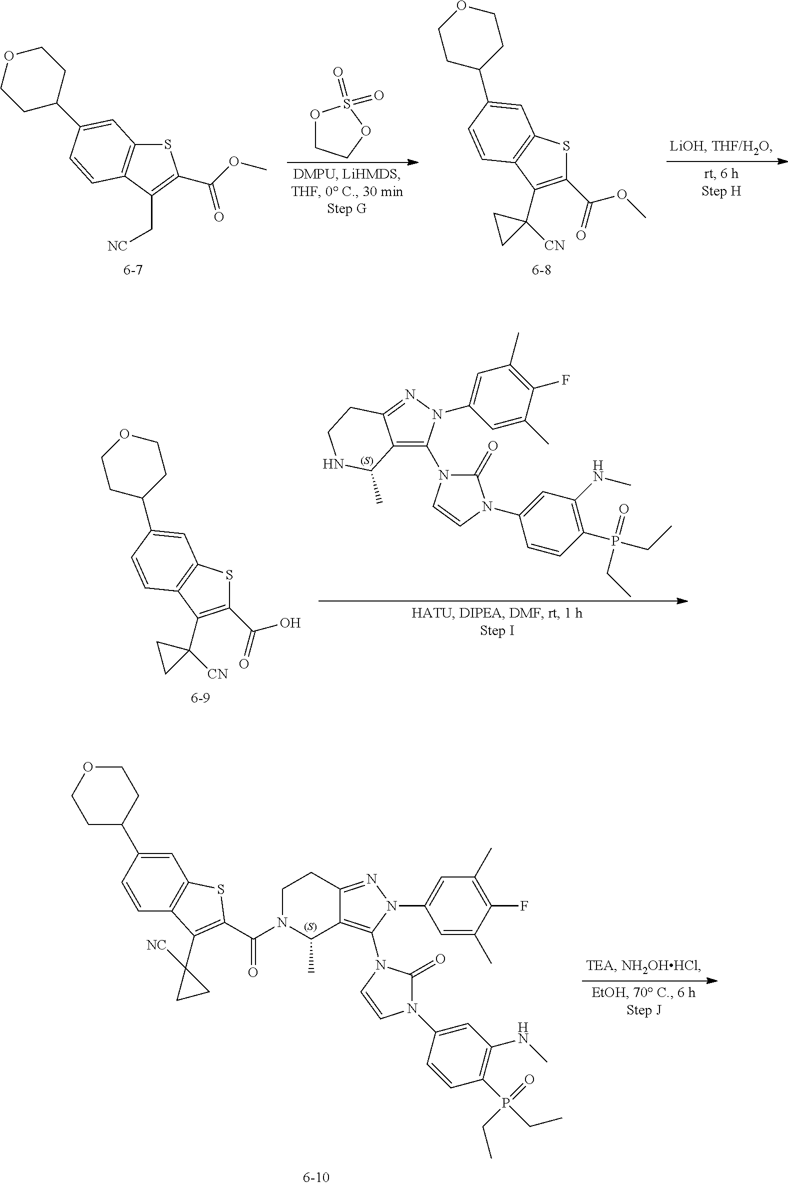

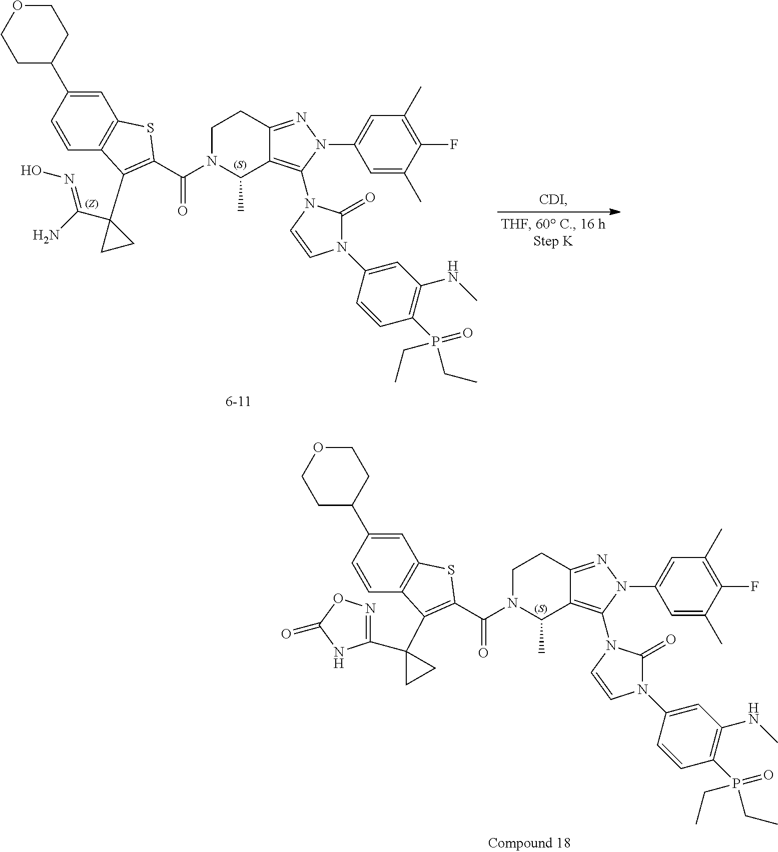

### Step A methyl 3-amino-6-bromobenzo[b]thiophene-2-carboxylate

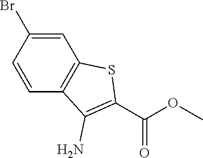

To the ice water cooled solution of 4-bromo-2-fluorobenzonitrile (10 g, 50 mmol) and methyl 2-mercaptoacetate (6.4 g, 60 mmol) in DMF (120 mL), was added sodium tert-butoxide (7.21 g, 75 mmol) in portions, kept the internal temperature below 0° C. After addition, the reaction mixture was stirred at 0° C. for 2 h. Then the reaction mixture was poured into cold water (200 mL) and extracted with EtOAc (200 mL×2). The combined organic layer was dried over anhydrous Na₂SO₄, filtered and concentrated. The residue was purified by flash silica gel chromatography (120 g SepaFlash® Silica Flash Column, Eluent of 0-10% EtOAc/PE gradient @100 mL/min) to give methyl 3-amino-6-bromobenzo[b]thiophene-2-carboxylate (11.2 g, 78.4% yield). LC-MS: m/z 285.9 (M+H)⁺.

### Step B methyl 3-amino-6-(3,6-dihydro-2H-pyran-4-yl)benzo[b]thiophene-2-carboxylate

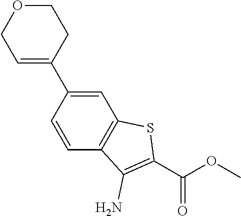 [SMILES: COC(=O)c1sc2cc(C3=CCOCC3)ccc2c1N]

To the mixture of methyl 3-amino-6-bromobenzo[b]thiophene-2-carboxylate (2.65 g, 12.63 mmol), 2-(3,6-dihydro-2H-pyran-4-yl)-4,4,5,5-tetramethyl-1,3,2-dioxaborolane (2.65 g, 12.63 mmol), and K₂CO₃ (4.36 g, 31.58 mmol) in dioxane (30 mL) and H₂O (3 mL), was added was added Pd(dppf)Cl₂·CH₂Cl₂ (1.05 g, 1.3 mmol). The reaction mixture was stirred at 90° C. for 3 h under nitrogen. After cooling, the mixture was filtered, the filtrate was concentrated. The residue was purified by flash silica gel chromatography (120 g SepaFlash® Silica Flash Column, Eluent of 0-10% MeOH/CH₂Cl₂ gradient @80 mL/min) to give methyl 3-amino-6-(3,6-dihydro-2H-pyran-4-yl)benzo[b]thiophene-2-carboxylate (1.9 g, 62.5% yield). LC-MS: m/z 290.1 (M+H)⁺.

### Step C methyl 3-amino-6-(tetrahydro-2H-pyran-4-yl)benzo[b]thiophene-2-carboxylate

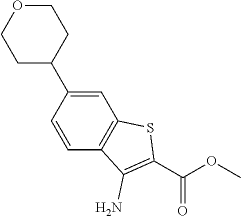 [SMILES: COC(=O)c1sc2cc(C3CCOCC3)ccc2c1N]

To the mixture of methyl 3-amino-6-(3,6-dihydro-2H-pyran-4-yl)benzo[b]thiophene-2-carboxylate (1.9 g, 6.57 mmol) in MeOH (20 mL) and DCM (20 mL), was added 5% Pd/C (3.8 g). After being degassed and charged with H₂ three times. The reaction mixture was stirred at room temperature under H₂ balloon for 6 h. After filtration, the filtrate was concentrated to give methyl 3-amino-6-(tetrahydro-2H-pyran-4-yl)benzo[b]thiophene-2-carboxylate (1.75 g, 91.2% yield). LC-MS: m/z 292.2 (M+H)⁺.

### Step D methyl 3-bromo-6-(tetrahydro-2H-pyran-4-yl)benzo[b]thiophene-2-carboxylate

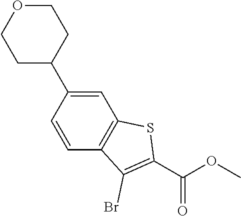 [SMILES: COC(=O)c1sc2cc(C3CCOCC3)ccc2c1Br]

To the mixture of methyl 3-amino-6-(tetrahydro-2H-pyran-4-yl)benzo[b]thiophene-2-carboxylate (1.65 g, 5.67 mmol) and CuBr (1.22 g, 8.5 mmol) in CH₃CN (20 mL) was added isopentyl nitrite (929 mg, 7.94 mmol) at 0° C. The reaction was stirred at room temperature for 16 h under nitrogen. The reaction mixture was filtered, diluted with H₂O (100 mL), extracted with EtOAc (30 mL×3). The combined organic layer was dried over anhydrous Na₂SO₄, filtered and concentrated. The residue was purified by flash silica gel chromatography (80 g SepaFlash® Silica Flash Column, Eluent of 0-10% EtOAc/PE gradient @50 mL/min) to give methyl 3-bromo-6-(tetrahydro-2H-pyran-4-yl)benzo[b]thiophene-2-carboxylate (900 mg, 45% yield). LC-MS: m/z 355.0 (M+H)⁺.

### Step E methyl 3-(isoxazol-4-yl)-6-(tetrahydro-2H-pyran-4-yl)benzo[b]thiophene-2-carboxylate

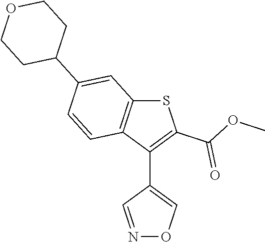 [SMILES: COC(=O)c1sc2cc(C3CCOCC3)ccc2c1-c1cnoc1]

To the mixture of methyl 3-bromo-6-(tetrahydro-2H-pyran-4-yl)benzo[b]thiophene-2-carboxylate (900 mg, 2.54 mmol), 4-(4,4,5,5-tetramethyl-1,3,2-dioxaborolan-2-yl)isoxazole (992 mg, 5.08 mmol) in dioxane (15 mL) and H₂O (1.5 mL), was added KF (737 mg, 12.71 mmol) and Pd(dppf)Cl₂·CH₂Cl₂ (186 mg, 0.25 mmol). The resulting mixture was stirred at 90° C. for 3 h under nitrogen. After cooling, the reaction mixture was concentrated and purified by flash silica gel chromatography (12 g SepaFlash® Silica Flash Column, Eluent of 0-30% EtOAc/PE gradient @50 mL/min) to give methyl 3-(isoxazol-4-yl)-6-(tetrahydro-2H-pyran-4-yl)benzo[b]thiophene-2-carboxylate (485 mg, 55.6% yield). LC-MS: m/z 344.1 (M+H)⁺.

### Step F methyl 3-(cyanomethyl)-6-(tetrahydro-2H-pyran-4-yl)benzo[b]thiophene-2-carboxylate

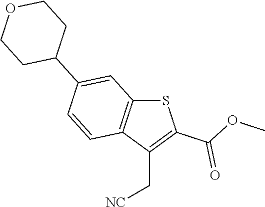 [SMILES: COC(=O)c1sc2cc(C3CCOCC3)ccc2c1CC#N]

The mixture of methyl 3-(isoxazol-4-yl)-6-(3,4,5,6-tetrahydro-2H-pyran-4-yl)benzothiophene-2-carboxylate (485 mg, 1.41 mmol) and KF (246 mg, 4.24 mmol) in MeOH (5 mL) and H₂O (1 mL) was heated at 100° C. for 2 h in a Biotage microwave reactor. After cooling, the reaction mixture was concentrated. The residue was purified by flash silica gel chromatography (12 g SepaFlash® Silica Flash Column, Eluent of 0-30% EtOAc/PE gradient @50 mL/min) to afford methyl 3-(cyanomethyl)-6-(3,4,5,6-tetrahydro-2H-pyran-4-yl)benzothiophene-2-carboxylate (290 mg, 65.1% yield). LC-MS: m/z 316.1 (M+H)⁺.

### Step G methyl 3-(cyanocyclopropyl)-6-(3,4,5,6-tetrahydro-2H-pyran-4-yl)benzo[b]thiophene-2-carboxylate

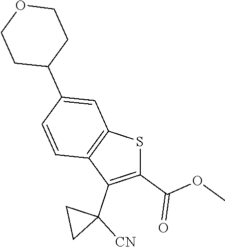 [SMILES: COC(=O)c1sc2cc(C3CCOCC3)ccc2c1C1(C#N)CC1]

To a solution of methyl 3-(cyanomethyl)-6-(3,4,5,6-tetrahydro-2H-pyran-4-yl)benzo[b]thiophene-2-carboxylate (290 mg, 0.92 mmol), 1,3,2-dioxathiolane 2,2-dioxide (228.2 mg, 1.84 mmol) and DMPU (235.7 mg, 1.84 mmol) in THF (6 mL) was added HMDSLi (2.5 mL, 2.5 mmol) at 0° C. The reaction mixture was stirred at 0° C. for 30 min. Then the mixture was quenched with sat. aq. NH₄Cl (5 mL), extracted with EtOAc (15 mL×3). The combined organic layer was dried over anhydrous Na₂SO₄, filtered and concentrated. The residue was purified by prep. TLC (PE/EtOAc=2/1) to afford methyl 3-(cyanocyclopropyl)-6-(3,4,5,6-tetrahydro-2H-pyran-4-yl)benzo[b]thiophene-2-carboxylate (182 mg, 58.0% yield). LC-MS: m/z 342.2 (M+H)⁺.

### Step H 3-(cyanocyclopropyl)-6-(3,4,5,6-tetrahydro-2H-pyran-4-yl)benzo[b]thiophene-2-carboxylic acid

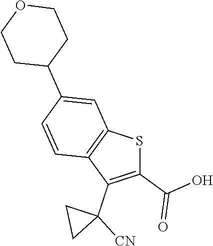 [SMILES: N#CC1(c2c(C(=O)O)sc3cc(C4CCOCC4)ccc23)CC1]

The mixture of methyl 3-(cyanocyclopropyl)-6-(3,4,5,6-tetrahydro-2H-pyran-4-yl)benzo[b]thiophene-2-carboxylate (182 mg, 0.53 mmol) and LiOH (44.8 mg, 1.07 mmol) in THF (2 mL) and H₂O (0.4 mL) was stirred at room temperature for 6 h. The reaction mixture was diluted with water (25 mL), acidified with 1M aq. HCl to pH=5.0, extracted with EtOAc (30 mL×3). The combined organic layer was washed with brine, dried over anhydrous Na₂SO₄, filtered and concentrated to give 3-(cyanocyclopropyl)-6-(3,4,5,6-tetrahydro-2H-pyran-4-yl)benzo[b]thiophene-2-carboxylic acid (180 mg, crude). LC-MS: m/z 328.1 (M+H)⁺.

### Step I (S)-1-(2-(3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-6-(tetrahydro-2H-pyran-4-yl)benzo[b]thiophen-3-yl)cyclopropane-1-carbonitrile

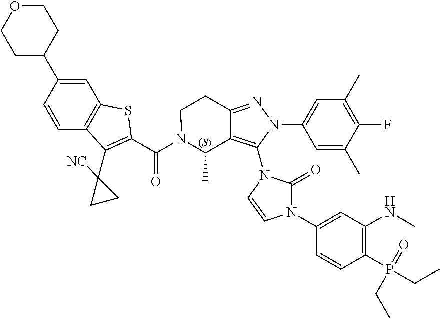

To the mixture of 3-(cyanocyclopropyl)-6-(3,4,5,6-tetrahydro-2H-pyran-4-yl)benzo[b]thiophene-2-carboxylic acid (170 mg, 0.52 mmol), (S)-1-(4-(diethylphosphoryl)-3-(methylamino)phenyl)-3-(2-(4-fluoro-3,5-dimethylphenyl)-4-methyl-4,5,6,7-tetrahydro-2H-pyrazolo[4,3-c]pyridin-3-yl)-1,3-dihydro-2H-imidazol-2-one (257 mg, 0.47 mmol) (HCl salt, preparation was described in WO2021155841A1) in DMF (4 mL), was added HATU (237 mg, 0.62 mmol) and DIPEA (134 mg, 1.04 mmol). The resulting mixture was stirred at room temperature for 1 h. The mixture was poured into water, extracted with EtOAc (20 mL×3). The combined organic layers were washed with brine (20 mL×2), dried over anhydrous Na₂SO₄, filtered and concentrated. The residue was purified by flash silica gel chromatography (12 g SepaFlash® Silica Flash Column, Eluent of 0-5% MeOH/CH₂Cl₂ gradient @50 mL/min) to obtain (S)-1-(2-(3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-6-(tetrahydro-2H-pyran-4-yl)benzo[b]thiophen-3-yl)cyclopropane-1-carbonitrile (137 mg, 30.7% yield). LC-MS: m/z 860.3 (M+H)⁺.

### Step J (S,Z)-1-(2-(3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-6-(tetrahydro-2H-pyran-4-yl)benzo[b]thiophen-3-yl)-N′-hydroxycyclopropane-1-carboximidamide

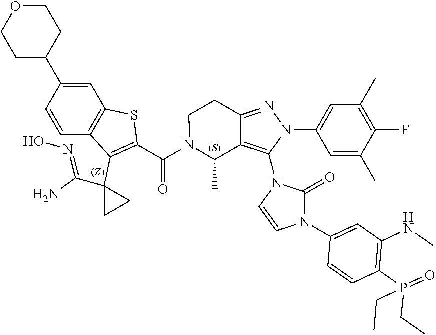

The mixture of (S)-1-(2-(3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-6-(tetrahydro-2H-pyran-4-yl)benzo[b]thiophen-3-yl)cyclopropane-1-carbonitrile (137 mg, 0.16 mmol), NH₂OH·HCl (33.2 mg, 0.478 mmol) and DIPEA (61.8 mg, 0.478 mmol) in EtOH (2 mL) was stirred at 70° C. for 6 h. After cooling, the reaction was quenched with H₂O (10 mL), extracted with ethyl acetate (10 mL×3). The combined organic layers were dried over anhydrous Na₂SO₄, filtered and concentrated to give (S,Z)-1-(2-(3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-6-(tetrahydro-2H-pyran-4-yl)benzo[b]thiophen-3-yl)-N′-hydroxycyclopropane-1-carboximidamide (82 mg, 57.6% yield). LC-MS: m/z 893.3 (M+H)⁺.

### Step K 3-(1-{2-[(4S)-3-{3-[4-(diethylphosphoryl)-3-(methylamino)phenyl]-2-oxo-2,3-dihydro-1H-imidazol-1-yl}-2-(4-fluoro-3,5-dimethylphenyl)-4-methyl-2H,4H,5H,6H,7H-pyrazolo[4,3-c]pyridine-5-carbonyl]-6-(oxan-4-yl)-1-benzothiophen-3-yl}cyclopropyl)-4,5-dihydro-1,2,4-oxadiazol-5-one (Compound 18)

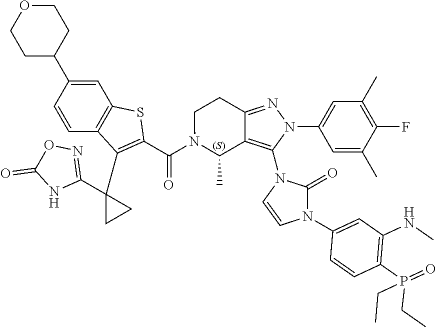

The mixture of (S,Z)-1-(2-(3-(3-(4-(diethylphosphoryl)-3-(methylamino)phenyl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-6-(tetrahydro-2H-pyran-4-yl)benzo[b]thiophen-3-yl)-N′-hydroxycyclopropane-1-carboximidamide (82 mg, 0.092 mmol) and CDI (29.8 mg, 0.184 mmol) in dioxane (1 mL) was stirred at 60° C. for 16 h. After cooling, the reaction mixture concentrated. The residue was purified by prep. HPLC (Column: Sunfire C18 19*250 mm*10 m; Mobile Phase A: Water (0.1% formic acid), Mobile Phase B: CH₃CN; Flow rate: 20 mL/min; Gradient: 64% B to 74% B, retention time: 8.7-9.6 min) to afford 3-(1-{2-[(4S)-3-{3-[4-(diethylphosphoryl)-3-(methylamino)phenyl]-2-oxo-2,3-dihydro-1H-imidazol-1-yl}-2-(4-fluoro-3,5-dimethylphenyl)-4-methyl-2H,4H,5H,6H,7H-pyrazolo[4,3-c]pyridine-5-carbonyl]-6-(oxan-4-yl)-1-benzothiophen-3-yl}cyclopropyl)-4,5-dihydro-1,2,4-oxadiazol-5-one (18.6 mg, 22.0% yield). LC-MS: m/z 919.4 (M+H)⁺. ¹H NMR (400 MHz, DMSO-d6): δ 11.93 (br s, 1H), 7.91 (s, 1H), 7.83 (s, 1H), 7.70 (s, 1H), 7.40-7.42 (m, 1H), 7.19-7.35 (m, 2H), 7.10-7.18 (m, 2H), 6.76-7.03 (m, 3H), 5.52-5.96 (m, 1H), 3.99 (d, J=10.8 Hz, 2H), 3.41-3.56 (m, 3H), 2.88-3.01 (m, 2H), 2.61-2.85 (m, 5H), 2.21 (s, 6H), 1.86-1.97 (m, 4H), 1.72-1.82 (m, 4H), 1.45-1.69 (m, 3H), 1.25-1.43 (m, 4H), 0.96-1.08 (m, 6H). ¹⁹F NMR (377 MHz, DMSO-d6): δ −122.22.

The compounds in Table 1 were, or can be, synthesized using a similar procedure described in the Examples above using the appropriate starting materials.

| Compound | Structure | LC-MS: m/z |
|---|---|---|
| 3 | 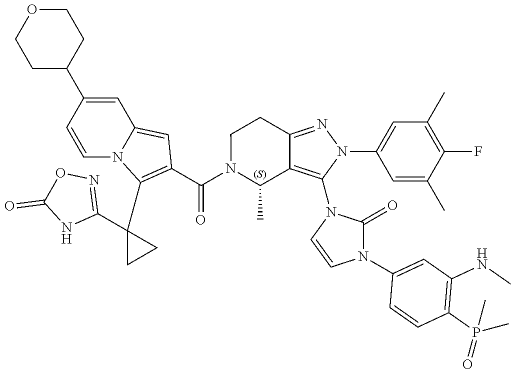 | 874.3 (M + H)⁺ |
| 4 | 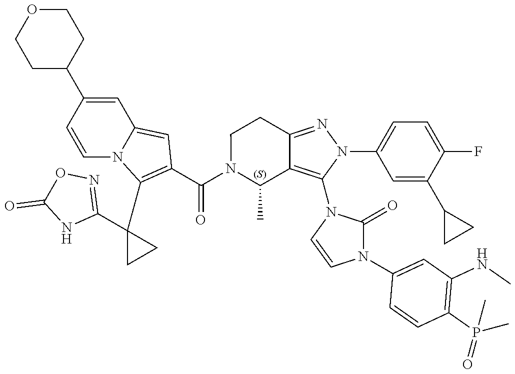 | 886.2 (M + H)⁺ |
| 5 | 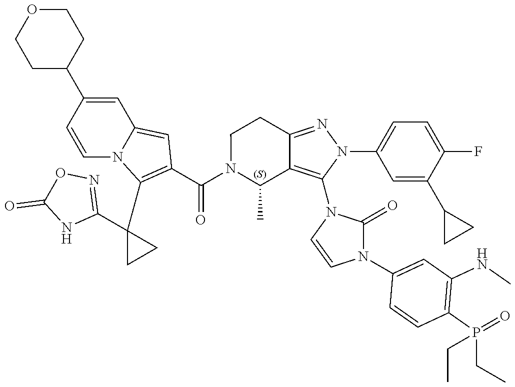 | 914.2 (M + H)⁺ |
| 6 | 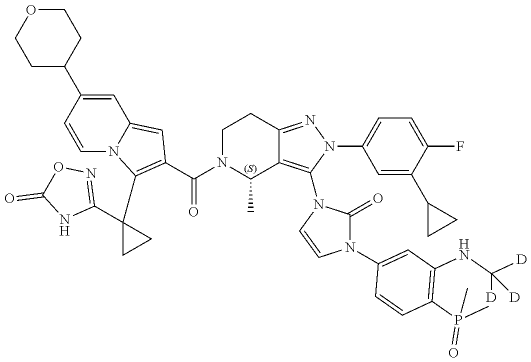 | 889.2 (M + H)⁺ |
| 18 | 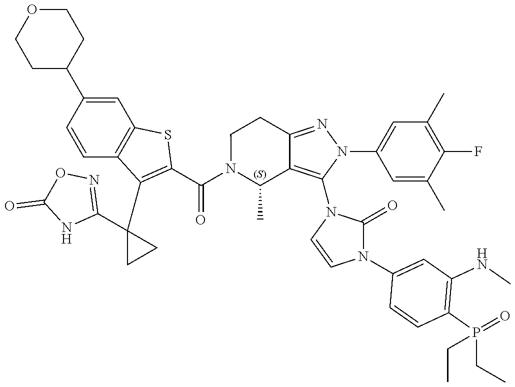 | 919.4 (M + H)⁺ |
| 19 | 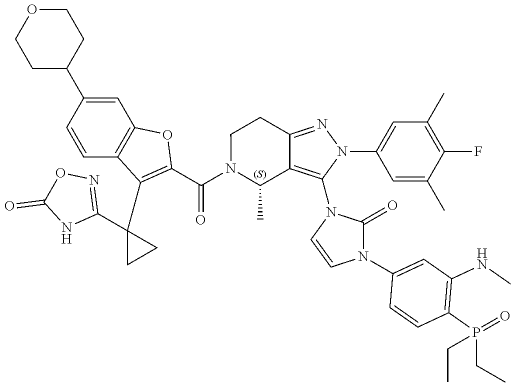 | 903.3 (M + H)⁺ |
| 20 | 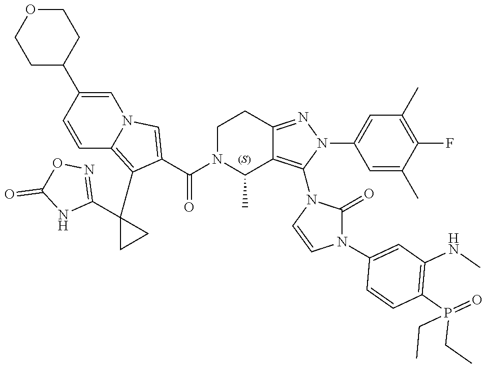 | 902.2 (M + H)⁺ |
| 21 | 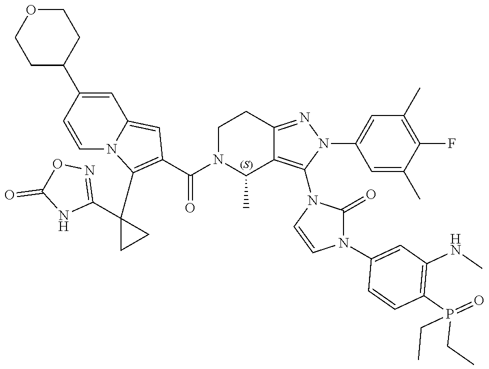 | 902.4 (M + H)⁺ |
| 22 | 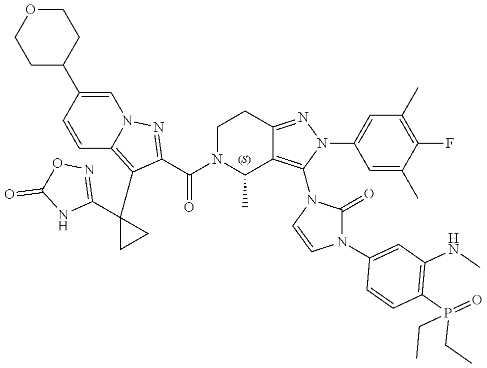 | 903.3 (M + H)⁺ |
| 24 | 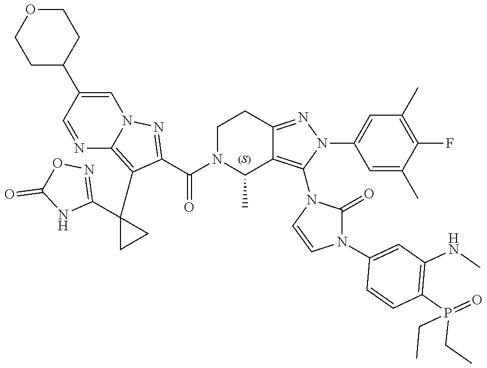 | 904.2 (M + H)⁺ |
| 26 | 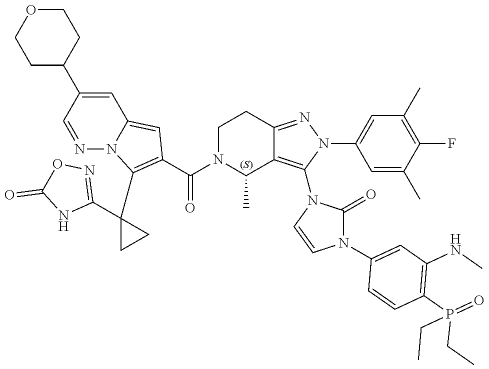 | 903.7 (M + H)⁺ |
| 27 | 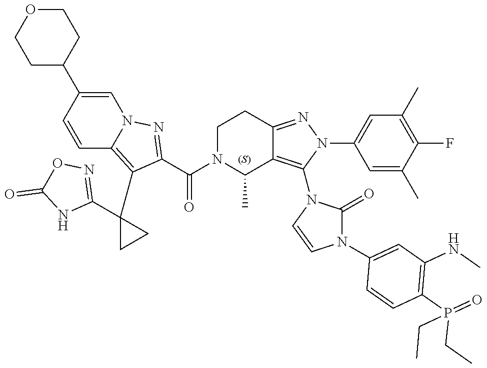 | 903.4 (M + H)⁺ |
| 28 | 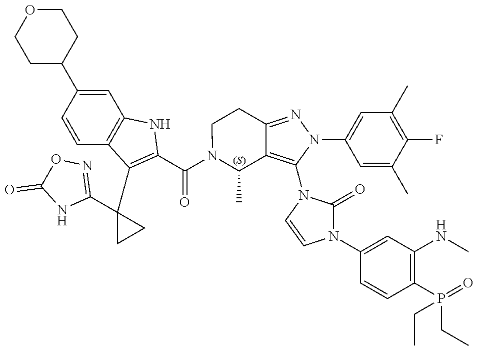 | 902.9 (M + H)⁺ |
| 29 | 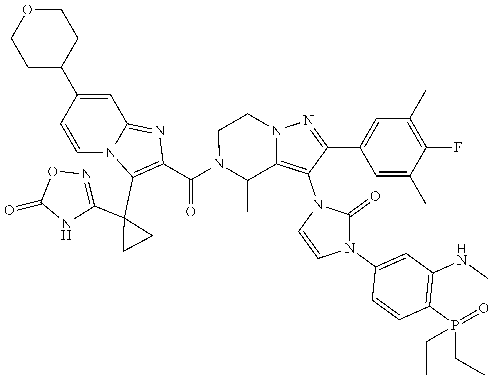 [SMILES: CCP(=O)(CC)c1ccc(-n2ccn(-c3c(-c4cc(C)c(F)c(C)c4)nn4c3C(C)N(C(=O)c3nc5cc(C6CCOCC6)ccn5c3C3(c5noc(=O)[nH]5)CC3)CC4)c2=O)cc1NC] | 903.5 (M + H)⁺ |
| 30 | 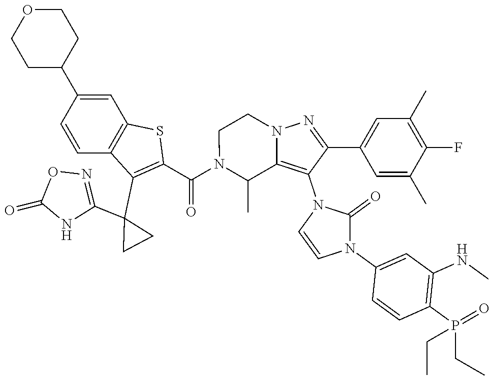 [SMILES: CCP(=O)(CC)c1ccc(-n2ccn(-c3c(-c4cc(C)c(F)c(C)c4)nn4c3C(C)N(C(=O)c3sc5cc(C6CCOCC6)ccc5c3C3(c5noc(=O)[nH]5)CC3)CC4)c2=O)cc1NC] | 919.4 (M + H)⁺ |

### Biological Assays

### cAMP Assays

Activation of GLP-1 receptor is known to stimulate cyclic AMP (cAMP) production in cells which indicates primary coupling to the Gas subunit of the G protein heterotrimeric complex. Evidence suggests signaling through G Gαs induced cAMP stimulation elicits the desired pharmacological response regarding insulin release from pancreatic β-cells.

Method 1: To optimize functional activity directed toward Gas coupling, a CHO-K1 cell line developed by DiscoverX stably expressing the GLP-1 Receptor is used. Cells expressing GLP-1 receptor are plated in a 384-well microtiter plates and incubated overnight at 37° C. with 5% CO₂ to allow the cells to attach and grow. Media is then aspirated from the cells and replaced with 15 μL Hanks Balanced Salt Solution (HBSS)/5 mM Hepes:0.5 mM IBMX 0.1% BSA (pH 7.4) [Stimulation Buffer]. Plated cells are resuspended in Stimulation buffer and five microliters (5 μL) of the suspension is added to previously generated compound sample stocks at 4× final concentration in Stimulation Buffer are then added to the cells and allowed to incubate at 37° C. for 30 or 60 minutes.

After incubation the assay signal is generated using a europium chelate-labeled cAMP tracer which competes with cAMP for binding sites on a cAMP-specific monoclonal antibody labeled with ULight™ dye. When antibodies are bound to the Eu-labeled cAMP tracer FRET transfer between the Eu chelate and ULight™ labeled antibodies is detected. Free cAMP produced from stimulated cells competes with the Eu-cAMP tracer for binding to the ULight™-mAb causing a decrease in the TR-FRET signal in a dose dependent manner.

The method for detection of cAMP using the Eu-chelate technology requires incubation with Eu-cAMP tracer solution+ULight™-anti-cAMP solution for one hour at room temperature. Microplates are read following signal generation with a PerkinElmer Envision instrument utilizing TR-FRET signal detection. Percentage activity is calculated using the following formula:

$$\% Activity = 100 \% \times ( mean RLU of test sample - mean RLU of vehicle control ) / ( mean RLU of MAX control - mean RLU of vehicle control ) )$$

Method 2: To optimize functional activity directed toward Gas coupling, a CHO-K1 cell line developed by DiscoverX stably expressing the GLP-1 Receptor is used. Cells expressing GLP-1 receptor are plated in a 384-well microtiter plates and incubated overnight at 37° C. with 5% CO₂ to allow the cells to attach and grow. Media is then aspirated from the cells and replaced with 15 μL 2:1 Hanks Balanced Salt Solution (HBSS)/10 mM Hepes: cAMP XS+Ab reagent. Five microliters (5 μL) of previously generated compound sample stocks at 4× final concentration in assay buffer are then added to the cells and allowed to incubate at 37° C. for 30 or 60 minutes.

After incubation the assay signal is generated using enzyme fragment complementation (EFC). In EFC, the enzyme B-galactosidase is split into two complementary portions (EA and ED). The fragment ED is fused to cAMP and in the assay format competes with endogenous cAMP for binding to a cAMP specific antibody. Activated B-Gal is formed when exogenous EA fragment binds to free ED-cAMP (not bound to cAMP specific antibody). Activated enzyme levels are detected through conversion of B-gal chemiluminescent substrate which generates a detectable luminescence signal and read on standard microtiter plate.

The methodology for detection of cAMP using EFC requires incubation with 20 μL of cAMP XS+ED/CL lysis cocktail for one hour followed by incubation with 20 μL cAMP XS+EA reagent for three hours at room temperature. Microplates are read following signal generation with a PerkinElmer Envision instrument utilizing chemiluminescent signal detection. Compound activity is analyzed using CBIS data analysis suite (ChemInnovation, CA). Percentage activity is calculated using the following formula:

$$\% Activity = 100 \% \times ( mean RLU of test sample - mean RLU of vehicle control ) / ( mean RLU of MAX control - mean RLU of vehicle control ) )$$

Method 3: Activation of GLP-1 receptor is known to stimulate cyclic AMP (cAMP) production in cells which indicates primary coupling to the Gas subunit of the G protein heterotrimeric complex. Evidence suggests signaling through Gas induced cAMP stimulation elicits the desired pharmacological response regarding insulin release from pancreatic β-cells.

To optimize functional activity directed toward Gas coupling, a HEK293/CRE-Luc cell line developed by HDB stably expressing the GLP-1 Receptor was used. 200× concentration of compound working solutions were prepared (Agilent Technologies Bravo) with ½ log serial dilution in 384-well Echo LDV plate (Labcyte, Cat #LP-0200). 50 nL/well 200× concentration of compound working solutions were moved to 384-well white low volume plate (Greiner, Cat #784075) using Labcyte ECHO550. 1×105 cells/mL HEK293/GLP1R/CRE-LUC(HD Biosciences) cell suspensions prepared with assay buffer [DPBS containing 0.5 mM IBMX (Sigma, Cat #15879) and 0.1% BSA (GENVIEW, Cat #FA016-100 g)], 10 μL cell suspensions were added to each well of previous generated assay plate which already contains 50 nL compound at 200×concentration using ThermoFisher Multidrop Combi (1000 cells/well). The plate was sealed and incubated at 37° C. with 5% CO₂ for 30 min.

After incubation, the cAMP assay signal was generated using cAMP dynamic 2 Kit (Cisbio). 5 μL cAMP-d2 working solution was added to each well, followed with 5 μL Anti-cAMP antibody-cryptate working solution was added to each well using ThermoFisher Multidrop Combi, and incubated at room temperature for 1 hour protected from light. Fluorescence was read at 665 and 615 nm with Reader PerkinElmer EnVision. Percentage activity was calculated using the following formula:

$$\% Activity = 100 \% \times ( mean RLU of test sample - mean RLU of vehicle control ) / ( mean RLU of MAX control - mean RLU of vehicle control ) )$$

Table 2 shows the biological activity of compounds in GLP-1R agonist cAMP stimulation assay (EC₅₀)

TABLE 2

| Compound | EC₅₀ (μM) |
|---|---|
| 3 | 0.00007 |
| 4 | 0.000085 |
| 5 | 0.00011 |
| 6 | 0.000089 |
| 18 | 0.000061 |
| 19 | 0.000049 |
| 20 | 0.00003 |
| 21 | 0.00002 |
| 22 | 0.000088 |
| 24 | 0.00018 |
| 26 | 0.000014 |
| 27 | 0.0025 |
| 28 | 0.000033 |
| 29 | 0.029 |
| 30 | 0.00032 |

The previous examples are included to demonstrate specific embodiments of the disclosure. It should be appreciated by those of skill in the art that the techniques disclosed herein represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

1. A compound of Formula I: 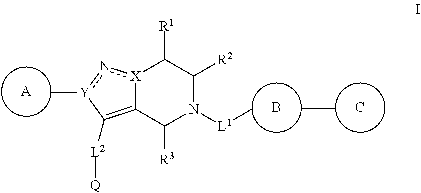
or a pharmaceutically acceptable salt, deuterated analog, or solvate thereof, wherein:
X is C and Y is N, or Y is C and X is N and the dashed lines represent an optional bond such that the moiety 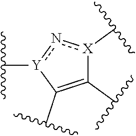
is 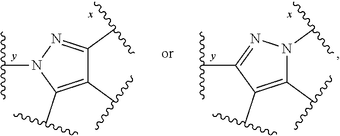 [SMILES: *c1nn(*)c(*)c1* ; *c1nn(*)c(*)c1*]
where bond y is attached to Y and bond x is attached to X;
Ring A is C₆₋₁₀ aryl, C₅₋₁₀ cycloalkyl, 5-10 membered heterocyclyl, or 5-10 membered heteroaryl, each of which is optionally substituted with from 1-5 substituents each independently selected from the group consisting of halo, C₁₋₆ alkyl, C₁₋₆ haloalkyl, C₁₋₆ alkoxy, and C₃₋₆ cycloalkyl;
R¹, R², and R³ are each independently selected from the group consisting of H and C₁₋₆ alkyl which is optionally substituted with from 1-6 substituents each independently selected from the group consisting of halo, —OH, and C₁₋₆ alkoxy;
L¹ is selected from the group consisting of —C(═O)—, —CH₂—, —CH(C₁₋₆ alkyl)-, and —S(═O)₂;
Ring B is selected from the group consisting of 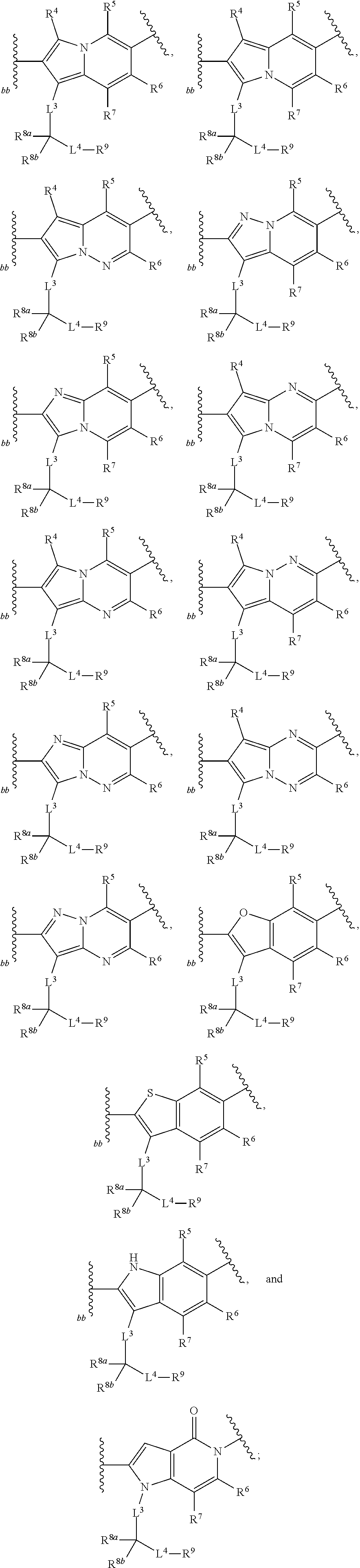
wherein bb represents point of attachment to L¹;
R⁴, R⁵, R⁶, and R⁷ are independently selected from the group consisting of H, halo, and C₁₋₆ alkyl;
L³ is a bond or C₁₋₃ alkylene;
L⁴ is a bond or C₁₋₅ alkylene;
R^{8a} and R^{8b} are independently selected from the group consisting of H and C₁₋₆ alkyl optionally substituted with one or more substituents independently selected from the group consisting of halo and C₃₋₁₅ cycloalkyl;
or R^{8a} and R^{8b} taken together with the carbon atom to which each is attached forms a C₃₋₁₅ cycloalkyl ring which is optionally substituted with from 1-3 independently selected C₁₋₆ alkyl, wherein the C₁₋₆ alkyl is optionally substituted with from 1-6 independently selected from R^{f};
or the moiety -L³-C(R^{8a}R^{8b})-L⁴-R⁹ is a phenyl substituted with C(═O)OH, and optionally 1-4 additional substituents independently selected from R^{f};
R⁹ is selected from the group consisting of C(═O)OH, C(═O)(OC₁₋₆ alkyl), C(═O)NR^{9a}R^{9b}, (IX-1), (IX-2), (IX-3), and (IX-4): 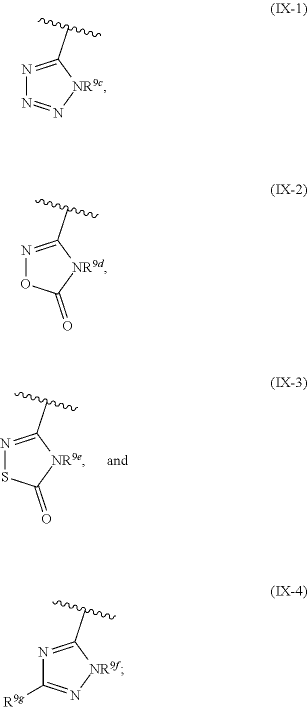
R^{9a} is H or C₁₋₆ alkyl;
R^{9b} is H, C₁₋₆ alkyl, C(═O)(C₁₋₆ alkyl), S(O)₀₋₂(C₁₋₆ alkyl), or cyano;
R^{9c}, R^{9d}, R^{9e}, R^{9f}, and R^{9g} are each independently selected from the group consisting of H, C₁₋₆ alkyl optionally substituted with from 1-6 independently selected halo and C₁₋₆ alkoxy, and C(═O)(C₁₋₆ alkyl);
Ring C is selected from the group consisting of 3-12 membered heterocyclyl, C₃₋₁₅ cycloalkyl, and 5-10 membered heteroaryl, each of which is optionally substituted with from 1-3 R^{Ca};
each R^{Ca} is independently selected from the group consisting of halo, C₁₋₆ alkyl, C₁₋₆ haloalkyl, C₁₋₆ alkoxy, and NR^{c}R^{d};
or a pair of R^{Ca} on the same or different ring atoms, taken together with the ring atom(s) to which each is attached, forms a carbocyclic ring including from 3-8 ring atoms;
L² is selected from the group consisting of 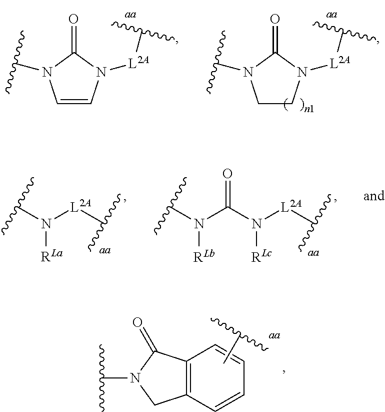
wherein aa represents the point of attachment to Q;
n1 is an integer from 1-3;
L^{2A} is a bond or C₁₋₁₀ alkylene;
R^{La} is selected from the group consisting of H, C₁₋₆ alkyl, and C(═O)(C₁₋₆ alkyl);
each of R^{Lb} and R^{Lc} is independently selected from the group consisting of H and C₁₋₆ alkyl;
Q is selected from the group consisting of C₁₋₁₀ alkyl; C₃₋₁₅ cycloalkyl; 3-12 membered heterocyclyl; 5-10 membered heteroaryl; and C₆₋₁₀ aryl, wherein Q is substituted with —P(═O)R^{a}R^{b}, and optionally substituted with from 1-6 additional substituents independently selected from R^{Q};
each R^{Q} is independently selected from the group consisting of
(a) halo;
(b) cyano;
(c) OH or oxo;
(d) —NR^{c}R^{d};
(e) —C(═O)NR^{c}R^{d} or —S(O)₂NR^{c}R^{d};
(f) —S(═O)₀₋₂R^{e};
(g) C₁₋₆ alkyl optionally substituted with from 1-6 independently selected R^{f};
(h) C₁₋₆ alkoxy optionally substituted with from 1-6 independently selected R^{f};
(i) 3-12 membered heterocyclyl optionally substituted with from 1-6 independently selected R^{g};
(j) C₆₋₁₀ aryl optionally substituted with from 1-6 independently selected R^{g};
(k) 5-10 membered heteroaryl optionally substituted with from 1-6 independently selected R^{g};
(l) C₃₋₈ cycloalkyl optionally substituted with from 1-6 independently selected R^{g};
(in) —P(═O)R^{a}R^{b}; and
(n) —(CR^{h}R^{h})_{q1}—S(O)₂-L^{e}-R^{e}, wherein q1 is 1, 2, or 3;
or Q is C₃₋₁₅ cycloalkyl; 3-12 membered heterocyclyl; 5-10 membered heteroaryl; and C₆₋₁₀ aryl, each of which is optionally substituted with from 1-6 substituents each independently selected from the group consisting of R^{QA} and R^{QB};
provided that one or both of (aa) or (bb) applies:
(aa) Q is substituted with at least one R^{QB}; or
(bb) Q is 4-12 membered heterocyclyl or 7-10 membered bicyclic heteroaryl, wherein Q comprises an endocyclic S(O)₂ group;
each R^{QA} is independently selected from the group consisting of
(a) halo;
(b) cyano;
(c) OH or oxo;
(d) —NR^{a}R^{b};
(e) —C(═O)NR^{c}R^{d};
(f) C₁₋₆ alkyl optionally substituted with from 1-6 independently selected R^{f};
(g) C₁₋₆ alkoxy optionally substituted with from 1-6 independently selected R^{f};
(h) 3-12 membered heterocyclyl optionally substituted with from 1-6 independently selected R^{g};
(i) C₆₋₁₀ aryl optionally substituted with from 1-6 independently selected R^{g};
(j) 5-10 membered heteroaryl optionally substituted with from 1-6 independently selected R^{g}; and
(k) C₃₋₈ cycloalkyl optionally substituted with from 1-6 independently selected R^{g};
each R^{QB} is independently selected from the group consisting of:
(a) -L^{a}-S(O)₂-L^{b}-R^{e}; and
(b) 4-12 membered heterocyclyl or 7-10 membered bicyclic heteroaryl, each of which comprises an endocyclic S(O)₂ group, wherein the heterocyclyl or heteroaryl is optionally substituted with from 1-6 independently selected R^{g};
L^{a} is selected from the group consisting of —N(H)—, —N(R^{c})—, and —(CR^{h}R^{h})_{q1};
L^{b} is selected from the group consisting of —O—, —N(H)—, —N(R^{c})—, and —(CR^{h}R^{h})_{q2}—;
q1 and q2 are independently 0, 1, 2, 3, or 4;
provided that when L^{a} is —(CR^{h}R^{h})_{q1}— and q1 is 0, then -L^{b}-R^{e} is other than unsubstituted C₁₋₃ alkyl;
R^{a} and R^{b} are independently selected from the group consisting of C₁₋₆ alkyl which is optionally substituted with from 1-6 substituents each independently selected from the group consisting of C₁₋₆ alkoxy, C₃₋₆ cycloalkyl, and halo; C₃₋₆ cycloalkyl optionally substituted with from 1-3 substituents each independently selected from the group consisting of C₁₋₃ alkyl and halo; and C₆₋₁₀ aryl optionally substituted with from 1-3 independently selected C₁₋₃ alkyl; or
R^{a} and R^{b} taken together with the phosphorous atom to which each is attached forms a ring including from 5-8 ring atoms, wherein from 0-2 ring atoms (in addition to the phosphorous attached to R^{a} and R^{b}) are heteroatoms each independently selected from the group consisting of O, S, and N, wherein the ring is optionally substituted with from 1-3 independently selected C₁₋₆ alkyl;
each R^{c} and R^{d} are independently selected from the group consisting of H, C₁₋₆ alkyl, C₃₋₆ cycloalkyl, C(═O)(C₁₋₆ alkyl), C(═O)(C₃₋₆ cycloalkyl), C(═O)O(C₁₋₆ alkyl), S(O)₁₋₂(C₁₋₆ alkyl), and S(O)₁₋₂(C₃₋₆ cycloalkyl), wherein the C₁₋₆ alkyl, C(═O)(C₁₋₆ alkyl), C(═O)(C₃₋₆ cycloalkyl), C(═O)O(C₁₋₆ alkyl), S(O)₁₋₂(C₁₋₃alkyl), and S(O)₁₋₂(C₃₋₆ cycloalkyl) are each optionally substituted with from 1-6 substituents independently selected from the group consisting of —OH, halo, and C₁₋₆ alkoxy;
R^{e} is H, or R^{c} is selected from the group consisting of C₁₋₆ alkyl, C₁₋₆ haloalkyl, C₁₋₆ alkoxy, C₁₋₆ haloalkoxy, C₃₋₆ cycloalkyl, and 3-8 membered heterocyclyl, each of which is optionally substituted with from 1-3 substituents each independently selected from the group consisting of halo, cyano, C₁₋₃ alkyl, C₁₋₃ haloalkyl, —OH, NH₂, NH(C₁₋₃ alkyl), N(C₁₋₃ alkyl)₂, C₁₋₃ alkoxy, and C₁₋₃ haloalkoxy;
L^{e} is a bond, NR^{c}, or O;
each R^{f} is independently selected from the group consisting of halo, —OH, NR^{c}R^{d}, C₁₋₆ alkoxy, C₁₋₆ haloalkoxy, and 3-12 membered heterocyclyl which is optionally substituted with from 1-4 substituents each independently selected from the group consisting of —OH, C₁₋₆ alkyl, and 3-12 membered heterocyclyl;
each R^{g} is independently selected from the group consisting of C₁₋₆ alkyl, C₁₋₆ haloalkyl, C₁₋₆ alkoxy, C₁₋₆ haloalkoxy, NR^{c}R^{d}, cyano, halo, C₃₋₆ cycloalkyl, and 3 to 12 membered heterocyclyl optionally substituted with one or more substituents each independently selected from the group consisting of C₁₋₆ alkyl and C(═O)C₁₋₆ alkyl; and
each occurrence of R^{h} is independently selected from the group consisting of H, C₁₋₆ alkyl, C₁₋₆ haloalkyl, C₃₋₆ cycloalkyl, and halo; or
a pair of R^{h} on the same or different carbon atom(s), taken together with the carbon atom(s) connecting them forms C₃₋₆ cycloalkyl or 4-8 membered heterocyclyl, each of which is optionally substituted with from 1-3 substituents each independently selected from the group consisting of halo, cyano, C₁₋₃ alkyl, C₁₋₃ haloalkyl, —OH, C₁₋₃ alkoxy, and C₁₋₃ haloalkoxy.

2. The compound of claim 1, wherein the moiety 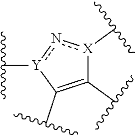
is 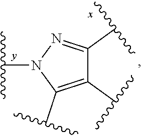 [SMILES: *c1nn(*)c(*)c1*]
where bond y is attached to Y and bond x is attached to X.

3. The compound of claim 1, wherein the moiety 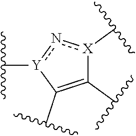
is 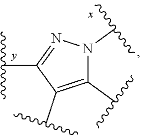 [SMILES: *c1nn(*)c(*)c1*]
where bond y is attached to Y and bond x is attached to X.

4. A compound of Formula II: 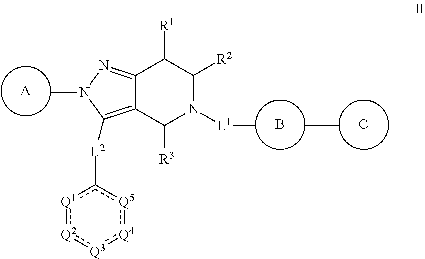
or a pharmaceutically acceptable salt thereof or a solvate thereof, wherein:
Q¹, Q², Q³, Q⁴, and Q⁵ are defined according to (AA) or (BB) below: [symbol]
(AA)
Q¹ and Q⁵ are independently selected from the group consisting of N, CH, and CR^{QC};
Q², Q³, and Q⁴ are independently selected from the group consisting of N, CH, CR^{QC}, and CR^{QD}, provided that at least one of Q², Q³, and Q⁴ is CR^{QD};
each [symbol] is a single bond or double bond, provided that the ring including Q¹-Q⁵ is aromatic;
(BB) [symbol]
Q¹ is a bond;
Q², Q³, Q⁴, and Q⁵ are independently selected from the group consisting of O, S, N, NH, NR^{c}, CH, CR^{QC}, and CR^{QD}, provided that at least one of Q², Q³, Q⁴, and Q⁵ is CR^{QD};
each [symbol] is a single bond or double bond, provided that the ring including Q¹-Q⁵ is aromatic;
R^{QD} is P(═O)R^{a}R^{b}, wherein R^{a} and R^{b} are independently selected from the group consisting of C₁₋₆ alkyl which is optionally substituted with from 1-6 substituents each independently selected from the group consisting of C₁₋₆ alkoxy, C₃₋₆ cycloalkyl, and halo; C₃₋₆ cycloalkyl optionally substituted with from 1-3 substituents each independently selected from the group consisting of C₁₋₃ alkyl and halo; and C₆₋₁₀ aryl optionally substituted with from 1-3 independently selected C₁₋₃ alkyl; or
R^{a} and R^{b} taken together with the phosphorous atom to which each is attached forms a ring including from 5-8 ring atoms, wherein from 0-2 ring atoms (in addition to the phosphorous attached to R^{a} and R^{b}) are heteroatoms each independently selected from the group consisting of O, S, and N, wherein the ring is optionally substituted with from 1-3 independently selected C₁₋₆ alkyl;
each R^{QC} is independently selected from the group consisting of (a) halo; (b) cyano; (c) OH; (d) —NR^{c}R^{d}; (e) C(═O)NR^{c}R^{d}; (f) S(═O)₀₋₂R^{e}; (g) C₁₋₆ alkyl optionally substituted with from 1-6 independently selected R^{f}; (h) C₁₋₆ alkoxy optionally substituted with from 1-6 substituents each independently selected from the group consisting of hydroxy, halo, and C₁₋₆ alkoxy; (i) 3-12 membered heterocyclyl optionally substituted with one or more substituents each independently selected from the group consisting of C₁₋₆ alkyl and C(═O)(C₁₋₆ alkyl); (j) C₆₋₁₀ aryl optionally substituted with from 1-3 independently selected C(═O)(C₁₋₆ alkyl); and (k) 5-10 membered heteroaryl optionally substituted with from 1-6 independently selected R^{g};
or a pair of R^{QC} on adjacent carbon atoms, taken together with the atom to which each is attached, forms a ring including from 5-8 ring atoms, wherein from 0-2 ring atoms are heteroatoms each independently selected from the group consisting of O, N, and S, wherein said ring is optionally substituted with from 1-2 independently selected R groups;
L² is selected from the group consisting of 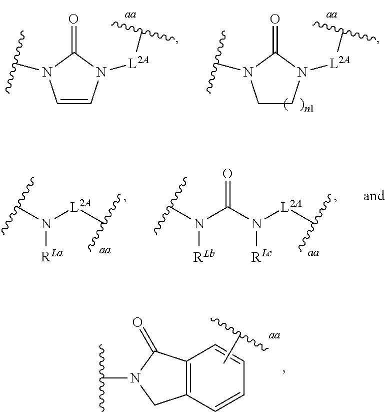
wherein aa represents the point of attachment to the ring containing Q¹-Q⁵;
n1 is an integer from 1-3;
L^{2A} is a bond or C₁₋₁₀ alkylene;
R^{La} is selected from the group consisting of H, C₁₋₆ alkyl, and C(═O)(C₁₋₆ alkyl);
each of R^{Lb} and R^{L}C is independently selected from the group consisting of H and C₁₋₆ alkyl;
Ring A is C₆₋₁₀ aryl, C₅₋₇ cycloalkyl, 5-7 membered heterocyclyl, or 5-10 membered heteroaryl, each of which is optionally substituted with from 1-5 substituents each independently selected from the group consisting of halo, C₁₋₆ alkyl, C₁₋₆ haloalkyl, C₁₋₆ alkoxy, and C₃₋₆ cycloalkyl;
R¹, R², and R³ are each independently selected from the group consisting of H and C₁₋₆ alkyl which is optionally substituted with from 1-6 substituents each independently selected from the group consisting of halo, —OH, and C₁₋₆ alkoxy;
L¹ is selected from the group consisting of —C(═O)—, —CH₂—, —CH(C₁₋₆ alkyl)-, and —S(═O)₂;
Ring B is selected from the group consisting of 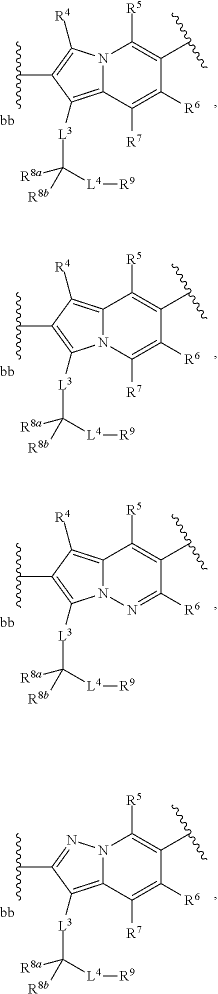 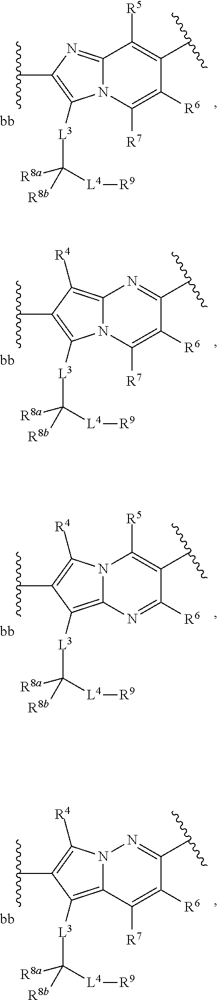 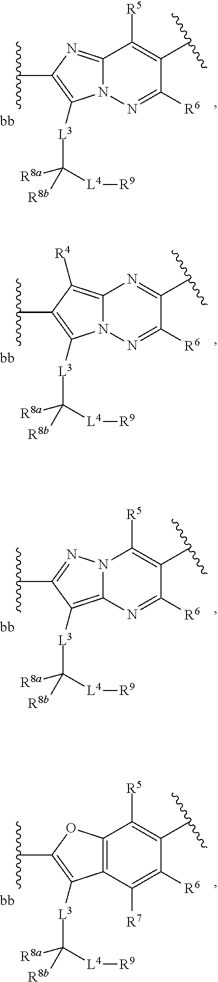 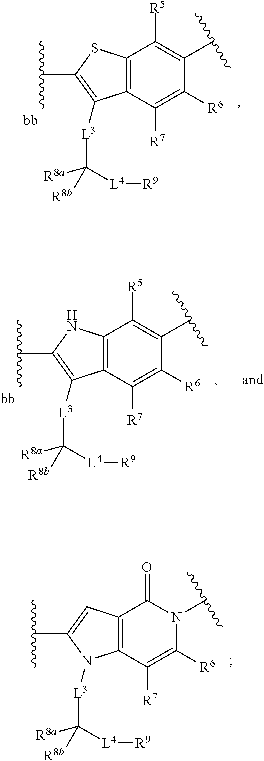
wherein bb represents point of attachment to L¹;
R⁴, R⁵, R⁶, and R⁷ are independently selected from the group consisting of H, halo, and C₁₋₆ alkyl;
L³ is a bond or C₁₋₃ alkylene;
L⁴ is a bond or C₁₋₅ alkylene;
R^{8a} and R^{8b} are independently selected from the group consisting of H and C₁₋₆ alkyl optionally substituted with one or more substituents independently selected from the group consisting of halo and C₃₋₁₅ cycloalkyl; or
R^{8a} and R^{8b} taken together with the carbon atom to which each is attached forms a C₃₋₁₅ cycloalkyl ring which is optionally substituted with from 1-3 independently selected C₁₋₆ alkyl, wherein the C₁₋₆ alkyl is optionally substituted with from 1-6 independently selected R^{f};
R⁹ is selected from the group consisting of C(═O)OH, C(═O)(OC₁₋₆ alkyl), C(═O)NR^{9a}R^{9b}, (IX-1), (IX-2), (IX-3), and (IX-4): 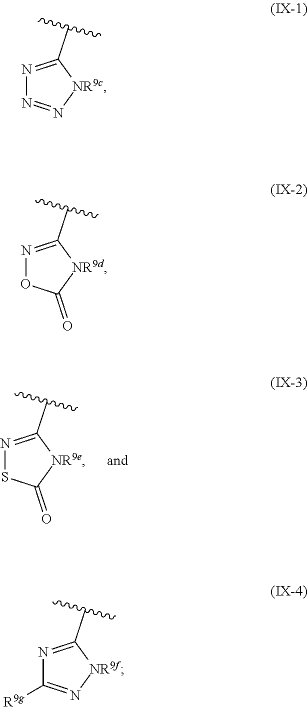
R^{9a} is H or C₁₋₆ alkyl;
R^{9b} is H, C₁₋₆ alkyl, C(═O)(C₁₋₆ alkyl), S(O)₀₋₂(C₁₋₆ alkyl), or cyano;
R^{9c}, R^{9d}, R^{9e}, R^{9f}, and R^{9g} are each independently selected from the group consisting of H;
C₁₋₆ alkyl optionally substituted with from 1-6 independently selected halo and C₁₋₆ alkoxy; and C(═O)(C₁₋₆ alkyl);
Ring C is selected from the group consisting of 3-12 membered heterocyclyl; C₃₋₁₅ cycloalkyl; and 5-10 membered heteroaryl, each of which is optionally substituted with from 1-3 R^{Ca};
each R^{Ca} is independently selected from the group consisting of halo, C₁₋₆ alkyl, C₁₋₆ haloalkyl, C₁₋₆ alkoxy, and NR^{c}R^{d};
or a pair of R^{Ca} on the same or different ring atoms, taken together with the ring atom(s) to which each is attached, forms a carbocyclic ring including from 3-8 ring atoms;
each R^{c} and R^{d} are independently selected from the group consisting of H, C₁₋₆ alkyl, C(═O)(C₁₋₆ alkyl), C(═O)(C₃₋₆ cycloalkyl), C(═O)O(C₁₋₆ alkyl), S(O)₁₋₂(C₁₋₆ alkyl), and S(O)₁₋₂(C₃₋₆ cycloalkyl), wherein the C₁₋₆ alkyl, C(═O)(C₁₋₆ alkyl), C(═O)(C₃₋₆ cycloalkyl), C(═O)O(C₁₋₆ alkyl), S(O)₁₋₂(C₁₋₆ alkyl), and S(O)₁₋₂(C₃₋₆ cycloalkyl) are each optionally substituted with from 1-6 substituents independently selected from the group consisting of —OH, halo, and C₁₋₆ alkoxy;
R^{c} is H, C₁₋₆ alkyl, or C₁₋₆ haloalkyl;
each R^{f} is independently selected from the group consisting of halo, —OH, NR^{c}R^{d}, C₁₋₆ alkoxy, C₁₋₆ haloalkoxy, and 3-12 membered heterocyclyl which is optionally substituted with from 1-4 substituents each independently selected from the group consisting of —OH, C₁₋₆ alkyl, and 3-12 membered heterocyclyl;
each R^{g} is independently selected from the group consisting of C₁₋₆ alkyl, C₁₋₆ alkoxy, NR^{c}R^{d}, and 3 to 12 membered heterocyclyl optionally substituted with one or more substituents each independently selected from the group consisting of C₁₋₆ alkyl and C(═O)C₁₋₆ alkyl; and
each R^{h} is independently selected from the group consisting of halo, cyano, C₁₋₆ alkyl, C₁₋₆ haloalkyl, —OH, NH₂, NH(C₁₋₃ alkyl), N(C₁₋₃ alkyl)₂, C₁₋₃ alkoxy, and C₁₋₃ haloalkoxy.

5. A compound of Formula III: 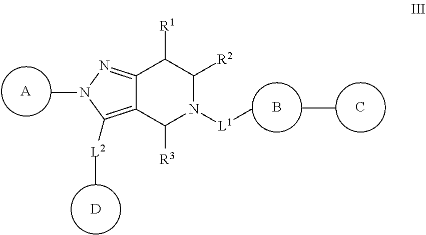
or a pharmaceutically acceptable salt, deuterated analog, or solvate thereof, wherein:
Ring D is selected from the group consisting of C₃₋₁₅ cycloalkyl; 3-12 membered heterocyclyl; 5-10 membered heteroaryl; and C₆₋₁₀ aryl, each of which is optionally substituted with from 1-6 substituents each independently selected from the group consisting of R^{QA} and R^{QB};
provided that one or both of (aa) or (bb) applies:
(aa) Ring D is substituted with at least one R^{QB}; or
(bb) Ring D is 4-12 membered heterocyclyl or 7-10 membered bicyclic heteroaryl, wherein Ring D comprises an endocyclic S(O)₂ group;
each R^{QA} is independently selected from the group consisting of
(a) halo;
(b) cyano;
(c) OH or oxo;
(d) —NR^{a}R^{b};
(e) —C(═O)NR^{c}R^{d};
(f) C₁₋₆ alkyl optionally substituted with from 1-6 independently selected R^{f};
(g) C₁₋₆ alkoxy optionally substituted with from 1-6 independently selected R^{f};
(h) 3-12 membered heterocyclyl optionally substituted with from 1-6 independently selected R^{g};
(i) C₆₋₁₀ aryl optionally substituted with from 1-6 independently selected R^{g};
(j) 5-10 membered heteroaryl optionally substituted with from 1-6 independently selected R^{g}; and
(k) C₃₋₈ cycloalkyl optionally substituted with from 1-6 independently selected R^{g};
each R^{QB} is independently selected from the group consisting of
(a) -L^{a}-S(O)₂-L^{b}-R^{e}; and
(b) 4-12 membered heterocyclyl or 7-10 membered bicyclic heteroaryl, each of which comprises an endocyclic S(O)₂ group, wherein the heterocyclyl or heteroaryl is optionally substituted with from 1-6 independently selected R^{g};
L^{a} is selected from the group consisting of —N(H)—, —N(R^{c})—, and —(CR^{h}R^{h})_{q1};
L^{b} is selected from the group consisting of —O—, —N(H)—, —N(R^{c})—, and —(CR^{h}R^{h})_{q2}—;
q1 and q2 are independently 0, 1, 2, 3, or 4;
provided that when L^{a} is —(CR^{h}R^{h})_{q1}— and q1 is 0, then -L^{b}-R^{e} is other than unsubstituted C₁₋₃ alkyl;
L² is selected from the group consisting of 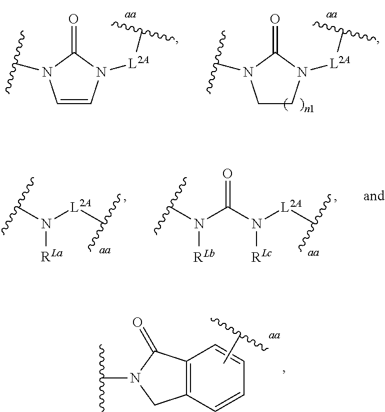
wherein aa represents the point of attachment to Ring D;
n1 is an integer from 1-3;
L^{2A} is a bond or C₁₋₁₀ alkylene;
R^{La} is selected from the group consisting of H, C₁₋₆ alkyl, and C(═O)(C₁₋₆ alkyl);
each of R^{Lb} and R^{Lc} is independently selected from the group consisting of H and C₁₋₆ alkyl;
Ring A is C₆₋₁₀ aryl, C₅₋₁₀ cycloalkyl, 5-10 membered heterocyclyl, or 5-10 membered heteroaryl, each of which is optionally substituted with from 1-5 substituents each independently selected from the group consisting of halo, C₁₋₆ alkyl, C₁₋₆ haloalkyl, C₁₋₆ alkoxy, and C₃₋₆ cycloalkyl;
R¹, R², and R³ are each independently selected from the group consisting of H and C₁₋₆ alkyl which is optionally substituted with from 1-6 substituents each independently selected from the group consisting of halo, —OH, and C₁₋₆ alkoxy;
L¹ is selected from the group consisting of —C(═O)—, —CH₂—, —CH(C₁₋₆ alkyl)-, and —S(═O)₂;
Ring B is selected from the group consisting of 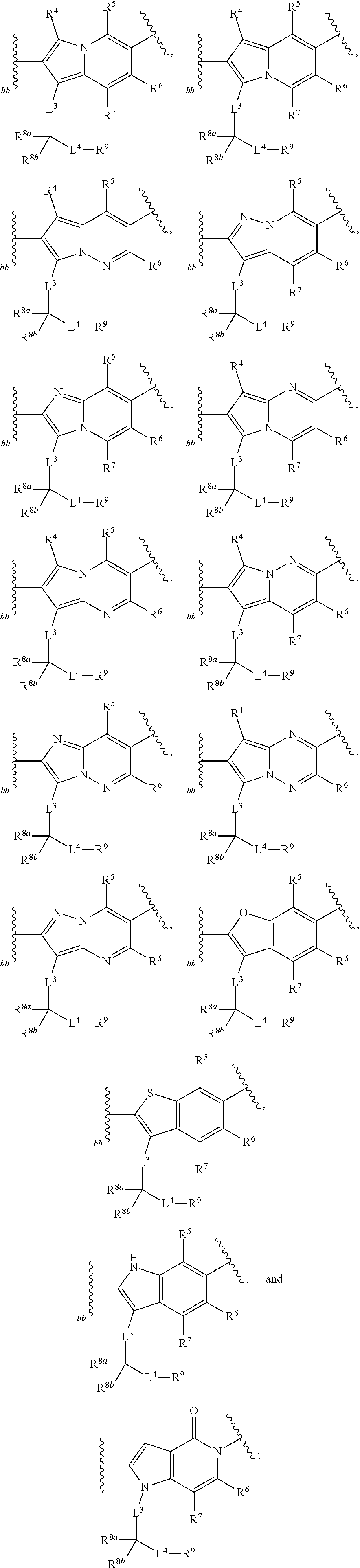
wherein bb represents point of attachment to L¹;
R⁴, R⁵, R⁶, and R⁷ are independently selected from the group consisting of H, halo, and C₁₋₆ alkyl;
L³ is a bond or C₁₋₃ alkylene;
L⁴ is a bond or C₁₋₅ alkylene;
R^{8a} and R^{8b} are independently selected from the group consisting of H and C₁₋₆ alkyl optionally substituted with one or more substituents independently selected from the group consisting of halo and C₃₋₁₅ cycloalkyl; or
R^{8a} and R^{8b} taken together with the carbon atom to which each is attached forms a C₃₋₁₅ cycloalkyl ring which is optionally substituted with from 1-3 independently selected C₁₋₆ alkyl, wherein the C₁₋₆ alkyl is optionally substituted with from 1-6 independently selected R^{f};
R⁹ is selected from the group consisting of C(═O)OH, C(═O)(OC₁₋₆ alkyl), C(═O)NR^{9a}R^{9b}(IX-1), (IX-2), (IX-3), and (IX-4): 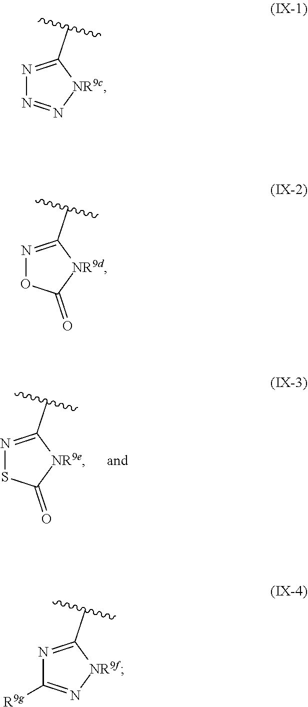
R^{9a} is H or C₁₋₆ alkyl;
R^{9b} is H, C₁₋₆ alkyl, C(═O)(C₁₋₆ alkyl), S(O)₀₋₂(C₁₋₆ alkyl), or cyano;
R^{9c}, R^{9d}, R^{9e}, R^{9f}, and R^{9g} are each independently selected from the group consisting of H; C₁₋₆ alkyl optionally substituted with from 1-6 independently selected halo and C₁₋₆ alkoxy; and C(═O)(C₁₋₆ alkyl);
Ring C is selected from the group consisting of 3-12 membered heterocyclyl; C₃₋₁₅ cycloalkyl; and 5-10 membered heteroaryl, each of which is optionally substituted with from 1-3 R^{Ca};
each R^{Ca} is independently selected from the group consisting of halo, C₁₋₆ alkyl, C₁₋₆ haloalkyl, C₁₋₆ alkoxy, and NR^{c}R^{d};
or a pair of R^{Ca} on the same or different ring atoms, taken together with the ring atom(s) to which each is attached, forms a carbocyclic ring including from 3-8 ring atoms;
each R^{a} and R^{b} are independently selected from the group consisting of H, C₁₋₆ alkyl, C₃₋₆ cycloalkyl, C(═O)(C₁₋₆ alkyl), C(═O)(C₃₋₆ cycloalkyl), and C(═O)O(C₁₋₆ alkyl), wherein the C₁₋₆ alkyl, C₃₋₆ cycloalkyl, C(═O)(C₁₋₆ alkyl), C(═O)(C₃₋₆ cycloalkyl), and C(═O)O(C₁₋₆ alkyl) are each optionally substituted with from 1-6 substituents independently selected from the group consisting of —OH, halo, and C₁₋₆ alkoxy;
each R^{c} and R^{d} are independently selected from the group consisting of H, C₁₋₆ alkyl, C₃₋₆ cycloalkyl, C(═O)(C₁₋₆ alkyl), C(═O)(C₃₋₆ cycloalkyl), C(═O)O(C₁₋₆ alkyl), S(O)₁₋₂(C₁₋₆ alkyl), and S(O)₁₋₂(C₃₋₆ cycloalkyl), wherein the C₁₋₆ alkyl, C₃₋₆ cycloalkyl, C(═O)(C₁₋₆ alkyl), C(═O)(C₃₋₆ cycloalkyl), C(═O)O(C₁₋₆ alkyl), S(O)₁₋₂(C₁₋₆ alkyl), and S(O)₁₋₂(C₃₋₆ cycloalkyl) are each optionally substituted with from 1-6 substituents independently selected from the group consisting of —OH, halo, and C₁₋₆ alkoxy;
R^{e} is H, or R^{e} is selected from the group consisting of C₁₋₆ alkyl, C₁₋₆ haloalkyl, C₁₋₆ alkoxy, C₁₋₆ haloalkoxy, C₃₋₆ cycloalkyl, and 3-8 membered heterocyclyl, each of which is optionally substituted with from 1-3 substituents each independently selected from the group consisting of halo, cyano, C₁₋₃ alkyl, C₁₋₃ haloalkyl, —OH, NH₂, NH(C₁₋₃ alkyl), N(C₁₋₃ alkyl)₂, C₁₋₃ alkoxy, and C₁₋₃ haloalkoxy;
each R^{f} is independently selected from the group consisting of halo, —OH, NR^{c}R^{d}, C₁₋₆ alkoxy, C₁₋₆ haloalkoxy, and 3-12 membered heterocyclyl which is optionally substituted with from 1-4 substituents each independently selected from the group consisting of —OH, C₁₋₆ alkyl, and 3-12 membered heterocyclyl;
each R^{g} is independently selected from the group consisting of C₁₋₆ alkyl, C₁₋₆ haloalkyl, C₁₋₆ alkoxy, C₁₋₆ haloalkoxy, NR^{c}R^{d}, cyano, halo, C₃₋₆ cycloalkyl, and 3 to 12 membered heterocyclyl optionally substituted with one or more substituents each independently selected from the group consisting of C₁₋₆ alkyl and C(═O)C₁₋₆ alkyl; and
each occurrence of R^{h} is independently selected from the group consisting of H, C₁₋₆ alkyl, C₁₋₆ haloalkyl, C₃₋₆ cycloalkyl, and halo; or
a pair of R^{h} on the same or different carbon atom(s), taken together with the carbon atom(s) connecting them forms C₃₋₆ cycloalkyl or 4-8 membered heterocyclyl, each of which is optionally substituted with from 1-3 substituents each independently selected from the group consisting of halo, cyano, C₁₋₃ alkyl, C₁₋₃ haloalkyl, —OH, C₁₋₃ alkoxy, and C₁₋₃ haloalkoxy.

6. A compound of Formula IV: 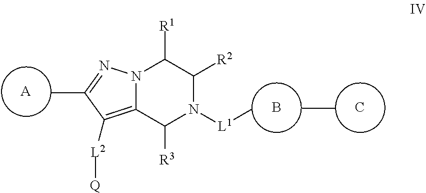
or a pharmaceutically acceptable salt, deuterated analog, or solvate thereof, wherein:
Ring A is C₆₋₁₀ aryl, C₅₋₁₀ cycloalkyl, 5-10 membered heterocyclyl, or 5-10 membered heteroaryl, each of which is optionally substituted with from 1-5 substituents each independently selected from the group consisting of halo, C₁₋₆ alkyl, C₁₋₆ haloalkyl, C₁₋₆ alkoxy, and C₃₋₆ cycloalkyl;
R¹, R², and R³ are each independently selected from the group consisting of H and C₁₋₆ alkyl which is optionally substituted with from 1-6 substituents each independently selected from the group consisting of halo, —OH, and C₁₋₆ alkoxy;
L¹ is selected from the group consisting of —C(═O)—, —CH₂—, —CH(C₁₋₆ alkyl)-, and —S(═O)₂;
Ring B is selected from the group consisting of 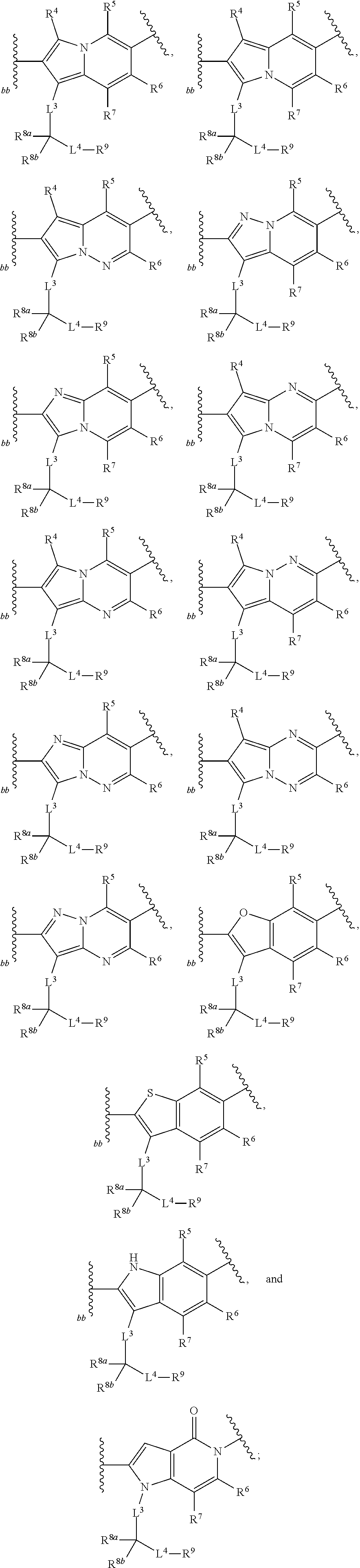
wherein bb represents point of attachment to L¹;
R⁴, R⁵, R⁶, and R⁷ are independently selected from the group consisting of H, halo, and C₁₋₆ alkyl;
L³ is a bond or C₁₋₃ alkylene;
L⁴ is a bond or C₁₋₅ alkylene;
R^{8a} and R^{8b} are independently selected from the group consisting of H and C₁₋₆ alkyl optionally substituted with one or more substituents independently selected from the group consisting of halo and C₃₋₁₅ cycloalkyl; or
R^{8a} and R^{8b} taken together with the carbon atom to which each is attached forms a C₃₋₁₅ cycloalkyl ring which is optionally substituted with from 1-3 independently selected C₁₋₆ alkyl, wherein the C₁₋₆ alkyl is optionally substituted with from 1-6 independently selected R^{f};
R⁹ is selected from the group consisting of C(═O)OH, C(═O)(OC₁₋₆ alkyl), C(═O)NR^{9a}R^{9b}, (IX-1), (IX-2), (IX-3), and (IX-4): 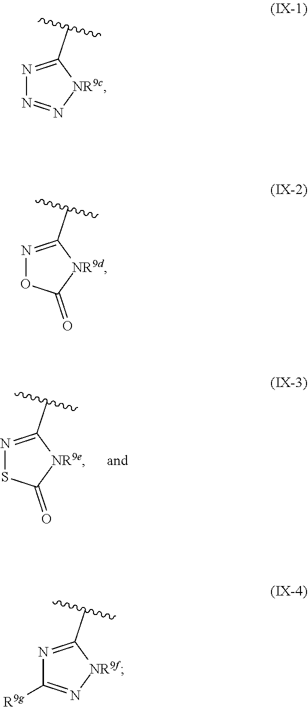
R^{9a} is H or C₁₋₆ alkyl;
R^{9b} is H, C₁₋₆ alkyl, C(═O)(C₁₋₆ alkyl), S(O)₀₋₂(C₁₋₆ alkyl), or cyano;
R^{9c}, R^{9d}, R^{9e}, R^{9f}, and R^{9g} are each independently selected from the group consisting of H; C₁₋₆ alkyl optionally substituted with from 1-6 independently selected halo and C₁₋₆ alkoxy; and C(═O)(C₁₋₆ alkyl);
Ring C is selected from the group consisting of 3-12 membered heterocyclyl; C₃₋₁₀ cycloalkyl; and 5-10 membered heteroaryl, each of which is optionally substituted with from 1-3 R^{Ca};
each R^{Ca} is independently selected from the group consisting of halo, C₁₋₆ alkyl, C₁₋₆ haloalkyl, C₁₋₆ alkoxy, and NR^{c}R^{d};
or a pair of R^{Ca} on the same or different ring atoms, taken together with the ring atom(s) to which each is attached, forms a carbocyclic ring including from 3-8 ring atoms;
L² is selected from the group consisting of 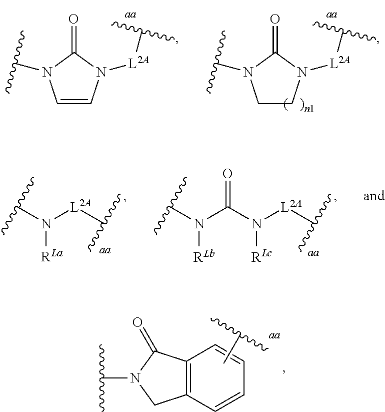
wherein aa represents the point of attachment to Q;
n1 is an integer from 1-3;
L^{2A} is a bond or C₁₋₁₀ alkylene;
R^{La} is selected from the group consisting of H, C₁₋₆ alkyl, and C(═O)(C₁₋₆ alkyl);
each of R^{Lb} and R^{Lc} is independently selected from the group consisting of H and C₁₋₆ alkyl;
Q is selected from the group consisting of C₁₋₁₀ alkyl; C₃₋₁₅ cycloalkyl; 3-12 membered heterocyclyl; 5-10 membered heteroaryl; and C₆₋₁₀ aryl, each of which is optionally substituted with from 1-6 independently selected R^{Q};
each R^{Q} is independently selected from the group consisting of
(a) halo;
(b) cyano;
(c) OH or oxo;
(d) —NR^{c}R^{d};
(e) —C(═O)NR^{c}R^{d} or —S(O)₂NR^{c}R^{d};
(f) —S(═O)₀₋₂R^{e};
(g) C₁₋₆ alkyl optionally substituted with from 1-6 independently selected R^{f};
(h) C₁₋₆ alkoxy optionally substituted with from 1-6 independently selected R^{f};
(i) 3-12 membered heterocyclyl optionally substituted with from 1-6 independently selected R^{g};
(j) C₆₋₁₀ aryl optionally substituted with from 1-6 independently selected R^{g};
(k) 5-10 membered heteroaryl optionally substituted with from 1-6 independently selected R^{g};
(l) C₃₋₈ cycloalkyl optionally substituted with from 1-6 independently selected R^{g};
(m) P(═O)R^{a}R^{b}; and
(n) —(CR^{h}R^{h})_{q}S(O)₂-L^{e}-R^{e}, wherein q1 is 1, 2, or 3;
R^{a} and R^{b} are independently selected from the group consisting of C₁₋₆ alkyl which is optionally substituted with from 1-6 substituents each independently selected from the group consisting of C₁₋₆ alkoxy, C₃₋₆ cycloalkyl, and halo; C₃₋₆ cycloalkyl optionally substituted with from 1-3 substituents each independently selected from the group consisting of C₁₋₃ alkyl and halo; and C₆₋₁₀ aryl optionally substituted with from 1-3 independently selected C₁₋₃ alkyl; or
R^{a} and R^{b} taken together with the phosphorous atom to which each is attached forms a ring including from 5-8 ring atoms, wherein from 0-2 ring atoms (in addition to the phosphorous attached to R^{a} and R^{b}) are heteroatoms each independently selected from the group consisting of O, S, and N, wherein the ring is optionally substituted with from 1-3 independently selected C₁₋₆ alkyl;
each R^{c} and R^{d} are independently selected from the group consisting of H, C₁₋₆ alkyl, C₃₋₆ cycloalkyl, C(═O)(C₁₋₆ alkyl), C(═O)(C₃₋₆ cycloalkyl), C(═O)O(C₁₋₆ alkyl), S(O)₁₋₂(C₁₋₆ alkyl), and S(O)₁₋₂(C₃₋₆ cycloalkyl), wherein the C₁₋₆ alkyl, C(═O)(C₁₋₆ alkyl), C(═O)(C₃₋₆ cycloalkyl), C(═O)O(C₁₋₆ alkyl), S(O)₁₋₂(C₁₋₆ alkyl), and S(O)₁₋₂(C₃₋₆ cycloalkyl) are each optionally substituted with from 1-6 substituents independently selected from the group consisting of —OH, halo, and C₁₋₆ alkoxy;
R^{e} is H, or R^{e} is selected from the group consisting of C₁₋₆ alkyl, C₁₋₆ haloalkyl, C₁₋₆ alkoxy, C₁₋₆ haloalkoxy, C₃₋₆ cycloalkyl, and 3-8 membered heterocyclyl, each of which is optionally substituted with from 1-3 substituents each independently selected from the group consisting of halo, cyano, C₁₋₃ alkyl, C₁₋₃ haloalkyl, —OH, NH₂, NH(C₁₋₃ alkyl), N(C₁₋₃ alkyl)₂, C₁₋₃ alkoxy, and C₁₋₃ haloalkoxy;
L^{e} is a bond, NR^{c}, or O;
each R^{f} is independently selected from the group consisting of halo, —OH, NR^{c}R^{d}, C₁₋₆ alkoxy, C₁₋₆ haloalkoxy, and 3-12 membered heterocyclyl which is optionally substituted with from 1-4 substituents each independently selected from the group consisting of —OH, C₁₋₆ alkyl, and 3-12 membered heterocyclyl;
each R^{g} is independently selected from the group consisting of C₁₋₆ alkyl, C₁₋₆ haloalkyl, C₁₋₆ alkoxy, C₁₋₆ haloalkoxy, NR^{c}R^{d}, cyano, halo, C₃₋₆ cycloalkyl, and 3 to 12 membered heterocyclyl optionally substituted with one or more substituents each independently selected from the group consisting of C₁₋₆ alkyl and C(═O)C₁₋₆ alkyl; and
each occurrence of R^{h} is independently selected from the group consisting of H, C₁₋₆ alkyl, C₁₋₆ haloalkyl, C₃₋₆ cycloalkyl, and halo; or
a pair of R^{h} on the same or different carbon atom(s), taken together with the carbon atom(s) connecting them forms C₃₋₆ cycloalkyl or 4-8 membered heterocyclyl, each of which is optionally substituted with from 1-3 substituents each independently selected from the group consisting of halo, cyano, C₁₋₃ alkyl, C₁₋₃ haloalkyl, —OH, C₁₋₃ alkoxy, and C₁₋₃ haloalkoxy.

7. The compound of claim 1, wherein Ring A is C₆₋₁₀ aryl, optionally substituted with from 1-5 substituents each independently selected from the group consisting of halo, C₁₋₆ alkyl, C₁₋₆ haloalkyl, C₁₋₆ alkoxy, and C₃₋₆ cycloalkyl.

8. The compound of claim 1, wherein Ring A is phenyl, optionally substituted with from 1-3 substituents each independently selected from the group consisting of fluoro, methyl, and cyclopropyl.

9. The compound of claim 1, wherein R¹ and R² are H.

10. The compound of claim 1, wherein R³ is C₁₋₆ alkyl.

11. The compound of claim 1, wherein R³ is methyl.

12. The compound of claim 1, wherein L¹ is —C(═O)—.

13. The compound of claim 1, wherein Ring B is:

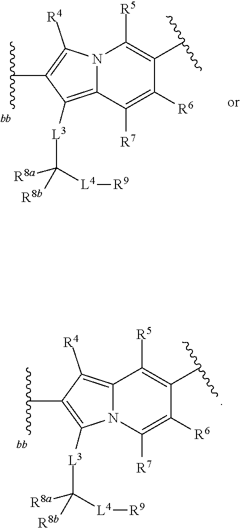

14. The compound of claim 1, wherein Ring B is:

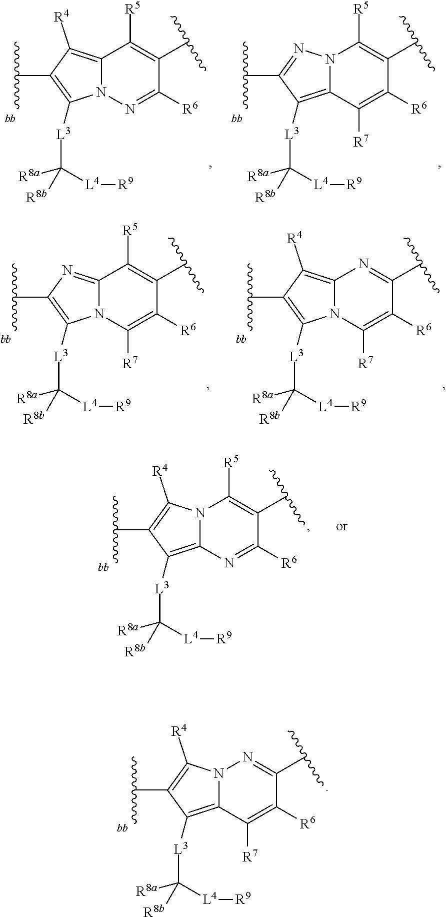

15. The compound of claim 1, wherein Ring B is:

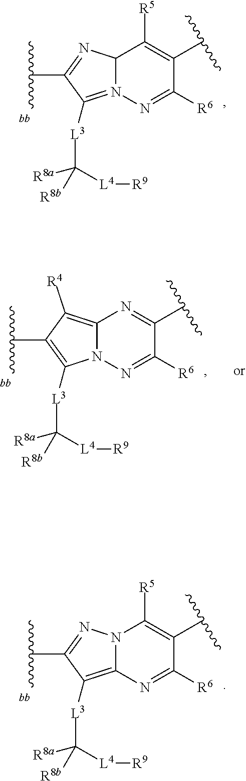

16. The compound of claim 1, wherein Ring B is:

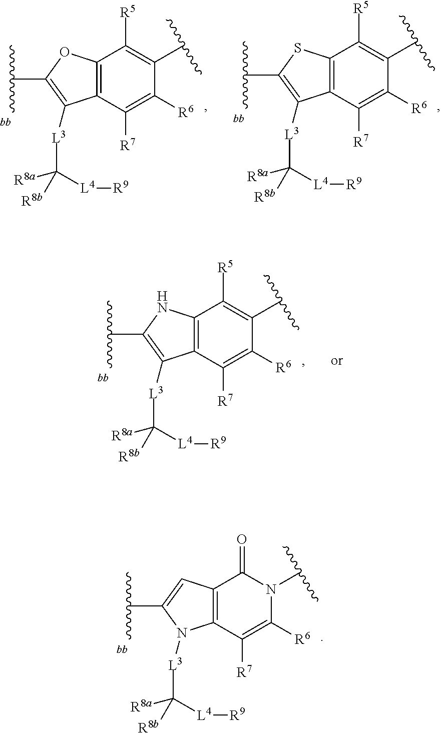

17. The compound of claim 1, wherein R⁴, R⁵, R⁶, and R⁷ are H.

18. The compound of claim 1, wherein L³ is a bond.

19. The compound of claim 1, wherein R^{8a} and R^{8b} together with the carbon atom to which each is attached form a C₃₋₁₅ cycloalkyl.

20. The compound of claim 1, wherein R^{8a} and R^{8b} together with the carbon atom to which each is attached form a cyclopropyl.

21. The compound of claim 1, wherein R⁹ is

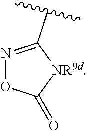

22. The compound of claim 1, wherein R^{9d} is H.

23. The compound of claim 1, wherein Ring C is 3-12 membered heterocyclyl.

24. The compound of claim 1, wherein Ring C is tetrahydropyranyl.

25. The compound of claim 1, wherein L² is 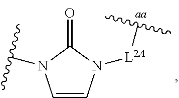
wherein aa represents the point of attachment to Q.

26. The compound of claim 1, wherein L^{2A} is a bond.

27. A compound of Formula IIE: 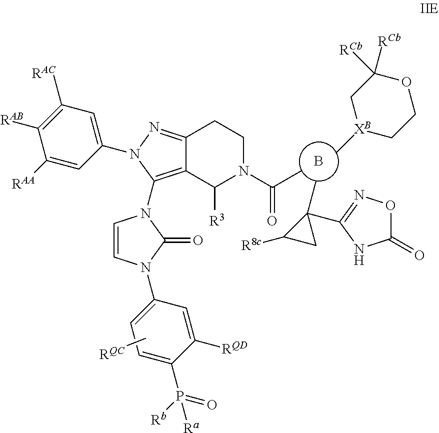
or a pharmaceutically acceptable salt, deuterated analog, or solvate thereof, wherein:
Ring B is selected from the group consisting of: 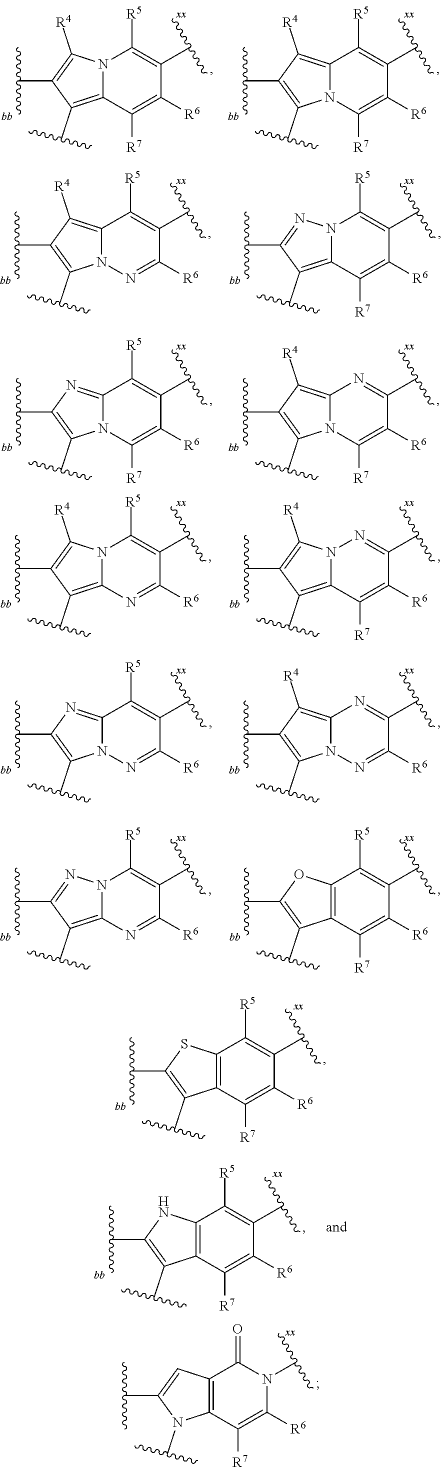
wherein bb represents the point of attachment to the —C(O)— and xx represents the point of attachment to X^{b};
R⁴, R⁵, R⁶, and R⁷ are independently selected from the group consisting of H, halo, and C₁₋₆ alkyl;
R^{AA}, R^{AB}, and R^{AC} are each independently hydrogen, halo, C₁₋₃ alkyl, or C₃₋₆ cycloalkyl;
R³ is H or C₁₋₃ alkyl;
R^{QC} is H or halo;
R^{QD} is selected from the group consisting of: (a) H; (b) —NH₂; (c) —NH(C₁₋₃ alkyl); (d) halo; (e) C₁₋₃ alkoxy optionally substituted with from 1-3 independently selected halo; and (f) C₁₋₃ alkyl optionally substituted with from 1-3 independently selected halo;
R^{a} and R^{b} are each independently selected from the group consisting of: C₁₋₄ alkyl and C₃₋₆ cycloalkyl; or
R^{a} and R^{b} taken together with the phosphorous atom to which each is attached form a ring including from 5-8 ring atoms, wherein from 0-1 ring atom (in addition to the phosphorous attached to R^{a} and R^{b}) is a heteroatom independently selected from the group consisting of: O, S, and N;
R^{8c} is H or C₁₋₃ alkyl;
X^{B} is CH or N; and
each R^{Cb} is independently selected from the group consisting of H and C₁₋₃ alkyl.

28. A compound selected from: 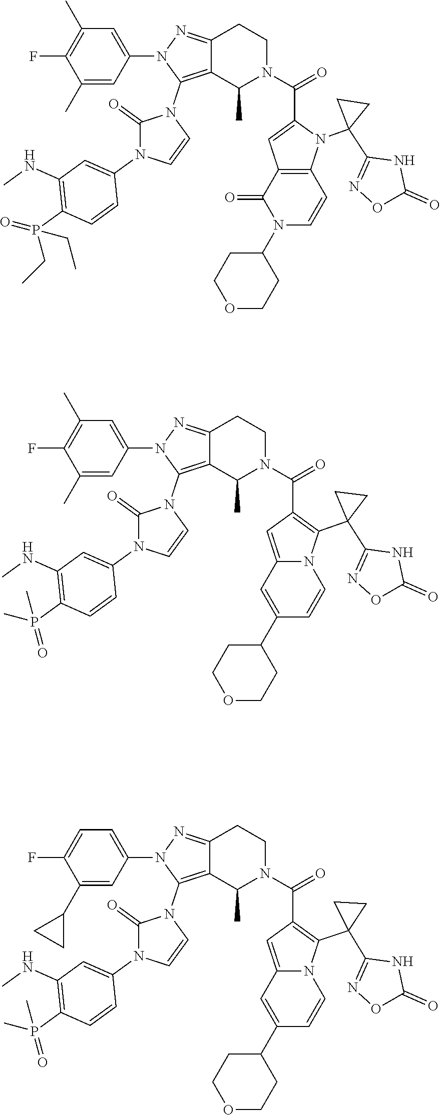 [SMILES: CCP(=O)(CC)c1ccc(-n2ccn(-c3c4c(nn3-c3cc(C)c(F)c(C)c3)CCN(C(=O)c3cc5c(=O)n(C6CCOCC6)ccc5n3C3(c5noc(=O)[nH]5)CC3)[C@H]4C)c2=O)cc1NC ; CNc1cc(-n2ccn(-c3c4c(nn3-c3cc(C)c(F)c(C)c3)CCN(C(=O)c3cc5cc(C6CCOCC6)ccn5c3C3(c5noc(=O)[nH]5)CC3)[C@H]4C)c2=O)ccc1P(C)(C)=O ; CNc1cc(-n2ccn(-c3c4c(nn3-c3ccc(F)c(C5CC5)c3)CCN(C(=O)c3cc5cc(C6CCOCC6)ccn5c3C3(c5noc(=O)[nH]5)CC3)[C@H]4C)c2=O)ccc1P(C)(C)=O] 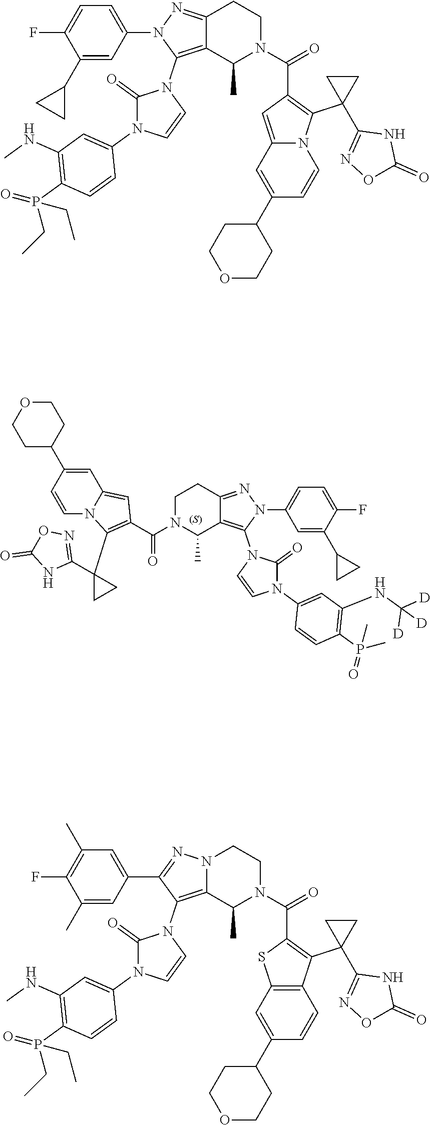 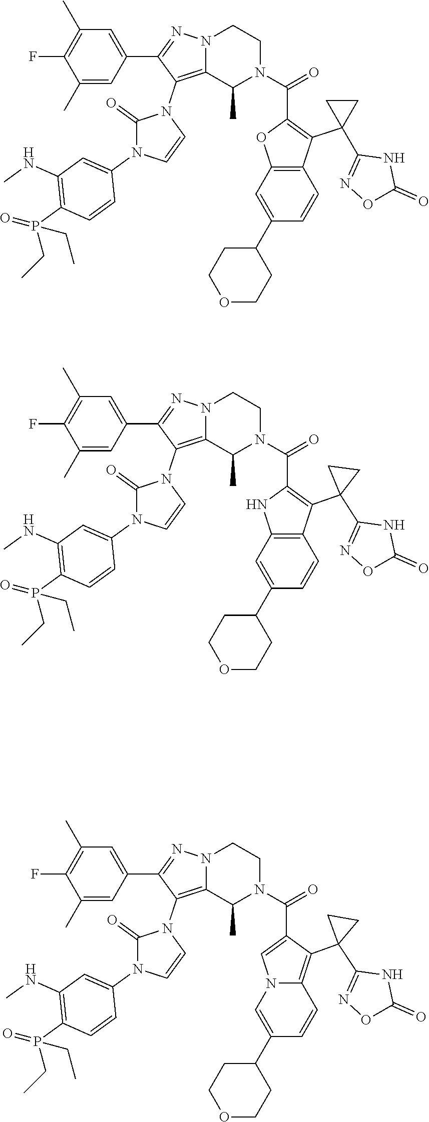 [SMILES: CCP(=O)(CC)c1ccc(-n2ccn(-c3c(-c4cc(C)c(F)c(C)c4)nn4c3[C@H](C)N(C(=O)c3oc5cc(C6CCOCC6)ccc5c3C3(c5noc(=O)[nH]5)CC3)CC4)c2=O)cc1NC ; CCP(=O)(CC)c1ccc(-n2ccn(-c3c(-c4cc(C)c(F)c(C)c4)nn4c3[C@H](C)N(C(=O)c3[nH]c5cc(C6CCOCC6)ccc5c3C3(c5noc(=O)[nH]5)CC3)CC4)c2=O)cc1NC ; CCP(=O)(CC)c1ccc(-n2ccn(-c3c(-c4cc(C)c(F)c(C)c4)nn4c3[C@H](C)N(C(=O)c3cn5cc(C6CCOCC6)ccc5c3C3(c5noc(=O)[nH]5)CC3)CC4)c2=O)cc1NC] 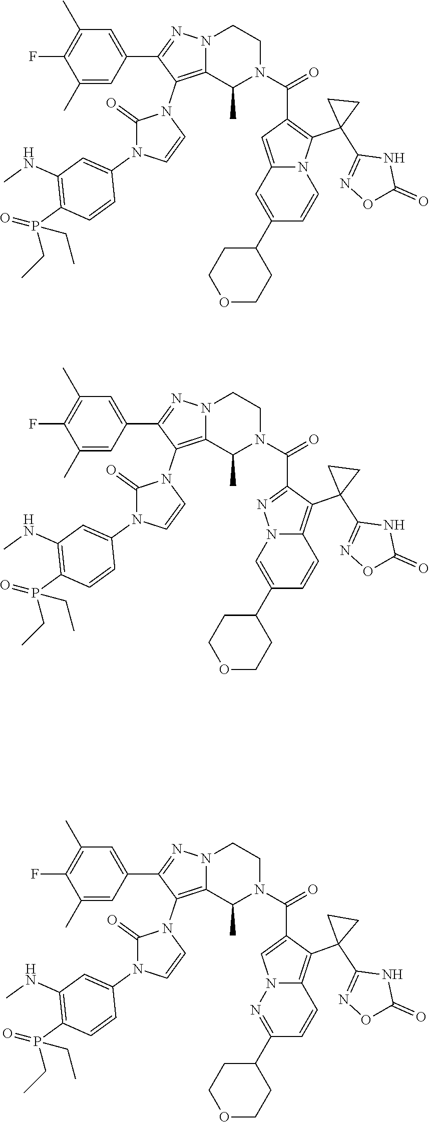 [SMILES: CCP(=O)(CC)c1ccc(-n2ccn(-c3c(-c4cc(C)c(F)c(C)c4)nn4c3[C@H](C)N(C(=O)c3cc5cc(C6CCOCC6)ccn5c3C3(c5noc(=O)[nH]5)CC3)CC4)c2=O)cc1NC ; CCP(=O)(CC)c1ccc(-n2ccn(-c3c(-c4cc(C)c(F)c(C)c4)nn4c3[C@H](C)N(C(=O)c3nn5cc(C6CCOCC6)ccc5c3C3(c5noc(=O)[nH]5)CC3)CC4)c2=O)cc1NC ; CCP(=O)(CC)c1ccc(-n2ccn(-c3c(-c4cc(C)c(F)c(C)c4)nn4c3[C@H](C)N(C(=O)c3cn5nc(C6CCOCC6)ccc5c3C3(c5noc(=O)[nH]5)CC3)CC4)c2=O)cc1NC] 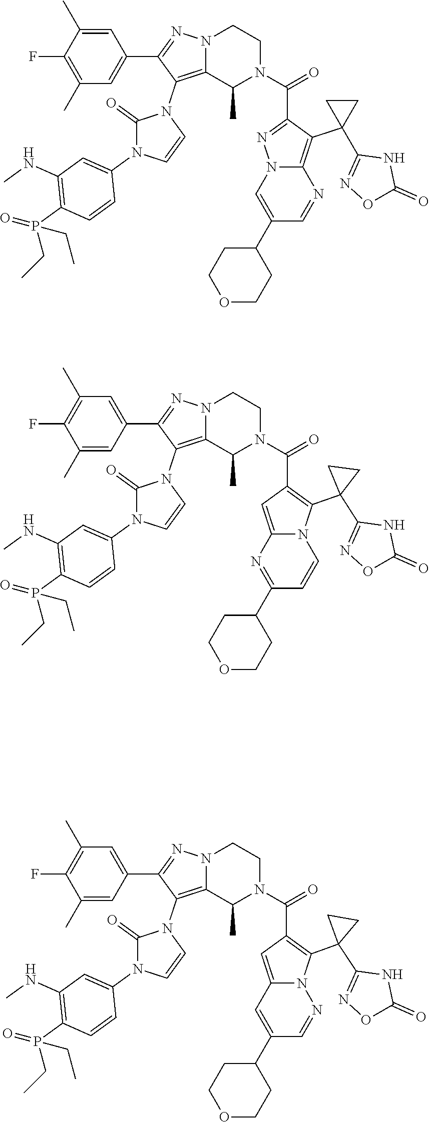 [SMILES: CCP(=O)(CC)c1ccc(-n2ccn(-c3c(-c4cc(C)c(F)c(C)c4)nn4c3[C@H](C)N(C(=O)c3nn5cc(C6CCOCC6)cnc5c3C3(c5noc(=O)[nH]5)CC3)CC4)c2=O)cc1NC ; CCP(=O)(CC)c1ccc(-n2ccn(-c3c(-c4cc(C)c(F)c(C)c4)nn4c3[C@H](C)N(C(=O)c3cc5nc(C6CCOCC6)ccn5c3C3(c5noc(=O)[nH]5)CC3)CC4)c2=O)cc1NC ; CCP(=O)(CC)c1ccc(-n2ccn(-c3c(-c4cc(C)c(F)c(C)c4)nn4c3[C@H](C)N(C(=O)c3cc5cc(C6CCOCC6)cnn5c3C3(c5noc(=O)[nH]5)CC3)CC4)c2=O)cc1NC] 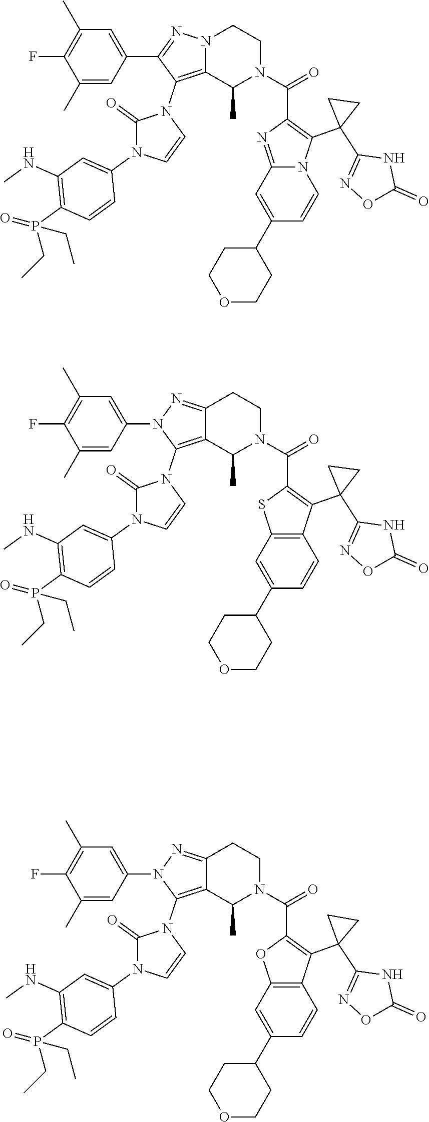 [SMILES: CCP(=O)(CC)c1ccc(-n2ccn(-c3c(-c4cc(C)c(F)c(C)c4)nn4c3[C@H](C)N(C(=O)c3nc5cc(C6CCOCC6)ccn5c3C3(c5noc(=O)[nH]5)CC3)CC4)c2=O)cc1NC ; CCP(=O)(CC)c1ccc(-n2ccn(-c3c4c(nn3-c3cc(C)c(F)c(C)c3)CCN(C(=O)c3sc5cc(C6CCOCC6)ccc5c3C3(c5noc(=O)[nH]5)CC3)[C@H]4C)c2=O)cc1NC ; CCP(=O)(CC)c1ccc(-n2ccn(-c3c4c(nn3-c3cc(C)c(F)c(C)c3)CCN(C(=O)c3oc5cc(C6CCOCC6)ccc5c3C3(c5noc(=O)[nH]5)CC3)[C@H]4C)c2=O)cc1NC] 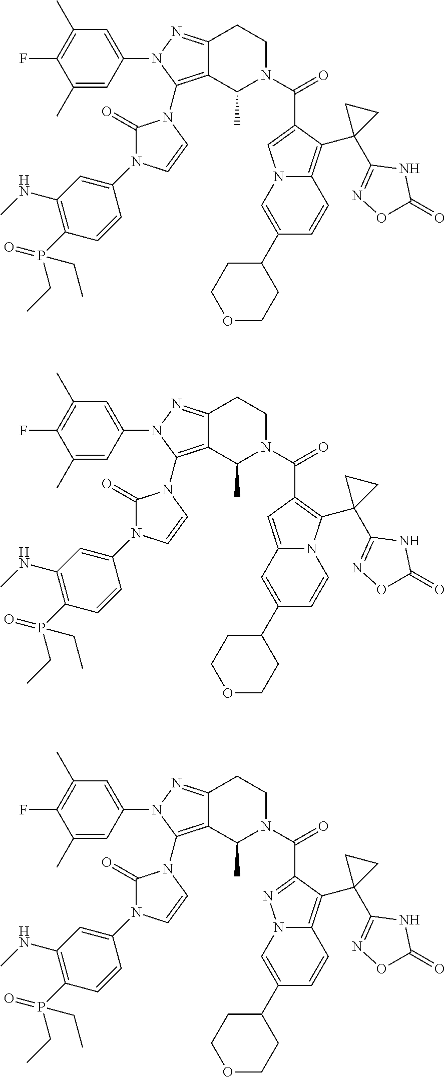 [SMILES: CCP(=O)(CC)c1ccc(-n2ccn(-c3c4c(nn3-c3cc(C)c(F)c(C)c3)CCN(C(=O)c3cn5cc(C6CCOCC6)ccc5c3C3(c5noc(=O)[nH]5)CC3)[C@@H]4C)c2=O)cc1NC ; CCP(=O)(CC)c1ccc(-n2ccn(-c3c4c(nn3-c3cc(C)c(F)c(C)c3)CCN(C(=O)c3cc5cc(C6CCOCC6)ccn5c3C3(c5noc(=O)[nH]5)CC3)[C@H]4C)c2=O)cc1NC ; CCP(=O)(CC)c1ccc(-n2ccn(-c3c4c(nn3-c3cc(C)c(F)c(C)c3)CCN(C(=O)c3nn5cc(C6CCOCC6)ccc5c3C3(c5noc(=O)[nH]5)CC3)[C@H]4C)c2=O)cc1NC] 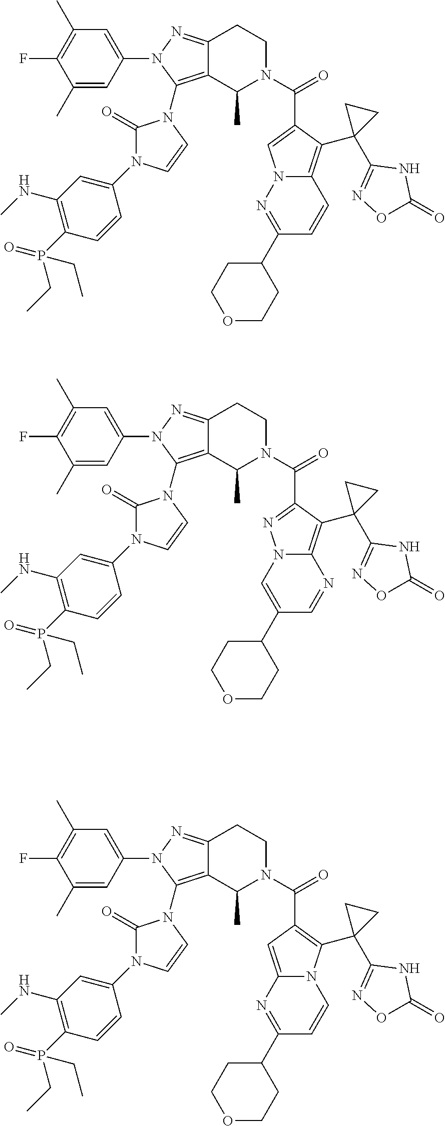 [SMILES: CCP(=O)(CC)c1ccc(-n2ccn(-c3c4c(nn3-c3cc(C)c(F)c(C)c3)CCN(C(=O)c3cn5nc(C6CCOCC6)ccc5c3C3(c5noc(=O)[nH]5)CC3)[C@H]4C)c2=O)cc1NC ; CCP(=O)(CC)c1ccc(-n2ccn(-c3c4c(nn3-c3cc(C)c(F)c(C)c3)CCN(C(=O)c3nn5cc(C6CCOCC6)cnc5c3C3(c5noc(=O)[nH]5)CC3)[C@H]4C)c2=O)cc1NC ; CCP(=O)(CC)c1ccc(-n2ccn(-c3c4c(nn3-c3cc(C)c(F)c(C)c3)CCN(C(=O)c3cc5nc(C6CCOCC6)ccn5c3C3(c5noc(=O)[nH]5)CC3)[C@H]4C)c2=O)cc1NC] 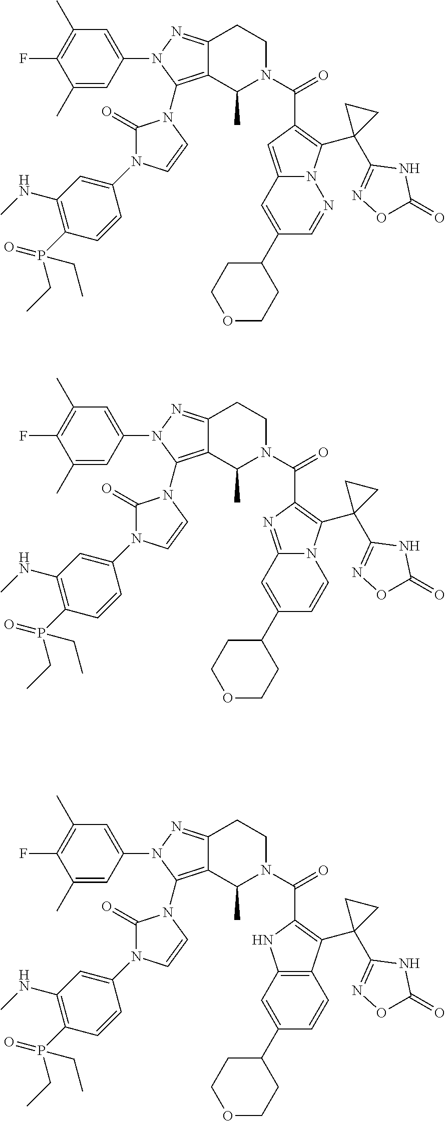 [SMILES: CCP(=O)(CC)c1ccc(-n2ccn(-c3c4c(nn3-c3cc(C)c(F)c(C)c3)CCN(C(=O)c3cc5cc(C6CCOCC6)cnn5c3C3(c5noc(=O)[nH]5)CC3)[C@H]4C)c2=O)cc1NC ; CCP(=O)(CC)c1ccc(-n2ccn(-c3c4c(nn3-c3cc(C)c(F)c(C)c3)CCN(C(=O)c3nc5cc(C6CCOCC6)ccn5c3C3(c5noc(=O)[nH]5)CC3)[C@H]4C)c2=O)cc1NC ; CCP(=O)(CC)c1ccc(-n2ccn(-c3c4c(nn3-c3cc(C)c(F)c(C)c3)CCN(C(=O)c3[nH]c5cc(C6CCOCC6)ccc5c3C3(c5noc(=O)[nH]5)CC3)[C@H]4C)c2=O)cc1NC] 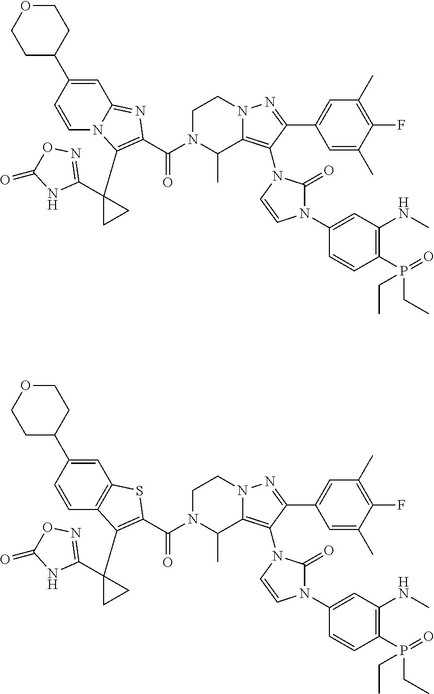 [SMILES: CCP(=O)(CC)c1ccc(-n2ccn(-c3c(-c4cc(C)c(F)c(C)c4)nn4c3C(C)N(C(=O)c3nc5cc(C6CCOCC6)ccn5c3C3(c5noc(=O)[nH]5)CC3)CC4)c2=O)cc1NC ; CCP(=O)(CC)c1ccc(-n2ccn(-c3c(-c4cc(C)c(F)c(C)c4)nn4c3C(C)N(C(=O)c3sc5cc(C6CCOCC6)ccc5c3C3(c5noc(=O)[nH]5)CC3)CC4)c2=O)cc1NC]
or a pharmaceutically acceptable salt, deuterated analog, or solvate thereof.

29. A pharmaceutical composition comprising a compound of claim 1, or a pharmaceutically acceptable salt, deuterated analog, or solvate thereof, and a pharmaceutically acceptable excipient.

30. A method for treating a GLP-1 associated disease, disorder, or condition, the method comprising administering to a patient in need thereof an effective amount of a compound of claim 1, or a pharmaceutically acceptable salt, deuterated analog, or solvate thereof.

31. The method of claim 30, wherein the disease, disorder, or condition is selected from the group consisting of type 1 diabetes mellitus, type 2 diabetes mellitus, early onset type 2 diabetes mellitus, idiopathic type 1 diabetes mellitus (Type 1b), youth-onset atypical diabetes (YOAD), maturity onset diabetes of the young (MODY), latent autoimmune diabetes in adults (LADA), obesity (including hypothalamic obesity and monogenic obesity), weight gain from use of other agents, idiopathic intracranial hypertension, Wolfram syndrome, gout, excessive sugar craving, hypertriglyceridemia, dyslipidemia, malnutrition-related diabetes, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, traumatic brain injury, peripheral vascular disease, endothelial dysfunction, impaired vascular compliance, vascular restenosis, thrombosis, hypertension, pulmonary hypertension, restenosis after angioplasty, intermittent claudication, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, macular degeneration, cataract, glomerulosclerosis, arthritis, osteoporosis, treatment of addiction, cocaine dependence, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), ulcerative colitis, inflammatory bowel disease, colitis, irritable bowel syndrome, Crohn's disease, short bowel syndrome, Parkinson's, Alzheimer's disease, impaired cognition, schizophrenia, Polycystic Ovary Syndrome (PCOS), or any combination thereof.

32. A method of treating type 2 diabetes mellitus in a patient in need thereof, the method comprising administering to a patient in need thereof an effective amount of a compound of claim 1, or a pharmaceutically acceptable salt, deuterated analog, or solvate thereof.

33. A method for modulating insulin levels in a patient in need of such modulating, the method comprising administering to a patient in need thereof an effective amount of a compound of claim 1, or a pharmaceutically acceptable salt, deuterated analog, or solvate thereof.

34. A method for modulating glucose levels in a patient in need of such modulating, the method comprising administering to a patient in need thereof an effective amount of a compound of claim 1, or a pharmaceutically acceptable salt, deuterated analog, or solvate thereof.

35. The method of claim 30, further comprising administering an additional therapy or therapeutic agent to the patient.

36. The method of claim 35, wherein the additional therapy or therapeutic agent is selected from the group consisting of an anti-diabetic agent, an anti-obesity agent, a GLP-1 receptor agonist, an anti-emetic agent, an agent to treat non-alcoholic steatohepatitis (NASH), gastric electrical stimulation, dietary monitoring, physical activity, or a combination thereof.
